(19) United States
(12) Patent Application Publication
(10) Pub. No.: US 2021/0071258 A1
(43) Pub. Date: Mar. 11, 2021

(54) GENE EXPRESSION AND ASSESSMENT OF RISK OF DEVELOPING TOXICITY FOLLOWING CELL THERAPY

(71) Applicant: Juno Therapeutics, Inc., Seattle, WA (US)

(72) Inventors: Robert F. DUBOSE, Seattle, WA (US); Nels Eric OLSON, Seattle, WA (US); Seamus RAGAN, Seattle, WA (US); Claire L. SUTHERLAND, Seattle, WA (US)

(73) Assignee: Juno Therapeutics, Inc., Seattle, WA (US)

(21) Appl. No.: 16/643,403

(22) PCT Filed: Aug. 31, 2018
(86) PCT No.: PCT/US2018/049283

§ 371 Date: Feb. 28, 2020

Related U.S. Application Data
(60) Provisional application No. 62/679,759, filed on Jun. 1, 2018.
(60) Provisional application No. 62/672,562, filed on May 16, 2018.
(60) Provisional application No. 62/584,725, filed on Nov. 10, 2017.
(60) Provisional application No. 62/563,639, filed on Sep. 26, 2017.
(60) Provisional application No. 62/553,790, filed on Sep. 1, 2017.

(51) Int. Cl. C12Q 1/6886; G01N 33/50

(52) U.S. Cl. CPC C12Q 1/6886; G01N 33/5014; C12Q 2600/158; C12Q 2600/106; G01N 33/5088

(57) ABSTRACT

Provided herein are methods for determining if a subject is at risk for developing a toxicity, e.g., neurotoxicity, following administration of a therapy, such as an immunotherapy or cell therapy, e.g., a chimeric antigen receptor (CAR) T cell therapy based on the expression, in a sample obtained from the subject, of one or more genes or gene products that are associated with and/or correlate to a risk of developing toxicity following administration of the therapy. In some aspects, the sample is a sample obtained from the subject prior to receiving the therapy. Also provided are methods for treating a subject having a disease or condition, such as acute lymphoblastic leukemia (ALL), according to a particular treatment regimen, in some cases involving administration of the immunotherapy or cell therapy, based on assessment of risk of developing a toxicity following administration of the therapy. Also provided herein are reagents and kits for performing the methods.

### CROSS-REFERENCE TO RELATED APPLICATIONS

This application claims priority from U.S. provisional applications No. 62/553,790, filed Sep. 1, 2017, entitled “GENE EXPRESSION AND ASSESSMENT OF RISK OF DEVELOPING TOXICITY FOLLOWING CELL THERAPY,” No. 62/563,639, filed Sep. 26, 2017, entitled “GENE EXPRESSION AND ASSESSMENT OF RISK OF DEVELOPING TOXICITY FOLLOWING CELL THERAPY,” No. 62/584,725, filed Nov. 10, 2017, entitled “GENE EXPRESSION AND ASSESSMENT OF RISK OF DEVELOPING TOXICITY FOLLOWING CELL THERAPY,” No. 62/672,562, filed May 16, 2018, entitled “GENE EXPRESSION AND ASSESSMENT OF RISK OF DEVELOPING TOXICITY FOLLOWING CELL THERAPY,” and No. 62/679,759, filed Jun. 1, 2018, entitled “GENE EXPRESSION AND ASSESSMENT OF RISK OF DEVELOPING TOXICITY FOLLOWING CELL THERAPY,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042011340SeqList.TXT, created August 27, 2018, which is 930,586 bytes in size. The information in the electronic format of the Sequence Listing is incorporated by reference in its entirety.

### FIELD

The present disclosure provides methods for determining if a subject is at risk for developing a toxicity, e.g., neurotoxicity, following administration of a therapy, such as an immunotherapy or cell therapy, e.g., a chimeric antigen receptor (CAR) T cell therapy, based on the expression, in a sample obtained from the subject, of one or more genes or gene products that are associated with and/or correlate to a risk of developing toxicity following administration of the therapy. In some aspects, the sample is a sample obtained from the subject prior to receiving the therapy. The present disclosure also provides methods for treating a subject having a disease or condition, such as acute lymphoblastic leukemia (ALL), according to a particular treatment regimen, in some cases involving administration of the immunotherapy or cell therapy, based on assessment of risk of developing a toxicity following administration of the therapy. Also provided herein are reagents and kits for performing the methods.

### BACKGROUND

Immunotherapies such a chimeric antigen receptors (CAR) T cell therapies have shown great promise for treating subjects with cancers, including relapsed and refractory B-cell neoplasms, such as acute lymphoblastic leukemia, chronic lymphocytic leukemia, and non-Hodgkin lymphomas. However, despite their success, immunotherapies such as CAR-T cell therapies can be accompanied by adverse effects and toxicity, such as cytokine release syndrome and neurotoxicity. The mechanisms underlying these toxicities are not completely understood. The present disclosure provides tools and techniques to identify and assess a risk of toxicity associated with therapeutic cell compositions, as well as tools and techniques to administer therapeutic cell compositions in a manner that reduces or prevents a risk of toxicity.

### SUMMARY

Provided herein are methods, gene signatures or panels, kits and articles of manufacture for use in assessing or predicting whether a subject may have a risk of developing a neurotoxicity, such as severe neurotoxicity, such as whether the subject has a high or low risk of developing severe neurotoxicity, following administration of an immunotherapy, such as a cell therapy containing cells expressing a recombinant receptor (e.g. CAR-T cells). In some aspects, the neurotoxicity is a severe neurotoxicity, such as a specified grade or severity of neurotoxicity, for example grade 4 or grade 5 neurotoxicity. In some aspects, the methods include assessing a sample, such as a biological sample, from a subject prior to the subject having receiving an immunotherapy and/or from a biological sample that does not contain cells genetically engineered with the recombinant receptors, such that the provided embodiments can be used for identifying subjects at risk prior to receiving the immunotherapy, e.g. cell therapy. In some aspects, the provided embodiments offer methods, reagents and kits for classifying or assessing risk of development of a neurotoxicity following administration of an immunotherapy, such as a cell therapy, based on patient-specific characteristics as opposed to drug product characteristics.

Provided herein is a method of assessing a risk of a toxicity or a toxicity-related outcome, following administration of an immunotherapy, which is optionally a cell therapy, in which the method comprises assessing the presence, absence or level of expression of one or more gene products or portions thereof in a sample from a subject that is a candidate for receiving a cell therapy for treatment of a disease or condition, said cell therapy comprising a dose of genetically engineered cells expressing a recombinant receptor, wherein the one or more gene products is associated with a risk of developing neurotoxicity following administration of the cell therapy; and the sample does not comprise cells genetically engineered with the recombinant receptor and/or is obtained from the subject prior to receiving the cell therapy; and comparing the presence, absence or level of expression of the one or more gene products or portions thereof to a gene reference value, wherein the comparison indicates the risk or likely risk of the subject developing a neurotoxicity, optionally a specified grade or severity of neurotoxicity, following administration of the therapy to the subject. In some aspects, each of the one or more gene products is individually compared to a gene reference value for the respective gene product. In some embodiments, the disease or condition is acute lymphoblastic leukemia (ALL) or a subtype thereof.

In some embodiments of any of the provided methods, (a) at least one of the one or more gene products is from a first group of gene products that negatively correlate to a risk of developing neurotoxicity; and/or (b) at least one of the one or more gene products is from a second group of gene products that positively correlates to a risk of developing neurotoxicity. In some embodiments, the at least one gene product is from (a) and is selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, or WNT9A, ENG, SELE, ICAM3, or IL6R, or is a portion or fragment thereof. In some embodiments, the at least one gene product is from (b) and is selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or is a portion or fragment thereof. In some embodiments, the disease or condition is acute lymphoblastic leukemia (ALL) or a subtype thereof.

Provided herein is a method of assessing a risk of toxicity following administration of an immunotherapy or a cell therapy, in which the method includes (1) assessing the presence, absence or level of expression of one or more gene products or a portion thereof in a sample from a subject that is a candidate for receiving a cell therapy for treating a disease or condition, wherein (a) at least one of the one or more gene products is selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or (b) at least one of the one or more gene products is selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or is a portion or fragment thereof; and (2) comparing the presence, absence or level of expression of the one or more gene product to a gene reference value, wherein the comparison indicates whether the subject is or is likely at risk of developing a neurotoxicity or grade or severity thereof following administration of the cell therapy when administered to the subject. In some embodiments, one or more gene products positively correlates to a risk of developing neurotoxicity. In some embodiments of such methods, each of the one or more gene products is individually compared to a gene reference value for the respective gene product. In some embodiments, the sample, which can be a biological sample from the subject, does not comprise cells genetically engineered with the recombinant receptor. In some embodiments, the sample, such as a biological sample, does not contain cells genetically engineered with the recombinant receptor and/or is obtained from the subject prior to receiving the cell therapy.

In some embodiments of any of the provided methods, the presence, absence or level of the one or more gene products is associated with a risk of developing neurotoxicity following administration of the cell therapy or immunotherapy, such as a low or high risk of developing neurotoxicity following administration of a cell therapy or immunotherapy. In some embodiments, expression of the at least one or more gene products from (a) negatively correlate to a risk, such as a high risk, of developing neurotoxicity, such as severe neurotoxicity, for example grade 4 or 5 neurotoxicity. In some embodiments, expression of the at least one or more gene products from (b) positively correlates to a risk, such as a high risk, of developing neurotoxicity, such as severe neurotoxicity, for example grade 4 or 5 neurotoxicity.

In some of any such embodiments, the comparison indicates the subject is or is likely at risk of developing neurotoxicity, such as is at a high risk of developing neurotoxicity, if the at least one gene product of (a) is at or below a gene reference value and/or the at least one gene product of (b) is at or above a gene reference value; or the comparison indicates the subject is not or is likely not at risk of developing neurotoxicity, such as has a low risk of developing neurotoxicity, if the at least one gene product of (a) is above a gene reference value and/or the at least one gene product of (b) is below a gene reference value.

In some of any such embodiments, if the comparison indicates the subject is or is likely to develop neurotoxicity, the method includes selecting the subject for administration of a therapeutic regimen. In some aspects, the therapeutic regimen comprising administering to the subject: an agent or other treatment capable of treating, preventing, delaying, reducing or attenuating the development or risk of development of a toxicity and the cell therapy, wherein administration of the agent is to be administered (i) prior to, (ii) within one, two, or three days of, (iii) concurrently with and/or (iv) at first fever following, the initiation of administration of the cell therapy to the subject; ii. the cell therapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the cell therapy; and/or iii. the cell therapy in an in-patient setting and/or with admission to the hospital for one or more days, optionally wherein the cell therapy is otherwise to be administered to subjects on an outpatient basis or without admission to the hospital for one or more days; or iv. an alternative therapeutic treatment other than the cell therapy.

In some of any such embodiments, if the comparison indicates the subject is not or is likely not at risk of developing neurotoxicity, the method includes selecting the subject for administration of a therapeutic regimen. In some embodiments, the therapeutic regimen comprises administering to the subject: i. the cell therapy, optionally at a non-reduced dose, optionally on an outpatient basis or without admission to the hospital for one or more days; ii. the cell therapy, wherein administration of the cell therapy does not comprise administering, prior to or concurrently with administering the cell therapy and/or prior to the development of a sign or symptom of toxicity other than fever, an agent or treatment capable of treating, preventing, delaying, or attenuating the development of the toxicity; or iii. the cell therapy in an outpatient setting and/or without admission of the subject to the hospital overnight or for one or more consecutive days and/or is without admission of the subject to the hospital for one or more days.

In some of any such embodiments, following selection of the subject, the method further comprises administering the therapeutic regimen to the selected subject.

Provided herein is a method of treating comprising administering a therapeutic regimen to a subject that is a candidate for receiving a cell therapy comprising a dose of genetically engineered cells expressing a recombinant receptor for treatment of a disease or condition, wherein the administration is carried out following or based on the results of assessing the presence, absence or level of expression, from a sample from the subject, of one or more gene products or portion thereof, wherein: (a) at least one of the one or more gene products is selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or (b) at least one of the one or more gene products is selected from ASAP2, ATP9A, CCNA1, CDHR3, CECR2, DLX1, DPYSL3, EHD4, FMNL2, GGA2, HHIPL1, HMX3, IGF2BP1, IL3RA, IRX5, KCNIP1, KIAA1644, LINC00092, LINC01483, MIB1, MMP14, NOM1, OTOA, PCDHGA12, PCDHGA4, PCDHGA6, PCDHGB5, PCDHGB6, PINLYP, PPM1E, PRKD1, PROKR2, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TNKS1BP1, TTC28, PCDHGA9, FMNL1, or ZNF415 or is a portion or fragment thereof. In some embodiments, the sample, such as a biological sample, does not comprise cells genetically engineered with the recombinant receptor. In some embodiments, the sample, such as a biological sample, is obtained from the subject prior to receiving the cell therapy and/or the sample does not comprise cells expressing the recombinant receptor. In some embodiments, the sample is obtained from the subject prior to receiving the cell therapy and/or the sample does not comprise cells expressing the recombinant receptor.

In some embodiments, the results of assessing the presence, absence or level of expression of the one or more gene products or portions thereof comprises a comparison to a gene reference value, wherein the comparison indicates the risk or likely risk of the subject developing neurotoxicity following administration of the cell therapy when administered to the subject. In some of any of such embodiments, the one or more gene products are individually compared to a gene reference value for the respective gene product.

In some embodiments of any of the provided methods, the presence, absence or level of the one or more gene products is associated with a risk of developing neurotoxicity following administration of the cell therapy or immunotherapy, such as a low or high risk of developing neurotoxicity following administration of a cell therapy or immunotherapy. In some embodiments, expression of the at least one or more gene products from (a) negatively correlate to a risk, such as a high risk, of developing neurotoxicity, such as severe neurotoxicity, for example grade 4 or 5 neurotoxicity. In some embodiments, expression of the at least one or more gene products from (b) positively correlates to a risk, such as a high risk, of developing neurotoxicity, such as severe neurotoxicity, for example grade 4 or 5 neurotoxicity.

In some of any such embodiments, the sample is obtained from the subject prior to receiving the cell therapy and/or the sample does not comprise cells expressing the recombinant receptor. In some of any such embodiments, the presence, absence or level of the one or more gene products is associated with a risk of developing neurotoxicity following administration of the cell therapy.

In some embodiments of the provided methods, wherein the results of assessing the presence, absence or level of expression of the one or more gene products or portions thereof comprises a comparison to a gene reference value, wherein the comparison indicates the risk or likely risk of the subject developing neurotoxicity following administration of the cell therapy when administered to the subject. In some embodiments of the provided methods, each of the one or more gene products is individually compared to a gene reference value for the respective gene product. In some embodiments of the provided methods: expression of the at least one or more gene products from (a) negatively correlate to a risk that the subject is or is likely to develop neurotoxicity following administration of the cell therapy when it is administered; and/or expression of the at least one or more gene products from (b) positively correlates to a risk that the subject is or is likely to develop neurotoxicity following administration of the cell therapy when it is administered.

In some embodiments of the provided methods, if the assessing indicates the subject is or is likely to develop neurotoxicity following administration of the immunotherapy, e.g. cell therapy, the therapeutic regimen comprises administering to the subject: i. an agent or other treatment capable of treating, preventing, delaying, reducing or attenuating the development or risk of development of a toxicity and the immunotherapy, e.g. cell therapy, wherein administration of the agent is to be administered (i) prior to, (ii) within one, two, or three days of, (iii) concurrently with and/or (iv) at first fever following, the initiation of administration of the immunotherapy, e.g. cell therapy, to the subject; ii. The immunotherapy, e.g. cell therapy, at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunothearapy, e.g. cell therapy; and/or iii. the immunotherapy, e.g. cell therapy, in an in-patient setting and/or with admission to the hospital for one or more days, optionally wherein the cell therapy is otherwise to be administered to subjects on an outpatient basis or without admission to the hospital for one or more days; or iv. an alternative therapeutic treatment other than the immunotherapy, e.g. cell therapy.

In some embodiments, provided herein is a therapeutic regimen involving an immunotherapy, such as a cell therapy, e.g. comprising cells engineered with a recombinant receptor, for use in treating a disease or condition in a subject that has been identified as being at risk of developing neurotoxicity or likely being at risk of developing neurotoxicity, e.g. in which the subject's risk is identified or determined in accord with the embodiments herein, such as by assessing the presence, absence or level of expression of one or more gene products as described and comparing the presence, absence or level to a reference value. In some embodiments, the disease or condition is ALL. In some embodiments, the therapeutic regimen comprises administering to the subject: i. an agent or other treatment capable of treating, preventing, delaying, reducing or attenuating the development or risk of development of a toxicity and the immunotherapy, e.g. cell therapy, wherein administration of the agent is to be administered (i) prior to, (ii) within one, two, or three days of, (iii) concurrently with and/or (iv) at first fever following, the initiation of administration of the immunotherapy, e.g. cell therapy, to the subject; ii. the immunotherapy, e.g. cell therapy, at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy, e.g. cell therapy; and/or iii. the immunotherapy, e.g. cell therapy, in an in-patient setting and/or with admission to the hospital for one or more days, optionally wherein the immunotherapy, e.g. cell therapy, is otherwise to be administered to subjects on an outpatient basis or without admission to the hospital for one or more days; or iv. an alternative therapeutic treatment other than the immunotherapy, e.g. cell therapy.

In some embodiments, provided herein is an agent or other treatment capable of treating, preventing, delaying, reducing or attenuating the development or risk of development of a neurotoxicity for use in treating, preventing, delaying, reducing or attenuating the development or risk of development of a neurotoxicity following administration of an immunotherapy, e.g. cell therapy, in a subject at has been identified as being at risk of developing neurotoxicity or likely being at risk of developing neurotoxicity, e.g. in which the subject's risk is identified or determined in accord with the embodiments herein, such as by assessing the presence, absence or level of expression of one or more gene products as described and comparing the presence, absence or level to a reference value. In some embodiments, the disease or condition is ALL. In some embodiments, the use of the agent is for administration of the agent (i) prior to, (ii) within one, two or three days of, (iii) concurrently with and/or (iv) at a first fever following the initiation of administration of the immunotherapy, e.g. cell therapy, to the subject.

In some embodiments of the provided methods, if the assessing indicates the subject is not or is likely not to develop neurotoxicity following administration of the cell therapy, the therapeutic regimen comprises administering to the subject: i. the immunotherapy, e.g. cell therapy, optionally at a non-reduced dose, optionally on an outpatient basis or without admission to the hospital for one or more days; ii. the immunotherapy, e.g. cell therapy, wherein administration of the immunotherapy, e.g. cell therapy, does not comprise administering, prior to or concurrently with administering the immunotherapy, e.g. cell therapy, and/or prior to the development of a sign or symptom of toxicity other than fever, an agent or treatment capable of treating, preventing, delaying, or attenuating the development of the toxicity; or iii. the immunotherapy, e.g. cell therapy, in an outpatient setting and/or without admission of the subject to the hospital overnight or for one or more consecutive days and/or is without admission of the subject to the hospital for one or more days.

In some embodiments, provided herein is a therapeutic regimen involving an immunotherapy, such as a cell therapy, e.g. comprising cells engineered with a recombinant receptor, for use in treating a disease or condition in a subject that has been identified as not being at risk of developing neurotoxicity or likely not at risk of developing neurotoxicity, such as in which the subject's risk has been identified or determined in accord with the embodiments herein, such as by assessing the presence, absence or level of expression of one or more gene products as described and comparing the presence, absence or level to a reference value. In some embodiments, the disease or condition is ALL. In some embodiments, the therapeutic regimen comprises administering to the subject: i. the immunotherapy, e.g. cell therapy, optionally at a non-reduced dose, optionally on an outpatient basis or without admission to the hospital for one or more days; ii. the cell therapy, wherein administration of the cell therapy does not comprise administering, prior to or concurrently with administering the cell therapy and/or prior to the development of a sign or symptom of toxicity other than fever, an agent or treatment capable of treating, preventing, delaying, or attenuating the development of the toxicity; or iii. the cell therapy in an outpatient setting and/or without admission of the subject to the hospital overnight or for one or more consecutive days and/or is without admission of the subject to the hospital for one or more days.

In some of any such embodiments, the disease or condition is any disease or condition described herein, optionally a cancer, optionally a myeloma, lymphoma or leukemia. In some of any such embodiments, the at least one gene product is from (a) and is a gene product associated with a PH+ or Ph-like molecular subtype of ALL. In some of any such embodiments, the at least one gene product is selected from ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, LO645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A, or is a portion or fragment of any of the foregoing.

Provided herein is a method of treatment, the method comprising: selecting a subject that exhibits a Philadelphia chromosome (Ph+) and/or Ph chromosome-like (Ph-like) molecular subtype of acute lymphoblastic leukemia (ALL); and administering to the subject a cell therapy comprising a dose of cells expressing a recombinant receptor that binds to an antigen associated with the ALL.

Also provided herein is a cell therapy for use in treating ALL in a subject that exhibits or has a Philadelphia chromosome (Ph+) and/or Ph chromosome-like (Ph-like) molecular subtype of acute lymphoblastic leukemia (ALL), said cell therapy comprising a dose of cells expressing a recombinant receptor that binds to an antigen associated with ALL.

In some of any such embodiments, the subject is selected based on results of cytogenetic or molecular genetic analysis. In some of any such embodiments, the analysis comprises karyotype analysis, fluorescence in situ hybridization (FISH), multicolor FISH, polymerase chain reaction (PCR), a tyrosine kinase inhibitor assay, gene expression profiling or microarray or an immunoassay, optionally an ELISA. In some of any such embodiments: the selected subject exhibits one or more of the (9;22)(q34;q11) chromosomal abnormality; deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or comprises a Ph-like gene expression signature; the subject is selected based on one or more of the presence of the (9;22)(q34;q11) chromosomal abnormality, deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or the presence of a Ph-like gene expression signature.

In some of any such embodiments, the presence of the Ph-like gene signature is based on comparison of the presence, absence or level of expression, in a sample from the subject, of at least one gene product to a reference gene value, said at least one gene product is selected from (a) ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, LO645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A or a portion or fragment of any of the foregoing and/or said at least one gene product is selected from (b) ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing, whereby the comparison indicates whether the subject exhibits a Ph-like molecular subtype of ALL.

In some of any such embodiments, each of the one or more gene products is individually compared to a gene reference value for the respective gene product. In some of any such embodiments, wherein the subject exhibits a Ph-like molecular subtype of ALL if the comparison indicates the at least one gene product of (a) is above a gene reference value and/or the at least one gene product of (b) is below a gene reference value. In some of any such embodiments, the at least one gene product selected from (a) is ADGRF1, BMPR1B, CA6, CD99, CHN2, CRLF2, DENND3, ENAM, GBP5, GLI2, IFITM1, IGJ (JCHAIN), LDB3, LO645744, MDF1C, MUC4, NRXN3, PON2, S100Z, SEMA6A, SLC37A3, SLC2A5, SPATS2L, TMEM154, TP53INP1, TTYH2 or WNT9A, or is a portion of fragment of any of the foregoing. In some of any such embodiments, the at least one gene product selected from (a) is ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A or is a portion or fragment of any of the foregoing. In some of any such embodiments, the at least one gene product selected from (b) is ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing.

In some of any such embodiments, the subject is a human and/or the one or more gene products are human. In some of any such embodiments, at least one of the one or more gene products is from (a) and at least one of the one or more gene products is from (b),In some of any such embodiments, the one or more genes comprises at least one gene from (a) that is IGJ (JCHAIN), MUC4, CA6, WNT9A, ADGRF1 or CCL17, or a portion or fragment of any of the foregoing. In some of any such embodiments, the one or more genes comprises at least one gene from (a) that is CCL17 or a portion or fragment thereof.4 In some of any such embodiments, the one or more genes comprises at least one gene from (b) that is PINLYP, ASAP2, FMNL2, PTCH1, TTC28, PCDHGA6, PCDHGB6 or PCDHGA12, or a portion or fragment of any of the foregoing. In some of any such embodiments, the one or more genes comprises at least one gene from (b) that is PINLYP or PCDHGA12 or a portion or fragment of any of the foregoing.

In some of any such embodiments: greater than or greater than about 30%, 35%, 40% , or 50% of the subjects treated according to the method do not exhibit any grade of cytokine release syndrome (CRS) or neurotoxicity; and/or at least at or about 45, 50, 60, 65, 70, 75, 80, 85, 90, 95% or about 100% of subjects treated according to the method do not exhibit severe CRS, optionally grade 3 or higher, prolonged grade 3 or higher or grade 4 or 5 CRS; and/or at least at or about 45, 50, 60, 65, 70, 75, 80, 85, 90, 95% or about 100% of subjects treated according to the method do not exhibit severe neurotoxicity, optionally grade 3 or higher, prolonged grade 3 or higher or grade 4 or 5 neurotoxicity; and/or at least at or about 45, 50, 60, 65, 70, 75, 80, 85, 90, 95% or about 100% of subjects treated according to the method do not exhibit cerebral edema.

In some of any such embodiments: prior to initiation of administration of the dose of cells, the subject has not been administered an agent or treatment capable of treating, preventing, delaying, reducing or attenuating the development or risk of development of a toxicity; and/or the subject is not administered an agent or treatment for the treatment or prevention or reduction or attenuation of a neurotoxicity and/or a cytokine release syndrome or risk thereof, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or the subject is not administered an agent or treatment for the treatment or prevention or reduction or attenuation of a neurotoxicity and/or a cytokine release syndrome or risk thereof, following administration of the dose, prior to or unless the subject exhibits a sign or symptom of the toxicity and/or prior to or unless the subject exhibits a sign or symptom of the toxicity other than a fever, optionally wherein the fever is not a sustained fever or the fever is or has been reduced or reduced by more than 1° C. after treatment with an antipyretic; and/or the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

In some of any such embodiments: the administration is carried out on an outpatient basis and/or without requiring admission to or an overnight stay at a hospital; and if the subject exhibits a sustained fever or a fever that is or has not been reduced or not reduced by more than 1° C. after treatment with an antipyretic, the subject is admitted to the hospital or to an overnight stay at a hospital and/or is administered an agent or treatment for the treatment or prevention or reduction or attenuation of a neurotoxicity and/or a cytokine release syndrome or risk thereof.

In some of any such embodiments, the neurotoxicity comprises severe neurotoxicity, optionally at or above grade 4 or grade 5 or at least prolonged grade 3 neurotoxicity. In some of any such embodiments, the neurotoxicity is associated with cerebral edema. In some of any such embodiments, the sample is a tumor sample and/or the sample comprises or is likely to comprise tumor cells. In some of any such embodiments, the sample is or comprises a bone marrow sample, blood sample, plasma sample, or serum sample. In some of any such embodiments, the sample is or comprises a bone marrow sample. In some of any such embodiments, is or comprises a bone marrow aspirate. In some of any such embodiments, the presence, absence or level of expression of one, two, three, four, five, six, seven, eight, nine, ten or more gene products is assessed or compared. In some of any such embodiments, the one or more gene products or portion or fragment thereof is a polynucleotide or a portion thereof. In some of any such embodiments, the polynucleotide is an RNA. In some of any such embodiments, the one or more gene products or portions thereof is a messenger RNA (mRNA) transcript or a partial transcript thereof.

In some of any such embodiments, the one or more gene products is assessed from complementary DNA (cDNA), optionally based on the mRNA transcript or partial transcript thereof. In some of any such embodiments, the cDNA is prepared by PCR amplification, optionally by RT-PCR or quantitative PCR, of the mRNA transcript or partial transcript thereof. In some of any such embodiments, the presence, absence or level of expression of the one or more gene products or portions thereof is assessed by polymerase chain reaction (PCR), northern blotting, microarray, and/or a sequencing technique.

In some of any such embodiments, the one or more gene products or portions thereof comprise a protein or a portion thereof. In some embodiments, the one or more gene products is a gene product selected from CCL17, ENG, SELE, ICAM3, or IL6R. In some of any such embodiments, the presence, absence or level of expression of the one or more gene products or portions thereof is measured by an immunoassay, nucleic acid-based or protein-based aptamer techniques, high precision liquid chromatography (HPLC), peptide sequencing, and/or mass spectrometry. In some aspects, the methods include an immunoassay-based method, such as an immunoassay performed on the Meso Scale Development (MSD) or Luminex platforms.

In some of any such embodiments, the presence, absence or level of the one or more gene products or portions thereof is measured by immunoassay and the immunoassay is selected from enzyme-linked immunosorbent assay (ELISA), enzyme immunoassay (EIA), radioimmunoassay (RIA), surface plasmon resonance (SPR), Western Blot, Lateral flow assay, immunohistochemistry, protein array or immuno-PCR iPCR). In some of any such embodiments, the gene reference value, or each of the gene reference values individually for each of the at least one or more gene product, is determined by application of an algorithm to the level, concentration or amount of expression in a control sample, or the average of such level, concentration or amount of expression among a plurality of control samples.

In some of any such embodiments, the gene reference value for the at least one gene product of (a), or each of the gene reference values individually for each of the at least one or more gene product of (a), is a value that: i) is within 25%, within 20%, within 15%, within 10%, or within 5% above the average level, concentration or amount, and/or is within a standard deviation above the average level, concentration or amount, of the at least one gene product in a plurality of control samples; ii) is above the highest level, concentration or amount of the at least one gene product, optionally within 50%, within 25%, within 20%, within 15%, within 10%, or within 5% above such highest level, concentration or amount, measured in at least one sample from among a plurality of control samples; and/or iii) is above the highest level, concentration or amount of the at least one gene product as measured among more than 75%, 80%, 85%, 90%, or 95%, or 98% of samples from a plurality of control samples; wherein the plurality of control samples are a plurality of biological samples obtained from a group of subjects prior to receiving a cell therapy for treating a disease or condition, optionally ALL, said cell therapy containing cells genetically engineered with a recombinant receptor, wherein each of the subjects of the group went on to develop severe neurotoxicity, optionally grade 3 or higher, prolonged grade 3 or higher or grade 4 or 5 neurotoxicity, after receiving the cell therapy for treating the same disease or condition.

In some of any such embodiments, the gene reference value for the at least one gene product of (a), or each of the gene reference values individually for each of the at least one or more gene product of (a), is: below the lowest level, concentration, or amount, optionally within 50%, within 25%, within 20%, within 15%, within 10%, or within 5% below the lowest level, concentration or amount, of the at least one gene product observed in a sample from among a second plurality of control samples; and/or below the level, concentration or amount of the at least one gene product measured as measured in more than 75%, 80%, 85%, 90%, 95%, or 98% of samples from among a second plurality of control samples wherein the second plurality of control samples is obtained from a group of subjects prior to receiving the same cell therapy for treating the same disease or condition, wherein each of the subjects of the group did not develop severe neurotoxicity, optionally wherein each of the subjects developed grade 3 or less, grade 2 or less, or grade 1 or 0 neurotoxicity, after receiving the cell therapy for treating the same disease or condition.

In some of any such embodiments, the gene reference value for the at least one gene product of (a), or each of the gene reference values individually for each of the at least one or more gene product of (a), is a value that: i) is within 25%, within 20%, within 15%, within 10%, or within 5% above the average level, concentration or amount, and/or is within a standard deviation above the average level, concentration or amount, of the at least one gene product in a plurality of control samples, ii) is above the highest level, concentration or amount of the at least one gene product, optionally within 50%, within 25%, within 20%, within 15%, within 10%, or within 5% above such highest level, concentration or amount, as measured in at least one sample from among a plurality of control samples; and/or iii) is above the highest level, concentration or amount of the at least one gene product measured among more than 75%, 80%, 85%, 90%, 95%, or 98% of samples from a plurality of control samples; wherein the plurality of control samples are a plurality of biological samples obtained from a group of subjects prior to receiving a cell therapy for treating a disease or condition, optionally ALL, wherein each of the subjects of the group has Philadelphia chromosome positive (PH+) or Philadelphia-like (Ph-like) subtype of ALL.

In some of any such embodiments, the gene reference value for the at least one gene product of (a), or each of the gene reference values individually for each of the at least one or more gene product of (a), is: below the lowest level, concentration, or amount, optionally within 50%, within 25%, within 20%, within 15%, within 10%, or within 5% below the lowest level, concentration or amount, of the at least one gene product observed in a sample from among a second plurality of control samples; and/or below the level, concentration or amount measured in more than 75%, 80%, 85%, 90%, 95%, or 98% of samples from among a plurality of control samples, wherein the second plurality of control samples is obtained from a group of subjects prior to receiving the same cell therapy for treating the same disease or condition, wherein each of the subjects has ALL that is not the Ph+ or Ph-like subtype of ALL.

In some of any such embodiments, the gene reference value for the at least one gene product of (b), or each of the gene reference values individually for each of the at least one or more gene product of (b), is a value that: i) is within 25%, within 20%, within 15%, within 10%, or within 5% below the average level, concentration or amount, and/or is within a standard deviation below the average level, concentration or amount, of the at least one gene product in a plurality of control samples; ii) is below the lowest level, concentration or amount of the at least one gene product, optionally within 50%, within 25%, within 20%, within 15%, within 10%, or within 5% below the lowest level, concentration or amount, as measured in at least one sample from among a plurality of control samples; iii) is below the lowest level, concentration or amount of the at least one gene product measured among more than 75%, 80%, 85%, 90%, 95%, or 98% of samples from a plurality of control samples; wherein the plurality of control samples are a plurality of biological samples obtained from a group of subjects prior to receiving a cell therapy for treating a disease or condition, said cell therapy containing cells genetically engineered with a recombinant receptor, wherein each of the subjects of the group went on to develop severe neurotoxicity, optionally grade 3 or higher, prolonged grade 3 or higher or grade 4 or 5 neurotoxicity, after receiving the cell therapy for treating the same disease or condition.

In some of any such embodiments, the gene reference value for the at least one gene product of (b), or each of the gene reference values individually for each of the at least one or more gene product of (b) is above the highest level, concentration, or amount of the at least one gene product, optionally within 50%, within 25%, within 20%, within 15%, within 10%, or within 5% above such level, concentration or amount, measured in at least one sample from among a second plurality of control samples; and/or above the level, concentration or amount of the at least one gene product measured in more than 75%, 80%, 85%, 90%, 95%, or 98% of samples from among a second plurality of control samples, wherein the second plurality of control samples are a plurality of control samples obtained from a group of subjects prior to receiving the cell therapy for treating the disease or condition, wherein each of the subjects of the group did not develop severe neurotoxicity, optionally wherein each of the subjects developed grade 3 or less, grade 2 or less, or grade 1 or 0 neurotoxicity, after receiving the cell therapy for treating the same disease or condition.

In some of any such embodiments, the gene reference value for the at least one gene product of (b), or each of the gene reference values individually for each of the at least one or more gene product of (b), is a value that: i) is within 25%, within 20%, within 15%, within 10%, or within 5% below the average level, concentration or amount, and/or is within a standard deviation below the average level, concentration or amount, of the at least one gene product in a plurality of control samples; ii) is below the lowest level, concentration or amount of the at least one gene product, optionally within 50%, within 25%, within 20%, within 15%, within 10%, or within 5% below such lowest level, concentration or amount, as measured in at least one sample from among a plurality of control samples; iii) is below the lowest level, concentration or amount of the at least one gene product measured among more than 75%, 80%, 85%, 90%, 95%, or 98% of samples from a plurality of control samples; wherein the plurality of control samples are a plurality of biological samples obtained from a group of subjects prior to receiving a cell therapy for treating a disease or condition, optionally ALL, said cell therapy containing cells genetically engineered with a recombinant receptor, wherein each of the subjects of the group has the PH+or Ph-like subtype of ALL.

In some of any such embodiments, the gene reference value for the at least one gene product of (b), or each of the gene reference values individually for each of the at least one or more gene product of (b) is: above the highest level, concentration, or amount of the at least one gene product, optionally within 50%, within 25%, within 20%, within 15%, within 10%, or within 5% above such level, concentration or amount, measured in at least one sample from among a second plurality of control samples; and/or is above the level, concentration or amount of the at least one gene product measured among more than 75%, 80%, 85%, 90%, 95%, or 98% of samples from a second plurality of control samples, wherein the second plurality of control samples are a plurality of control samples obtained from a group of subjects prior to receiving the cell therapy for treating the same disease or condition, wherein each of the subjects do not exhibit the Ph+ or Ph-like subtype of ALL.

In some of any such embodiments, the control sample or the plurality of control samples are obtained from one or more subjects who have ALL. In some of any such embodiments, the control sample or each of the plurality of control samples is from the same biological sample being assessed, optionally is a bone marrow sample. In some of any such embodiments, the plurality of control samples comprises at least 3, at least 10, at least 20, at least 50, or at least 100 control samples.

Provided herein are methods of assessing a risk of a toxicity or a toxicity-related outcome, following administration of an immunotherapy, the method including assessing the level or amount of one or more proteins or portions thereof in a biological sample from a subject that is a candidate for receiving a immunotherapy for treatment of a disease or condition, wherein the disease or condition is acute lymphoblastic leukemia (ALL) or a subtype thereof, wherein at least one of the one the one or more proteins or portions thereof are selected from CCL17, ENG, SELE, ICAM3, or IL6R; and comparing the level or amount of the one or more proteins or portions thereof to a reference value, wherein the comparison indicates the subject is or is likely at risk of developing neurotoxicity if the at least one of the one or more proteins or portions thereof is at or below the reference value; or the comparison indicates the subject is not or is likely not at risk of developing neurotoxicity if at least one of the one or more protein or portions thereof is above the reference value. In some embodiments, the biological sample is a plasma sample. In some embodiments, at least one of the one or more proteins or portions thereof is CCL17. In some embodiments, the immunotherapy is a cell therapy or is a T cell-engaging therapy, optionally wherein the cell therapy contains cells engineered to express a recombinant receptor. In some embodiments, the sample does not include the immunotherapy, and/or is obtained from the subject prior to receiving the immunotherapy.

In some embodiments, the reference value for the one or more protein or portion thereof, or each of the reference values individually for each of the one or more protein or portion thereof, is a value that is within 25%, within 20%, within 15%, within 10%, or within 5% above the average level, concentration or amount, and/or is within a standard deviation above the average level, concentration or amount, of the one or more protein or portion thereof in a plurality of control samples; is above the highest level, concentration or amount of the one or more protein or portion thereof, optionally within 50%, within 25%, within 20%, within 15%, within 10%, or within 5% above such highest level, concentration or amount, measured in at least one sample from among a plurality of control samples; and/or is above the highest level, concentration or amount of the one or more protein or portion thereof as measured among more than 75%, 80%, 85%, 90%, or 95%, or 98% of samples from a plurality of control samples; wherein the plurality of control samples are a plurality of biological samples obtained from a group of subjects prior to receiving a immunotherapy for treating ALL, wherein (1) each of the subjects of the group went on to develop severe neurotoxicity, optionally grade 3 or higher, prolonged grade 3 or higher or grade 4 or 5 neurotoxicity, after receiving the immunotherapy for treating the same disease or condition; or (2) each of the subjects of the group has ALL that is not Philadelphia chromosome positive (Ph+) or Philadelphia-like (Ph-like) subtype of ALL.

In some embodiments, the reference value for the one or more protein or portion thereof, or each of the gene reference values individually for each of the one or more protein or portion thereof, is below the lowest level, concentration, or amount, optionally within 50%, within 25%, within 20%, within 15%, within 10%, or within 5% below the lowest level, concentration or amount, of the one or more protein or portion thereof observed in a sample from among a second plurality of control samples; and/or below the level, concentration or amount of the one or more protein or portion thereof as measured in more than 75%, 80%, 85%, 90%, 95%, or 98% of samples from among a second plurality of control samples, wherein the second plurality of control samples is obtained from a group of subjects prior to receiving the same immunotherapy for treating the ALL, wherein (1) each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition, or (2) each of the subjects has ALL that is Philadelphia chromosome positive (Ph+) or Philadelphia-like (Ph-like) subtype of ALL.

In some embodiments,the control sample or each of the plurality of control samples is the same type of biological sample being assessed from the subject, optionally is a plasma sample. In some embodiments,the plurality of control samples includes at least 3, at least 10, at least 20, at least 50, or at least 100 control samples.

In some embodiments, if the comparison indicates the subject is or is likely to develop neurotoxicity, selecting the subject for administration of a therapeutic regimen, the therapeutic regimen comprising administering to the subject an agent or other treatment capable of treating, preventing, delaying, reducing or attenuating the development or risk of development of a toxicity and the immunotherapy, wherein administration of the agent is to be administered (i) prior to, (ii) within one, two, or three days of, (iii) concurrently with and/or (iv) at first fever following, the initiation of administration of the immunotherapy to the subject; the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy; the immunotherapy in an in-patient setting and/or with admission to the hospital for one or more days, optionally wherein the immunotherapy is otherwise to be administered to subjects on an outpatient basis or without admission to the hospital for one or more days; or an alternative therapeutic treatment other than the immunotherapy.

In some embodiments, if the comparison indicates the subject is not or is likely not at risk of developing neurotoxicity, selecting the subject for administration of a therapeutic regimen, the therapeutic regimen comprising administering to the subject the immunotherapy, optionally at a non-reduced dose, optionally on an outpatient basis or without admission to the hospital for one or more days; the immunotherapy, wherein administration of the immunotherapy does not include administering, prior to or concurrently with administering the immunotherapy and/or prior to the development of a sign or symptom of toxicity other than fever, an agent or treatment capable of treating, preventing, delaying, or attenuating the development of the toxicity; or the immunotherapy in an outpatient setting and/or without admission of the subject to the hospital overnight or for one or more consecutive days and/or is without admission of the subject to the hospital for one or more days.

In some embodiments, the method includes further administering the therapeutic regimen to the selected subject.

Provided herein are methods of assessing a risk of a toxicity or a toxicity-related outcome, following administration of an immunotherapy, the method including assessing the level or amount of one or more proteins or portions thereof in a biological sample from a subject that received an immunotherapy for treatment of a disease or condition, wherein at least one of the one the one or more proteins or portions thereof are selected from CCL27, ENG, FAS, I-309, ICAM3, NSE, P-Selectin, Resistin, S100β, Thrombomodulin or vWF; and comparing the level or amount of the one or more proteins or portions thereof to a reference value, wherein the comparison indicates the subject is or is likely at risk of developing neurotoxicity if the at least one of the one or more proteins or portions thereof is at or below the reference value; or the comparison indicates the subject is not or is likely not at risk of developing neurotoxicity if at least one of the one or more protein or portions thereof is above the reference value. In some embodiments, the biological sample is a plasma sample.

In some embodiments, the biological sample is obtained or collected from the subject no more than 4 days, no more than 3 days, no more than 2 days or no more than 1 day, after initiation of administration of the immunotherapy and/or before the subject exhibits a sign or symptom of the toxicity and/or before the subjects develops a sustained fever. In some embodiments, at least one of the one or more proteins or portions thereof is ENG or ICAM3.

In some embodiments, the immunotherapy is a cell therapy or is a T cell-engaging therapy, optionally wherein the cell therapy includes cells engineered to express a recombinant receptor.

In some embodiments, the reference value for the one or more protein or portion thereof, or each of the reference values individually for each of the one or more protein or portion thereof, is a value that is within 25%, within 20%, within 15%, within 10%, or within 5% above the average level, concentration or amount, and/or is within a standard deviation above the average level, concentration or amount, of the one or more protein or portion thereof in a plurality of control samples; is above the highest level, concentration or amount of the one or more protein or portion thereof, optionally within 50%, within 25%, within 20%, within 15%, within 10%, or within 5% above such highest level, concentration or amount, measured in at least one sample from among a plurality of control samples; and/or is above the highest level, concentration or amount of the one or more protein or portion thereof as measured among more than 75%, 80%, 85%, 90%, or 95%, or 98% of samples from a plurality of control samples; wherein the plurality of control samples are a plurality of biological samples obtained from a group of subjects after receiving a immunotherapy for treating ALL, wherein (1) each of the subjects of the group went on to develop severe neurotoxicity, optionally grade 3 or higher, prolonged grade 3 or higher or grade 4 or 5 neurotoxicity, after receiving the immunotherapy for treating the same disease or condition; or (2) each of the subjects of the group has ALL that is not Philadelphia chromosome positive (Ph+) or Philadelphia-like (Ph-like) subtype of ALL.

In some embodiments, the reference value for the one or more protein or portion thereof, or each of the gene reference values individually for each of the one or more protein or portion thereof, is below the lowest level, concentration, or amount, optionally within 50%, within 25%, within 20%, within 15%, within 10%, or within 5% below the lowest level, concentration or amount, of the one or more protein or portion thereof observed in a sample from among a second plurality of control samples; and/or below the level, concentration or amount of the one or more protein or portion thereof as measured in more than 75%, 80%, 85%, 90%, 95%, or 98% of samples from among a second plurality of control samples, wherein the second plurality of control samples is obtained from a group of subjects after receiving the same immunotherapy for treating the ALL, wherein (1) each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition, or (2) each of the subjects has ALL that is Philadelphia chromosome positive (Ph+) or Philadelphia-like (Ph-like) subtype of ALL.

In some embodiments, the control sample or each of the plurality of control samples is the same type of biological sample being assessed from the subject, optionally is a plasma sample. In some embodiments,the control sample or each of the plurality of control samples had been obtained or collected from the subject no more than 4 days, no more than 3 days, no more than 2 days or no more than 1 day, after initiation of administration of the immunotherapy and/or before the subject exhibits a sign or symptom of the toxicity and/or before the subjects develops a sustained fever. In some embodiments, the plurality of control samples includes at least 3, at least 10, at least 20, at least 50, or at least 100 control samples.

In some embodiments, if the comparison indicates the subject is or is likely to develop neurotoxicity, administering to the subject an agent or other treatment capable of treating, preventing, delaying, reducing or attenuating the development or risk of development of a toxicity and the immunotherapy. In some embodiments, administration of the agent is to be administered within one, two, or three days of and/or at first fever following, the initiation of administration of the immunotherapy to the subject.

In some of any such embodiments, the risk or likely risk of the subject developing neurotoxicity following administration of the cell therapy is further based on the value of a parameter that indicates or correlates with the degree of recombinant receptor-dependent, optionally CAR-dependent, activity of the composition, wherein if the value of the parameter is at or greater than a threshold value the subject is at risk of developing neurotoxicity following administration of the cell therapy when administered to the subject.

In some of any such embodiments, the recombinant receptor-dependent activity comprises a measure of the production or accumulation of one or more of a proinflammatory cytokine, or a normalized value thereof. In some of any such embodiments, the proinflammatory cytokine is TNF-alpha, IFN-gamma, IL-2, IL-10, or a combination thereof. In some of any such embodiments, the measure is in an assay involving culture or incubation for a fixed time, optionally 24 hours, of cells of the cell therapy or sample thereof in the presence of an antigen that binds to the recombinant receptor, cells expressing an antigen that binds to the recombinant receptor, and/or an agent that binds to a recombinant receptor. In some of any such embodiments, the assay is an ELISA. In some of any such embodiments, the measure of the proinflammatory cytokine is: (i) a concentration, relative concentration, amount, or relative amount of the cytokine; or (ii) an amount or relative amount of the cytokine per number of cells of the therapeutic T cell composition, optionally the number of CAR+ T cells of the therapeutic T cell composition; or (iii) an amount or relative amount of the cytokine per unit of input cells of the given composition per unit of time, optionally one hour; or(iv) a level indicative of any of (i)-(iii).

In some of any such embodiments, the threshold value of the recombinant receptor-dependent activity is: i) within 25%, within 20%, within 15%, within 10%, or within 5% below the measure of the recombinant receptor-dependent activity, and/or is within a standard deviation below the such measure, in a plurality of reference compositions; ii) is below the lowest measure of the recombinant receptor-dependent activity, optionally within 50%, within 25%, within 20%, within 15%, within 10%, or within 5% below such lowest measure, in a composition from among a plurality of reference compositions; iii) is below the measure of the lowest recombinant receptor-dependent activity among more than 75%, 80%, 85%, 90%, 95%, or 98% of samples from a plurality of reference compositions; wherein the plurality of reference compositions are a plurality of compositions of the cell therapy comprising T cells expressing the recombinant receptor, optionally the CAR, from among a group of subjects that went on to receive the cell therapy for treating the same a disease or condition, optionally the same disease or condition, optionally ALL, those subjects in the group that went on to develop severe neurotoxicity, optionally grade 3 or higher, prolonged grade 3 or higher or grade 4 or 5 neurotoxicity.

In some embodiments, the immunotherapy specifically binds to an antigen associated with the disease or condition or expressed in cells of the environment of a lesion associated with the disease or condition. In some embodiments, the antigen is CD19, CD20, CD22 or CD123.

In some embodiments, the immunotherapy is a T cell-engaging therapy comprising a bispecific antibody, wherein at least one binding portion specifically binds to a T cell antigen and a second binding portion binds to the antigen associated with the disease or condition or expressed in cells of the environment of a lesion associated with the disease or condition. In some embodiments, the T cell antigen is CD3. In some embodiments, the second binding portion binds CD19. In some embodiments, the bispecific antibody is blinatumomab.

In some embodiments, the immunotherapy is a cell therapy, wherein the cell therapy includes genetically engineered cells expressing a recombinant receptor. In some embodiments, the genetically engineered cells include T cells or NK cells. In some embodiments,the engineered cells include T cells. In some embodiments, the immunotherapy is a T cell therapy comprising genetically engineered T cells expressing a recombinant receptor.

In some embodiments, the T cells include CD4+ and/or CD8+ T cells. In some embodiments, the recombinant receptor is a T cell receptor or a functional non-T cell receptor. In some embodiments, the recombinant receptor is a chimeric antigen receptor (CAR). In some embodiments, the recombinant receptor is an anti-CD19 CAR. In some embodiments, the CAR includes an extracellular antigen-recognition domain that specifically binds to the antigen and an intracellular signaling domain comprising an ITAM, wherein optionally, the intracellular signaling domain includes an intracellular domain of a CD3-zeta (CD3ζ) chain; and/or wherein the CAR further includes a costimulatory signaling region, which optionally includes a signaling domain of CD28 or 4-1BB.

In some embodiments, the risk or likely risk of the subject developing neurotoxicity following administration of the cell therapy is further based on the value of a parameter that indicates or correlates with the degree of recombinant receptor-dependent, optionally CAR-dependent, activity of the composition, wherein if the value of the parameter is at or greater than a threshold value the subject is at risk of developing neurotoxicity following administration of the immunotherapy when administered to the subject. In some embodiments, the recombinant receptor-dependent activity includes a measure of the production or accumulation of one or more of a proinflammatory cytokine, or a normalized value thereof. In some embodiments, the proinflammatory cytokine is TNF-alpha, IFN-gamma, IL-2, IL-10, or a combination thereof.

In some of any such embodiments, the recombinant receptor is any recombinant receptor described herein, optionally a CAR. In some of any such embodiments, the cell therapy comprises the administration of from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some of any such embodiments, the cell therapy comprises the administration of no more than 1×10⁸ total recombinant receptor-expressing cells, total T cells, or total peripheral blood mononuclear cells (PBMCs), no more than 1×10⁷ total recombinant receptor-expressing cells, total T cells, or total peripheral blood mononuclear cells (PBMCs), no more than 0.5×10⁷ total recombinant receptor-expressing cells, total T cells, or total peripheral blood mononuclear cells (PBMCs), no more than 1×10⁶ total recombinant receptor-expressing cells, total T cells, or total peripheral blood mononuclear cells (PBMCs), no more than 0.5×10⁶ total recombinant receptor-expressing cells, total T cells, or total peripheral blood mononuclear cells (PBMCs).

In some embodiments, the immunotherapy is a cell therapy, said cell therapy comprising genetically engineered cells expressing a recombinant receptor, and the subject is administered a dose that is from or from about 2×10⁶ to 5×10⁷ total recombinant receptor-expressing cells, inclusive, or that is from or from about 2×10⁵ cells/kg to 5×10⁵ cells/kg total recombinant receptor-expressing cells, inclusive. In some embodiments, the immunotherapy is a cell therapy, said cell therapy comprising genetically engineered cells expressing a recombinant receptor, and the subject is administered a dose that is from or from about 1×10⁷ to 2.0×10⁸ total recombinant receptor-expressing cells, inclusive, or that is from or from about 1×10⁶ cells/kg to 2×10⁶ cells/kg total recombinant receptor-expressing cells, inclusive. In some embodiments, the immunotherapy is a cell therapy, said cell therapy comprising genetically engineered cells expressing a recombinant receptor, and the subject is administered a dose that is from or from about 1×10⁷ to 5×10⁷ total recombinant receptor-expressing cells, inclusive, or the subject is administered a dose that is from or from about 1×10⁶ cells/kg to 2×10⁶ cells/kg total recombinant receptor-expressing cells, inclusive. In some embodiments, the subject is an adult human subject. In some embodiments, the subject is a pediatric human subject.

Provided herein is a kit, comprising reagents for detecting the expression of two or more gene products or portions thereof in a sample, wherein the two or more gene products are encoded by two or more of ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, ZNF415, ENG, SELE, ICAM3, PCDHGA9, FMNL1, or IL6R, or a portion or a fragment of any of the forgoing.

In some of any such embodiments, the two or more gene products are human gene products. In some of any such embodiments, the kit comprises reagents for detecting the expression of at least 3, at least 4, at least 5, at least 6, at least 7, at least 8, at least 9, at least 10, at least 11, at least 12, at least 13, at least 14, at least 15, at least 20, at least 25, at least 30, at least 40, or at least 50 gene products. In some of any such embodiments, the kit comprises reagents for detecting the expression of at least 3 gene products. In some of any such embodiments, the kit comprises reagents for detecting the expression of at least 5 gene products.

In some of any such embodiments, at least one of the two or more gene products is from a first group of gene products that negatively correlate to a risk of developing neurotoxicity, wherein the first group comprises gene products encoded by ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, and IL6R, and portions or a fragments of any of the forgoing.

In some of any such embodiments, at least one of the two or more gene products is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing. In some of any such embodiments, at least one of the two or more gene products is a gene product encoded by JCHAIN, MUC4, CA6, WNT9A, ADGRF1 or CCL17, or a portion or fragment of any of the foregoing. In some embodiments, at least one of the two or more gene products is a gene product encoded by CCL17, ENG, SELE, ICAM3, or IL6R, and portions or a fragments of any of the forgoing. In some of any such embodiments, at least one of the two or more gene products is a gene product encoded by CCL17 or is a portion or fragment thereof.

In some of any such embodiments, at least one of the two or more gene products is from a second group of gene products that positively correlate to a risk of developing neurotoxicity, wherein the second group comprises gene products encoded by ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRXS, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, and ZNF415, or portions or a fragments of any of the forgoing.

In some of any such embodiments, at least one of the two or more gene products is a gene product encoded by ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

In some of any such embodiments, at least one of the two or more gene products is a gene product encoded by PINLYP, ASAP2, FMNL2, PTCH1, TTC28, PCDHGA6, PCDHGB6 or PCDHGA12, or a portion or fragment of any of the foregoing. In some of any such embodiments, at least one of the two or more gene products is a gene product encoded by PINLYP or PCDHGA12, or a portion or fragment of any of the foregoing. In some of any such embodiments, at least one of the two or more gene products is a gene product, or a portion or fragment thereof, from the first group of gene products that negatively correlate to a risk of developing neurotoxicity and at least one of the gene products is a gene product, or a portion or fragment thereof, from the second group of gene products that positively correlate to a risk of developing neurotoxicity.

In some embodiments, at least one of the two or more gene products is a gene product, or a portion or fragment thereof, from a first group of gene products that negatively correlate to a risk of developing neurotoxicity selected from CCL17, ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, and portions or a fragments of any of the forgoing; and at least one of the gene products is a gene product, or a portion or fragment thereof, from a second group of gene products that positively correlate to a risk of developing neurotoxicity selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, and ZNF415, or portions or a fragments of any of the forgoing.

In some of any such embodiments, the at least one of the gene products from the first group is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing. In some of any such embodiments, the at least one of the gene products from the first group is a gene product encoded by JCHAIN, MUC4, CA6, WNT9A, ADGRF1 or CCL17, or a portion or fragment of any of the foregoing. In some of any such embodiments, the at least one of the gene products from the first group is a gene product encoded by CCL17 or is a portion or fragment thereof.

In some of any such embodiments, the at least one of the gene products from the second group is a gene product encoded by ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

In some of any such embodiments, the at least one of the gene products from the second group is a gene product encoded by ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

In some of any such embodiments, the at least one of the gene products from the second group is a gene product encoded by PINLYP, ASAP2, FMNL2, PTCH1, TTC28, PCDHGA6, PCDHGB6 or PCDHGA12, or a portion or fragment of any of the foregoing.

In some of any such embodiments, the at least one of the gene products from the second group is a gene product encoded by PINLYP or PCDHGA12, or a portion or fragment of any of the foregoing. In some of any such embodiments, the at least one of the gene products from the first group is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing; and wherein the at least one of the gene products from the second group is a gene product encoded by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

In some of any such embodiments, the at least one of the gene products from the first group is a gene product encoded by JCHAIN, MUC4, CA6, WNT9A, ADGRF1 or CCL17, or a portion or fragment of any of the foregoing; and wherein the at least one of the gene products from the second group is a gene product encoded by PINLYP, ASAP2, FMNL2, PTCH1, TTC28, PCDHGA6, PCDHGB6 or PCDHGA12, or a portion or fragment of any of the foregoing. In some of any such embodiments, the at least one of the gene products from the first group is a gene product encoded by CCL17 or is a portion or fragment thereof; and wherein the at least one of the gene products from the second group is a gene product encoded by PINLYP or PCDHGA12, or a portion or fragment thereof.

In some embodiments, the two or more gene products are or include mRNA. In some embodiments, the reagents include one or more oligonucleotide and/or polynucleotide probes that are to, bind to, and/or are capable of binding to the one or more mRNA gene products. In some embodiments, the two or more gene products are or include proteins or variants or fragments thereof.

In some embodiments, the two or more gene products are selected from CCL17, ENG, SELE, ICAM3, or IL6R, and portions or a fragments of any of the forgoing. In some embodiments, the reagents are or include antibodies or antigen binding fragments or variants thereof, wherein the antibodies or the antigen binding fragments or variants thereof bind to and/or are capable of binding to the protein gene products. In some embodiments, the kit further contains an immunotherapy. In some embodiments, the immunotherapy is a cell therapy or is a T cell-engaging therapy, optionally wherein the cell therapy includes cells engineered to express a recombinant receptor. In some embodiments, the kit is for use in connection with any of the methods provided herein.

Provided herein is an article of manufacture, comprising any of the kits provided herein, and instructions for using the reagents to assay a biological sample from a subject that is a candidate for treatment, optionally with a cell therapy, said cell therapy optionally comprising a dose or composition of genetically engineered cells expressing a recombinant receptor. In some embodiments, the instructions specify carrying out any of the methods provided herein.

In some of any such embodiments, the instructions specify assessing the presence, absence or level of expression of the two or more gene products or portions thereof in the sample, and comparing the presence, absence, or level of expression of the two or more gene products or portions thereof to gene reference values, wherein the comparison indicates the risk or likely risk of the subject developing neurotoxicity following administration of the cell therapy when administered to the subject. In some of any such embodiments, wherein the instructions specify each of the one or more gene products is individually compared to a gene reference value for the respective gene product. In some of any such embodiments, the instructions specify the sample does not comprise cells genetically engineered with the recombinant receptor and/or is obtained from the subject prior to receiving the cell therapy.

In some of any such embodiments, the instructions specify the comparison indicates the subject is or is likely at risk of developing neurotoxicity if the level of expression of the at least one gene product from the first group of gene products that negatively correlate to a risk of developing neurotoxicity is at or below a gene reference value and/or the level of expression of the at least one gene product from the second group of gene products that positively correlate to a risk of developing neurotoxicity is at or above a gene reference value; or the instructions specify the comparison indicates the subject is not or is likely not at risk of developing neurotoxicity if the level of expression of the at least one gene product of (a) is above a gene reference value and/or the level of expression of the at least one gene product of (b) is below a gene reference value.

In some of any such embodiments, the instructions specify if the comparison indicates the subject is or is likely to develop neurotoxicity, selecting the subject for administration of a therapeutic regimen, the therapeutic regimen comprising administering to the subject: i. an agent or other treatment capable of treating, preventing, delaying, reducing or attenuating the development or risk of development of a toxicity and (2) the cell therapy, wherein administration of the agent is to be administered (i) prior to, (ii) within one, two, or three days of, (iii) concurrently with and/or (iv) at first fever following, the initiation of administration of the cell therapy to the subject; ii. the cell therapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the cell therapy; and/or iii. the cell therapy in an in-patient setting and/or with admission to the hospital for one or more days, optionally wherein the cell therapy is otherwise to be administered to subjects on an outpatient basis or without admission to the hospital for one or more days; or iv. an alternative therapeutic treatment other than the cell therapy.

### BRIEF DESCRIPTION OF THE DRAWINGS

FIGS. 1A-1D show a volcano plot and heat maps displaying the expression levels of genes across 31 bone marrow aspirate (BMA) samples collected from subjects with acute lymphoblastic leukemia (ALL). FIG. 1A shows a volcano plot comparing gene expression in samples from subjects having grades 0 or 1 neurotoxicity with samples from subjects having grades 4 or 5 neurotoxicity. For the individual genes, the Log 2 fold change in gene expression between samples associated with grades 0-1 and 4-5 neurotoxicity (x-axis) and the −log 10 of the adjusted p value (y-axis) is plotted. FIGS. 1B-1C show heat maps displaying the expression levels of exemplary genes positively and negatively correlated to the degree of neurotoxicity (NTX) across the individual BMA samples (X axis). The neurotoxicity grade associated with each sample is shown along the X axis. The BMA samples are arranged into 3 groups: the group 1 samples show high levels of expression of genes negatively correlated to toxicity (“A” genes) and low expression of genes positively correlated to toxicity (“B” genes), the group 2 samples show high expression of genes positively correlated to neurotoxicity genes and low expression of genes negatively correlated to toxicity genes, and the Group 3 samples show mixed expression of genes negatively and positively correlated to neurotoxicity. FIG. 1D shows a heat map displaying the expression of genes negatively correlated to toxicity (“Low NTX Genes (Ph-like genes)”; “A” genes) and genes positively correlated to toxicity (“High NTX Genes” (Non Ph-like genes)”; “B” genes) for samples associated with maximum observed neurotoxicity grades of 0-1 and 4-5.

FIGS. 2A and 2B show multidimensional analyses illustrating the relationship between the expression level of genes in BMA samples taken from subjects and the in vitro antigen-stimulated TNF-alpha release of therapeutic T cell compositions that were administered to the subjects with the degree of neurotoxicity experienced by the subjects.

FIG. 2A shows a two dimensional scatter plot that depicts the expression of the PINLYP and CCL17 genes as log 2-transformed quantile-normalized FPKM (FPKQ (log 2)). The level of in vitro antigen-stimulated TNF-alpha release of the therapeutic cell composition is indicated by size of the circles marking the data points, with larger circles indicating a high degree of TNF-alpha release, and smaller circles indicating a lesser release of TNF-alpha. The numbers adjacent to the circles indicate the grade of the associated neurotoxicity experienced by the subject.

FIG. 2B shows a two dimensional scatter plot that depicts the expression levels of the PCDHGA12 and CCL17 genes, expressed as FPKQ (log 2). The level of in vitro antigen-stimulated TNF-alpha release of the therapeutic cell composition is indicated by size of the circles marking the data points, with larger circles indicating a high degree of TNF-alpha release, and smaller circles indicating a lesser release of TNF-alpha. The numbers adjacent to the circles indicate the grade of the associated neurotoxicity experienced by the subject.

FIG. 3 displays a multidimensional analyses illustrating the data presented in FIG. 2B. A two dimensional scatter plot is shown that depicts the expression level of the CCL17 gene expressed as FPKQ (log 2) and the antigen-stimulated TNF-alpha release by the therapeutic cell composition. The level of PCDHGA12 expression is indicated by size of the circles marking the data points, with larger circles indicating a high degree of PCDHGA12 expression, and smaller circles indicating a low degree of PCDHGA12 expression. The numbers adjacent to the circles indicate the grade of the associated neurotoxicity experienced by the subject.

FIG. 4 shows a heat map displaying the expression levels of exemplary Group A and Group B genes (Y axis) across 223 BMA samples collected from subject with pediatric ALL. The BCR-ABL1 status and IKZF copy number status of each sample are shown on the X axis.

FIGS. 5A and 5B show graphs displaying the levels of CCL17 detected in an immunoassay in plasma samples collected from subjects. Subjects were grouped according to the grade of neurotoxicity that developed following administration of the CAR+ T cells. FIG. 5A shows a graph displaying the concentration of CCL17 in plasma samples associated with neurotoxicity grades 0-1, 2-3, and 4-5 collected from subjects prior to administration of anti-CD19 CAR+ T cells. FIG. 5B shows a whisker plot graph displaying the levels of CCL17 in plasma samples associated with neurotoxicity grades (NTX Gr) 0-1 and 4-5 collected from subjects 1 day prior and 2, 4, and 7 days subsequent to administration of anti-CD19 CAR T cells.

FIGS. 6-8 show whisker plot graphs displaying the levels of Endoglin (FIG. 6), E-Selectin (FIG. 7) or ICAM-3 (FIG. 8), respectively, detected by an immunoassay in plasma samples (collected from subjects 1 day prior and 2, 4, and 7 days subsequent to administration of anti-CD19 CAR-T cells) in groups of subjects grouped according to grade of development of neurotoxicity (NTX Gr) (subjects developing grades 0-1 vs 4-5 neurotoxicity) following administration of the CAR+ T cells. Numbers above the X-axis indicate number of assessed subjects for each time point. Y axis shows arbitrary units of the assay signal, with higher values reflecting higher detection of the protein. Horizontal lines of the whisker plots indicate median values, and diamonds indicate mean values. Circles indicate individual data points.

FIG. 9 shows a whisker plot graph displaying the levels IL-6 receptor (IL-6R) detected by an immunoassay in plasma samples (collected from subjects 1 day prior and 2, 4, and 7 days subsequent to administration of anti-CD19 CAR-T cell) in groups of subjects grouped according to the development of neurotoxicity (subjects developing grades (Gr) 0-1 vs 4-5 neurotoxicity) following administration of the CAR+ T cells. The count (number of assessed subjects), median expression, and number of outliers for each group are listed below the X axis. The Y axis shows arbitrary units of the assay signal, with higher values reflecting higher detection of the protein. Horizontal lines of the whisker plots indicate median values, and circles represent the outliers.

FIG. 10 shows a whisker plot graph displaying the levels of CCL7, E-selectin, endoglin, ICAM3, and soluble IL-6R (sIL6R) detected by a Luminex multiplex assay in plasma samples collected from subjects prior to administration of anti-CD19 CAR-T cells in groups of subjects grouped according to the development of neurotoxicity (subjects developing grades<2 vs >2 neurotoxicity) following administration of the CAR+ T cells. The count (number of assessed subjects), median expression, and number of outliers for each group are listed below the X axis. The Y axis shows arbitrary units of the assay signal, with higher values reflecting higher detection of the protein. Horizontal lines of the whisker plots indicate median values, and circles represent the outliers. P values (Pval) of the comparisons between groups for each protein are indicated at the top of the graph.

FIG. 11 shows a graph displaying (i) for each of a number of individual subjects, individual non-boxed dots indicate maximum PK measure (number of CAR+ T cells/μl of blood (PK measure) at peak (on an individual subject-by-subject basis, the time-point at which the highest number of CAR+ T cells was measured, among the time-points at which CAR+ T cell numbers were assessed following treatment of the subject with anti-CD19 CAR-expressing T cell compositions) (with dots also indicating the highest grade of neurotoxicity observed for each subject: grade 0-2 (square), grade 3 (star), prolonged grade 3 (diamond), grade 4 (pentagon with arrow), or grade 5 (circle) neurotoxicity and (ii) median levels, for those of such patients deemed to have had an early or late CAR+ T cell peak (indicated by upper and lower lines with boxed dots with range bars, respectively) of IL-15 (picograms/mL) in serum at various indicated time points prior to and following administration of such T cell compositions. The oval indicates subjects in which early, rapid expansion of CAR-T cells was observed, as compared to other subjects.

### DETAILED DESCRIPTION

Provided herein are methods of determining, assessing, and/or measuring a risk, probability, and/or likelihood of toxicity following administration of a therapy, e.g., a cell therapy and/or an immunotherapy, that include one or more steps for measuring, assessing, determining, and/or quantifying the presence, absence, or level of expression of one or more gene products, or portions thereof in a sample and comparing the presence, absence, or level of expression of the one or more gene products or portions thereof to a gene reference value, wherein the comparison indicates the risk, probability, or likelihood of the subject developing a toxicity following administration of and/or associated with the therapy, e.g. cell therapy. Also provided herein are methods of treatment that include one or more steps of administering to a subject a dose of a therapy, e.g. a cell therapy and/or an immunotherapy, for treatment of a disease or condition following and/or based on the results of assessing the presence, absence, or level of expression, from a sample from the subject, of one or more gene products or portion thereof, wherein the assessment indicates the risk, probability, or likelihood of the subject developing a toxicity following administration of and/or associated with the therapy, e.g. cell therapy.

In particular embodiments, the one or more gene products are associated with and/or correlated to a risk, probability, and/or likelihood of developing and/or experiencing toxicity following administration of and/or associated with the therapy. In some embodiments, the sample is taken or obtained from a subject that is a candidate for receiving the therapy. In some embodiments, the sample does not contain or include the therapy, e.g., genetically engineered cells of a cell therapy. In certain embodiments, the sample does not include genetically engineered cells, e.g., genetically engineered cells from a therapy.

Also provided herein are methods of treatment including one or more steps of selecting a subject that exhibits a Philadelphia chromosome (Ph+) and/or Philadelphia chromosome-like (Ph-like) molecular subtype of acute lymphoblastic leukemia (ALL) and administering to the subject a therapy, e.g. a cell therapy and/or immunotherapy, comprising a dose of therapy for the treatment, alleviation, and/or amelioration of ALL.

Immunotherapies, such as adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies), can be effective in treating cancer and other diseases and disorders. In certain contexts, available approaches to immunotherapy, such as adoptive cell therapy, may not always be entirely satisfactory. In some aspects, the provided embodiments are based on observations that the efficacy of adoptive cell therapy may be limited in some context by the development of, or risk of developing, toxicity or one or more toxic outcomes in the subject. In some cases, such toxicities can be severe. For example, in some cases, administering a dose of cells expressing a recombinant receptor, e.g. a CAR, can result in toxicity or risk thereof, such as CRS or neurotoxicity.

Predicting the risks of adverse outcomes or toxicity associated with the administration of a therapy, e.g., an immunotherapy such as a cell therapy, in individual subjects is useful for evaluating, monitoring, and/or tailoring current or potential therapies for individual subjects, particularly in the context of minimizing potential life-threatening side effects (e.g. cytokine release syndrome or severe neurotoxicity). In some aspects, the methods provided herein may be used to minimize, mitigate, and/or avoid the risk of toxicity and other such undesirable outcomes of a therapy, e.g. an immunotherapy such as a cell therapy or CAR-T cell therapy.

In some embodiments, the methods provided herein contain one or more steps of determining the gene signatures in a sample, e.g., a bone marrow aspirate sample or a serum sample, by assessing one or more specific genes products as provided herein in order to predict or assess the degree of a risk for toxicity associated with a therapy in subjects. In certain embodiments, the one or more specific gene products are from among two sets of genes: (1) genes identified as having high expression in low-risk subjects (e.g. grade 0-2 neurotoxicity) and low expression in high-risk subjects (e.g. severe neurotoxicity, such as grade 3 or higher, for example grade 4 or 5 neurotoxicity), and/or (2) genes identified as having high expression in high-risk subjects and low expression in low-risk subjects. In some embodiments, the gene signatures combining the expression of two or more gene products from one or both sets of genes are useful for predicting the degree of the risk, probability, or likelihood of a toxicity, for example severe neurotoxicity. In some embodiments, high-risk neurotoxicity is or is characterized by grade 4 or 5 neurotoxicity or is severe neurotoxicity. In particular embodiments, the therapy is an immunotherapy and/or a cell therapy, such as a CAR-T cell therapy. In certain embodiments, the analysis of the gene signature in the sample may be combined with measurements of a parameter of the therapy, such as an activity readout of engineered T cells of a CAR-T cell therapy, e.g., TNF-alpha secretion by cells of a cell therapy, to access the risk of toxicity in individual subjects in a manner that is strongly predictive.

In certain embodiments, the methods provided herein assess the gene signatures of the sample collected prior to any administration of the therapy. Thus, in such embodiments, the sample does not contain the therapy and has not been affected by or exposed to the therapy. The sample may contain tumor or cancer cells, or may contain gene products that are released or secreted by tumor cells. Thus, in certain embodiments, the methods provided herein provide an assessment and/or determination of the subject's risk to a toxicity associated with a therapy based on properties that are intrinsic to the subject and/or the subject's disease. Without being bound by theory, the predictive nature of the gene signatures provided herein may suggest that toxicity, e.g., neurotoxicity, results from an interaction or relationship between the disease or cancer and the therapy, e.g., the CAR-T cell therapy.

In particular embodiments, provided herein are methods for assessing the risk of a subject for toxicity following administration of a therapy, e.g., an immunotherapy and/or a cell therapy, by assessing, measuring, determining, and/or quantifying the gene signature and/or the expression of one or more genes or gene products that are associated with and/or correlate to toxicity in a sample. In certain embodiments, the sample is taken and/or obtained from the subject prior to any treatment or administration of the therapy, e.g. a cell therapy. Thus, in some embodiments, the gene signature, e.g., the expression of the one or more genes or gene products in the sample, is not a response to the therapy but reflects a preexisting disposition of the subject and/or of the particular strain of the subjects' cancer to the therapy. In some embodiments, an advantage of the methods provided herein is that a subject at risk for toxicity may be identified before the therapy is administered, so that the subject may be assigned close monitoring during and following treatment of the therapy, receive treatment of the therapy in a hospital setting, and/or in some embodiments may receive an intervention that prevents, treats, and/or ameliorates toxicity prior to receiving the therapy or before any symptoms of a toxicity are present. In certain embodiments, the at risk subject may be selected to receive a low dose of the therapy, or in some cases, to receive an alternative therapy. In some embodiments, the alternative therapy is a therapy that treats the disease or condition other than the immunotherapy, e.g. cell therapy, for treating the disease or condition.

In certain embodiments, the risk, probability, and/or likelihood of toxicity following administration of or associated with the therapy is assessed in a subject with minimal invasiveness by utilizing samples, such as bone marrow aspirates and/or tumor samples or serum samples, that are collected at a screening session, such as a routine biopsy or blood draw to confirm and/or identify the condition or disease in the subject. For example, in some embodiments, a bone marrow sample is collected or obtained from a subject having or suspecting of having a leukemia, such as ALL, at an initial screening, such as when a treatment regimen is still being planned or considered. Thus, in some embodiments, the methods provided herein allow for the risk of toxicity following administration of a therapy to be assessed in a subject without the need for additional procedures, e.g., additional biopsies, beyond the normal procedures leading up to the therapy.

In particular embodiments, provided herein are the identities of genes with expression that is negatively or positively correlated to and/or associated with a risk of developing a toxicity following administration of a therapy, e.g., an immunotherapy and/or a cell therapy. For example, in some embodiments, provided herein are genes that have been identified through gene expression analysis, e.g., RNA-seq analysis, of samples obtained from subjects in a study, e.g., a clinical study, prior to treatment with the therapy and analyzed for a correlation to the incidence and/or the degree of toxicity experienced by the subjects during the study. In some embodiments, further analysis has demonstrated that the expression of one or more genes identified herein can be analyzed in samples taken from subjects prior to a therapy, such as a cell therapy, to accurately predict the risk of toxicity following administration of the therapy, e.g. a cell therapy. In particular aspects as described, at least one of the one or more gene products is selected from a gene listed in Table 1 or Table E2A and/or at least one of the one or more of the gene products is selected from a gene listed in Table 2 or Table E2B (e.g. a subset of genes in the relevant Table which as a recited SEQ ID NO, and/or a Uniprot ID). In some embodiments, the presence, absence, or level of expression of the one or more gene products are compared to reference values of the one or more gene products.

In certain embodiments, the gene signature of the sample may reflect and/or identify a molecular or genetic subtype of the subject's disease or cancer. In some embodiments, some or all of the gene signatures provided herein of bone marrow or blood samples obtained from subjects with ALL may reflect and/or identify a molecular or genetic subtype of the subject's ALL. For example, in some embodiments, it is found herein that the high expression of certain genes that negatively correlate to the degree and/or risk of neurotoxicity following administration of a therapy, e.g., an immunotherapy and/or a cell therapy, have been identified in bone marrow and peripheral blood samples as components of a genetic signature for a subtype of ALL, e.g., a molecular subtype, such as an ALL subtype termed “Ph-like” or “BCR-ABL-like” (Harvey et al., Blood 122: 826 (2013); PCT App. No. PCT/US2012/069228) and an ALL subtype termed “R8” (Harvey et al., Blood 116(23): 4874-4884 (2010)). Thus, in some embodiments, the gene signatures associated with a high or low risk of toxicity following administration of a therapy may, at least in some cases, identify distinct subtypes and/or populations of ALL.

In particular embodiments, provided herein are molecular subtypes of ALL and/or B-ALL that are negatively or positively correlated to and/or associated with a risk of developing a toxicity following administration of a therapy, e.g., an immunotherapy and/or a cell therapy. For example, in some embodiments, provided herein are molecular subtypes of ALL that were identified in samples obtained from subjects in a study, e.g., a clinical study, prior to treatment with the therapy and analyzed for a correlation to the incidence and/or the degree of toxicity experienced by the subjects during the study. In certain embodiments, subjects that are identified as Ph+ and/or Ph-like have a lower risk of developing toxicity, e.g., neurotoxicity, than non Ph− and/or Ph-like subjects. Thus, in some embodiments, identification of a subject's molecular subtype or gene expression signature of All and/or B-ALL is used to assess the risk and/or probability for experiencing a toxicity associated with a cell therapy, such as an anti-CD19 CAR T cell therapy.

In some embodiments, information obtained from the gene signature of a sample from a subject may be combined with other assessments and/or measurements to accurately (or more accurately) assess or determine the risk of a subject for developing a toxicity following administration of a therapy, e.g., a cell therapy and/or an immunotherapy. In particular embodiments, the gene signature of the sample may be analyzed in conjunction with one or more parameters of the cells of a cell therapy. In some embodiments, a parameter of the therapy may be assessed in conjunction with the gene signatures to determine if and/or the dose that the therapy may be administered to the subject, for example to minimize or further reduce the risk, probability or likelihood that the subject will experience toxicity following administration of and/or associated with the therapy. For example, in some embodiments, one or more parameters of the cells of the cell therapy that relate to cellular activity and/or immune function are measured and combined in the analysis of the gene signature to assess or determine a subjects risk for developing a toxicity following administration of the therapy. In particular embodiments, therapy contains cells expressing a recombinant receptor, e.g., a CAR, and the parameter relates to recombinant receptor-dependent cellular activity and/or immune function of the therapy. In some embodiments, the combined analysis of the gene signature and the parameter might increase the accuracy and/or the predictive power of the assessment for toxicity as opposed to an analysis of either the gene signature or the parameter alone.

In all of the provided embodiments, a preferred immunotherapy is CAR-T cell therapy targeting an antigen associated with ALL, and the disease or condition to be treated is ALL. In such preferred embodiments, the CAR-T cell therapy is targets CD19, CD20, CD22 or CD23. In some embodiments of a preferred embodiment, the CAR-T cell therapy is an anti-CD19 CAR-T cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS OF ASSESSING RISK TOXICITY

In particular embodiments, provided herein are methods to assess, predict, infer, and/or estimate a risk of toxicity, for example to a therapy such as an immunotherapy or cell therapy. In some embodiments, the subject is administered, will be administered, or is a candidate to be administered a therapy, e.g., an immunotherapy and/or a cell therapy. In some embodiments, the toxicity is a toxicity following administration of the therapeutic treatment. In certain embodiments, the toxicity is triggered, caused, and/or associated with a therapy. In particular embodiments, methods for the assessment, prediction, inference, and/or estimate of the risk or probability include one or more steps for measuring, assessing, and/or determining the expression products of one or more genes (hereinafter one or more “gene products”), e.g., a gene expression profile. In certain embodiments, the expression of the one or more gene products is predictive of, correlated with, and/or associated with a risk of toxicity following administration of therapy, e.g., the immunotherapy and/or the cell therapy. In certain embodiments, the expression of one or more gene products is measured in a sample, e.g., a sample taken, collected, and/or obtained from the subject.

Particular embodiments contemplate that the expression of one or more gene products, e.g., a gene expression profile, of a sample is predictive of, correlated with, and/or associated with a risk, e.g., a high risk or a low risk, of toxicity, e.g., toxicity following administration of or associated with a therapy. In some embodiments, the gene expression profile of a sample is or includes the expression the one or more gene products that are correlated with and/or associated with a risk and/or an incidence of a toxicity. In certain embodiments, the gene expression profile of a sample is or includes the expression of one or more gene products that are negatively correlated with and/or negatively associated with a risk and/or an incidence of a particular grade or grades of a toxicity, e.g. a severe toxicity such as grade 4 and/or grade 5 neurotoxicity. In certain embodiments, the gene expression profile of a sample is or includes the expression of one or more gene products that are negatively correlated with and/or negatively associated with a risk and/or an incidence of a toxicity. In particular embodiments, the gene expression profile of a sample is or includes the expression of one or more gene products that are positively correlated with and/or positively associated with a risk and/or an incidence of a toxicity. In particular embodiments, the gene expression profile of a sample is or includes the expression of one or more gene products that are positively correlated with and/or positively associated with an incidence of a particular grade or grades of a low-risk toxicity, e.g. grade 0 or grade 1 neurotoxicity. In particular embodiments, the gene expression profile of a sample is or includes the expression of one or more gene products that are positively correlated with and/or positively associated with a risk and/or an incidence of a particular grade or grades of high-risk toxicity, e.g. a severe toxicity such as grade 4 or grade 5 neurotoxicity. In certain embodiments the toxicity is to and/or associated with a cell therapy. In particular embodiments, the toxicity is to and/or associated with an immunotherapy.

In particular embodiments, the methods to assess, predict, infer, and/or estimate a risk of toxicity, for example to a therapy such as an immunotherapy, include one or more steps of assessing a gene expression profile in a sample. In some embodiments, the one or more steps include comparing the expression of one or more gene products of the gene expression profile to one or more gene reference values. In certain embodiments, comparing the expression of one or more gene products from the gene expression profile with the one or more reference values indicates the risk, probability, and/or likelihood that a subject will experience a toxicity following administration of the therapy. In some embodiments, the sample is obtained from a subject that is administered, will be administered, or is a candidate to be administered a therapy, e.g., an immunotherapy and/or a cell therapy. In particular embodiments, the methods to assess, predict, infer, and/or estimate a risk of toxicity, include one or more steps for taking, collecting, and/or obtaining a sample from a subject; detecting, measuring, and/or obtaining a gene expression profile of the sample; and/or comparing the expression of one or more gene products of the gene expression profile to determine, estimate, or predict the risk or likelihood of toxicity.

A. Sample

In certain embodiments, the expression of one or more gene products are measured, assessed, and/or determined in a sample. In particular embodiments, the sample is a biological sample that is taken, collected, and/or obtained from a subject. In certain embodiments, the subject has a disease or condition and/or is suspected of having a disease or condition. In some embodiments, subject has received, will receive, or is a candidate to receive a therapy. In some embodiments, the therapy is an administration of a cell therapy. In particular embodiments, the therapy is an immunotherapy. In certain embodiments, the cell therapy treats and/or is capable of treating the disease or condition. In some embodiments, the therapy is a cell therapy that contains one or more engineered cells. In some embodiments, the engineered cells express a recombinant receptor. In particular embodiments, the recombinant receptor is a chimeric antigen receptor (CAR). In particular embodiments, the sample is taken, collected, and/or obtained from a subject who has been, who will be, or is a candidate to be administered a therapy. In particular embodiments, the sample is taken, collected, and/or obtained prior to treatment or administration with the therapy, e.g., the cell therapy.

In particular embodiments, the sample is taken, collected, and/or obtained from a subject who has been, who will be, or is a candidate to be administered a therapy. In particular embodiments, the sample is taken, collected, and/or obtained prior to treatment or administration with the therapy, e.g., the cell therapy. In accord with methods, kits and articles of manufacture described herein, the sample can be assessed for one or more gene products that is associated with and/or correlate to toxicity or risk of toxicity. Exemplary gene products that are associated with and/or correlated with a risk of developing toxicity based on expression in a sample collected or obtained from a subject prior to receiving an immunotherapy are described in Section I.B and/or Table 1, Table E2A, Table 2 or Table E2B (e.g. a subset of these genes in the relevant Table which has a recited SEQ ID NO, and/or a Uniprot ID). Thus, in some aspects, the provided methods relate to identifying subjects, prior to receiving an immunotherapy, such as a cell therapy (e.g. CAR-T cells), who may be at risk of developing a toxicity, e.g. neurotoxicity, such as grade 3 or higher or grade 4 or 5. As described elsewhere herein, the methods can be used to determine if the subject should be closely monitored following the administration of the immunotherapy, is a candidate for outpatient therapy or should receive treatment of the therapy in a hospital setting and/or is a candidate for receiving an intervention of preventing, treating or ameliorating a risk of a toxicity.

In some embodiments, the sample is taken, collected, and/or obtained subsequent to treatment or administration with the therapy, e.g., the cell therapy. In accord with methods, kits and articles of manufacture described herein, the sample can be assessed for one or more gene products that are associated with and/or correlate to toxicity or risk of toxicity after receiving the immunotherapy. Exemplary gene products that are associated with and/or correlated with a risk of developing toxicity based on expression in a sample collected or obtained from a subject subsequent to receiving an immunotherapy are described in Table E10. In some embodiments, the sample is collected within or about within or about 30 minutes, 1 hours, 2 hours, 3 hours, 4 hours, 5 hours, 6 hours, 8 hours, 10 hours, 12 hours, 14 hours or more following initiation of administration of the immunotherapy, e.g. the cell therapy. In some aspects, the sample is collected prior to the subject exhibiting a sign or symptom of a toxicity following administration of the immunotherapy, e.g. cell therapy, such as severe CRS or grade 3 CRS or greater, e.g. grade 4 or 5 CRS, and/or severe neurotoxicity or grade 3 neurotoxicity or greater, e.g. grade 4 or 5 neurotoxicity. In some embodiments, the sample is collected from the subject at a time in which the subject exhibits grade 2 or lower CRS, e.g. grade 0 or grade 1 CRS, and/or grade 2 or lower neurotoxicity, e.g. grade 0 or grade 2 neurotoxicity following administration of the immunotherapy, e.g. cell therapy. In some embodiments, the sample is collected prior to the subject developing a first sustained fever following administration of the cell therapy, e.g. immunotherapy, or at a time in which is within 1 hours, 2 hours, 3 hours, 4 hours, 5 hours, 6 hours, 7 hours, 8 hours, 9 hours, 10 hours, 12 hours, 18 hours or 24 hours of a fever, such as a sustained fever, that develops following administration of the immunotherapy, e.g. cell therapy.

In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyretic. An antipyretic may include any agent, e.g., compound, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the sample is taken, collected, and/or obtained from a subject that has or is suspected of having a condition or disease. In some embodiments, the subject has or is suspected of having a cancer or proliferative disease. In particular embodiments, the subject has a disease or condition, or is suspected of having a disease or condition, that is associated with an antigen and/or is associated with diseased cells that express the antigen. In some embodiments, the disease or condition, e.g., a cancer or proliferative disorder, is associated with αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4) epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erbB2), Her3 (erb-B3), Her4 (erb-B4), erbB dimers, human high molecular weight-melanoma-associated antigen (HMW-MAA), hepatitis B surface antigen, Human leukocyte antigen Al (HLA-A1), human leukocyte antigen A2 (HLA-A2), IL-22 receptor alpha(IL-22Ra), IL-13 receptor alpha 2 (IL-13Rα2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, melanoma-associated antigen (MAGE)-A1, MAGE-A3, MAGE-A6,MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, preferentially expressed antigen of melanoma (PRAME), progesterone receptor, a prostate specific antigen, prostate stem cell antigen (PSCA), prostate specific membrane antigen (PSMA), receptor tyrosine kinase like orphan receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms tumor 1 (WT-1), and/or a pathogen-specific or pathogen expressed antigen. In some embodiments, the antigen is a viral antigen (such as a viral antigen from HIV, HCV, HBV, etc.), bacterial antigens, and/or parasitic antigens. In certain embodiments, the subject has a disease or condition, or is suspected of having a disease or condition, that is associated with CD19 and/or is associated with diseased cells that express CD19.

In some embodiments, the sample is taken, collected, and/or obtained from a subject that has or is suspected of having a cancer or proliferative disease that is a B cell malignancy or hematological malignancy. In some embodiments, the cancer or proliferative disease is a myeloma, e.g., a multiple myeloma (MM), a lymphoma or a leukemia, lymphoblastic leukemia (ALL), non-Hodgkin's lymphoma (NHL), chronic lymphocytic leukemia (CLL), a diffuse large B-cell lymphoma (DLBCL), and/or acute myeloid leukemia (AML). In some embodiments, the cancer or proliferative disorder is ALL. In some embodiments, the subject has, or is suspected of having ALL. In some embodiments, the ALL is adult ALL. In particular embodiments, the ALL is pediatric ALL.

In certain embodiments, the sample is a biological sample. In certain embodiments, the sample is a tissue sample. In particular embodiments, the sample is or includes a tissue affected, or suspected of being affected, by a disease or condition. In some embodiments, the sample is or includes a tissue affected, or suspected of being affected by a cancer or a proliferative disease. In some embodiments, the sample is a biopsy.

In certain embodiments, the sample is collected from a tissue having or suspected of having a tumor. In particular embodiments, the sample is or includes a tumor and/or a tumor microenvironment. In particular embodiments, the tumor is precancerous or cancerous, or is suspected of being cancerous or precancerous. In certain embodiments, the tumor is a primary tumor, i.e., the tumor is found at the anatomical site where the lesion initially developed or appeared. In some embodiments, the tumor is a secondary tumor, e.g., a cancerous tumor that originated from a cell within a primary tumor located within a different site in the body. In some embodiments, the sample contains one or more cells that are cancer cells and/or tumor cells.

In particular embodiments, the sample is collected from a lesion and/or a tumor that is associated with or caused by, or is suspected of being associated with or caused by, a non-hematologic cancer, e.g., a solid tumor. In some embodiments, the tumor is associated with or caused by, or is suspected of being associated with or caused by, a bladder, a lung, a brain, a melanoma (e.g. small-cell lung, melanoma), a breast, a cervical, an ovarian, a colorectal, a pancreatic, an endometrial, an esophageal, a kidney, a liver, a prostate, a skin, a thyroid, or a uterine cancer. In some embodiments, the lesion is associated with or caused by a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma. In certain embodiments, the sample contains one or more cancer cells. In some embodiments, the sample contains one or more cells that are suspected of being cancerous.

In some embodiments, the sample is collected from a lesion or tumor that is associated with or caused by a B cell malignancy or hematological malignancy. In some embodiments, the lesion or tumor is associated with a myeloma, e.g., a multiple myeloma (MM), a lymphoma or a leukemia, lymphoblastic leukemia (ALL), non-Hodgkin's lymphoma (NHL), chronic lymphocytic leukemia (CLL), a diffuse large B-cell lymphoma (DLBCL), and/or acute myeloid leukemia (AML). In some embodiments, the lesion or tumor is associated with or caused by ALL, e.g., adult ALL or pediatric ALL.

In some embodiments, the sample is a tissue sample, e.g., a tissue biopsy. In particular embodiments, the sample is obtained, collected, or taken from connective tissue, muscle tissue, nervous tissue, or epithelial tissue. In certain embodiments, the lesion is present on the heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, urethra, lymphatic system, skin, muscle, brain, spinal cord, nerves, ovaries, uterus, testes, prostate, pharynx, larynx, trachea, bronchi, lungs, diaphragm, bone, cartilage, ligaments, or tendons. In particular embodiments, the sample is obtained, collected, or taken from bone marrow. In some embodiments, the sample is a bone marrow aspirate.

In some embodiments, the sample is a body fluid from the subject. In some embodiments, the sample is a blood, serum, plasma or urine sample. In some embodiments, the sample is a plasma sample.

In particular embodiments, the sample does not contain the therapy, e.g., the immunotherapy and/or the cell therapy. In particular embodiments, the sample does not contain any cells, e.g., engineered cells, of a cell therapy. In particular embodiments, the therapy is a T cell therapy and the sample does not contain any engineered T cells and/or any T cells of the therapy. In particular embodiments, the sample does not contain any engineered cells that express a recombinant receptor, e.g., a CAR. In some embodiments, the sample does not contain cells expressing a CAR. In certain embodiments, the sample does not contain any therapy or components of a therapy described herein, such as in Section II-A, Section II-B, or Section III-B.

In any of the provided embodiments, the sample is a bone marrow aspirate from a subject with ALL, or a subject that is likely or suspected of having ALL, and the gene product is a polynucleotide, such as RNA, e.g. mRNA. In any of the provided embodiments, the sample is a bone marrow aspirate from a subject with ALL, or that is likely or suspected of having ALL, and the gene product is a protein.

In any of the provided embodiments, the sample is a body fluid sample from a subject with ALL, or a subject that is likely or suspected of having ALL, and the gene product is a protein. In a preferred embodiment, the body fluid sample is a plasma sample.

B. Gene Products

In some embodiments, the methods provided herein include one or more steps to measure, assess, determine, and/or quantify the expression of one or a more genes (interchangeably referred to herein as one or more “gene products”), e.g., to determine a gene expression profile, of a sample to assess, predict, infer, and/or estimate a risk, likelihood, and/or probability of toxicity. In some embodiments, the sample is taken, collected, and/or obtained from subject that is administered, will be administered, or is a candidate to be administered a therapy, e.g., an immunotherapy or a T cell therapy. In particular embodiments, the sample is from a subject prior to receiving the immunotherapy, such as within 0 to 7 days prior to receiving the immunotherapy, such as cell therapy (e.g. CAR-T cells), e.g. within 1 day, 2 days, 3 days, 4 days, 5 days, 6 days or 7 days prior to receiving the immunotherapy. In certain embodiments, the expression of the one or more genes is predictive of, correlated with, and/or associated with a risk, likelihood, and/or probability of the subject developing a toxicity following administration of a therapeutic treatment.

Particular embodiments contemplate that the expression of one or more genes in a sample, e.g. from a subject prior to receiving an immunotherapy, e.g. cell therapy, is predictive of, correlated with, and/or associated with a risk, e.g., a high risk or a low risk, of toxicity, e.g., toxicity following administration of a therapeutic treatment. In certain embodiments, the expression of the one or more genes in the sample e.g. from a subject prior to receiving an immunotherapy, e.g. cell therapy, includes one or more genes that are negatively correlated with and/or negatively associated with a risk and/or an incidence of a toxicity. In particular embodiments, the expression of the one or more genes in the sample, e.g. from a subject prior to receiving an immunotherapy, e.g. cell therapy, includes one or more genes that are positively correlated with and/or positively associated with a risk and/or an incidence of a toxicity.

In certain embodiments, elevated, increased, or high amounts or levels of expression of one or more genes that are negatively correlated to and/or negatively associated with a risk and/or an incidence of toxicity in a sample, e.g. from a subject prior to receiving an immunotherapy, e.g. cell therapy, are predictive of and/or associated with a low, reduced, or decreased risk, likelihood, and/or probability of toxicity, e.g., neurotoxicity or severe neurotoxicity. In particular embodiments, reduced, decreased, or low amounts or levels of expression of one or more genes that are negatively correlated to a risk of toxicity in a sample, e.g. from a subject prior to receiving an immunotherapy, e.g. cell therapy, are predictive of and/or associated with a high, increased, or elevated risk of neurotoxicity.

In certain embodiments, elevated, increased, or high amounts or levels of expression of one or more genes that are positively correlated to and/or positively associated with a risk and/or an incidence of toxicity in a sample obtained from a subject are predictive of and/or associated with a high, increased, or elevated risk of neurotoxicity. In particular embodiments, reduced, decreased, or low amounts or levels of expression of one or more genes that are positively correlated to a risk of toxicity in a sample obtained from a subject are predictive of and/or associated with a low or reduced risk of toxicity, e.g., neurotoxicity or severe neurotoxicity.

In certain embodiments, the expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at leave seventy, at least eighty, at least ninety or at least one hundred genes that are that are negatively correlated to and/or negatively associated with a risk and/or an incidence of a toxicity are assessed, measured, detected, and/or quantified. In some embodiments, 1, 2, 3, 4, 5, 6, 7, 8, 9, 10, 11, 12, 13, 14, 15, 16, 17, 18, 19, 20, 21, 22, 23, 34, 25 or more genes that are negatively correlated to and/or negatively associated with a risk and/or and an incidence of a toxicity are assessed, measured, detected, and/or quantified. In particular embodiments, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least forty, at least fifty, at least sixty, at least seventy, at least eighty, at least ninety, or at least one hundred genes that are that are positively correlated to and/or positively associated with a risk and/or an incidence of a toxicity are assessed, measured, detected, and/or quantified. In some embodiments, 1, 2, 3, 4, 5, 6, 7, 8, 9, 10, 11, 12, 13, 14, 15, 16, 17, 18, 19, 20, 21, 22, 23, 34, 25 or more genes that are positively correlated to and/or negatively associated with a risk and/or and an incidence of a toxicity are assessed, measured, detected, and/or quantified. In some embodiments, one or more genes as described that is positively correlated with a risk and/or an incidence of a toxicity are assessed, measured, detected, and/or quantified and one or more genes as described that are negatively correlated with a risk and/or an incidence of a toxicity are assessed, measured, detected, and/or quantified.

In some embodiments, an expression of a gene that is negatively correlated to or negatively associated with a risk, likelihood, and/or probability of toxicity has, is likely to have, or has been determined to negatively correlative to the risk, likelihood, and/or probability of a toxicity that is CRS or neurotoxicity. In particular embodiments, the toxicity is severe neurotoxicity, e.g., a neurotoxicity of a grade 4 or 5 or of a prolonged grade 3 or greater. In some embodiments, the negative correlation between the expression of the gene and the risk of toxicity has, is likely to have, or has been determined to have a correlation coefficient (R) value of at or below −0.25, at or below −0.3, at or below −0.4, at or below −0.5, at or below −0.55, at or below −0.6, at or below −0.65, at or below −0.7, at or below −0.75, at or below −0.8, at or below −0.85, at or below −0.90, at or below −0.95, at or below −0.97, at or below −0.98, at or below −0.99, or about −1.0.

In some embodiments, an expression of a gene that is identified as negatively correlated to or negatively associated with a risk of toxicity has been identified based on data from a study, e.g., a clinical study. In some embodiments, the expression of the gene has been negatively correlated with an incidence of the toxicity. In certain embodiments, the toxicity is neurotoxicity. In some embodiments, the toxicity is severe neurotoxicity, e.g., a neurotoxicity of a grade 4 or 5 or of a prolonged grade 3 or greater. In some embodiments, the toxicity is CRS. In particular embodiments, the negative correlation between the expression of the gene and the incidence of toxicity has, is likely to have, or has been determined to have a correlation coefficient (R) of at or below −0.25, at or below −0.3, at or below −0.4, at or below −0.5, at or below −0.55, at or below −0.6, at or below −0.65, at or below −0.7, at or below −0.75, at or below −0.8, at or below −0.85, at or below −0.90, at or below −0.95, at or below −0.97, at or below −0.98, at or below −0.99, or about −1.0.

In some embodiments, an expression of a gene that is negatively correlated to or negatively associated with a risk or toxicity includes the expression of one or more of ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R. In certain embodiments, an expression of a gene that is negatively correlated to or negatively associated with a risk of toxicity includes the expression of one or more of ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, and WNT9A. In particular embodiments, an expression of a gene that is negatively correlated to or negatively associated with a risk of toxicity includes the expression of one or more of CCL17, CA6, JCHAIN, PTP4A3, IFITM1, CRLF2, ENAM, GBP5, ABCA9, SV2C, SLC37A3, IL15, IL2RA, and SEMA6A. In some embodiments, an expression of a gene that is negatively correlated to or negatively associated with a risk of toxicity includes the expression of one or more of CCL17, CCR6, GAS6, GL12, PTP4A3, or IL2RA. The full name, alternative symbols, and polynucleotide and polypeptide sequences of the gene products of genes with expression negatively correlated to toxicity, e.g., neurotoxicity or severe neurotoxicity, are shown in Table 1.

TABLE 1
Genes with expression negatively correlated to toxicity

| | | Alternative Names | | | |
|---|---|---|---|---|---|
| Gene Symbol | Full Name | and Symbols | Uniprot ID | mRNA | Protein |
| CA6 | carbonic anhydrase 6 | CA-VI; GUSTIN | P23280 | SEQ ID | SEQ ID |
| | | | | NOS: | NOS: |
| | | | | 1-4 | 49-52 |
| CCL17 | C-C motif chemokine ligand | TARC; ABCD-2; | Q92583 | SEQ ID | SEQ ID |
| | 17 | SCYA17; A-152E5.3 | | NO: 5 | NO: 53 |
| CCR6 | C-C motif chemokine | BN-1; DCR2; DRY6; | P51684 | SEQ ID | SEQ ID |
| | receptor 6 | CCR-6; CD196; | | NO: 6 | NO: 54 |
| | | CKRL3; GPR29; | | | |
| | | CKR-L3; CMKBR6; | | | |
| | | GPRCY4; STRL22; | | | |
| | | CC-CKR-6; C-C | | | |
| | | CKR-6 | | | |
| ENAM | enamelin | ADAI; AI1C; AIH2 | Q9NRM1 | SEQ ID | SEQ ID |
| | | | | NO: 7 | NO: 55 |
| GAS6 | growth arrest specific 6 | AXSF; AXLLG | Q14393 | SEQ ID | SEQ ID |
| | | | | NO: 8 | NO: 56 |
| GBP5 | guanylate binding protein 5 | GBP-5 | Q96PP8 | SEQ ID | SEQ ID |
| | | | | NO: 9 | NO: 57 |
| GLI2 | GEI family zinc finger 2 | CJS; HPE9; PHS2; | P10070 | SEQ ID | SEQ ID |
| | | THP1; THP2 | | NO: 10 | NO: 58 |
| ADGRF1 | adhesion G protein-coupled | PGR19; GPR110; | Q5T601 | SEQ ID | SEQ ID |
| | receptor F1 | KPG_012; hGPCR36 | | NOS: | NOS: |
| | | | | 11-12 | 59-60 |
| IFITM1 | interferon induced | 9-27; CD225; IFI17; | P13164 | SEQ ID | SEQ ID |
| | transmembrane protein 1 | LEU13; DSPA2a | | NO: 13 | NO: 61 |
| JCHAIN | joining chain of multimeric | IGJ; JCH; IGCJ | P01591 | SEQ ID | SEQ ID |
| | IgA and IgM | | | NO: 14 | NO: 62 |
| MUC4 | Mucin 4 | ASGP; MUC-4; | Q99102 | SEQ ID | SEQ ID |
| | | HSA276359 | | NOS: | NOS: |
| | | | | 15-18 | 63-66 |
| PON2 | paraoxonase 2 | A-esterase 2; aromatic | Q15165 | SEQ ID | SEQ ID |
| | | esterase 2; arylesterase | | NOS: | NOS: |
| | | 2; paraoxonase nirs; | | 19 and | 67-68 |
| | | serum aryldialkyl- | | 20 | |
| | | phosphatase 2 | | | |
| PTP4A3 | protein tyrosine phosphatase | PRL3; PRL-3; PRL-R | O75365 | SEQ ID | SEQ ID |
| | type IVA, member 3 | | | NOS: | NOS: |
| | | | | 21-22 | 69-70 |
| SEMA6A | semaphorin 6A | VIA; SEMA; HT018; | Q9H2E6 | SEQ ID | SEQ ID |
| | | SEMAQ; SEMA6A1 | | NO: 23 | NO: 71 |
| SLC37A3 | solute carrier family 37 | Sugar phosphate | Q8NCC5 | SEQ ID | SEQ ID |
| | member 3 | exchanger 3 | | NO: 24 | NO: 72 |
| SPATS2L | spermatogenesis associated | SPATS2-like protein; | Q9NUQ6 | SEQ ID | SEQ ID |
| | serine rich 2 like | SGNP; DNAPTP6 | | NO: 25 | NO: 73 |
| TMEM154 | transmembrane protein 154 | | Q6P9G4 | SEQ ID | SEQ ID |
| | | | | NO: 26 | NO: 74 |
| TP53INP1 | tumor protein p53 inducible | SIP; Teap; p53DINP1; | Q96A56 | SEQ ID | SEQ ID |
| | nuclear protein 1 | TP53DINP1; | | NOS: | NOS: |
| | | TP53INP1A; | | 27-28 | 75-76 |
| | | TP53INP1B | | | |
| WNT9A | Wnt family member 9A | | O14904 | SEQ ID | SEQ ID |
| | | | | NO: 29 | NO: 77 |
| IL2RA | Interleukin-2 receptor subunit | TAC antigen; p55; | P01589 | SEQ ID | SEQ ID |
| | alpha | CD25 | | NOS: | NOS: |
| | | | | 97-99 | 120-122 |
| ABCA9 | ATP-binding cassette sub- | | Q8IUA7 | | |
| | family A member 9 | | | | |
| ADAMTSL4 | ADAMTS-like protein 4 | | Q6UY14 | | |
| ADGRA2 | Adhesion G protein-coupled | | Q96PE1 | | |
| | receptor A2 | | | | |
| AK5 | Adenylate kinase isoenzyme | | Q9Y6K8 | | |
| | 5 | | | | |
| APOL1 | Apolipoprotein L1 | | O14791 | | |
| ARHGAP27 | Rho GTPase-activating | | Q6ZUM4 | | |
| | protein 27 | | | | |
| ARID3B | AT-rich interactive domain- | | Q8IVW6 | | |
| | containing protein 3B | | | | |
| CABP7 | Calcium-binding protein 7 | | Q86V35 | | |
| CCDC152 | Coiled-coil domain- | | Q4G0S7 | | |
| | containing protein 152 | | | | |
| CCR1 | C-C chemokine receptor type | | P32246 | | |
| | 1 | | | | |
| CEP85L | Centrosomal protein of 85 | | Q5SZL2 | | |
| | kDa-like | | | | |
| CISH | Cytokine-inducible SH2- | | Q9NSE2 | | |
| | containing protein | | | | |
| CR2 | Complement receptor type 2 | | P20023 | | |
| EPHA4 | Ephrin type-A receptor 4 | | P54764 | | |
| ENPP2 | Ectonucleotide | | Q13822 | | |
| | pyrophosphatase/phospho- | | | | |
| | diesterase family member 2 | | | | |
| FTH1P11 | ferritin heavy chain 1 | | | | |
| | pseudogene 11 | | | | |
| FTH1P2 | ferritin heavy chain 1 | | | | |
| | pseudogene 2 | | | | |
| FTH1P8 | ferritin heavy chain 1 | | | | |
| | pseudogene 8 | | | | |
| GADD45A | Growth arrest and DNA | | P24522 | | |
| | damage-inducible protein | | | | |
| | GADD45 alpha | | | | |
| GBP3 | Guanylate-binding protein 3 | | Q9H0R5 | | |
| GBP6 | Guanylate-binding protein 6 | | Q6ZN66 | | |
| GIMAP1- | GIMAP1-GIMAP5 | | A0A087 | | |
| GIMAP5 | readthrough | | WTJ2 | | |
| GPA33 | Cell surface A33 antigen | | Q99795 | | |
| GPRIN3 | G protein-regulated inducer | | Q6ZVF9 | | |
| | of neurite outgrowth 3 | | | | |
| HSPA1A | Heat shock 70 kDa protein | | P0DMV8 | | |
| | 1A | | | | |
| IFITM3 | Interferon-induced | | Q01628 | | |
| | transmembrane protein 3 | | | | |
| IL15 | Interleukin-15 | | P40933 | | |
| KIAA1257 | Uncharacterized protein | | Q9ULG3 | | |
| | KIAA1257 | | | | |
| LA16c-390H2.4 | LOC101929732 | | | | |
| LAMB1 | Laminin subunit beta-1 | | P07942 | | |
| LDB3 | LIM domain-binding protein | | O75112 | | |
| | 3 | | | | |
| LINC00623 | long intergenic non-protein | | | | |
| | coding RNA 623 | | | | |
| LST1 | Leukocyte-specific transcript | | O00453 | | |
| | 1 protein | | | | |
| LTB | Lymphotoxin-beta | | Q06643 | | |
| LY6E | Lymphocyte antigen 6E | | Q16553 | | |
| MAS1 | Proto-oncogene Mas | | P04201 | | |
| NLRC3 | NLR family CARD domain- | | Q7RTR2 | | |
| | containing protein 3 | | | | |
| PLXNA4 | Plexin-A4 | | Q9HCM2 | | |
| PTGES3P1 | prostaglandin E synthase 3 | | — | | |
| | pseudogene 1 | | | | |
| RNU1-1 | RNA, U1 small nuclear 1 | | — | | |
| RP11-345J4.6 | — | | — | | |
| RP11-421N8.1 | — | | — | | |
| RP11-51J9.5 | — | | — | | |
| RP11-51O6.1 | — | | — | | |
| RP11-552F3.9 | — | | — | | |
| RP11-686D22.9 | — | | — | | |
| RP11-723D22.3 | — | | — | | |
| RP11-723O4.6 | — | | — | | |
| RP13-512J5.1 | — | | — | | |
| RP4-620F22.2 | — | | — | | |
| RP5-940J5.9 | — | | — | | |
| RP6-109B7.5 | — | | — | | |
| RPL21P75 | — | | — | | |
| RYR2 | Ryanodine receptor 2 | | Q92736 | | |
| SAMD9L | Sterile alpha motif domain- | | Q8IVG5 | | |
| | containing protein 9-like | | | | |
| SNRPEP4 | small nuclear | | — | | |
| | ribonucleoprotein polypeptide | | | | |
| | E pseudogene 4 | | | | |
| SOCS1 | Suppressor of cytokine | | O15524 | | |
| | signaling 1 | | | | |
| SPON1 | Spondin-1 | | Q9HCB6 | | |
| SV2C | Synaptic vesicle glycoprotein | | Q496J9 | | |
| | 2C | | | | |
| TNF | Tumor necrosis factor | | P01375 | | |
| TRIM47 | Tripartite motif-containing | | Q96LD4 | | |
| | protein 47 | | | | |
| UST | Uronyl 2-sulfotransferase | | Q9Y2C2 | | |
| ENG | Endoglin | | P17813 | | |
| SELE | E-Selectin | | P16581 | | |
| ICAM3 | Intercellular adhesion | | P32942 | | |
| | molecule 3 | | | | |
| IL6R | Interleukin-6 receptor subunit | | P08887 | | |
| | alpha | | | | |
| CRLF2 | Cytokine receptor-like factor | | Q9HC73 | | |
| | 2 | | | | |

In certain embodiments, the expression of the one or more genes is positively correlated to a risk of toxicity following administration of a therapeutic treatment. In some embodiments, reduced, decreased, or low amounts or levels of expression of one or more genes that are positively correlated to a risk of toxicity in a sample obtained from a subject are predictive of and/or associated with a low or reduced risk of toxicity, e.g., neurotoxicity or severe neurotoxicity. In some embodiments, elevated, increased, or high amounts or levels of expression of one or more genes that are positively correlated to a risk of toxicity in a sample obtained from a subject are predictive of and/or associated with a high, increased, or elevated risk of neurotoxicity.

In certain embodiments, an expression of a gene that is identified as positively correlated to or positively associated with a risk of toxicity has, is likely to have, or has been determined to have a gene expression with a positive correlation to the risk of the toxicity. In certain embodiments, the toxicity is neurotoxicity. In particular embodiments, the toxicity is severe neurotoxicity, e.g., a neurotoxicity of a grade 4 or 5 or of a prolonged grade 3. In particular embodiments, the positive correlation has, is likely to have, or has been determined to have a positive correlation to the risk of the toxicity with a correlation coefficient (R) of at least 0.25, at least 0.3, at least 0.4, at least 0.5, at least 0.55, at least 0.6, at least 0.65, at least 0.7, at least 0.75, at least 0.8, at least 0.85, at least 0.90, at least 0.95, at least 0.97, at least 0.98, at least 0.99, or about 1.0.

In some embodiments, an expression of a gene that is identified as positively correlated to or positively associated with a risk of toxicity has been identified based on data from a study, e.g., a clinical study. In some embodiments, the expression of the gene has been positively correlated with an incidence of the toxicity, such as CRS or neurotoxicity. In certain embodiments, the toxicity is neurotoxicity. In some embodiments, the toxicity is severe neurotoxicity, e.g., a neurotoxicity of a grade 4 or 5 or of a prolonged grade 3 or greater. In some embodiments, the positive correlation has, is likely to have, or has been determined to have a positive correlation to the incidence of the toxicity with a correlation coefficient (R) of at least 0.25, at least 0.3, at least 0.4, at least 0.5, at least 0.55, at least 0.6, at least 0.65, at least 0.7, at least 0.75, at least 0.8, at least 0.85, at least 0.90, at least 0.95, at least 0.97, at least 0.98, at least 0.99, or about 1.0.

In some embodiments, an expression of a gene that is identified as positively correlated to or positively associated with a risk of toxicity includes the expression of one or more of ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCN1P1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415. In certain embodiments, an expression of a gene that is identified as positively correlated to or positively associated with a risk of toxicity includes the expression of one or more of ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28. In certain embodiments, an expression of a gene that is identified as positively correlated to or positively associated with a risk of toxicity includes the expression of one or more of PCDHGA12, PCDHGB6, PCDHGB5, PCDHGA9, PINLYP, ASAP2, TTC28, PTCH1, and FMNL1. The full name, alternative symbols, and polynucleotide and polypeptide sequences of the gene products of genes with expression negatively correlated to toxicity, e.g., neurotoxicity or severe neurotoxicity, are shown in Table 2.

TABLE 2
Genes with expression positive correlated to toxicity

| | | Alternative Names | | | |
|---|---|---|---|---|---|
| Gene Symbol | Full Name | and Symbols | Uniprot ID | mRNA | Protein |
| ASAP2 | ArfGAP with SH3 | Development and | O43150 | SEQ ID | SEQ ID |
| | domain, ankyrin | differentiation- | | NO: 30 | NO: 78 |
| | repeat and PH | enhancing factor 2; | | | |
| | domain 2 | Paxillin-associated | | | |
| | | protein with ARF | | | |
| | | GAP activity 3; | | | |
| | | PAP; PAG3; | | | |
| | | AMAP2; DDEF2; | | | |
| | | SHAG1; CENTB3; | | | |
| | | Pap-alpha | | | |
| FMNL2 | formin like 2 | FHOD2 | Q96PY5 | SEQ ID | SEQ ID |
| | | | | NO: 31 | NO: 79 |
| GPR176 | G protein-coupled | HB-954 | Q14439 | SEQ ID | SEQ ID |
| | receptor 176 | | | NOS: 32-34 | NOS: 80-82 |
| MDFI | MyoD family | I-MF; I-mfa | Q99750 | SEQ ID | SEQ ID |
| | inhibitor | | | NOS: 35-36 | NOS: 83-84 |
| PCDHGA12 | protocadherin | FIB3; CDH21; | O60330 | SEQ ID | SEQ ID |
| | gamma subfamily | PCDH-GAMMA- | | NOS: 37-38 | NOS: 85-86 |
| | A, 12 | A12 | | | |
| PCDHGA6 | protocadherin | PCDH-GAMMA-A6 | Q9Y5G7 | SEQ ID | SEQ ID |
| | gamma subfamily | | | NOS: 39-40 | NOS: 87-88 |
| | A, 6 | | | | |
| PCDHGB6 | protocadherin | PCDH-GAMMA-B6 | Q9Y5G7 | SEQ ID | SEQ ID |
| | gamma subfamily | | | NOS: 41-42 | NOS: 89-90 |
| | B, 6 | | | | |
| PINLYP | phospholipase A2 | 2310033E01Rik | A6NC86 | SEQ ID | SEQ ID |
| | inhibitor and | | | NOS: 43-44 | NOS: 91-92 |
| | LY6/PLAUR | | | | |
| | domain containing | | | | |
| PTCH1 | Protein patched | PTC; BCNS; HPE7; | Q13635 | SEQ ID | SEQ ID |
| | homolog 1 | PTC1; PTCH; | | NOS: 45-47 | NOS: 93-95 |
| | | NBCCS; PTCH11 | | | |
| TTC28 | tetratricopeptide | TPR repeat- | Q96AY4 | SEQ ID | SEQ ID |
| | repeat domain 28 | containing big gene | | NO: 48 | NO: 96 |
| | | cloned at Keio; | | | |
| | | TPRBK; | | | |
| | | KIAA1043; | | | |
| ATP9A | ATPase | ATPIIA | Q2NLD0 | SEQ ID | SEQ ID |
| | phospholipid | | | NO: 100 | NO: 123 |
| | transporting 9A | | | | |
| HMX3 | H6 family | Homeobox protein | A6NHT5 | SEQ ID | SEQ ID |
| | homeobox 3 | H6 family member | | NO: 101 | NO: 124 |
| | | 3; NKX5.1; Nkx5-1; | | | |
| | | NKX-5.1 | | | |
| DPYSL3 | dihydropyrimidinase | DRP3; ULIP; | Q14195 | SEQ ID | SEQ ID |
| | like 3 | CRMP4; DRP-3; | | NOS: 102- | NOS: 125- |
| | | LCRMP; CRMP-4; | | 103 | 126 |
| | | ULIP-1 | | | |
| ZNF415 | zinc finger protein | ZfLp | Q09FC8 | SEQ ID | SEQ ID |
| | 415 | | | NOS: 104- | NOS: 127- |
| | | | | 112 | 135 |
| IRX5 | iroquois homeobox | Iroquois-class | P78411 | SEQ ID | SEQ ID |
| | 5 | homeodomain | | NOS: 113- | NOS: 136- |
| | | protein IRX-5; | | 114 | 137 |
| | | HMMS; IRXB2; | | | |
| | | IRX-2a | | | |
| TMPRSS15 | transmembrane | Enteropeptidase; | P98073 | SEQ ID | SEQ ID |
| | protease, serine 15 | Enterokinase; Serine | | NO: 115 | NO: 138 |
| | | protease 7 | | | |
| | | ENTK; PRSS7 | | | |
| IL3RA | interleukin 3 | IL3R; CD123; | P26951 | SEQ ID | SEQ ID |
| | receptor subunit | IL3RX; IL3RY; | | NOS: 116- | NOS: 139- |
| | alpha | IL3RAY; hIL-3Ra | | 117 | 140 |
| IGF2BP1 | insulin like growth | CRD-BP; CRDBP; | Q9NZI8 | SEQ ID | SEQ ID |
| | factor 2 mRNA | IMP-1; IMP1; | | NOS: 118- | NOS: 141- |
| | binding protein 1 | VICKZ1; ZBP1 | | 119 | 142 |
| PCDHGB5 | protocadherin | PCDH-GAMMA-B5 | Q9NZI8 | SEQ ID | SEQ ID |
| | gamma subfamily B, | | | NOS: 143- | NOS: 145- |
| | 5 | | | 144 | 146 |
| ATP8B1 | Phospholipid- | | O43520 | | |
| | transporting ATPase | | | | |
| | IC | | | | |
| CCNA1 | Cyclin-A1 | | P78396 | | |
| CDHR3 | Cadherin-related | | Q6ZTQ4 | | |
| | family member 3 | | | | |
| CECR2 | Cat eye syndrome | | Q9BXF3 | | |
| | critical region | | | | |
| | protein 2 | | | | |
| CELF4 | CUGBP Elav-like | | Q9BZC1 | | |
| | family member 4 | | | | |
| DLX1 | Homeobox protein | | P56177 | | |
| | DLX-1 | | | | |
| EHD4 | EH domain- | | Q9H223 | | |
| | containing protein 4 | | | | |
| GGA2 | ADP-ribosylation | | Q9UJY4 | | |
| | factor-binding | | | | |
| | protein GGA2 | | | | |
| HHIPL1 | HHIP-like protein 1 | | Q96JK4 | | |
| HOXA7 | Homeobox protein | | P31268 | | |
| | Hox-A7 | | | | |
| IRX3 | Iroquois-class | | P78415 | | |
| | homeodomain | | | | |
| | protein IRX-3 | | | | |
| KCNIP1 | Kv channel- | | Q9NZI2 | | |
| | interacting protein 1 | | | | |
| KIAA1644 | Uncharacterized | | Q3SXP7 | | |
| | protein KIAA1644 | | | | |
| LINC00092 | long intergenic non- | | — | | |
| | protein coding RNA | | | | |
| | 92 | | | | |
| LINC01483 | long intergenic non- | | — | | |
| | protein coding RNA | | | | |
| | 1483 | | | | |
| MIB1 | E3 ubiquitin-protein | | Q86YT6 | | |
| | ligase MIB1 | | | | |
| MMP14 | Matrix | | P50281 | | |
| | metalloproteinase- | | | | |
| | 14 | | | | |
| NOM1 | Nucleolar MIF4G | | Q5C9Z4 | | |
| | domain-containing | | | | |
| | protein 1 | | | | |
| OTOA | Otoancorin | | Q7RTW8 | | |
| PCDHGA4 | Protocadherin | | Q9Y5G9 | | |
| | gamma-A4 | | | | |
| PCDHGB1 | Protocadherin | | Q9Y5G3 | | |
| | gamma-B1 | | | | |
| PPM1E | Protein phosphatase | | Q8WY54 | | |
| | 1E | | | | |
| PRKD1 | Serine/threonine- | | Q15139 | | |
| | protein kinase D1 | | | | |
| PROKR2 | Prokineticin | | Q8NFJ6 | | |
| | receptor 2 | | | | |
| PRSS12 | Neurotrypsin | | P56730 | | |
| PRTG | Protogenin | | Q2VWP7 | | |
| RFX8 | DNA-binding | | Q6ZV50 | | |
| | protein RFX8 | | | | |
| RP11-146B14.1 | | | | | |
| RP11-3P17.5 | | | | | |
| RP11-41O4.1 | | | | | |
| RP11-713N11.4 | | | | | |
| RP4-568B10.1 | | | | | |
| SERF1A | Small EDRK-rich | | O75920 | | |
| | factor 1 | | | | |
| SEZ6L | Seizure 6-like | | Q9BYH1 | | |
| | protein | | | | |
| SMURF1 | E3 ubiquitin-protein | | Q9HCE7 | | |
| | ligase SMURF1 | | | | |
| TBC1D30 | TBC1 domain | | Q9Y2I9 | | |
| | family member 30 | | | | |
| TCF12 | Transcription factor | | Q99081 | | |
| | 12 | | | | |
| TCP11 | T-complex protein | | Q8WWU5 | | |
| | 11 homolog | | | | |
| TM9SF3 | Transmembrane 9 | | Q9HD45 | | |
| | superfamily member | | | | |
| | 3 | | | | |
| TMSB15A | Thymosin beta-15A | | P0CG34 | | |
| TNKS1BP1 | 182 kDa tankyrase- | | Q9C0C2 | | |
| | 1-binding protein | | | | |
| TREM2 | Triggering receptor | | Q9NZC2 | | |
| | expressed on | | | | |
| | myeloid cells 2 | | | | |
| PCDHGA9 | Protocadherin | PCDH-gamma-A9 | Q9Y5G4 | | |
| | gamma-A9; | | | | |
| FMNL1 | Formin-like protein | CLL-associated | O95466 | | |
| | 1 | antigen KW-13; | | | |
| | | Leukocyte formin; | | | |
| | | C17orf1, C17orf1B, | | | |
| | | FMNL, FRL1 | | | |

In some embodiments, provided herein are panels, profiles, and/or arrays for use in the measurement, assessment, and/or determination of one or more gene products in a sample e.g. from a subject prior to receiving an immunotherapy, e.g. cell therapy, to assess risk, probability, and/or likelihood of toxicity following administration of and/or associated with a therapy. In certain embodiments, the panels, profiles, and/or arrays are suitable for use to measure, assess, detect, and/or quantify the level and/or amount of one or more gene products in a sample, e.g., a BMA sample or a serum sample, such as from a subject prior to receiving an immunotherapy, e.g. cell therapy. In certain embodiments, the gene products are proteins and/or polypeptides. In some embodiments, the gene products are polynucleotides, e.g., mRNA or cDNA derived from mRNA. In particular embodiments, the panels, profiles, and/or arrays include the measurements, assessments, and/or quantifications of at least one, two, three, four, five, six, seven, eight, nine, ten, more than ten, or more than twenty gene products. In some embodiments, the gene products include one or more of the genes listed in Table 1, Table 2, Table 3, Table E2A and/or Table E2B.

In particular embodiments, the panels, profiles, and/or arrays include the measurements, assessments and/or quantifications of gene products from one or more genes listed in Table 1 and/or Table E2A and/or one or more genes listed in Table 2 and/or Table E2B. In some embodiments, the panels, profiles, and/or arrays include the measurements, assessments and/or quantifications of gene products from one or more of ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, and/or one or more of ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415.

In certain embodiments, the panels, profiles, and/or arrays include the measurements, assessments and/or quantifications of gene products from one or more of ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, and WNT9A and/or one or more of ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28.

In particular embodiments, the panels, profiles, and/or arrays include the measurements, assessments and/or quantifications of gene products from one or more of CCL17, CA6, JCHAIN, PTP4A3, IFITM1, CRLF2, ENAM, GBP5, ABCA9, SV2C, SLC37A3, IL15, IL2RA, and SEMA6A and/or one or more of PCDHGA12, PCDHGB6, PCDHGB5, PCDHGA9, PINLYP, ASAP2, TTC28, PTCH1, and FMNL1.

In certain embodiments, the panels, profiles, and/or arrays include the measurements, assessments and/or quantifications of gene products from one or more of IGJ (JCHAIN), MUC4, CA6, WNT9A, ADGRF1 and CCL17 and/or one or more of PINLYP, ASAP2, FMNL2, PTCH1, TTC28, PCDHGA6, PCDHGB6 and PCDHGA12. In particular embodiments, the panels, profiles, and/or arrays include the measurements, assessments and/or quantifications of gene products from one or more of IGJ (JCHAIN), MUC4, CA6, WNT9A, ADGRF1 and CCL17 and/or one or more of PINLYP and PCDHGA12.

In certain embodiments, the sample is obtained, collected, or taken from the subject prior to treatment with the therapy, e.g., an immunotherapy and/or a cell therapy. In particular embodiments, the sample is obtained, collected, and/or taken from the subject prior to a treatment with a cell therapy. In particular embodiments, the cell therapy is a T cell therapy. In certain embodiments, the T cell therapy contains one or more engineered cells. In particular embodiments, the therapeutic T cell therapy contains cells that express a recombinant receptor, e.g., a CAR. In some embodiments, the sample does not contain any engineered cells, cells expressing a recombinant receptor, or cells expressing a CAR.

In certain embodiments, one or more gene products are measured in a biological sample. In particular embodiments, the sample is or contains bone marrow. In some embodiments, the sample is or contains bone marrow aspirates. In some embodiments, the bone marrow sample contains, or is suspected of containing, at least one diseased cell or cancer cell. In particular embodiments, the diseased cell or cancer cell is a B cell. In particular embodiments, the bone marrow sample contains one or more gene products of one or more genes listed in Table 1, Table 2, Table E2A, Table E2B, or table E4. In certain embodiments, the gene product is a polynucleotide and/or polypeptide. In some embodiments, the gene product is mRNA.

In certain embodiments, the biological sample is or is derived or taken from bone marrow, e.g., bone marrow aspirate. In particular embodiments, the one or more gene products are mRNA or proteins selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, and WNT9A or a portion or fragment thereof and/or are selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or a portion or fragment thereof.

In some embodiments, the sample is a blood sample. In certain embodiments, the sample is a serum sample. In some embodiments, the sample is a peripheral blood sample. In some embodiments, the blood sample contains, or is suspected of containing, at least one diseased cell or cancer cell. In particular embodiments, the diseased cell or cancer cell is a B cell. In some embodiments, the blood or serum sample contains one or more gene products of one or more genes listed in Table 1, Table 2, Table E2A, Table E2B, or table E4. In certain embodiments, the gene product is a polypeptide.

In certain embodiments, one or more gene products are measured, assessed, quantified or detected in a biological sample that is taken or is derived from blood, e.g., a plasma or serum sample. In certain embodiments, one or more proteins are measured. In particular embodiments, the one or more proteins are whole portions, and/or variations of, versions of, isoforms of, or fragments of proteins selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or a portion or fragment thereof and/or are selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or a portion or fragment thereof. In certain embodiments, the one or more proteins measured in the biological sample taken are soluble, lack a transmembrane domain, and/or are cleaved at the cell surface. In certain embodiments, the one or more proteins are expressed on vascular endothelial cells and/or are associated with endothelial cell activation, vascular permeability and/or angiogenesis. In some embodiments, increased levels of the one or more proteins is associated with an increased risk of developing a toxicity or a severe form thereof, such as grade 3, grade 4, or higher neurotoxicity. In particular embodiments, the one or more proteins are whole portions, and/or variations of, versions of, isoforms of, or fragments of proteins selected from CCL17, ENG, SELE, ICAM3, and IL6R.

In some aspects, also provided herein are methods of measuring, assessing, determining, and/or quantifying the expression of one or a more gene products from a sample from a subject collected after the subject has received or been administered the immunotherapy, such as cell therapy, e.g. CAR-T cells. As shown herein, expression of certain gene products obtained from a sample from a subject subsequent to administration of the immunotherapy, such as cell therapy (e.g. CAR-T cells) is associated with and/or correlates with a risk of developing toxicity. In some aspects, the sample, e.g. serum or plasma sample, is obtained or collected from the subject within or about within 1 day, 2 days, 3 days, 4 days, 5 days, 6 days, 7 days, 8 days, 10 days, 12 days or 14 days after initiation of administration of the immunotherapy. In some aspects, the sample, e.g. serum or plasma sample, is obtained or collected from the subject no more than 4 days, such as no more than 3 days, no more than 2 days or no more than 1 day, after initiation of administration of the immunotherapy and/or before the subject exhibits a sign or symptom of the toxicity and/or before the subjects develops a sustained fever. In some embodiments, a gene product that is assessed in a sample from a subject collected after the subject has received or been administered the immunotherapy is selected from Endoglin, ICAM-3, CCL27, FAS, 1-309, NSE, P-Selectin, Resistin, S100β, Thrombomodulin and/or vWF, or a portion or fragment thereof. In some embodiments, such one or more gene products can be employed in provided methods to assess or monitor a risk of developing a toxicity, e.g. grade 3 or higher such as grade 4 or 5 neurotoxicity, subsequent to receiving the immunotherapy, such as cell therapy.

C. Measuring Gene Expression or Gene Products

In certain embodiments, the methods provided herein include one or more steps of assessing, measuring, determining, and/or quantifying the expression of one or more genes in a sample. In some embodiments, the expression of a gene, e.g., a gene with an expression that positively or negatively correlates with a risk for toxicity, is or includes assessing, measuring, determining, and/or quantifying a level, amount, or concentration of a gene product in the sample. In some embodiments, gene expression is or includes a process by which information of the gene is used in the synthesis of a gene product. Thus, in some embodiments, a gene product is any biomolecule that is assembled, generated, and/or synthesized with information encoded by a gene, and may include polynucleotides and/or polypeptides. In particular embodiments, assessing, measuring, and/or determining gene expression is or includes determining or measuring the level, amount, or concentration of the gene product. In certain embodiments, the level, amount, or concentration of the gene product may be transformed (e.g., normalized) or directly analyzed (e.g., raw). In some embodiments, the gene product is a protein that is encoded by the gene. In certain embodiments, the gene product is a polynucleotide, e.g., an mRNA or a protein, that is encoded by the gene.

In some embodiments, the gene product is a polynucleotide that is expressed by and/or encoded by the gene. In certain embodiments, the polynucleotide is an RNA. In some embodiments, the gene product is a messenger RNA (mRNA), a transfer RNA (tRNA), a ribosomal RNA, a small nuclear RNA, a small nucleolar RNA, an antisense RNA, long non-coding RNA, a microRNA, a Piwi-interacting RNA, a small interfering RNA, and/or a short hairpin RNA. In particular embodiments, the gene product is an mRNA.

In particular embodiments, the amount or level of a polynucleotide in a sample may be assessed, measured, determined, and/or quantified by any suitable means known in the art. For example, in some embodiments, the amount or level of a polynucleotide gene product can be assessed, measured, determined, and/or quantified by polymerase chain reaction (PCR), including reverse transcriptase (rt) PCR, droplet digital PCR, real-time and quantitative PCR (qPCR) methods (including, e.g., TAQMAN®, molecular beacon, LIGHTUP™, SCORPION™ SIMPLEPROBES®; see, e.g., U.S. Pat. Nos.5,538,848; 5,925,517; 6,174,670; 6,329,144; 6,326,145 and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art, such as discussed in Shendure et al., Nat. Rev. Genet. 5:335-44 (2004) or Nowrousian, Euk. Cell 9(9): 1300-1310 (2010), including such specific platforms as HELICOS®, ROCHE® 454, ILLUMINA®/SOLEXA®, ABI SOLiD®, and POLONATOR® sequencing. In particular embodiments, the levels of nucleic acid gene products are measured by quantitative PCR (qPCR) methods, such qRT-PCR. In some embodiments, the qRT-PCR uses three nucleic acid sets for each gene, where the three nucleic acids comprise a primer pair together with a probe that binds between the regions of a target nucleic acid where the primers bind—known commercially as a TAQMAN® assay.

In particular embodiments, assessing, measuring, determining, and/or quantifying amount or level of an RNA gene product includes a step of generating, polymerizing, and/or deriving a cDNA polynucleotide and/or a cDNA oligonucleotide from the RNA gene product. In certain embodiments, the RNA gene product is assessed, measured, determined, and/or quantified by directly assessing, measuring, determining, and/or quantifying a cDNA polynucleotide and/or a cDNA oligonucleotide that is derived from the RNA gene product.

In some embodiments, one or more oligonucleotide primers is contacted to an RNA gene product and/or a cDNA polynucleotide or oligonucleotide derived from the RNA gene product, to assess, measure, determine, and/or quantify the level, amount, or concentration of the RNA gene product. In some embodiments, provided herein are oligonucleotide primers that are suitable for assessing, measuring, detecting, and/or quantifying the level, amount, or concentration of an RNA gene product (or a cDNA derived therefrom). In certain embodiments, the oligonucleotide primers hybridize, and/or are capable of hybridizing to an RNA gene product and/or a cDNA derived therefrom. In certain embodiments, the oligonucleotide hybridizes and/or is capable of hybridizing to an RNA gene product, or cDNA derived therefrom, that is expressed and/or encoded by a gene listed in Table 1 or Table 2. In particular embodiments, the oligonucleotide primer hybridizes and/or is capable of hybridizing to an RNA gene product, a portion or partial transcript thereof, or a cDNA derived therefrom, that is at least 75%, 80%, 85%, 90%, 95%, 97%, 98%, 99%, about 100%, or 100% identical to a nucleotide sequence set forth in SEQ ID NOS: 1-48. In some embodiments, sets of oligonucleotide primers may be prepared for any of RNA gene products that are encoded by any of the genes listed in Table 1 or Table 2, or described anywhere in the application. In some embodiments, the oligonucleotide primers can readily be designed using ordinary skill in the art of molecular biology to arrive at primers that are specific for a given RNA gene product. In some embodiments, the oligonucleotide primer has a length of about 10-100 nucleotides, e.g., about 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100 nucleotides, or more) and a sequence (i.e., the particular portion of a sequence set for in SEQ ID NOS: 1-48, or a portion complementary thereto) of the primers can readily be adjusted to achieve a desired melting temperature (“Tm”; e.g., about 45-72° C., e.g., about 45, 50, 55, 60, 65, 70, 72° C. or more) and specificity. One of skill in the art will readily account for factors such as secondary structures, primer dimers, salt concentrations, nucleic acid concentrations, et cetera. Oligonucleotide primers provided herein may consist of (or consist essentially of) naturally occurring deoxribonucleotides or, optionally, may include modifications such as non-natural nucleotides, artificial backbones (such as PNAs), and detectable labels, such as florescent labels. In particular embodiments, a florescent label is attached, e.g., covalently attached, to the oligonucleotide primer.

In particular embodiments, the expression of two or more of the genes are measured or assessed simultaneously. In certain embodiments, a multiplex PCR, e.g., a multiplex rt-PCR assessing or a multiplex quantitative PCR (qPCR) for, measuring, determining, and/or quantifying the level, amount, or concentration of two or more gene products. In some embodiments, microarrays (e.g., AFFYMETRIX®, AGILENT® and ILLUMINA®-style arrays) are used for assessing, measuring, determining, and/or quantifying the level, amount, or concentration of two or more gene products. In some embodiments, microarrays are used for assessing, measuring, determining, and/or quantifying the level, amount, or concentration of a cDNA polynucleotide that is derived from an RNA gene product.

In some embodiments, the expression of one or more gene products, e.g., polynucleotide gene products, is determined by sequencing the gene product and/or by sequencing a cDNA polynucleotide that is derived from the from the gene product. In some embodiments, the sequencing is performed by a non-Sanger sequencing method and/or a next generation sequencing (NGS) technique. Examples of Next Generation Sequencing techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

In some embodiments, the NGS technique is RNA sequencing (RNA-Seq). In particular embodiments, the expression of the one or more polynucleotide gene products is measured, determined, and/or quantified by RNA-Seq. RNA-Seq, also called whole transcriptome shotgun sequencing determines the presence and quantity of RNA in a sample. RNA sequencing methods have been adapted for the most common DNA sequencing platforms [HiSeq systems (IIlumina), 454 Genome Sequencer FLX System (Roche), Applied Biosystems SOLiD (Life Technologies), IonTorrent (Life Technologies)]. These platforms require initial reverse transcription of RNA into cDNA. Conversely, the single molecule sequencer HeliScope (Helicos BioSciences) is able to use RNA as a template for sequencing. A proof of principle for direct RNA sequencing on the PacBio RS platform has also been demonstrated (Pacific Bioscience). In some embodiments, the one or more RNA gene products are assessed, measured, determined, and/or quantified by RNA-seq.

In some embodiments, the RNA-seq is a tag-based RNA-seq. In tag-based methods, each transcript is represented by a unique tag. Initially, tag-based approaches were developed as a sequence-based method to measure transcript abundance and identify differentially expressed genes, assuming that the number of tags (counts) directly corresponds to the abundance of the mRNA molecules. The reduced complexity of the sample, obtained by sequencing a defined region, was essential to make the Sanger-based methods affordable. When NGS technology became available, the high number of reads that could be generated facilitated differential gene expression analysis. A transcript length bias in the quantification of gene expression levels, such as observed for shotgun methods, is not encountered in tag-based methods. All tag-based methods are by definition strand specific. In particular embodiments, the one or more RNA gene products are assessed, measured, determined, and/or quantified by tag-based RNA-seq.

In some embodiments, the RNA-seq is a shotgun RNA-seq. Numerous protocols have been described for shotgun RNA-seq, but they have many steps in common: fragmentation (which can occur at RNA level or cDNA level, conversion of the RNA into cDNA (performed by oligo dT or random primers), second-strand synthesis, ligation of adapter sequences at the 3′ and 5′ ends (at RNA or DNA level) and final amplification. In some embodiments, RNA-seq can focus only on polyadenylated RNA molecules (mainly mRNAs but also some lncRNA, snoRNA, pseudogenes and histones) if poly(A)⁺ RNAs are selected prior to fragmentation, or may also include non-polyadenylated RNAs if no selection is performed. In the latter case, ribosomal RNA (more than 80% of the total RNA pool) needs to be depleted prior to fragmentation. It is, therefore, clear that differences in capturing of the mRNA part of the transcriptome lead to a partial overlap in the type of detected transcripts. Moreover, different protocols may affect the abundance and the distribution of the sequenced reads. This makes it difficult to compare results from experiments with different library preparation protocols.

In some embodiments, RNA from each sample, such as each BMA sample, is obtained, fragmented and used to generate complementary DNA (cDNA) samples, such as cDNA libraries for sequencing Reads may be processed and aligned to the human genome and the expected number of mappings per gene/isoform are estimated and used to determine read counts. In some embodiments, read counts are normalized by the length of the genes/isoforms and number of reads in a library to yield FPKM normalized, e.g., by length of the genes/isoforms and number of reads in the library, to yield fragments per kilobase of exon per million mapped reads (FPKM) according to the gene length and total mapped reads. In some aspects, between—sample normalization is achieved by normalization, such as 75th quantile normalization, where each sample is scaled by the median of 75th quantiles from all samples, e.g., to yield quantile-normalized FPKM (FPKQ) values. The FPKQ values may be log-transformed (log 2).

In some embodiments, techniques and methods involving nucleotide aptamers are used to measure, assess, quantify, and/or determine the level, amount, or concentration of a polynucleotide gene product. Suitable nucleotide aptamers are known, and include those described in Cox and Ellington, Bioorganic & Medicinal Chemistry. (2001) 9 (10): 2525-2531; Cox et al., Combinatorial Chemistry & High Throughput Screening. (2002) 5 (4): 289-29; Cox et al., Nucleic Acids Research. (2002) 30(20): e108.

In some embodiments, RNA-seq is performed to sequence total RNA, e.g., the total RNA of a sample. In particular embodiments, the RNA-seq is performed to sequence one or more of mRNA, tRNA, ribosomal RNA, small nuclear RNA, small nucleolar RNA, antisense RNA, long non-coding RNA, microRNA, Piwi-interacting RNA, small interfering RNA, and/or a short hairpin RNA. In certain embodiments, the RNA-seq is performed to sequence only mRNA, tRNA, ribosomal RNA, small nuclear RNA, small nucleolar RNA, antisense RNA, long non-coding RNA, microRNA, Piwi-interacting RNA, small interfering RNA, and/or a short hairpin RNA. In particular embodiments, the RNA-seq is performed to sequence mRNA gene products.

In some embodiments, the gene product is or includes a protein, i.e., a polypeptide, that is encoded by and/or expressed by the gene. In particular embodiments, the gene product encodes a protein that is localized and/or exposed on the surface of a cell. In some embodiments, the protein is a soluble protein. In certain embodiments, the protein is secreted by a cell.

In particular embodiments, the gene expression is the amount, level, and/or concentration of a protein that is encoded by the gene. In certain embodiments, one or more protein gene products are measured by any suitable means known in the art. Suitable methods for assessing, measuring, determining, and/or quantifying the level, amount, or concentration or more or more protein gene products include, but are not limited to detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include, but are not limited to, quantitative immunocytochemisty or immunohistochemisty, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), enzyme immunoassay (EIA), RIA (radioimmunoassay), and SPR (surface plasmon resonance).

In some embodiments, the gene expression product is a protein. In particular embodiments, the gene expression product is a fraction, portion, variant, version, and/or isoform of a protein, e.g., a protein encoded by a gene listed Table 1, Table 2, Table 3, Table E2A, and/or Table E2B. In particular embodiments, the fraction, portion, variant, version, and/or isoform of the protein is soluble. In some embodiments, the fraction, portion, variant, version, and/or isoform of the protein lacks a transmembrane domain. In certain embodiments, the fraction, portion, variant, version, and/or isoform of a protein is not expressed on or within the surface of a cell. In some embodiments, the fraction, portion, variant, version, and/or isoform of the protein has been cleaved from the surface of a cell.

The practice of the methods, kits, and compositions provided herein may also employ conventional biology methods, software and systems. For example, means for measuring the expression level of transcripts or partial transcripts of genes, e.g., genes listed in Table 1, Table 2, Table 3, Table E2A, and/or Table E2B; means for correlating the expression level with a classification of risk, probability, and/or likelihood of toxicity following administration of and/or associated with the therapy; and means for outputting the risk, probability, and/or likelihood may employ conventional biology methods, software and systems as described herein or as otherwise known. Computer software products for use with the provided methods, compositions, and kits, typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

In some embodiments, the methods provided herein include a step of assessing one or more genes in a sample by assessing, measuring, determining, and/or quantifying the amount of the corresponding one or more gene products in the sample. In certain embodiments, the expression of one or more genes in a sample that negatively correlates and/or is negatively associated with a toxicity, e.g., neurotoxicity or severe neurotoxicity, is measured by determining the amount or level of one of more corresponding gene products in the sample. In certain embodiments, the gene expression in a sample is the level, amount, or concentration of a gene product that is encoded by the gene.

In particular embodiments, the expression of one or more genes that negatively correlate and/or are negatively associated with a toxicity, e.g., neurotoxicity or severe neurotoxicity, are measured in a sample. In some embodiments, the expression of one or more genes that negatively correlate and/or are negatively associated with a toxicity is assessed, measured, determined, and/or quantified by determining the amount or level of a product encoded, produced, and/or expressed by the gene. In some embodiments, the one or more gene products are encoded, produced, and/or expressed by one or more genes listed in Table 1 and/or Table E2A. In certain embodiments, the gene product is one of two or more isoforms that are encoded by a gene. In particular embodiments, the one or more gene products are products of one or more of ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A or a portion thereof. In some embodiments, the one or more gene product is a product of IGJ (JCHAIN), MUC4, CA6, WNT9A, ADGRF1 or CCL17, or a portion thereof. In various embodiments, the one or more gene products are products of one or more of CCL17, CA6, JCHAIN, PTP4A3, IFITM1, CRLF2, ENAM, GBP5, ABCA9, SV2C, SLC37A3, IL15, IL2RA, and SEMA6A or a portion thereof. In some embodiments, the one or more gene product is a product of IGJ (JCHAIN), MUC4, CA6, WNT9A, ADGRF1 or CCL17 or a portion thereof.

In particular embodiments, the expression of one or more genes that negatively correlate and/or are negatively associated with a toxicity are assessed, measured, determined, and/or quantified by determining the amount or level of an RNA product encoded, produced, and/or expressed by the one or more genes. In certain embodiments, the gene product is an mRNA. In certain embodiments, the one or more gene products are mRNA produced or encoded by one or more genes listed in Table 1 and/or Table E2A. In some embodiments, the one or more gene products are mRNA product encoded by one or more of ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A. In some embodiments, the one or more gene products are mRNA produced or encoded by one or more of IGJ (JCHAIN), MUC4, CA6, WNT9A, ADGRF1 or CCL17.

In some embodiments, the one or more products or gene products are one or more mRNA with at least 15, at least 20, at least 25, at least 50, at least 75, at least 100, at least 150, at least 200, at least 250, at least 300, at least 350, at least 400, at least 450, at least 500, or at least 1,000 contiguous nucleotides of one or more sequences that are at least 75%, at least 80%, at least 85%, at least 90%, at least 95%, at least 97%, at least 98%, at least 99%, or at least 99.5% identical to one or more sequences set forth in SEQ ID NOS: 1-29 or 97-99. In particular embodiments, the one or more products or gene products are one or more mRNA with at least 15, at least 20, at least 25, at least 50, at least 75, at least 100, at least 150, at least 200, at least 250, at least 300, at least 350, at least 400, at least 450, at least 500, or at least 1,000 contiguous nucleotides of one or more sequences set forth in SEQ ID NOS: 1-29 or 97-99. In particular embodiments, the one or more products or gene products are mRNA that are at least 75%, at least 80%, at least 85%, at least 90%, at least 95%, at least 97%, at least 98%, at least 99%, or at least 99.5% identical to one or more sequences set forth in SEQ ID NOS: 1-29 or 97-99. In some embodiments, the gene products are one or more mRNA or a portion or a partial transcript thereof of one or more sequences that are set forth in SEQ ID NOS: 1-29 or 97-99. In particular embodiments, the one or more gene products are mRNA of one or more sequences that are set forth in SEQ ID NOS: 1-29 or 97-99.

In particular embodiments, the expression of one or more genes that negatively correlate and/or are negatively associated with a toxicity are assessed, measured, determined, and/or quantified by determining the amount or level of a protein encoded by or expressed by the one or more genes. In some embodiments, the one or more gene products are proteins, or portions or variants thereof, that are encoded, produced, and/or expressed by one or more genes listed in Table 1 and/or Table E2A. In some embodiments, the one or more gene products are proteins encoded by one or more genes selected from ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A. In some embodiments, the one or more proteins are encoded by one or more genes selected from IGJ (JCHAIN), MUC4, CA6, WNT9A, ADGRF1 or CCL17.

In some embodiments, the one or more gene products are proteins encoded by one or more genes selected from CCL17, CA6, JCHAIN, PTP4A3, IFITM1, CRLF2, ENAM, GBP5, ABCA9, SV2C, SLC37A3, IL15, IL2RA, and SEMA6A. In some embodiments, the one or more proteins are encoded by one or more genes selected from PCDHGA12, PCDHGB6, PCDHGBS, PCDHGA9, PINLYP, ASAP2, TTC28, PTCH1, and FMNL1.

In some embodiments, the one or more products or gene products are proteins with at least 15, at least 20, at least 25, at least 50, at least 75, at least 100, at least 125, at least 150, at least 175, at least 200, at least 250, at least 300, at least 350, at least 400, at least 450, or at least 500 contiguous amino acids of one or more polypeptide sequences that are at least 75%, at least 80%, at least 85%, at least 90%, at least 95%, at least 97%, at least 98%, at least 99%, or at least 99.5% identical to a polypeptide sequence set forth in SEQ ID NOS: 49-77 and 120-122. In particular embodiments, the one or more products or gene products are proteins with at least 15, at least 20, at least 25, at least 50, at least 75, at least 100, at least 125, at least 150, at least 175, at least 200, at least 250, at least 300, at least 350, at least 400, at least 450, or at least 500 contiguous amino acids of a polypeptide sequence set forth in SEQ ID NOS: 49-77 and 120-122. In certain embodiments, the one or more products or gene products are proteins that are at least 75%, at least 80%, at least 85%, at least 90%, at least 95%, at least 97%, at least 98%, at least 99%, or at least 99.5% identical to a polypeptide sequence set forth in SEQ ID NOS: 49-77 and 120-122. In some embodiments, the one or more gene products are proteins with one or more polypeptide sequences set forth in SEQ ID NOS: 49-77 and 120-122.

In particular embodiments, the expression of one or more genes that positively correlate and/or are positively associated with a toxicity, e.g., neurotoxicity or severe neurotoxicity, are measured in a sample. In some embodiments, the expression of one or more gene that positively correlate and/or are positively associated with a toxicity are assessed, measured, determined, and/or quantified by determining the amount or level of a product encoded, produced, and/or expressed by the gene. In some embodiments, the one or more gene products are encoded, produced, and/or expressed by a gene listed in Table 2 or Table E2B. In certain embodiments, the gene product is one of two or more isoforms that are encoded by a gene. In particular embodiments, the one or more gene products are products of one or more ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28. In certain embodiments, the one or more gene products are products of one or more of PINLYP, ASAP2, FMNL2, PTCH1, TTC28, PCDHGA6, PCDHGB6 or PCDHGA12. In certain embodiments, the one or more gene products are a product of PINLYP or PCDHGA12.

In particular embodiments, the expression of one or more genes that positively correlate and/or are positively associated with a toxicity are assessed, measured, determined, and/or quantified by determining the amount or level of an RNA product encoded, produced, and/or expressed by the one or more genes. In certain embodiments, the one or more gene products are one or more of an mRNA or a portion or partial transcript thereof of one or more genes listed in Table 2 and/or Table E2B. In certain embodiments, the one or more genes product are mRNA or a portion or partial transcript thereof that are produced or encoded by one or more of ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28. In some embodiments, the one or more gene products are mRNA or a portion or partial transcript thereof that are produced or encoded by one or more of PINLYP, ASAP2, FMNL2, PTCH1, TTC28, PCDHGA6, PCDHGB6 or PCDHGA12. In certain embodiments, the one or more genes product are mRNA or a portion or partial transcript thereof that are produced or encoded by one or more of PINLYP or PCDHGA12.

In some embodiments, the one or more gene products are mRNA with at least 15, at least 20, at least 25, at least 50, at least 75, at least 100, at least 150, at least 200, at least 250, at least 300, at least 350, at least 400, at least 450, at least 500, or at least 1,000 contiguous nucleotides of a sequence that is at least 75%, at least 80%, at least 85%, at least 90%, at least 95%, at least 97%, at least 98%, at least 99%, or at least 99.5% identical to one or more sequences set forth in SEQ ID NOS: 30-48, 100-119, 143-144. In particular embodiments, the one or more gene products are mRNA with at least 15, at least 20, at least 25, at least 50, at least 75, at least 100, at least 150, at least 200, at least 250, at least 300, at least 350, at least 400, at least 450, at least 500, or at least 1,000 contiguous nucleotides of one or more sequences set forth in SEQ ID NOS: 30-48, 100-119, 143-144. In some embodiments, the one or more gene products are mRNA that is at least 75%, at least 80%, at least 85%, at least 90%, at least 95%, at least 97%, at least 98%, at least 99%, or at least 99.5% identical to a sequence set forth in SEQ ID NOS: 30-48, 100-119, 143-144. In certain embodiments, the one or more gene products are an mRNA or a portion or partial transcription of a sequence set forth in SEQ ID NOS: 30-48, 100-119, 143-144. In some embodiments, the one or more gene products are RNA with a sequence set forth in SEQ ID NOS: 30-48, 100-119, 143-144.

In particular embodiments, the expression of one or more genes that positively correlate and/or are positively associated with a toxicity are assessed, measured, determined, and/or quantified by determining the amount or level of a protein encoded by or expressed by the gene. In some embodiments, the gene product is a protein encoded, produced, and/or expressed by a gene listed in Table 2 and/or Table E2B. In some embodiments, the gene product is a protein encoded, produced, and/or expressed by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28. PINLYP, ASAP2, FMNL2, PTCH1, TTC28, PCDHGA6, PCDHGB6 or PCDHGA12. In certain embodiments, the one or more genes product are mRNA or a portion or partial transcript thereof that is produced or encoded by one or more of PINLYP or PCDHGA12.

In certain embodiments, the one or more gene products are one or more proteins with at least 15, at least 20, at least 25, at least 50, at least 75, at least 100, at least 125, at least 150, at least 175, at least 200, at least 250, at least 300, at least 350, at least 400, at least 450, or at least 500 contiguous amino acids of one or more polypeptide sequences that are at least 75%, at least 80%, at least 85%, at least 90%, at least 95%, at least 97%, at least 98%, at least 99%, or at least 99.5% identical to a polypeptide sequence set forth in SEQ ID NOS: 78-96, 123-142, or 145-146. In particular embodiments, the one or more gene products are one or more proteins with at least 15, at least 20, at least 25, at least 50, at least 75, at least 100, at least 125, at least 150, at least 175, at least 200, at least 250, at least 300, at least 350, at least 400, at least 450, or at least 500 contiguous amino acids of one or more polypeptide sequences set forth in SEQ ID NOS: 78-96, 123-142, or 145-146. In some embodiments, the one or more gene products are a protein that is at least 75%, at least 80%, at least 85%, at least 90%, at least 95%, at least 97%, at least 98%, at least 99%, or at least 99.5% identical to a polypeptide sequence set forth in SEQ ID NOS: 78-96, 123-142, or 145-146. In some embodiments, the one or more gene product are proteins or a portions of one or more polypeptide sequences set forth in SEQ ID NOS: 78-96, 123-142, or 145-146. In certain embodiments, the one or more gene products are one or more proteins with a polypeptide sequence set forth in SEQ ID NOS: 78-96, 123-142, or 145-146.

In certain embodiments, one or more gene products of gene that negatively correlate and/or are negatively associated with toxicity following administration of a therapy, e.g., an immunotherapy and/or a cell therapy, and one or more genes that are positively correlated and/or are positively associated with the therapy are measured in a sample. In certain embodiments, the gene products of one or more genes listed in Table 1 and/or Table E2A and one or more genes listed in Table 2 and/or Table E2A are measured in a sample. In some embodiments, one or more gene products of ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, and one or more gene products of ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 are measured in a sample.

In some embodiments, one or more gene products expressed by ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A and one or more gene products expressed by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28 are measured in a sample. In various embodiments, one or more gene products expressed by CCL17, CA6, JCHAIN, PTP4A3, IFITM1, CRLF2, ENAM, GBP5, ABCA9, SV2C, SLC37A3, IL15, IL2RA, and SEMA6A and one or more gene products expressed by PCDHGA12, PCDHGB6, PCDHGB5, PCDHGA9, PINLYP, ASAP2, TTC28, PTCH1, and FMNL1 are measured in a sample. In certain embodiments, one or more gene products expressed by IGJ (JCHAIN), MUC4, CA6, WNT9A, ADGRF1 or CCL17 and one or more gene products expressed by PINLYP, ASAP2, FMNL2, PTCH1, TTC28, PCDHGA6, PCDHGB6 or PCDHGA12 are measured in a sample. In particular embodiments, one or more gene products expressed by IGJ (JCHAIN), MUC4, CA6, WNT9A, ADGRF1 or CCL17 and one or more gene products expressed by PINLYP or PCDHGA12 are measured in a sample.

In some embodiments, measuring, assessing, determining, and/or quantifying one or more of the gene products in a sample is not predictive, and/or is not associated or correlated with toxicity, e.g., neurotoxicity, at the time at which the sample is collected from the subject. In some embodiments, the gene expression profile of any of the genes listed in Table 1, Table 2, Table 3, Table E2A, Table E2B, or Table E4, are not predictive, correlated with, and/or associated with toxicity when the sample is collected during or after the subject has received treatment with an immunotherapy, e.g., a cell therapy containing CAR-T cells.

1. Normalization to Control Values

In some embodiments, the assessment, determination, measurement, and/or quantification of a gene product, e.g., an RNA or protein gene product, of a sample is normalized to a control value. In certain embodiments, normalization to one or more control values may be performed to analyze, assess, or determine if an amount or level of the gene product indicates if the expression of the gene is elevated or decreased, and/or high or low. In particular embodiments, normalization to control values may be used to compare the gene expression of a gene a sample to the gene expression of a different sample.

In particular embodiments, the control value is a measurement, or a value of a measurement, of a different gene product. In some embodiments, the different gene product is a gene product of a housekeeping gene. In certain embodiments, the housekeeping gene is a constitutively active gene, e.g., a gene that is required for maintenance of basic cellular function. Examples of suitable housekeeping genes are known in the art, and include, but are not limited to, genes encoding ACTB (Beta-actin), B2M (Beta-2-microglobulin), GAPDH (Glyceraldehyde 3-phosphate dehydrogenase), RPLP0 (60S acidic ribosomal protein P0), GUSB (beta-glucuronidase), HMBS (Hydroxymethyl-bilane synthase), HPRT1 (Hypoxanthine phosphoribosyl-transferase 1), RPL13A (Ribosomal protein L13a), SDHA, succinate dehydrogenase complex subunit A), TBP (TATA box binding protein), TFRC (transferring receptor 1), and UBC (Ubiquitin C). In some embodiments, the control value is measured in the same sample as the gene product.

In certain embodiments, the gene product is compared and/or normalized to a control value that is a measurement, or a value of a measurement, of the gene product from the same gene. In some embodiments, the control value is a measurement, or a value of a measurement, that is obtained from one or more control samples. In certain embodiments, the gene product and the control value are measured in different samples. In some embodiments, the one or more control samples have an identical, a same, or a similar tissue composition and/or cellular composition as the sample. In some embodiments, the sample and control sample are different samples from the same, similar, and/or identical tissue from the same subject. In particular embodiments, the sample and the control sample different samples from the same tissue in different subjects. In particular embodiments, the sample and control sample are different samples from the same, similar, and/or identical tissue from different subjects. In certain embodiments, the control sample is obtained from a subject that does not have a condition and/or a cancer. In particular embodiments, the control sample is obtained from a subject that does not have ALL. In some embodiments, the control sample does not have and/or is not suspected of having one or more tumor cells. In particular embodiments, the control sample does not have and/or is not suspected of having one or more tumor cells. In some embodiments, the one or more samples are bone marrow samples and the one or more control samples are bone marrow samples. In particular embodiments, the one or more samples are blood samples, e.g., peripheral blood samples, and the one or more control samples are blood samples.

In certain embodiments, the assessment, determination, measurement, and/or quantification of a gene product, e.g., an RNA or protein gene product, of a sample is normalized to and/or compared to two or more control values. In some embodiments, the two or more control values include a control value that is a measurement, or a value of a measurement, that of the same gene product and a control value that is a measurement, or a value of a measurement, that of a different gene product.

In some embodiments, the control value has been previous determined. In certain embodiments, the one or more control values are measured or obtained in parallel with the assessment, measurement, determination, and/or quantification of the one or gene products in the sample.

In particular embodiments, the control value is an average or a median amount or level of expression of the one or more gene products obtained from a plurality of control samples. In some embodiments, the plurality of control samples is obtained from individual control subjects. In particular embodiments, the plurality of individual control subjects are subjects that do not have and/or are not suspected of having a condition or a disease. In some embodiments, the plurality of individual control subjects are subjects that do not have and/or are not suspected of having a cancer. In some embodiments, the plurality of individual control subjects are subjects that do not have and/or are not suspected of having ALL. In some embodiments, the plurality of individual control subjects are subjects that do not have and/or are not suspected of having a specific subtype of ALL. In particular embodiments, the subtype of ALL is the Philadelphia-like (Ph-like) subtype of ALL.

In certain embodiments, the plurality of individual control subjects is a plurality of subjects that have and/or are suspected of having a cancer. In some embodiments, the plurality of individual control subjects is or includes subjects that have and/or are suspected of having ALL. In some embodiments, the plurality of individual control subjects is or includes subjects that have and/or are suspected of having a specific subtype of ALL. In some embodiments, the plurality of individual control subjects is or includes subjects that have and/or are suspected of having ALL, but not a specific subtype of ALL. In particular embodiments, the subtype of ALL is the Philadelphia chromosome positive (Ph+) and/or Philadelphia-like (Ph-like) subtype of ALL. In some embodiments, the plurality of individual control subjects is or includes Ph+ and/or Ph-like subjects.

In some embodiments, the control value is obtained from a plurality of control samples. In certain embodiments, the plurality of control samples contains at least 2, at least 3, at least 5, at least 10, at least 15, at least 20, at least 25, at least 30, at least 40, at least 50, at least 100, at least 200, at least 300, at least 400, or at least 500 control samples.

In certain embodiments, an assessment, measurement, determination, or quantification of gene expression, e.g., the amount or level of one or more gene products, can be analyzed by any means in the art. In some embodiments, prior to an analysis, raw gene expression data, e.g., the value of the measured and/or quantified level, amount, or concentration of the gene product can be normalized or transformed, e.g., log-normalized, expressed as an expression ratio, percentile-ranked, and/or quantile-scaled. In some embodiments, gene expression data may further be modified by any nonparametric data scaling approach. In some embodiments, the transformation of the measurement or assessment of the expression of the one or more gene products occurs prior to any normalization to a control. In certain embodiments, the transformation of the measurement or assessment of the expression of the one or more gene products occurs after a normalization to a control value. In some embodiments, the transformation is a logarithmic transformation, a power transformation, or a logit transformation. In some embodiments, the logarithmic transformation is a common log (log₁₀(x)), a natural log (ln(x)), or a binary log (log₂(x)).

Expression patterns can be evaluated and classified by a variety of means, such as general linear model (GLM), ANOVA, regression (including logistic regression), support vector machines (SVM), linear discriminant analysis (LDA), principal component analysis (PCA), k-nearest neighbor (kNN), neural network (NN), nearest mean/centroid (NM), and Bayesian covariate predictor (BCP). A model, such as SVM, can be developed using any of the subsets and combinations of genes described herein based on the teachings of the invention. In more particular embodiments, an expression pattern is evaluated as the mean of log-normalized expression levels of the genes.

In some embodiments, a combination of one or more genes that positively correlate and one or more genes that negatively correlate are measured to determine a risk of a toxicity.

In certain embodiments, an expression profile and/or a gene expression profile is or is indicated by assessing, measuring, determining, and/or quantifying the expression of at least two genes. For example, in some embodiments, assessing or determining the gene expression profile of a sample may include assessing, measuring, determining, and/or quantifying of at least two genes that are associated with and/or correlated to a risk of developing a toxicity, e.g., a neurotoxicity. In certain embodiments, a gene expression profile is obtained by measuring, determining, and/or quantifying the expression of two or more genes, e.g., by measuring, determining, and/or quantifying the gene products of two or more genes, that are positively correlated with risk of developing a toxicity.

D. Molecular Subtypes of a Disease

In particular embodiments, provided herein are methods of treating a subject with a subtype, e.g., a molecular subtype, of a disease with a therapy, e.g., a cell therapy or an immunotherapy. In some embodiments, the subtype of the disease is associated with a low, reduced, and/or decreased risk, probability, and/or likelihood of toxicity following administration of and/or associated with the therapy. In certain embodiments, the methods include one or more steps of determining and/or identifying the subtype of the disease of the subject, and for example, by measuring or assessing the expression of one or more genes, e.g., a gene listed in Table 1 or table E2A. In certain embodiments, the methods include one or more steps of administering the therapy to a subject that has been determined to have a subtype of a disease that is associated with a reduced, decreased, or low probability of toxicity following administration of and/or associated with the therapy. In particular embodiments, the methods include one or more steps of administering an alternative therapy or a reduced dose of the therapy to a subject that had been determined not to have a subtype of the disease that is associated with a reduced, decreased, or low probability of toxicity following administration of and/or associated with the therapy.

In some embodiments, the therapy is administered to treat a disease, e.g., a proliferative disease and/or a cancer. The disease may be associated with and/or contain one or more sub-types of the disease. Particular embodiments contemplate that different disease subtypes may be associated with different risks, probabilities, likelihoods, of toxicity following administration of and/or associated with the therapy. In some embodiments, a subtype of a disease is associated with a least a portion of the symptoms of the disease, but may differ in one or more characteristics of the disease. In some embodiments, the disease is a proliferative disease and/or a cancer, and the sub-type of the proliferative disease or cancer includes or involves diseased cells of the same cell-type. In certain embodiments, the subtype of the proliferative disease or cancer is characterized by a chromosomal abnormality that is not present in other sub-types of the same disease. In some embodiments, the subtype of the cancer or proliferative disease is characterized by a particular positive, high, negative, and/or low expression of one or more genes, e.g., a gene signature. In some embodiments, the subtype of the proliferative disease is identified by measuring, assessing, determining, and/or quantifying the expression of one or more genes listed in Section I-B or in Tables 1, 2, 3, E2A, E2B, or E4. (e.g. a subset of these genes in the relevant Table which has a recited SEQ ID NO, and/or a Uniprot ID).

In certain embodiments, provided herein are methods of treatment for subjects with a subtype of a proliferative disease and/or a cancer. In certain embodiments, the subtype is a subtype of a B cell malignancy or hematological malignancy. In some embodiments, the subtype is a subtype of a myeloma, e.g., a multiple myeloma (MM), lymphoma or a leukemia, acute lymphoblastic leukemia (ALL), non-Hodgkin's lymphoma (NHL), chronic lymphocytic leukemia (CLL), diffuse large B-cell lymphoma (DLBCL), or acute myeloid leukemia (AML). In some embodiments, the subtype is a subtype of ALL.

In particular embodiments, provided herein are methods of treating a subject having a subtype of leukemia. In certain embodiments, the methods include one or more steps for treating a subject having a subtype of ALL. In some embodiments, the subtype of ALL is associated with a low, reduced, and/or decreased risk, probability, and/or likelihood of toxicity following administration of and/or associated with the therapy. In certain embodiments, the methods include one or more steps of administering the therapy to a subject that has been determined to have a subtype of ALL that is associated with a reduced, decreased, or low probability of toxicity following administration of and/or associated with the therapy. In particular embodiments, the methods include one or more steps of administering an alternative therapy or a reduced dose of the therapy to a subject that had been determined not to have the subtype of ALL that is associated with a reduced, decreased, or low probability of toxicity following administration of and/or associated with the therapy.

In particular embodiments, the subtype of ALL is the Philadelphia chromosome positive (Ph+) subtype of ALL. This subtype is characterized, in part, by poor outcomes with treatments of standard chemotherapy. The Philadelphia chromosome is present in 3-4% of pediatric acute lymphoblastic leukemia (PH+ ALL), and about 25% of adult ALL cases. In certain embodiments, the Philadelphia chromosome is contains a translocation, t(9;22)(q34;q1 1), that results in a novel chimeric gene and protein which fuses the BCR gene on chromosome 22 with the gene encoding the Abelson tyrosine kinase (ABL1) on chromosome 9. The resulting BCR-ABL1 fusion transcript and protein is a constitutively activated tyrosine kinase which activates various signaling pathways to promote leukemic transformation in hematopoietic stem cells. In certain embodiments, subjects having the PH+ subtype of ALL have one or more cells that have a Philadelphia chromosome. In certain embodiments, the cells are bone marrow cells. In certain embodiments, the Ph+ subtype of ALL is associated with a low, reduced, and/or decreased risk, probability, and/or likelihood of toxicity following administration of and/or associated with a therapy, e.g., an immunotherapy and/or a cell therapy. In particular embodiments, the provided methods include one or more steps of administering a therapy, e.g., an immunotherapy and/or a cell therapy, to a subject determined to be Ph+.

In some embodiments, subtype of ALL is identified by assessing and/or analyzing the chromosomal structures for abnormalities, e.g., the Philadelphia chromosome. For example, in some embodiments, the chromosomes are analyzed by karyotyping, e.g., a G-banding technique. G-banding produces an individual's karyotype, whereby giemsa stain is used to produce a series of dark and light bands, with each chromosome displaying a unique banding pattern under light microscope. Each chromosome can be further distinguished by the position of its centromere (metacentric, submetacentric, acrocentric), dividing it into a shorter arm, the p (petite) arm and a longer arm, called the q arm. Chromosomes are then arranged with pairs side by side to detect abnormalities including deletions, duplications, or other structural rearrangements. This technique is relatively inexpensive and is a good first-line test for anomalies, but a limitation of this technique is the inability to detect small deletions or rearrangements.

Other techniques include fluorescent In-situ Hybridization (FISH) and multicolor FISH which uses a fluorescently-labeled probes to detect the presence or absence of a particular chromosome segment or gene. FISH and multicolor FISH can detect small deletions, duplications and/or subtle chromosomal rearrangements. FISH and multicolor FISH analysis can be performed on the same specimens obtained for chromosome analysis. In some embodiments, the PH+ subtype is identified and/or detected by FISH and/or multicolor FISH.

In particular embodiments, the subtype of ALL is the Philadelphia-like (Ph-like) subtype of ALL. In some embodiments, the Ph-like subtype is characterized by related gene expression signatures variously referred to as “cluster group R8,” “Philadelphia Chromosome (Ph)-like,” “Ph-like,” “BCR-ABL1-like,” or an “activated tyrosine kinase gene expression signature.” These gene expression signatures have been shown to be highly similar to gene expression profiles measured in PH+ ALL subjects, despite the fact that, in some embodiments, Ph-like subjects to not have the Philadelphia chromosome translocation or the BCR-ABL1 fusion transcript. The prevalence of the Ph-like subtype is approximately 12% in children, 21% in adolescents (16-20 years of age), and 20% to 24% in adults older than 40 years, with a peak (27%) in young adults 21 to 39 years old. It occurs more often in male individuals and patients with Down syndrome. Ph-like ALL is overrepresented in those with Hispanic ethnicity and is associated with inherited genetic variants in GATA3 (rs3824662). It is a clinically and biologically heterogeneous subtype of B-ALL.

In some embodiments, “Philadelphia chromosome-like” or “BCR-ABL1 -like” gene expression signatures have a deletion of the IKAROS or LKZFI transcription factor that regulates B cell development. In some embodiments, a subject with Ph-like ALL has one or more cells with a rearrangement involving one or more of ABL1, ABL2, CRLF2, CSF1R, EPOR, JAK2, NTRK3, PDGFRB, PTK2B, TSLP, and TYK2. In particular embodiments, a subject with Ph-like ALL has one or more cells with sequence mutations involving FLT3, IL7R, or SH2B3. In certain embodiments, subjects having the Ph-like subtype of ALL have one or more cells with a Ph-like gene expression profile. In certain embodiments, the cells are bone marrow cells. In certain embodiments, the Ph-like subtype of ALL is associated with a low, reduced, and/or decreased risk, probability, and/or likelihood of toxicity following administration of and/or associated with a therapy, e.g., an immunotherapy and/or a cell therapy. In particular embodiments, the provided methods include one or more steps of administering a therapy, e.g., an immunotherapy and/or a cell therapy, to a subject determined have a Ph-like subtype of ALL.

In some embodiments, the subtype, e.g., the molecular subtype, of ALL is a not a PH+ or Ph-like subtype. In particular embodiments, molecular subtypes of ALL that are not Ph+ or Ph-like subtypes include, but are not limited to, subtypes associated with a TCF3-PBX1 fusion, a ETV6-RUNX1 fusion, a EP300-ZNF384 fusion, a KMT2A-AFF1 fusion, hyperploidy, or a dic(9;20) chromosome abnormality, e.g., dic(9;20)(p13.2;q11.2).

Particular embodiments include methods of administering an immunotherapy to a subject that has or is suspected of having a Ph-like or PH+ subtype of ALL. In certain embodiments, such methods include one or more steps of selecting and/or identifying a subject having a Ph-like or PH+ subtype of ALL. In certain embodiments, the methods include one or more steps of identifying or detecting a Ph-like or PH+ subtype of ALL in a subject. In particular embodiments, subjects with ALL that lack a Ph-like or PH+ subtype are not administered the immunotherapy. In particular embodiments, the immunotherapy is an immunotherapy that is described in Section-II, such as a T cell engager described in Section II-A or a cell therapy containing CAR expressing cells such as described in Section II-B.

Methods and techniques of identifying and/or determining a PH+ and/or a Ph-like ALL subtype include, but are not limited to, karyotype analysis, fluorescence in situ hybridization (FISH), multicolor FISH, polymerase chain reaction (PCR), tyrosine kinase inhibitor assays, gene expression profiling, microarrays, e.g., immunoassays, e.g., immunocytochemistry, Western blot analysis, and ELISA. Methods and techniques of identifying and/or determining a PH+ and/or a Ph-like ALL subtypes have been described (see, e.g., Roberts et al., N Engl J Med (2014) 371(11): 1005-1015; Roberts et al. Cancer Cell (2012) 14; 22(2):153-66; Perez-Andreu et al. Nature Genetics (2013) 45(12): 1494-1498; Yap et al. Leuk Lymphoma (2017) 58(4): 950-958; Roberts et al. J Clin Oncol (2017) 35(4): 394-401; Harvey et al. Blood (2013) 122:826; Harvey et al., Blood (2010) 116(23): 4874-4884; and PCT App. No. WO 2013/090419, hereby incorporated by reference in their entirety).

In certain embodiments, a sample obtained and/or taken from a PH+ and/or Ph-like subject contains positive, high, elevated, and/or increased expression of one or more genes listed in Table 1, Table E2A, and/or Table 3. In certain embodiments, a sample obtained and/or taken from a PH+ and/or Ph-like subject contains negative, low, reduced, and/or decreased expression of one or more genes listed in Table 2 and/or Table E2B. In particular embodiments, a sample obtained and/or taken from a PH+ and/or Ph-like subject contains positive, high, elevated, and/or increased expression of one or more of ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, LO645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A. In some embodiments, a sample obtained and/or taken from a PH+ and/or Ph-like subject contains positive, high, elevated, and/or increased expression of one or more of ADGRF1, BMPR1B, CA6, CD99, CHN2, CRLF2, DENND3, ENAM, GBP5, GLI2, IFITM1, IGJ (JCHAIN), LDB3, L0645744, MDF1C, MUC4, NRXN3, PON2, S100Z, SEMA6A, SLC37A3, SLC2A5, SPATS2L, TMEM154, TP53INP1, TTYH2 or WNT9A. In certain embodiments, a sample obtained and/or taken from a PH+ and/or Ph-like subject contains positive, high, elevated, and/or increased expression of one or more of ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A. In particular embodiments, a sample obtained and/or taken from a PH+ and/or Ph-like subject contains positive, high, elevated, and/or increased expression of one or more of CCL17, CA6, JCHAIN, PTP4A3, IFITM1, CRLF2, ENAM, GBP5, ABCA9, SV2C, SLC37A3, IL15, IL2RA, and SEMA6A. In some embodiments, the sample is a BMA sample. In certain embodiments, the sample is a plasma sample.

In particular embodiments, a sample obtained and/or taken from a PH+ and/or Ph-like subject contains negative, low, reduced, and/or decreased expression of one or more of ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28. In particular embodiments, a sample obtained and/or taken from a PH+ and/or Ph-like subject contains positive, high, elevated, and/or increased expression of one or more of ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, LO645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A; and also contains negative, low, reduced, and/or decreased expression of one or more of ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28. In various embodiments, a sample obtained and/or taken from a PH+ and/or Ph-like subject contains positive, high, elevated, and/or increased expression of one or more of CCL17, CA6, JCHAIN, PTP4A3, IFITM1, CRLF2, ENAM, GBP5, ABCA9, SV2C, SLC37A3, IL15, IL2RA, and SEMA6A; and also contains negative, low, reduced, and/or decreased expression of one or more of PCDHGA12, PCDHGB6, PCDHGBS, PCDHGA9, PINLYP, ASAP2, TTC28, PTCH1, and FMNL1. In particular embodiments, the sample is a BMA sample. In certain embodiments, the sample is a plasma sample.

Table 3 lists exemplary genes with positive, high, elevated, and/or increased expression in samples taken and/or obtained from PH+ and/or Ph-like subjects.

TABLE 3
Exemplary genes associated with Ph+ and Ph-like subtype of ALL

| Gene Symbol | Full Name | Uniprot No. |
|---|---|---|
| CRLF2 | Cytokine receptor-like factor 2 | Q9HC73 |
| NRXN3 | Neurexin-3-beta | Q9HDB5 |
| BMPR1B | Bone morphogenetic protein receptor | O00238 |
| | type-1B | |
| CHN2 | Beta-chimaerin | P52757 |
| SLC2A5 | Solute carrier family 2, facilitated | P22732 |
| | glucose transporter member 5 | |
| S100Z | Protein S100-Z | Q8WXG8 |
| CD99 | CD99 antigen | P14209 |
| MDFIC | MyoD family inhibitor domain-containing | Q9P1T7 |
| | protein | |
| TTYH2 | Protein tweety homolog 2 | Q9BSA4 |
| DENND3 | DENN domain-containing protein 3 | A2RUS2 |
| LOC645744 | — | — |

In some embodiments, samples, e.g., BMA samples, that contain high levels of one or more genes that are negatively correlated and/or are negatively associated with toxicity, e.g., a gene listed in Table 1 or Table E2A, have a higher probability of containing a deletion at CDKN2A (9p21), IKZF1 (7p12), KLHL22 (22q11), STARD3NL (7p14), or a BCR-ABL1 or ETV6-RUNX1 fusion than samples that do not contain high levels of one or more the negatively correlated genes. In some embodiments, deletions at CDKN2A (9p21), IKZF1 (7p12), KLHL22 (22q11), STARD3NL (7p14), or a BCR-ABL1 or ETV6-RUNX1 fusion are positively associated with and/or positively correlated to the genes that are negatively correlated and/or are negatively associated with toxicity, e.g., a gene listed in Table 1 or Table E2A. In some embodiments, samples collected from individuals of Hispanic descent have an increased probability of having positive or elevated expression of one or more of the genes that are negatively correlated and/or are negatively associated with toxicity.

In some embodiments, provided herein are panels, profiles, and/or arrays for use in the detection and/or identification of PH+ and/or Ph-like subtype of ALL. In certain embodiments, the panels, profiles, and/or arrays are suitable for use to measure, assess, detect, and/or quantify the level and/or amount of one or more gene products in a sample, e.g., a BMA sample or a serum sample. In certain embodiments, the gene products are proteins and/or polypeptides. In some embodiments, the gene products are polynucleotides, e.g., mRNA or cDNA derived from mRNA. In particular embodiments, the panels, profiles, and/or arrays include the measurements, assessments, and/or quantifications of at least one, two, three, four, five, six, seven, eight, nine, ten, more than ten, or more than twenty gene products. In some embodiments, the gene products include one or more of the genes listed in Table 1, Table 2, Table 3, Table E2A and/or Table E2B. In particular embodiments, the gene products are of one or more of ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, L0645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A. In certain embodiments, the gene products include one or more of CCL17, CCR6, GAS6, GLI2, PTP4A3, and IL2RA. Gene products may be measured, assessed, detected, and/or quantified by any known means, including but not limited to the techniques described in Section-IC.

E. Gene Reference Value

In some embodiments, the comparison of a measurement of one or more gene products to a reference value of the one or more gene products allows for the assessment, measurement, and/or determination of the risk, probability, and/or likelihood of toxicity following administration of and/or associated with a therapy. In some embodiments, the expression of a gene product in a sample is compared to a reference value, e.g., a gene reference value. In some embodiments, the gene reference value is a value of a level, amount, or concentration of the gene product, and/or a transformation thereof. In some embodiments, the gene reference value is or is derived from an amount or level of an RNA gene product or a protein gene product. In particular embodiments, the gene reference value is an amount or level of the gene product, or a transformation thereof, that is a boundary between or a threshold value that separates the amounts or levels of the gene product, or transformations thereof, that indicate a likelihood to develop toxicity and/or an increased, elevated, or high risk of toxicity following administration of a therapy and values or measurements of gene expression that a indicate an absent or low likelihood and/or a decreased, reduced, or low risk of toxicity following administration of a therapy. In some embodiments, the gene reference value is a boundary, divide, and/or threshold value between the amounts or levels of the gene product where a majority of toxicities take place or have previously taken place and amounts or levels of the gene product where a minority of the toxicities take place or previously taken place.

In certain embodiments, the gene reference value is an amount or level of the gene product, or a transformation thereof, that is a boundary between or a threshold value that separates the amounts or levels of the gene product, or transformations thereof, associated with a disease subtypes from amounts or levels associated with other subtypes of the same disease. In particular embodiments, the gene reference value is an amount or level of the gene product, or a transformation thereof, that is a boundary between or a threshold value that separates the amounts or levels of the gene product, or transformations thereof, associated with PH+ and/or Ph-like subtypes of ALL from the amounts or levels that are associated with subtypes other subtypes of ALL.

In some embodiments, the reference value is or is derived from the minimal level, amount, or concentration that can be detected, such as by one or more methods described in Section-IC. In certain embodiments, when a comparison indicates that the measurement in a sample of the level, amount, or concentration, of a gene product is below the reference value, then the sample is negative for expression of the gene product. In particular embodiments, when a comparison indicates that the measurement in a sample of the level, amount, or concentration, of a gene product is above the reference value, then the sample is positive for expression of the gene product.

In some embodiments, the expression of a gene product is compared to a reference value and/or a gene reference value and an elevated, increased and/or high risk of toxicity is indicated. In particular embodiments, the expression of a gene product is compared to a gene reference value and a reduced, decreased and/or low risk of toxicity is indicated. In certain embodiments, the expression of a gene product that has been normalized to a control is compared to a reference value and/or a gene reference value and an elevated, increased and/or high risk of toxicity is indicated. In particular embodiments, the expression of a gene product that has been normalized to a control is compared to a gene reference value and a reduced, decreased and/or low risk of toxicity is indicated. In certain embodiments, a value of the expression of a gene product that have been normalized or transformed is compared to a reference value and/or a gene reference value and an elevated, increased and/or high risk of toxicity is indicated. In particular embodiments, the value of the expression of a gene product that have been normalized or transformed is compared to a gene reference value and a reduced, decreased and/or low risk of toxicity is indicated.

In some embodiments, the expression of a gene product that is negatively correlated to and/or negatively associated with a risk of toxicity is compared to a gene reference value. In some embodiments, when the expression of a gene product that is negatively correlated to and/or negatively associated with a risk of toxicity is greater than, over, and/or above the gene reference value, then a decreased, reduced, and/or low risk of toxicity is indicated. In some embodiments, when the expression of a gene product that is negatively correlated to and/or negatively associated with a risk of toxicity is less than, under, and/or below the gene reference value, then an elevated, increased, and/or high risk of toxicity is indicated. In certain embodiments, when the expression of a gene product listed in Table 1 or Table E2A is greater than, over, and/or above the gene reference value, then a decreased, reduced, and/or low risk of toxicity is indicated. In particular embodiments, when the expression of a gene product listed in Table 1 or Table E2A is less than, under, and/or below the gene reference value, then an elevated, increased, and/or high risk of toxicity is indicated.

In particular embodiments, the expression of a gene product that is positively correlated to and/or positively associated with a risk of toxicity is compared to a gene reference value. In some embodiments, when the expression of a gene product that is positively correlated to and/or positively associated with a risk of toxicity is greater than, over, and/or above the gene reference value, than an elevated, increased, and/or high risk of toxicity is indicated. In some embodiments, when the expression of a gene product that is positively correlated to and/or positively associated with a risk of toxicity is less than, under, and/or below the gene reference value, then a decreased, reduced, and/or low risk of toxicity is indicated. In some embodiments, when the expression of a gene product listed in Table 2 or Table E2B is greater than, over, and/or above the gene reference value, than an increased, elevated, and/or high risk of toxicity is indicated. In certain embodiments, when the expression of a gene product listed in Table 2 or Table E2B is less than, under, and/or below the gene reference value, then a decreased, reduced, and/or low risk of toxicity is indicated.

In some embodiments, the gene reference value is a predetermined value. In particular embodiments, the gene reference value has been calculated and/or derived from data from a study. In some embodiments, the study is a clinical study. In particular embodiments, the clinical study is a completed clinical study. In certain embodiments, the data from the study included gene expression, e.g., expression of a gene product, in samples taken or obtained from subjects in the study. In particular embodiments, the data from the study includes the number of instances and the degree of severity of toxicities experienced by subjects during the study. In certain embodiments, the subjects in the clinical study had or have a disease or condition. In some embodiments, the disease or condition is cancer. In some embodiments, the cancer is ALL.

In some embodiments, the reference value is or reflects a minimum detectable level, value, or amount of gene expression, e.g., a value that serves as a boundary of positive or negative expression. In certain embodiments, a measurement of the expression of a gene product is compared to a reference value that is or reflects a minimum detectable level, value, or amount of gene expression, e.g., expression of a gene product, and the gene is determined to be positively expressed if the measurement is a value above the reference value, and/or the gene is determined to be negatively expressed if the measurement is a value that is below the reference value.

In particular embodiments, the expression of a gene product in a sample taken or obtained from a subject is compared to a gene reference value that was calculated and/or derived from a study that included subjects with the same disease or condition as the subject. In certain embodiments, the same disease or condition is a cancer. In particular embodiments the same disease or condition is ALL.

In some embodiments, the gene reference value is determined by the application of an algorithm to the level, concentration, or amount of expression in a control sample or a plurality of control samples. In some embodiments, the control sample or plurality of control samples is obtained from a subject or group of subjects of a completed study, e.g., a completed clinical trial, where the subjects were monitored for outcome and signs of toxicity, e.g., as described in Section III-A. In particular embodiments, the sample or the plurality of samples were collected prior to the subjects receiving the therapy. In some embodiments, the subject or group of subjects went on to develop signs of toxicity after the therapy was administered. In certain embodiments, the subject or group of subjects developed and/or experienced a grade of 0 or greater, 1 or greater, 2 or greater, 3 or greater, prolonged 3 or greater, 4 or greater, or 5. In some embodiments, the subject or group of subjects developed and/or experienced a grade of 5 or less, 4 or less, prolonged 3 or less, 3 or less, 2 or less, 1 or less, or 0. In certain embodiments, the subject or group of subjects developed and/or experienced a grade of between 3 and 5, between prolonged 3 and 5, between 4 and 5, between 0 and prolonged 3, between 0 and 3, between 0 and 2, or between 0 and 1. In some embodiments, the gene reference value is determined by the application of an algorithm to two or more control samples or pluralities that are obtained from two or more different subjects or different groups of subjects.

In certain embodiments, illustrative algorithms include but are not limited to methods that reduce the number of variables such as principal component analysis algorithms, partial least squares methods, and independent component analysis algorithms. Illustrative algorithms further include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques include bagging procedures, boosting procedures, random forest algorithms, and combinations thereof. In some embodiments of the present invention a support vector machine (SVM) algorithm, a random forest algorithm, or a combination thereof is used for classification of microarray data or RNA-seq data. In some embodiments, identified markers that distinguish samples or subtypes are selected based on statistical significance. In some cases, the statistical significance selection is performed after applying a Benjamini Hochberg correction for false discovery rate (FDR). In certain embodiments, the algorithmic techniques may be applied to the expression profiles of one or more gene products in a sample, such as gene products listed in Table 1, Table 2, Table 3, Table E2A, and/or Table E2B.

In some embodiments, the algorithm may be supplemented with a meta-analysis approach such as that described by Fishel and Kaufman et al. 2007 Bioinformatics 23(13): 1599-606. Also, the classifier algorithm may be supplemented with a meta-analysis approach such as a repeatability analysis. In some cases, the repeatability analysis selects markers that appear in at least one predictive expression product marker set.

In some embodiments, the gene reference value is an amount or level of the gene product, or a transformation thereof, that is a boundary between or a threshold value that separates the amounts or levels of the gene product, or transformations thereof, where all or a majority of toxicities take place or have previously taken place, from amounts or levels of the gene product, or transformations thereof, where a minority of the toxicities take place or previously taken place. In particular embodiments, the gene reference value partitions or separates values or measurements of the gene expression associated with more than half, and/or greater than 50%, 60%, 70%, 80%, 90%, 95%, or at or about 100% of the instances of toxicity, e.g., severe neurotoxicity that occurred in a study. In some embodiments, the instance of toxicity is neurotoxicity of grade 2 or greater, grade 3 or greater, grade of prolonged 3 or greater, grade 4 or greater, or grade 5. In some embodiments, the instance of toxicity is neurotoxicity of a grade of between 3 and 5, between prolonged 3 and 5, between 4 and 5, or grade 5 neurotoxicity. In some embodiments, the gene reference value partitions or separates values or measurements of the gene expression that are associated with at least a 25%, at least a 30%, at least a 40%, at least a 45%, at least a 50%, at least a 55%, at least a 60%, at least a 65%, at least a 70%, at least a 75%, at least a 80%, at least a 85%, at least a 90%, at least a 95%, or at or about a 100% frequency of toxicity, such as a neurotoxicity of grade 2 or greater, grade 3 or greater, grade of prolonged 3 or greater, grade 4 or greater, or grade 5; or a neurotoxicity with a grade between 3 and 5, between prolonged 3 and 5, between 4 and 5, or grade 5 neurotoxicity.

In particular embodiments, the gene reference value is within 25%, within 20%, within 15%, within 10% or within 5% of the gene expression in a control sample. In some embodiments, the gene reference value is within 25%, within 20%, within 15%, within 10% or within 5% an average or median level, concentration or amount of the gene expression in a plurality of control samples. In particular embodiments, the gene reference value is within 2, 1.5, 1.25, 1, 0.75, 0.5, 0.25, or 0.1 standard deviations of an average or median level, concentration or amount of the gene expression in a plurality of control samples, wherein each of the subjects of the group went on to develop severe neurotoxicity, optionally grade 3 or higher, extended grade 3 or higher or grade 4 or 5 neurotoxicity, after receiving the cell therapy for treating the same disease or condition.

In some embodiments, the reference value is obtained from and/or derived from control samples that were obtained from subjects prior to the administration of the therapy, wherein the subjects of the group went on to develop severe neurotoxicity, such as grade 3 or higher, extended grade 3 or higher, grade 4 or 5, or grade 5 neurotoxicity. In some embodiments, the gene reference value is a value of a gene product of a gene that negatively correlates and/or is negatively associated with toxicity following administration of and/or associated with a therapy, e.g., an immunotherapy or cell therapy, such as a gene product listed in Table 1 and/or E1A (e.g. a subset of these genes in the relevant Table which has a recited SEQ ID NO, and/or a Uniprot ID). In certain embodiments, the gene reference value is within 2-fold, within 1.5 fold, 1.0 fold, within 100%, within 50%, within 40%, within 30%, within 25%, within 20%, within 15%, within 10%, or within 5% above the average level, concentration or amount, and/or is within 2.0, 1.5, 1.25, 1.0, 0.75, 0.5, or 0.25 standard deviations above the average level, concentration, or amount of the gene product in a plurality of the control samples. In certain embodiments, the gene reference value is above the highest level, concentration or amount of the gene product observed in a sample from among the plurality of control samples. In particular embodiments, the gene reference value is within 100%, 75%, 50%, 40%, 30%, 25%, 20%, 10%, or 5% above the highest level, concentration or amount of the gene product observed in a sample from among the plurality of control samples. In some embodiments, the reference value is above the level, concentration or amount observed in at least 50%, at least 60%, at least 70%, at least 75%, at least 80%, at least 85%, at least 90%, or at least 95%, or at least 98% of samples from among a plurality of control samples.

In some embodiments, the gene reference value is a value of a gene product of a gene that negatively correlates and/or is negatively associated with toxicity following administration of and/or associated with a therapy, and the plurality of control samples are obtained from a group of subjects prior to receiving therapy, wherein each of the subjects of the group did not develop severe neurotoxicity. In some embodiments, the subjects developed grade 3 or less, grade 2 or less, or grade 1 or 0 neurotoxicity, after receiving the therapy. In some embodiments, the value is below the lowest level, concentration, or amount, of the at least one gene product observed in a sample from among a plurality of control samples. In certain embodiments, the reference value is below the lowest level, concentration, or amount of the gene product observed in a sample from among the plurality of control samples that did not experience severe neurotoxicity. In some embodiments, the reference value is within 50%, within 40%, within 30%, within 25%, within 20%, within 15%, within 10%, or within 5% below the lowest level, concentration, or amount of the gene product observed in a sample from among the plurality of control samples that did not experience severe neurotoxicity. In some embodiments, the reference value is below the level, concentration, or amount observed in at least 75%, at least 80%, at least 85%, at least 90%, or at least 95%, or at least 98% of samples from among a plurality of control samples obtained from a group of subjects prior to receiving a cell therapy that did not experience severe toxicity.

In some embodiments, the reference value is obtained from and/or derived from control samples that were obtained from subjects prior to the administration of the therapy, wherein the each of the subjects of the group has Philadelphia chromosome positive (PH+) or Philadelphia-like (Ph-like) subtype of ALL. In some embodiments, the gene reference value is a value of a gene product of a gene that is positively associated, such as genes that are expressed or highly expressed in samples from Ph-like or PH+subjects, and/or a gene listed in Table 1 and/or E1A (e.g. a subset of these genes in the relevant Table which has a recited SEQ ID NO, and/or a Uniprot ID). In certain embodiments, the gene reference value is within 2-fold, within 1.5 fold, 1.0 fold, within 100%, within 50%, within 40%, within 30%, within 25%, within 20%, within 15%, within 10%, or within 5% above the average level, concentration or amount, and/or is within 2.0, 1.5, 1.25, 1.0, 0.75, 0.5, or 0.25 standard deviations above the average level, concentration, or amount of the gene product in a plurality of the control samples. In certain embodiments, the gene reference value is above the highest level, concentration or amount of the gene product observed in a sample from among the plurality of control samples. In particular embodiments, the gene reference value is within 100%, 75%, 50%, 40%, 30%, 25%, 20%, 10%, or 5% above the highest level, concentration or amount of the gene product observed in a sample from among the plurality of control samples. In some embodiments, the reference value is above the level, concentration or amount observed in at least 50%, at least 60%, at least 70%, at least 75%, at least 80%, at least 85%, at least 90%, or at least 95%, or at least 98% of samples from among a plurality of control samples obtained from a group of subjects prior to receiving a cell therapy that did not experience severe toxicity or that are not associated with Ph-like or PH+ ALL.

In some embodiments, the gene reference value is a value of a gene product of a gene that is positively associated with PH+ or Ph-like ALL, and the plurality of control samples are obtained from a group of subjects that have ALL, but not the PH+ or Ph-like ALL. In some embodiments, the gene reference value is above the highest level, concentration, or amount, of the at least one gene product observed in a sample from among a plurality of control samples. In certain embodiments, the reference value is below the lowest level, concentration, or amount of the gene product observed in a sample from among the plurality of control samples that did not experience severe neurotoxicity. In some embodiments, the reference value is within 50%, within 40%, within 30%, within 25%, within 20%, within 15%, within 10%, or within 5% below the lowest level, concentration, or amount of the gene product observed in a sample from among the plurality of control samples that did not experience severe neurotoxicity. In some embodiments, the reference value is below the level, concentration, or amount observed in at least 75%, at least 80%, at least 85%, at least 90%, or at least 95%, or at least 98% of samples from among a plurality of control samples obtained from a group of subjects that do not have Ph-like or PH+ ALL.

In certain embodiments, the reference value is obtained from and/or derived from control samples that were obtained from subjects prior to the administration of the therapy, wherein the subjects of the group went on to develop severe neurotoxicity, such as grade 3 or higher, extended grade 3 or higher, grade 4 or 5, or grade 5 neurotoxicity. In some embodiments, the gene reference value is a value of a gene product of a gene that positively correlates and/or is positively associated with toxicity following administration of and/or associated with a therapy, e.g., an immunotherapy or cell therapy, such as a gene product listed in Table 2 and/or E2B. In certain embodiments, the gene reference value is within 2-fold, within 1.5 fold, 1.0 fold, within 100%, within 50%, within 40%, within 30%, within 25%, within 20%, within 15%, within 10%, or within 5% below the average level, concentration or amount, and/or is within 2.0, 1.5, 1.25, 1.0, 0.75, 0.5, or 0.25 standard deviations below the average level, concentration, or amount of the gene product in a plurality of the control samples. In certain embodiments, the gene reference value is below the lowest level, concentration or amount of the gene product observed in a sample from among the plurality of control samples. In particular embodiments, the gene reference value is within 100%, 75%, 50%, 40%, 30%, 25%, 20%, 10%, or 5% below the lowest level, concentration or amount of the gene product observed in a sample from among the plurality of control samples. In some embodiments, the reference value is below the level, concentration or amount observed in at least 50%, at least 60%, at least 70%, at least 75%, at least 80%, at least 85%, at least 90%, or at least 95%, or at least 98% of samples from among a plurality of control samples.

In some embodiments, the gene reference value is a value of a gene product of a gene that negatively correlates and/or is negatively associated with toxicity following administration of and/or associated with a therapy, and the plurality of control samples are obtained from a group of subjects prior to receiving a cell therapy containing cells genetically engineered with a recombinant receptor, wherein each of the subjects of the group did not develop severe neurotoxicity. In some embodiments, the subjects developed grade 3 or less, grade 2 or less, or grade 1 or 0 neurotoxicity, after receiving the cell therapy. In some embodiments, the value is below the lowest level, concentration, or amount, of the at least one gene product observed in a sample from among a plurality of control samples. In certain embodiments, the value is within 100%, within 75%, within 50%, within 40%, within 30%, within 25%, within 20%, within 10%, or within 5% below the lowest level, concentration, or amount, of the at least one gene product observed in a sample from among a plurality of control samples. In some embodiments, the reference value is above the level, concentration, or amount observed in at least 75%, at least 80%, at least 85%, at least 90%, or at least 95%, or at least 98% of samples from among a plurality of control samples obtained from a group of subjects prior to receiving a therapy that did not experience severe toxicity.

In some embodiments, the reference value is obtained from and/or derived from control samples that were obtained from subjects prior to the administration of the therapy, wherein the each of the subjects of the group has Philadelphia chromosome positive (PH+) or Philadelphia-like (Ph-like) subtype of ALL. In some embodiments, the gene reference value is a value of a gene product of a gene that is negatively associated, such as genes that are not expressed or expressed at low levels in samples from Ph-like or PH+subjects, and/or a gene listed in Table 2 and/or E2B. In certain embodiments, the gene reference value is within 2-fold, within 1.5 fold, 1.0 fold, within 100%, within 50%, within 40%, within 30%, within 25%, within 20%, within 15%, within 10%, or within 5% below the average level, concentration, or amount, and/or is within 2.0, 1.5, 1.25, 1.0, 0.75, 0.5, or 0.25 standard deviations above the average level, concentration, or amount of the gene product in a plurality of the control samples. In certain embodiments, the gene reference value is below the lowest level, concentration or amount of the gene product observed in a sample from among the plurality of control samples. In particular embodiments, the gene reference value is within 100%, 75%, 50%, 40%, 30%, 25%, 20%, 10%, or 5% below the lowest level, concentration or amount of the gene product observed in a sample from among the plurality of control samples. In some embodiments, the reference value is below the level, concentration, or amount observed in at least 50%, at least 60%, at least 70%, at least 75%, at least 80%, at least 85%, at least 90%, or at least 95%, or at least 98% of samples from among a plurality of control samples.

In some embodiments, the expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-five, at least forty, at least fifty, at least sixty, at least seventy, at least eighty, at least ninety, or at least one hundred gene products in a sample obtained from a subject are compared to corresponding gene reference values, e.g., gene reference values to the same gene, to determine the probability, risk, or likelihood, that the subject will experience a toxicity, e.g., a severe neurotoxicity. In some embodiments, the subject is at an elevated, increased, and/or high risk of toxicity if comparison of the expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-five, at least forty, at least fifty, at least sixty, at least seventy, at least eighty, at least ninety, or at least one hundred gene products with the corresponding gene reference value indicate that the expression is associated with an elevated, increased, and/or high risk of toxicity.

In some embodiments, the expression of at least one gene product that positively correlates and/or is positively associated with a toxicity and the expression of at least one gene product that negatively correlates to and/or is negatively associated with the toxicity are compared to the corresponding reference values to determine the probability, risk, or likelihood, that the subject will experience a toxicity, e.g., a severe neurotoxicity. In certain embodiments, the expression of at least one gene product listed in Table 1 or Table E2A (e.g. a subset of these genes in the relevant Table which as a recited SEQ ID NO, and/or a Uniprot ID) and the expression of at least one gene product listed in Table 2 or Table E2B (e.g. a subset of these genes in the relevant Table which as a recited SEQ ID NO, and/or a Uniprot ID) are compared to the corresponding reference values to determine the probability, risk, or likelihood, that the subject will experience a toxicity. In some embodiments, the expression of at least one, two, three, four, five, six, seven, eight, nine, ten, eleven, twelve, thirteen, fourteen, fifteen, sixteen, seventeen, eighteen, nineteen, twenty, or more than twenty gene products listed in Table 1 and/or Table E2A (e.g. a subset of these genes in the relevant Table which as a recited SEQ ID NO, and/or a Uniprot ID); and/or the expression of one, two, three, four, five, six, seven, eight, nine, ten, eleven, twelve, thirteen, fourteen, fifteen, sixteen, seventeen, eighteen, nineteen, twenty, or more than twenty gene products listed in Table 2 and/or Table E2B (e.g. a subset of these genes in the relevant Table which as a recited SEQ ID NO, and/or a Uniprot ID) are compared to the corresponding reference values to determine the probability, risk, or likelihood, that the subject will experience a toxicity.

In particular embodiments, a subject is and/or is considered to have a high, elevated, and/or increased risk of developing a toxicity, e.g., neurotoxicity, to a therapy, e.g., a cell therapy, if the expression of one or more gene products that are negatively correlated to and/or negatively associated with the toxicity, e.g., gene products listed in Table 1 and/or Table E2A (e.g. a subset of these genes in the relevant Table which as a recited SEQ ID NO, and/or a Uniprot ID), are below the reference value. In certain embodiments, a subject is and/or is considered to have a high, elevated, and/or increased risk of developing a toxicity, e.g., neurotoxicity, to a therapy if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, or at least one hundred gene products that are negatively correlated to and/or negatively associated with the toxicity are below the reference value.

In some embodiments, a subject is and/or is considered to have a high, elevated, and/or increased risk of developing a toxicity, e.g., neurotoxicity, to a therapy, e.g., a cell therapy, if the expression of one or more gene products that are positively correlated to and/or positively associated with the toxicity, e.g., gene products listed in Table 2 and/or Table E2B (e.g. a subset of these genes in the relevant Table which as a recited SEQ ID NO, and/or a Uniprot ID), are above the reference value. In certain embodiments, a subject is and/or is considered to have a high, elevated, and/or increased risk of developing a toxicity, e.g., neurotoxicity, to a therapy if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, or at least one hundred gene products that are negatively correlated to and/or negatively associated with the toxicity are above the reference value.

In particular embodiments, a subject is and/or is considered to have a low, decreased, and/or reduced risk of developing a toxicity, e.g., neurotoxicity, to a therapy, e.g., a cell therapy, if the expression of one or more gene products that are negatively correlated to and/or negatively associated with the toxicity, e.g., gene products listed in Table 1, are above the reference value. In certain embodiments, a subject is and/or is considered to have a low, decreased, and/or reduced risk of developing a toxicity, e.g., neurotoxicity, to a therapy if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, or at least one hundred gene products that are negatively correlated to and/or negatively associated with the toxicity are above the reference value.

In some embodiments, the gene product is a protein, e.g., a protein measured from a plasma sample that is obtained from a subject prior to or subsequent to administration of a cell therapy, and the gene referetable e4nce value is a concentration of the protein in serum. In certain embodiments, protein is a gene product that is negatively correlated to and/or negatively associated with toxicity, e.g., a gene product listed in Table 1. In certain embodiments, the plasma sample is obtained from a subject prior to administration of the cell therapy, such as 1 day prior. In certain embodiments, the plasma sample is obtained from a subject subsequent to administration of the cell therapy, such as 2 days, 4 days, or 7 days after administration of the cell therapy.

In certain embodiments, the plasma sample is obtained from a subject prior to administration of the cell therapy. In certain embodiments, the protein is a protein or portion of a gene product listed in Table 1. In certain embodiments, the gene product is a CCL17, Endoglin, E-selectin, ICAM-3, or IL-GR protein or portion thereof. In some embodiments, the gene reference value is a concentration of the gene product, e.g., the protein or portion thereof, in serum.

In some embodiments, the plasma sample is collected from the subject prior to administration of the cell therapy, e.g., 1 day prior, and the protein or portion thereof is CCL17. In some embodiments, the gene reference value for CCL17 is a plasma concentration of CCL17 of between or between about 1 pg/mL and 1,000 pg/mL, 5 pg/mL and 250 pg/mL, 20 pg/mL and 100 pg/mL. In some embodiments, the gene reference values are or are about 20 pg/mL, 25 pg/mL, 64 pg/mL, or 100 pg/mL CCL17 in plasma.

In some embodiments, the plasma sample is collected from the subject prior to administration of the cell therapy, e.g., 1 day prior, and the protein or portion thereof is Endoglin. In some embodiments, the gene reference value for Endoglin is a plasma concentration of Endoglin of between or between about 0.5 pg/mL and 2.5 pg/mL, or 1 pg/mL and 2 pg/mL, each inclusive. In some embodiments, the gene reference values are or are about 1 pg/mL, 1.5 pg/mL, or 2 pg/ml Endoglin in plasma.

In some embodiments, the plasma sample is collected from the subject prior to administration of the cell therapy, e.g., 1 day prior, and the protein or portion thereof is E-selectin. In some embodiments, the gene reference value for E-selectin is a plasma concentration of E-selectin of between or between about 1 pg/mL and 10 pg/mL, 4 pg/mL and 8 pg/mL, or 6 pg/mL and 8 pg/mL, each inclusive. In some embodiments, the gene reference values are or are about 6 pg/mL or 8 pg/ml E-selectin in plasma.

In some embodiments, the plasma sample is collected from the subject prior to administration of the cell therapy, e.g., 1 day prior, and the protein or portion thereof is ICAM-3. In some embodiments, the gene reference value for ICAM-3 is a plasma concentration of ICAM 3 of between or between about 0.01 pg/mL and 0.35 pg/mL, 0.1 pg/mL and 0.30 pg/mL, or 0.125 pg/mL and 0.25 pg/mL, each inclusive. In some embodiments, the gene reference values are or are about 0.20 pg/mL, 0.25 pg/mL, or 0.30 pg/ml ICAM-3 in plasma.

In some embodiments, the plasma sample is collected from the subject prior to administration of the cell therapy, e.g., 1 day prior, and the protein or portion thereof is IL-6R. In some embodiments, the gene reference value for IL-6R is a plasma concentration of IL-6R of between or between about 10 pg/mL and 40 pg/mL, 20 pg/mL and 35 pg/mL, or 30 pg/mL and 35 pg/mL, each inclusive. In some embodiments, the gene reference values are or are about 25 pg/mL, 30 pg/mL, or 35 pg/ml IL-6R in plasma.

In some embodiments, the plasma sample is collected from the subject subsequent to administration of the cell therapy, e.g., 2 days, 4 days, or 7 days after the cell therapy, and the protein or portion thereof is CCL17. In some embodiments, the gene reference value for CCL17 is a plasma concentration of CCL17 of between or between about 50 pg/mL and 1,000 pg/mL, 100 pg/mL and 600 pg/mL, 150 pg/mL and 500 pg/mL. In some embodiments, the gene reference values are or are about 100 pg/mL, 250 pg/mL, or 500 pg/mL CCL17 in plasma.

In some embodiments, the plasma sample is collected from the subject subsequent to administration of the cell therapy, e.g., 2 days, 4 days, or 7 days after the cell therapy, and the protein or portion thereof is Endoglin. In some embodiments, the gene reference value for Endoglin is a plasma concentration of Endoglin of between or between about 0.5 pg/mL and 2.5 pg/mL, or 1 pg/mL and 2 pg/mL, each inclusive. In some embodiments, the gene reference values are or are about 1 pg/mL, 1.5 pg/mL, or 2 pg/ml Engoglin in plasma.

In some embodiments, the plasma sample is collected from the subject subsequent to administration of the cell therapy, e.g., 2 days, 4 days, or 7 days after the cell therapy, and the protein or portion thereof is E-selectin. In some embodiments, the gene reference value for E-selectin is a plasma concentration of E-selectin of between or between about 1 pg/mL and 10 pg/mL, 4 pg/mL and 8 pg/mL, or 6 pg/mL and 8 pg/mL, each inclusive. In some embodiments, the gene reference values are or are about 6 pg/mL or 8 pg/ml E-selectin in plasma.

In some embodiments, the plasma sample is collected from the subject subsequent to administration of the cell therapy, e.g., 2 days, 4 days, or 7 days after the cell therapy, and the protein or portion thereof is ICAM-3. In some embodiments, the gene reference value for ICAM-3 is a plasma concentration of ICAM 3 of between or between about 0.01 pg/mL and 0.35 pg/mL, 0.1 pg/mL and 0.30 pg/mL, or 0.125 pg/mL and 0.25 pg/mL, each inclusive. In some embodiments, the gene reference values are or are about 0.20 pg/mL, 0.25 pg/mL, or 0.30 pg/ml ICAM-3 in plasma.

In some embodiments, the plasma sample is collected from the subject subsequent to administration of the cell therapy, e.g., 2 days, 4 days, or 7 days after the cell therapy, and the protein or portion thereof is IL-6R. In some embodiments, the gene reference value for IL-6R is a plasma concentration of IL-6R of between or between about 20 pg/mL and 50 pg/mL, 20 pg/mL and 40 pg/mL, or 25 pg/mL and 35 pg/mL, each inclusive. In some embodiments, the gene reference values are or are about 20 pg/mL, 25 pg/mL, or 30 pg/ml IL-6R in plasma.

In some embodiments, a subject is and/or is considered to have a low, decreased, and/or reduced risk of developing a toxicity, e.g., neurotoxicity, to a therapy, e.g., a cell therapy, if the expression of one or more gene products that are positively correlated to and/or positively associated with the toxicity, e.g., gene products listed in Table 2, are below the reference value. In certain embodiments, a subject is and/or is considered to have a high, elevated, and/or increased risk of developing a toxicity, e.g., neurotoxicity, to a therapy if the expression of expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least fifty, or at least one hundred gene products that are negatively correlated to and/or negatively associated with the toxicity are below the reference value.

In certain embodiments, the subject is and/or is considered to have a high, elevated, and/or increased risk of developing a toxicity, e.g., neurotoxicity, to a therapy, e.g., a cell therapy if the expression of one or more gene products that are negatively correlated to and/or negatively associated with the toxicity, gene products listed in Table 1, are above the reference value and the expression of one or more gene products that is positively correlated to and/or positively associated with the toxicity, e.g., gene products listed in Table 2, are above the reference value. In particular embodiments, the subject is and/or is considered to have a low, reduced and/or decreased risk of developing a toxicity following administration of a therapy, e.g., a cell therapy if the expression of one or more gene products that are negatively correlated to and/or negatively associated with the toxicity, e.g., gene products listed in Table 1, are above the reference value and the expression of one or more gene products that is positively correlated to and/or positively associated with the toxicity, e.g., gene products listed in Table 2, are below the reference value.

### II. IMMUNOTHERAPIES AND CELL THERAPIES

The methods provided herein include on or more steps of assessing, predicting, and/or determining the risk, likelihood, or probability a subject will experience or develop a toxicity following administration of and/or associated with a therapy. In particular embodiments, the risk, likelihood, and/or probability a subject will experience or develop a toxicity following administration of and/or associated with is determined, assessed, or predicted by one or more methods provided in Section I. In some embodiments, the therapy is an immunotherapy. In particular embodiments, the immunotherapy is or contains an immune system activator or stimulator. In some embodiments, the immunotherapy is a T cell engaging therapy, such as a bispecific T cell engaging therapy. In some embodiments, the therapy is a cell therapy, e.g., an autologous cell therapy. In particular embodiments, the cell therapy includes one or more cells that express a recombinant receptor, such as a T cell receptor (TCR) or a chimeric antigen receptor (CAR). In some embodiments, the immunotherapy, e.g. engineered to contain an engineered receptor, such as a chimeric antigen receptor (CAR), is specific to an antigen associated with ALL, such as CD19, CD22, CD20 or CD123. In some embodiments, the immunotherapy is specific to CD19.

A. Immunotherapy

In certain embodiments, the immunotherapy is or contains an immune system activator or stimulator. In certain embodiments, the immune system stimulator is an agent or therapy that activates at least one immune cell. In some embodiments, the immune cell is a T cell. In certain embodiments, the immune cell activator is IL-2, e.g., Proleukin; rhu-IFN-alpha-2a and/or rhu-IFN-alpha-2b, e.g., Pegasys, Roferon-A, Intron-A, and PEG intron; Anti-CD3 monoclonal antibody, e.g., Muromonab-CD3 and/or Orthoclone OKT 3; TGN-1412; and/or Blinatumomab, e.g., anti-CD3xCD19 BiTE.

In some embodiments, the immunotherapy is or contains a T cell-engaging therapy that is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the T cell-engaging therapy is or comprises an antibody or antigen-binding fragment. In some embodiments, the T cell-engaging therapy is a bispecific antibody containing at least one antigen-binding domain binding to an activating component of the T cell (e.g. a T cell surface molecule, e.g. CD3 or CD2) and at least one antigen-binding domain binding to a surface antigen on a target cell, such as a surface antigen on a tumor or cancer cell, for example any of the listed antigens as described herein, e.g. CD19. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

Among such exemplary bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). In some embodiments, the T-cell engaging therapy is blinatumomab or AMG 330. Any of such T cell-engagers can be used in used in the provided methods, compositions or combinations.

The immune system stimulator and/or the T cell engaging therapy can be administered by any suitable means, for example, by bolus infusion, by injection, e.g., intravenous or subcutaneous injections, intra-ocular injection, peri-ocular injection, sub-retinal injection, intra-vitreal injection, trans-septal injection, sub-scleral injection, intra-choroidal injection, intra-cameral injection, sub-conjunctival injection, sub-conjunctival injection, sub-Tenon's injection, retro-bulbar injection, peri-bulbar injection, or posterior juxta-scleral delivery. In some embodiments, the immunotherapy is administered by parenteral, intrapulmonary, and intranasal, and, if desired for local treatment, intra-lesional administration. Parenteral infusions include intramuscular, intravenous, intraarterial, intraperitoneal, intrathoracic, intracranial, or subcutaneous administration.

In certain embodiments, if the risk, probability, and/or likelihood that the subject will experience and/or develop a toxicity following administration of or associated with the immunotherapy is determined to be high, increased, and/or elevated by one or more methods provided in Section-I, then the subject is administered the immunotherapy in an in-patient setting and/or with admission to the hospital for one or more days, optionally wherein the immunotherapy is otherwise to be administered to subjects on an outpatient basis or without admission to the hospital for one or more days if the risk, probability, and/or likelihood of is determined to be low, reduced, or decreased according to the results of a method provided in Section-I. In particular embodiments, and immunotherapy, optionally at a non-reduced dose, optionally on an outpatient basis or without admission to the hospital for one or more days, is administered to the subject that is determined to have a low, decreased, and/or reduced risk of toxicity according to the results of a method provided in Section-I.

In certain embodiments, if the risk, probability, and/or likelihood that the subject will experience and/or develop a toxicity following administration of or associated with the immunotherapy is determined to be high, increased, or elevated by one or more methods provided in Section-I, then the subject is administered a standard dose of the immunotherapy in an in-patient setting and/or with admission to the hospital for one or more days. In particular embodiments, if the risk, probability, and/or likelihood that the subject will experience and/or develop a toxicity following administration of or associated with the immunotherapy is determined to be high, increased, or elevated by one or more methods provided in Section-I, then the subject is administered a reduced dose of the immunotherapy in an in-patient setting and/or with admission to the hospital for one or more days. In certain embodiments, if the risk, probability, and/or likelihood that the subject will experience and/or develop a toxicity following administration of or associated with the immunotherapy is determined to be low, decreased, or reduced by one or more methods provided in Section-I, then the subject is administered a standard dose of the immunotherapy on an outpatient basis or without admission to the hospital for one or more days.

In certain embodiments, if the risk, probability, and/or likelihood that the subject will experience and/or develop a toxicity following administration of or associated with the immunotherapy is determined to be high, increased, or elevated by one or more of the methods provided in Section-I, then the subject is then the subject is administered a reduced dose of the immunotherapy. In certain embodiments, if the risk, probability, and/or likelihood that the subject will experience and/or develop a toxicity following administration of or associated with the immunotherapy is determined to be low, reduced, or decreased by one or more of the methods provided in Section-I, then the subject is administered a reduced dose of the immunotherapy.

In some embodiments, a reduced dose of the immunotherapy is less than or equal to 90%, 85%, 80%, 75%, 70%, 65%, 60%, 55%, 50%, 45%, 40%, 35%, 30%, 25%, 20%, 15%, 10%, 5%, 3%, 1%, 0.5%, 0.1%, 0.05%, 0.01%, 0.001%, or 0.0001% of a standard dose of the immunotherapy. In some embodiments, the immunotherapy is a T cell engaging therapy.

In certain embodiments, if the risk, probability, and/or likelihood that the subject will experience and/or develop a toxicity following administration of or associated with the immunotherapy is determined to be high, increased, or elevated by one or more methods provided in Section-I, then the subject is not administered the immunotherapy. In such embodiments, the subject may be administered an alternative therapy, such as a different immunotherapy or a cell therapy.

In certain embodiments, one or more doses of a T cell engaging therapy and/or an immune system stimulator are administered. In particular embodiments, a standard dose of between 0.001m to 5,000m of the T cell engaging therapy and/or an immune system stimulator are administered. In particular embodiments, a standard dose of between 0.001μg to 1,000 μg, 0.001 μg to 1 μg, 0.01 μg to 1 μg, 0.1 μg to 10 μg, 0.01 μg to 1 μg, 0.1 μg to 5 μg, 0.1 μg to 50 μg, 1 μg to 100 μg, 10 ∥g to 100 μg, 50 μg to 500 μg, 100 μg to 1,000 μg, 1,000 μg to 2,000 μg, or 2,000 μg to 5,000 μg of the T cell engaging therapy is administered. In some embodiments, the standard dose of the T cell engaging therapy is or includes between 0.01 μg/kg and 100 mg/kg, between 0.1 μg/kg and 10 μg/kg, between 10 μg/kg and 50 μg/kg, between 50 mg/kg and 100 mg/kg, between 0.1 mg/kg and 1 mg/kg, between 1 mg/kg and 10 mg/kg, between 10 mg/kg and 100 mg/kg, between 100 mg/kg and 500 mg/kg, between 200 mg/kg and 300 mg/kg, between 100 mg/kg and 250 mg/kg, between 200 mg/kg and 400 mg/kg, between 250 mg/kg and 500 mg/kg, between 250 mg/kg and 750 mg/kg, between 50 mg/kg and 750 mg/kg, between 1 mg/kg and 10 mg/kg, or between 100 mg/kg and 1,000 mg/kg (amount of the lymphodepleting agent over body weight). In some embodiments, the standard dose of the T cell engaging therapy is at least or at least about or is about 0.1 mg/kg, 0.5 mg/kg, 1 mg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1,000 mg/kg. In particular embodiments, the T cell engaging therapy is administered orally, intravenously, intraperitoneally, transdermally, intrathecally, intramuscularly, intranasally, transmucosally, subcutaneously, or rectally.

B. Engineering Cells for Adoptive Cell Therapy

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8+ or CD4+ cells are enriched or selected. Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

The cells generally express recombinant receptors, such as antigen receptors including functional non-TCR antigen receptors, e.g., chimeric antigen receptors (CARs), and other antigen-binding receptors such as transgenic T cell receptors (TCRs) or antigen-binding fragments thereof. Also among the receptors are other chimeric receptors.

1. Chimeric Antigen Receptors (CARs)

In some embodiments of the provided methods and uses, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos.: 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416,and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No.: 7,446,190, and those described in International Patent Application Publication No.: WO/2014055668 A1. Examples of the CARs include CARs as disclosed in any of the aforementioned publications, such as WO2014031687, U.S. Pat. No. 8,339,645, U.S. Pat. No. 7,446,179, US 2013/0149337, U.S. Pat. No.: 7,446,190, U.S. Pat. No.: 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also Application Nos. WO 2014031687 and US 2013/0149337 and U.S. Pat. Nos. 7,446,190, 8,339,645, 7,446,179, and 8,389,282.

The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen is a tumor antigen or cancer marker. In some embodiments, the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, 0-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen Al (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-Al, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some embodiments, the antigen is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgG1 antibody raised against Nalm-1 and −16 cells expressing CD19 of human origin (Ling, N. R., et al. (1987). Leucocyte typing III. 302). The FMC63 antibody comprises CDRH1 and H2 set forth in SEQ ID NOS: 184, 185 respectively, and CDRH3 set forth in SEQ ID NOS: 186 or 200 and CDRL1 set forth in SEQ ID NOS: 181 and CDR L2 set forth in SEQ ID NOS:182 or 201 and CDR L3 sequences set forth in SEQ ID NOS:183 or 180. The FMC63 antibody comprises the heavy chain variable region (V_{H}) comprising the amino acid sequence of SEQ ID NO: 187 and the light chain variable region (V_{L}) comprising the amino acid sequence of SEQ ID NO: 188. In some embodiments, the svFv comprises a variable light chain containing the CDRL1 sequence of SEQ ID NO 181, a CDRL2 sequence of SEQ ID NO:182, and a CDRL3 sequence of SEQ ID NO:183 and/or a variable heavy chain containing a CDRH1 sequence of SEQ ID NO:184, a CDRH2 sequence of SEQ ID NO:185, and a CDRH3 sequence of SEQ ID NO:186. In some embodiments, the scFv comprises a variable heavy chain region of FMC63 set forth in SEQ ID NO:187 and a variable light chain region of FMC63 set forth in SEQ ID NO:188. In some embodiments, the variable heavy and variable light chains are connected by a linker. In some embodiments, the linker is set forth in SEQ ID NO:202. In some embodiments, the scFv comprises, in order, a V_{H}, a linker, and a V_{L}. In some embodiments, the scFv comprises, in order, a V_{L}, a linker, and a V_{H}. In some embodiments, the svFc is encoded by a sequence of nucleotides set forth in SEQ ID NO:203 or a sequence that exhibits at least 85%, 86%, 87%, 88%, 89%, 90%, 91%, 92%, 93%, 94%, 95%, 96%, 97%, 98%, or 99% sequence identity to SEQ ID NO:203. In some embodiments, the scFv comprises the sequence of amino acids set forth in SEQ ID NO:189 or a sequence that exhibits at least 85%, 86%, 87%, 88%, 89%, 90%, 91%, 92%, 93%, 94%, 95%, 96%, 97%, 98%, or 99% sequence identity to SEQ ID NO:189.

In some embodiments the scFv is derived from SJ25C1. SJ25C1 is a mouse monoclonal IgG1 antibody raised against Nalm-1 and −16 cells expressing CD19 of human origin (Ling, N. R., et al. (1987). Leucocyte typing III. 302). The SJ25C1 antibody comprises CDRH1, H2 and H3 set forth in SEQ ID NOS: 193-195, respectively, and CDRL1, L2 and L3 sequences set forth in SEQ ID NOS: 190-192, respectively. The SJ25C1 antibody comprises the heavy chain variable region (V_{H}) comprising the amino acid sequence of SEQ ID NO: 196 and the light chain variable region (V_{L}) comprising the amino acid sequence of SEQ ID NO: 197. In some embodiments, the svFv comprises a variable light chain containing the CDRL1 sequence of SEQ ID NO:190, a CDRL2 sequence of SEQ ID NO: 191, and a CDRL3 sequence of SEQ ID NO:192 and/or a variable heavy chain containing a CDRH1 sequence of SEQ ID NO:193, a CDRH2 sequence of SEQ ID NO: 194, and a CDRH3 sequence of SEQ ID NO:195. In some embodiments, the scFv comprises a variable heavy chain region of SJ25C1 set forth in SEQ ID NO:196 and a variable light chain region of SJ25C1 set forth in SEQ ID NO:197. In some embodiments, the variable heavy and variable light chain are connected by a linker. In some embodiments, the linker is set forth in SEQ ID NO:198. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:199 or a sequence that exhibits at least 85%, 86%, 87%, 88%, 89%, 90%, 91%, 92%, 93%, 94%, 95%, 96%, 97%, 98%, or 99% sequence identity to SEQ ID NO:199.

In some embodiments, the antigen is CD20. In some embodiments, the scFv contains a VH and a VL derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a VH and a VL derived from an antibody or an antibody fragment specific to CD22. In some embodiments, the antibody or antibody fragment that binds CD22 is an antibody that is or is derived from m971, such as is m971 scFv.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 147), and is encoded by the sequence set forth in SEQ ID NO: 148. In some embodiments, the spacer has the sequence set forth in SEQ ID NO: 149. In some embodiments, the spacer has the sequence set forth in SEQ ID NO: 150. In some embodiments, the constant region or portion is of IgD. In some embodiments, the spacer has the sequence set forth in SEQ ID NO: 151. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 147, 149, 150 or 151. In some embodiments, the spacer has the sequence set forth in SEQ ID NOS: 168-179. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 168-179.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 153 or 162 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 153 or 162. An exemplary T2A linker sequence comprises the sequence of amino acids set forth in SEQ ID NO: 153 or 162 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 153 or 162.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 154 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 154; in some embodiments, the transmembrane-domain containing portion of the recombinant receptor comprises the sequence of amino acids set forth in SEQ ID NO: 155 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 156 or 157 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 156 or 157. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 158 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 158.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No.: 7,446,190 or U.S. Pat. No. 8,911,993. For example, in some embodiments, the intracellular signaling domain comprises the sequence of amino acids as set forth in SEQ ID NO: 159, 160, 161 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 159, 160, 161.

In some aspects, the spacer contains only a hinge region of an IgG, such as only a hinge of IgG4 or IgG1, such as the hinge only spacer set forth in SEQ ID NO: 147. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 150. In some embodiments, the spacer is an Ig hinge, e.g., an IgG4 hinge, linked to a CH3 domain only, such as set forth in SEQ ID NO: 149. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 152 or 163, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 152 or 163. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 16, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 152 or 163. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 167), equine rhinitis A virus (E2A, e.g., SEQ ID NO: 166), Thosea asigna virus (T2A, e.g., SEQ ID NO: 152 or 163), and porcine teschovirus-1 (P2A, e.g., SEQ ID NO: 164 or 165) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

2. Multi-Targeting

In some embodiments, the cells used in connection with the provided methods, uses, articles of manufacture and compositions include cells employing multi-targeting strategies. In some embodiments, the cells express multi-chain chimeric antigen receptors (CAR) or express two or more genetically engineered receptors on the cell, each recognizing the same of a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating or stimulatory signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains or regions of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

In some embodiments, the disease to be treated is a B cell malignancy and at least one CAR, e.g., activating CAR binds, to an antigen expressed on at least cells of the disease or condition to be treated. In some embodiments, the disease or condition is acute lymphoblastic leukemia (ALL). In some embodiments, the disease or condition is adult ALL. In some of such aspects, the antigens targeted by the receptors are B cell antigens, such as one or more of CD19, CD20 or CD20.

3. TCRs

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable f3 chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the (3-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Va and VP, such as by RT-PCR in samples, such as T cells, obtained from humans. In some embodiments, libraries, such as single-chain TCR (scTv) libraries, can be assembled from naïve Va and VP libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPredl (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPredl (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol. 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, and WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR a chain containing a variable a domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an a chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence a chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO:173). In some embodiments, the linker has the sequence GSADDAKKDAAKKDGKS (SEQ ID NO:174)

In some embodiments, the scTCR contains a covalent disulfide bond linking a residue of the immunoglobulin region of the constant domain of the a chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, La Jolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10,λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-C1, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector. Genetically Engineered Cells and Methods of Producing Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

4. Vectors and Methods for Genetic Engineering

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: W02014055668, and U.S. Patent No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments, the vector contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 153 or 162) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 153 or 162 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 153 or 162.

5. Cells and Preparation of Cells for Genetic Engineering

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF})_{,} memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood.1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, or CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al.(2012) J Immunother. 35(9): 651-660, Terakura et al. (2012) Blood.1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### III. COMPOSITIONS, FORMULATIONS, AND METHODS OF ADMINISTRATION

Provided herein are methods of administering a therapy, e.g., a cell therapy and/or an immunotherapy, to a subject. In some embodiments, the subjects risk, likelihood, and/or probability of experiencing a toxicity following administration of and/or associated with the therapy has been assessed, determined, and/or measured by one or more methods described in Section-I. In particular embodiments, the subject has been determined to have a low, reduced, and/or decreased risk, probability, or likelihood of experiencing toxicity following administration of and/or associated with the therapy and the subject is administered a standard dose or standard doses of the therapy. In certain embodiments, the subject has been determined to have a high, increased, and/or elevated risk, probability, or likelihood of experiencing toxicity following administration of and/or associated with the therapy, and the subject is administered a reduced dose of the therapy and/or an intervention to prevent or reduce toxicity, e.g., an intervention as described in Section IV. In certain embodiments, the subject is determined to have a high, increased, and/or elevated risk, probability and/or likelihood of developing toxicity following administration of the therapy and the subject is not administered the therapy and is instead administered an alternative therapy. In certain embodiments, the T cell therapy contains one or more cells that express a recombinant receptor, e.g., a CAR.

In some embodiments, the subject is determined to have a low risk, probability, and/or likelihood of developing a toxicity, e.g., a severe neurotoxicity, to a therapy, by one or more methods provided in Section I. In certain embodiments, the subject is determined to have a low risk, probability, and/or likelihood of developing a toxicity following administration of a cell therapy and/or an immunotherapy. In particular embodiments, the expression of one or more genes in a sample is assessed, measured, detected, and/or quantified in a sample taken and/or obtained from the subject, and the subject is determined to have a low risk, probability, and/or likelihood of developing a toxicity, e.g., a severe neurotoxicity, to the cell therapy. In certain embodiments, the therapy is a treatment with a cell therapy that includes CAR expressing cells. In certain embodiments, the subject is determined to have a risk, probability, and/or likelihood of developing a toxicity following administration of a therapy of at or below 50%, at or below 45%, at or below 40%, at or below 35%, at or below 30%, at or below 25%, at or below 20%, at or below 15%, at or below 10%, at or below 5%, at or below 4%, at or below 3%, at or below 2%, at or below 1%, at or below 0.1%, at or below 0.05%, at or below 0.01%, at or below 0.005%, at or below 0.001%, at or below 0.0001%, at or below 0.00001%, or at or below 0.000001%. In certain embodiments, the subject is determined to have a low risk, probability, and/or likelihood of developing a toxicity following administration of a therapy, and the therapy is administered to the subject at a standard dose.

In some embodiments, the subject is determined to have a high risk, probability, and/or likelihood of developing a toxicity, e.g., a severe neurotoxicity, to a therapy. In certain embodiments, the subject is determined to have a high risk, probability, and/or likelihood of developing a toxicity following administration of a therapy. In particular embodiments, the expression of one or more genes in a sample is assessed, measured, detected, and/or quantified in a sample taken and/or obtained from the subject, and the subject is determined to have a high risk, probability, and/or likelihood of developing a toxicity, e.g., a severe neurotoxicity, to the cell therapy. In certain embodiments, the therapy is a treatment with a therapy containing CAR expressing cells. In certain embodiments, the subject is determined to have a risk, probability, and/or likelihood of developing a toxicity following administration of a therapy is at least a at least a 5%, at least a 10%, at least a 15%, at least a 20%, at least a 25%, at least a 30%, at least a 40%, at least a 45%, at least a 50%, at least a 55%, at least a 60%, at least a 65%, at least a 70%, at least a 75%, at least a 80%, at least a 85%, at least a 90%, at least a 95%, at least a 97%, at least a 98%, at least a 99%, or about a 100% risk, probability or likelihood that a toxicity will occur in the subject, e.g., during or after the course of a therapy, such as a cell therapy. In certain embodiments, the subject is determined to have a high risk, probability, and/or likelihood of developing a toxicity following administration of a therapy, and the therapy is not administered to the subject at the standard dose. In some embodiments, the subject is determined to have a high risk, probability, and/or likelihood of developing a toxicity following administration of a therapy, and an alternative treatment is administered to the subject. In some embodiments, the alternative therapy is not a cell therapy. In particular embodiments, the alternative therapy is not a T-cell engaging therapy.

A. Measuring a Parameter of Activity in Cells of the Therapy

In certain embodiments, the activity of one or more cells of a cell therapy is measured or assessed by measuring, detecting, quantifying, and/or assessing a parameter, e.g., a phenotype or a characteristic, of the one or more cells. In certain embodiments, cells with a high activity and/or an increased or elevated activity are not administered to a subject that is determined to be at an elevated, increased, or high risk of toxicity following administration of the therapy by any of the methods described in Section I. In particular embodiments, a reduced dose, e.g., lower than a standard dose, of cells with a high activity and/or an increased or elevated activity are administered to a subject that is determined to be at an elevated, increased, or high risk of toxicity following administration of the cell therapy by any of the methods described in Section I.

In some embodiments, the subject is determined at a high, elevated, and/or increased risk of a toxicity following administration of a cell therapy by one or more methods provided in Section-I. In some embodiments, if the parameter of the cell composition indicates a high, elevated, and/or increased activity, then the therapeutic cell composition is not administered to the subject with the high, elevated, and/or increased risk, or is administered at a reduced dose, e.g., less than the dose. In certain embodiments, if the parameter of the cell composition does not indicate a high, elevated, and/or increased activity, then the therapeutic cell composition is administered to the subject at the standard dose.

In some embodiments, the parameter is assessed by a response to a stimulus, for example a stimulus that stimulates triggers, induces, stimulates, or prolongs an immune cell function. In certain embodiments, the cells are incubated in the presence of stimulating conditions or a stimulatory agent, the parameter is or includes the response to the stimulation. In particular embodiments, the parameter is or includes the production or secretion of a soluble factor in response to one or more stimulations. In some embodiments, the parameter is or includes a lack or production or secretion of a soluble factor in response to one or more stimulations. In certain embodiments, the soluble factor is a cytokine. In particular embodiments, the soluble factor is a proinflammatory cytokine.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the cells are stimulated and the parameter is determined by whether or not a soluble factor, e.g., a proinflammatory cytokine, is produced or secreted. In some embodiments, the stimulation is nonspecific, i.e., is not an antigen-specific stimulation. In some embodiments, cells are incubated in the presence of stimulating conditions or a stimulatory agent for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 48 hours, or for a duration of time between 1 hour and 4 hours, between 1 hour and 12 hours, between 12 hours and 24 hours, or for more than 24 hours.

In certain embodiments, the therapeutic cell composition contains cells that express a recombinant receptor, e.g., a CAR. In some embodiments, the cells of the composition are stimulated with an agent that is an antigen or an epitope thereof that is specific to the recombinant receptor, or is an antibody or fragment thereof that binds to and/or recognizes the recombinant receptor, or a combination thereof. Thus, in some embodiments, the measurement of the parameter is a measure of recombinant receptor-dependent activity. In some embodiments, the recombinant receptor is a CAR, and the agent is an antigen or an epitope thereof that is specific to the CAR, or is an antibody or fragment thereof that binds to and/or recognizes the CAR, or a combination thereof. In particular embodiments, the cells are stimulated by incubating the cells in the presence of target cells with surface expression of the antigen that is recognized by the CAR. In certain embodiments, the recombinant receptor is a CAR, and the agent is an antibody or an active fragment, variant, or portion thereof that binds to the CAR. In certain embodiments, the antibody or the active fragment, variant, or portion thereof that binds to the CAR is an anti-idiotypic (anti-ID) antibody.

In particular embodiments, the parameter is or includes the production or secretion of a proinflammatory cytokine in response to one or more of stimulations. The production and/or the secretion of cytokines contributes to immune responses, and is involved in different processes including the induction of anti-viral proteins and the induction of T cell proliferation. Cytokines are not pre-formed factors but are rapidly produced and secreted in response to cellular activation. The production or secretion of cytokines may be measured, detected, and/or quantified by any suitable known technique.

In some embodiments, the parameter is or includes the production of a proinflammatory cytokine. In certain embodiments, the parameter is or includes the production of more than one proinflammatory cytokine. In certain embodiments, the parameter is or includes the production, or lack thereof, of one or more of IL-2, IL-13, IFN-gamma, or TNF-alpha. In some embodiments, the parameter is the presence of a production, and/or the presence of a high level of production of the cytokine. In some embodiments, the parameter is a low, reduced, or absent production of a cytokine. In some embodiments, the proinflammatory cytokine is TNF-alpha.

In some embodiments, a measurement of the parameter is the amount of a proinflammatory cytokine that was released by cells of the cell composition. In some embodiments, the measurement of the parameter is a concentration of extracellular proinflammatory cytokine, e.g., TNF-alpha, that is released by cells of the cell composition. In certain embodiment, the measurement of the parameter is normalized to the number of cells examined. In some embodiments, the measurement of the parameter is normalized to the amount of time the cells were incubated under the stimulatory conditions. In particular embodiments, the measurement is normalized to the volume of the media where the cells were incubated.

In some embodiments, the measurement of the parameter is compared to a threshold or reference value of the parameter, e.g., a threshold value. In particular embodiments, the cells of the therapeutic cell composition are determined to have a high activity of the measurement of the parameter is above the threshold value. In some embodiments, the threshold value is a boundary between or a threshold value that separates the values of the parameter that are associated with a risk of toxicity and/or where all or a majority of toxicities take place or have previously taken place from values of the parameter associated with low risk of toxicity and or where a minority of the toxicities take place or previously taken place.

In some embodiments, the threshold value is a predetermined value. In particular embodiments, the threshold value has been calculated and/or derived from data from a study. In some embodiments, the study is a clinical study. In particular embodiments, the clinical study is a completed clinical study. In certain embodiments, the data from the study included measurements of the parameter from therapeutic cell compositions that were administered to subjects in the study. In particular embodiments, the data from the study includes the number of instances and the degree of severity of toxicities experienced by subjects who were administered the therapeutic cell compositions. In certain embodiments, the subjects in the clinical study had or have a disease or condition. In some embodiments, the disease or condition is cancer. In some embodiments, the cancer is ALL. In certain embodiments, the therapeutic cell compositions contained cells that expressed recombinant receptors. In particular embodiments, the recombinant receptor was a CAR.

In particular embodiments, the measurement of the parameter is compared to a threshold value that was calculated and/or derived from a study that included the same or similar therapeutic cell compositions. In particular embodiments, the parameter was measured in cells of the therapeutic cell composition that express the same recombinant receptor or CAR as the cell compositions administered in the clinical study.

In certain embodiments, the measurement of the parameter is compared to the threshold value. In some embodiments, the measurement of the parameter is a measure of recombinant receptor activity, e.g., CAR dependent activity. In certain embodiments, when the measurement of the parameter is greater than the reference value, then the cell composition is determined and/or considered to have a high/elevated, and/or increased cell activity. In some embodiments, when the measurement of the parameter is less than the reference value, then the cell composition is determined and/or considered to not to have a high/elevated, and/or increased cell activity. In some embodiments, when the measurement of the parameter is less than the reference value, then the cell composition is determined and/or considered to have a low, reduced and/or decreased cell activity.

In certain embodiments, the threshold value is determined from a measurement of the plurality of compositions, e.g., reference compositions, comprising T cells expressing the recombinant receptor, e.g., a CAR, that were derived from and administered to subjects of a group of subjects that went on to develop toxicity. In some embodiments, the toxicity was severe neurotoxicity, e.g., grade 3 or higher, prolonged grade 3 or higher, grade 4 or 5, or grade 5 neurotoxicity. In some embodiments, the threshold value is within 50%, within 40%, within 30%, within 25%, within 20%, within 15%, within 10%, or within 5% below the average measure of the parameter, e.g., recombinant receptor dependent activity, and/or is within 3, 2.5, 2, 1.5, 1.0, 0.75, 0.5, or 0.25 standard deviations below the average measure in the plurality of reference compositions. In some embodiments, the threshold value is below the lowest measure of the parameter, e.g., recombinant receptor-dependent activity, in a composition from among the plurality of reference compositions. In certain embodiments, the threshold value is within 50%, within 25%, within 20%, within 15%, within 10%, or within 5% below the lowest measure, in a composition from among the plurality of reference compositions. In particular embodiments, the threshold value is below the measure of the parameter, e.g., the recombinant receptor-dependent activity, among more than 75%, 80%, 85%, 90%, 95%, or 98% of samples from among a plurality of reference composition.

In some embodiments, the gene signature, e.g., the expression of one or more genes associated with and/or correlated to toxicity following administration of a cell therapy, e.g., genes listed in Table 1, Table 2, Table 3, Table E2A, Table E2B, or Table E4 (e.g. a subset of these genes in the relevant Table which as a recited SEQ ID NO, and/or a Uniprot ID), of a sample obtained from a subject indicates that the subject has or is likely to have a high, elevated, and/or increased risk of toxicity following administration of a therapeutic cell composition, and a parameter of the therapeutic cell composition is assessed, measured, and/or quantified. In some embodiments, the parameter is related to cell activity. In particular embodiments, the parameter is the release of TNF-alpha by stimulation of a recombinant receptor or CAR that is expressed by cells in the therapeutic cell composition. In some embodiments, measurement of the parameter is compared to a threshold value. In particular embodiments, if the measurement is greater than the threshold value then the therapeutic cell composition is determined to have a high, elevated, and/or increased activity. In some embodiments, if the measurement is less than the threshold value, then the therapeutic cell composition is determined to not to have a high, elevated, and/or increased activity. In particular embodiments, a therapeutic cell composition that is determined to have a high, elevated, and/or increased activity is not administered to the subject at the standard dose. In certain embodiments, a therapeutic cell composition that is determined to not have a high, elevated, and/or increased activity is administered to the subject at the standard dose.

In some embodiments, methods are provided herein to administer a therapeutic cell composition to a subject in need thereof, whereby a dose is administered to subjects with low risk and an alternative doses are administered to subjects with high risk.

B. Administration of a Cell Therapy

Provided herein are methods of administering a cell therapy, such as methods of selecting a subject and administering a dose of cell therapy, wherein the subject is selected based on the risk, probability, and/or likelihood of toxicity. In certain embodiments, the methods include steps to assess, determine, measure, and/or quantify a risk, probability, and/or likelihood that a subject will experience and/or develop a toxicity following administration of or associated with a cell therapy. In some embodiments, the subject's risk, probability, and/or likelihood of experiencing or developing the toxicity is assessed, determined, measured and/or quantified by a method described in Section-I. In particular embodiments, the subject is determined to have a low, reduced, and/or decreased risk, probability and/or likelihood of toxicity, and the subject is administered a standard dose of a cell therapy. In certain embodiments, the subject is determined to have a high, elevated, or increased risk of toxicity, and the subject is administered a dose of a cell therapy that is less than a standard dose of the cell therapy. In particular embodiments, the subject is determined to have a high, elevated, or increased risk of toxicity, and the subject is administered a standard dose of a cell therapy and an intervention that is provided in Section IV. In certain embodiments, the subject is determined to have a high, elevated, or increased risk of toxicity, and the subject is administered a dose of a cell therapy that is lower than the standard dose of the cell therapy and an intervention that is provided in Section IV.

1. Methods of Treatment

Also provided are methods of using and uses of the cells and compositions, such as those present in an output composition described herein, in the treatment of diseases, conditions, and disorders in which the antigen recognized by the recombinant receptor (e.g. CAR) is expressed. In certain embodiments of the methods provided herein, the subject is administered a standard dose of a therapy, e.g., an immunotherapy or a cell therapy. In certain embodiments, the risk, probability, and/or likelihood that the subject will experience and/or develop a toxicity following administration of or associated with the cell therapy is determined by one or more methods provided in Section-I. In certain embodiments, the subject is determined to be unlikely, or to have a low risk, probability, and/or likelihood, of developing a toxicity, e.g., a neurotoxicity and/or a severe neurotoxicity, by performing one or more methods described in Section I, and the subject is administered a standard dose of the cell therapy. In particular embodiments, the subject is administered a reduced dose of the therapeutic cell composition. In some embodiments, the subject is determined to have a high, elevated, and/or an increased risk, probability, and/or likelihood of developing a toxicity, e.g., a neurotoxicity, and the subject is administered a reduced dose of the therapeutic cell composition. In some embodiments, the cell therapy is a T cell therapy. In some embodiments, the T cell therapy contains cells that express a recombinant receptor. In some embodiments, the recombinant receptor is a CAR.

The provided methods and uses include methods and uses for adoptive cell therapy. Provided are methods of administering the engineered cells and compositions, and uses of such engineered cells and compositions to treat or prevent diseases, conditions, and disorders, including cancers. The disease or condition that is treated in some aspects can be any in which expression of an antigen is associated with, specific to, and/or expressed on a cell or tissue of a disease, disorder or condition and/or involved in the etiology of a disease, condition or disorder, e.g. causes, exacerbates or otherwise is involved in such disease, condition, or disorder. In some embodiments, the methods include administration of the engineered cells or a composition containing the cells, such as cells from an output composition as described, to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition, ameliorate one or more symptom of the disease or condition, such as by lessening tumor burden in a cancer expressing an antigen recognized by an engineered T cell.

The disease or condition that is treated in some aspects can be any in which expression of an antigen is associated with, specific to, and/or expressed on a cell or tissue of a disease, disorder or condition and/or involved in the etiology of a disease, condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen.

Exemplary antigens, which include antigens associated with various diseases and conditions that can be treated, are described above. In particular embodiments, the immunomodulatory polypeptide and/or recombinant receptor, e.g., the chimeric antigen receptor or TCR, specifically binds to an antigen associated with the disease or condition. In some embodiments, the subject has a disease, disorder or condition, optionally a cancer, a tumor, an autoimmune disease, disorder or condition, or an infectious disease.

In some embodiments, the disease, disorder or condition includes tumors associated with various cancers. The cancer can in some embodiments be any cancer located in the body of a subject, such as, but not limited to, cancers located at the head and neck, breast, liver, colon, ovary, prostate, pancreas, brain, cervix, bone, skin, eye, bladder, stomach, esophagus, peritoneum, or lung. For example, the anti-cancer agent can be used for the treatment of colon cancer, cervical cancer, cancer of the central nervous system, breast cancer, bladder cancer, anal carcinoma, head and neck cancer, ovarian cancer, endometrial cancer, small cell lung cancer, non-small cell lung carcinoma, neuroendocrine cancer, soft tissue carcinoma, penile cancer, prostate cancer, pancreatic cancer, gastric cancer, gall bladder cancer or espohageal cancer. In some cases, the cancer can be a cancer of the blood. In some embodiments, the disease, disorder or condition is a tumor, such as a solid tumor, lymphoma, leukemia, blood tumor, metastatic tumor, or other cancer or tumor type. In some embodiments, the disease, disorder or condition is selected from among cancers of the colon, lung, liver, breast, prostate, ovarian, skin, melanoma, bone, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, and/or mesothelioma.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma,diffuse large B-cell lymphoma (DLBCL) and multiple myeloma (MM), a B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen or ligand is a tumor antigen or cancer marker. In some embodiments, the antigen or ligand the antigen is or includes αvβ6 integrin (αvβ6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen Al (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some embodiments, the disease or condition is a B cell malignancy. In some embodiments, the B cell malignancy is a leukemia or a lymphoma. In some aspects, the disease or condition is acute lymphoblastic leukemia (ALL), adult ALL, chronic lymphoblastic leukemia (CLL), non-Hodgkin lymphoma (NHL), or Diffuse Large B-Cell Lymphoma (DLBCL). In some cases, the disease or condition is an NHL, such as or including an NHL that is an aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B). In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the disease or condition is acute lymphoblastic leukemia (ALL). In some embodiments, the disease or condition is adult ALL. In some of such aspects, the antigens targeted by the receptors are B cell antigens, such as one or more of CD19, CD20 or CD20. Chimeric antigen receptors for targeting such diseases are known, including any as described.

In particular embodiments, the immunomodulatory polypeptide and/or recombinant receptor, e.g., the chimeric antigen receptor or TCR, specifically binds to an antigen associated with the disease or condition. In some embodiments, the subject has a disease, disorder or condition, optionally a cancer, a tumor, an autoimmune disease, disorder or condition, or an infectious disease.

In certain embodiments, if the risk, probability, and/or likelihood that the subject will experience and/or develop a toxicity following administration of or associated with the cell therapy is determined to be high, increased, or elevated by one or more methods provided in Section-I, then the subject is administered the cell therapy in an in-patient setting and/or with admission to the hospital for one or more days, optionally wherein the cell therapy is otherwise to be administered to subjects on an outpatient basis or without admission to the hospital for one or more days if the risk, probability, and/or likelihood of is determined to be low, reduced, or decreased according to the results of a method provided in Section-I. In particular embodiments, a cell therapy, optionally at a non-reduced dose, optionally on an outpatient basis or without admission to the hospital for one or more days, is administered to the subject that is determined to have a low, decreased, and/or reduced risk of toxicity according to the results of a method provided in Section-I.

In certain embodiments, if the risk, probability, and/or likelihood that the subject will experience and/or develop a toxicity following administration of or associated with the cell therapy is determined to be high, increased, or elevated by one or more methods provided in Section-I, then the subject is administered a standard dose of the cell therapy in an in-patient setting and/or with admission to the hospital for one or more days. In particular embodiments, if the risk, probability, and/or likelihood that the subject will experience and/or develop a toxicity following administration of or associated with the cell therapy is determined to be high, increased, or elevated by one or more methods provided in Section-I, then the subject is administered a reduced dose of the cell therapy in an in-patient setting and/or with admission to the hospital for one or more days. In certain embodiments, if the risk, probability, and/or likelihood that the subject will experience and/or develop a toxicity following administration of or associated with the cell therapy is determined to be low, decreased, or reduced by one or more methods provided in Section-I, then the subject is administered a standard dose of the cell therapy on an outpatient basis or without admission to the hospital for one or more days.

In certain embodiments, if the risk, probability, and/or likelihood that the subject will experience and/or develop a toxicity following administration of and/or associated with the cell is determined to be high, increased, or elevated by one or more methods provided in Section-I, then the subject is not administered the cell therapy. In such embodiments, the subject may be administered an alternative therapy, such as a different cell therapy, or an immunotherapy.

In some embodiments, subjects with low, decreased, and/or reduced risk, probability, and/or likelihood to experience and/or develop a toxicity as determined by one or more methods provided in Section-I, are administered a standard dose of the cell therapy and at least 30%, at least 35%, at least 40%, at least 45%, at least 50%, at least 55%, at least 60%, at least 65%, at least 70%, at least 85%, at least 90%, at least 97%, at least 99%, or about 100% of the subjects do not develop or experience severe toxicity. In some embodiments, subjects with high, increased, or elevated risk, probability, and/or likelihood to experience and/or develop a toxicity as determined by one or more methods provided in Section-I, are administered a reduced dose of the cell therapy and at least 30%, at least 35%, at least 40%, at least 45%, at least 50%, at least 55%, at least 60%, at least 65%, at least 70%, at least 85%, at least 90%, at least 97%, at least 99%, or about 100% of the subjects do not develop or experience severe toxicity. In some embodiments, subjects with high, increased, or elevated risk, probability, and/or likelihood to experience and/or develop a toxicity as determined by one or more methods provided in Section-I, are administered a standard dose of the cell therapy and an intervention to prevent or reduce toxicity provided in Section IV, and at least 30%, at least 35%, at least 40%, at least 45%, at least 50%, at least 55%, at least 60%, at least 65%, at least 70%, at least 85%, at least 90%, at least 97%, at least 99%, or about 100% of the subjects do not develop or experience severe toxicity. In particular embodiments, subjects with high, increased, or elevated risk, probability, and/or likelihood to experience and/or develop a toxicity as determined by one or more methods provided in Section-I, are administered a reduced dose of the cell therapy and an intervention to prevent or reduce toxicity provided in Section IV, and at least 30%, at least 35%, at least 40%, at least 45%, at least 50%, at least 55%, at least 60%, at least 65%, at least 70%, at least 85%, at least 90%, at least 97%, at least 99%, or about 100% of the subjects do not develop or experience severe toxicity. In some embodiments, the severe toxicity is severe CRS of grade 3 or higher, grade 4 or higher, or grade 5. In some embodiments, the severe toxicity is severe neurotoxicity of grade 3 or higher, extended grade 3 or higher, grade 4 or higher, or grade 5.

In certain embodiments, a reduced dose of a therapeutic cell composition contains less than or equal to 90%, less than or equal to 80%, less than or equal to 70%, less than or equal to 60%, less than or equal to 50%, less than or equal to 40%, less than or equal to 30%, less than or equal to 20%, or less than or equal to 10%, or less than or equal to 1%, less than or equal to 0.1%, or less than or equal to 0.01% of the total amount of cells, CAR+cells, CAR+CD4+ cells, and/or CD8+CAR+ cells of a standard dose of the therapeutic cell composition. In some embodiments, a reduced dose of a therapeutic cell composition contains less than or equal to 90%, less than or equal to 80%, less than or equal to 70%, less than or equal to 60%, less than or equal to 50%, less than or equal to 40%, less than or equal to 30%, less than or equal to 20%, or less than or equal to 10%, or less than or equal to 1%, less than or equal to 0.1%, or less than or equal to 0.01% of the amount of CAR+cells of a standard dose of the therapeutic cell composition total amount of cells per kg of subject body weight, CAR+ cells per kg of subject body weight, CAR+CD4+ cells per kg of subject body weight, and/or CD8+CAR+ cells per kg of subject body weight.

In some embodiments, the methods include administration of the engineered cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition, ameliorate one or more symptom of the disease or condition.

In some embodiments, the cell-based therapy is or comprises administration of cells, such as T cells, that target a molecule expressed on the surface of a lesion, such as a tumor or a cancer. In some embodiments, the immune cells express a T cell receptor (TCR) or other antigen-binding receptor. In some embodiments, the immune cells express a recombinant receptor, such as a transgenic TCR or a chimeric antigen receptor (CAR). In some embodiments, the cells are autologous to the subject. In some embodiments, the cells are allogeneic to the subject.

Methods for administration of engineered cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al., (2013) Nat Biotechnol. 31(10): 928-933; Tsukahara et al., (2013) Biochem Biophys Res Commun 438(1): 84-9; Davila et al., (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. The cells can be administered by any suitable means. Dosing and administration may depend in part on whether the administration is brief or chronic. Various dosing schedules include but are not limited to single or multiple administrations over various time-points, bolus administration, and pulse infusion.

The cells can be administered by any suitable means, for example, by bolus infusion, by injection, e.g., intravenous or subcutaneous injections, intraocular injection, periocular injection, subretinal injection, intravitreal injection, trans-septal injection, sub-scleral injection, intra-choroidal injection, intra-cameral injection, sub-conjunctival injection, sub-conjunctival injection, sub-Tenon's injection, retro-bulbar injection, peri-bulbar injection, or posterior juxtascleral delivery. In some embodiments, they are administered by parenteral, intrapulmonary, and intranasal, and, if desired for local treatment, intra-lesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (−2 g/m²) of cyclophosphamide and 3 to 5 doses of 25 mg/m² fludarabine prior to the first or subsequent dose.

In some embodiments, the provided methods include one or more steps of administering to a subject cells of the output cell composition, such as a composition of cells described in Section I. In certain embodiments, the cells of the output cell composition include engineered CD4+ T cells and engineered CD8+ T cells. In some embodiments, the engineered CD4+ and CD8+ T cells express a T cell receptor (TCR) or other antigen-binding receptor. In some embodiments, the immune cells express a recombinant receptor, such as a transgenic TCR, a chimeric antigen receptor (CAR) or an antigen-binding fragment thereof. In some embodiments, the cells of the output cell composition are autologous to the subject. In some embodiments, the cells are allogeneic to the subject.

In certain embodiments, the CD4+ T cells and CD8+ T cells of the output cell composition are administered to the subject in the same composition, dose, or mixture. Thus, in some embodiments, the recombinant receptor expressing CD4+ T cells and recombinant receptor expressing CD8+ T cells, e.g., CAR+CD4+ and CAR+CD8+ are administered to the subject in the same composition, dose, or mixture.

2 Dosing

In some embodiments, the cell therapy, such as CAR-T cell therapy, is administered to a subject in a therapeutically effective amount to treat the disease, such as in an amount to reduce disease burden. In some embodiments, the dose is a flat dose that is administered as a total number of cells, such as total recombinant receptor (e.g., CAR)-expressing cells, T cells, or peripheral blood mononuclear cells (PBMCs),In some embodiments, the dose is given as a number of cells per kg body weight of the subject. In some embodiments, the subject is a human. In some embodiments, the subject is an adult, such as an adult human subject. In some embodiments, the subject is a pediatric human subject.

In some embodiments, the standard dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the standard dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1 x 10⁶ cells/kg, or no more than at or about 2×10⁶ cells/kg. In some embodiments, the standard dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells are administered to the subject at a standard dose. In particular embodiments, the standard dose is or contains a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 5×10⁸ total recombinant receptor (e.g., CAR)-expressing cells, T cells, or peripheral blood mononuclear cells (PBMCs). In some embodiments, for example, where the subject is a human, the standard dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁸ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×10⁷ to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In certain embodiments, if the risk, probability, and/or likelihood that the subject will experience and/or develop a toxicity following administration of or associated with the cell therapy is determined to be high, increased, or elevated by one or more of the methods provided in Section-I, then the subject is administered a reduced, e.g. low, dose of the cell therapy. In some embodiments, a reduced dose is less than or equal to 90%, 85%, 80%, 75%, 70%, 65%, 60%, 55%, 50%, 45%, 40%, 35%, 30%, 25%, 20%, 15%, 10%, 5%, 3%, 1%, 0.5%, 0.1%, 0.05%, 0.01%, 0.001%, or 0.0001% of a standard dose.

In certain embodiments, a reduced dose of a therapeutic cell composition contains less than or equal to 90%, less than or equal to 80%, less than or equal to 70%, less than or equal to 60%, less than or equal to 50%, less than or equal to 40%, less than or equal to 30%, less than or equal to 20%, or less than or equal to 10%, or less than or equal to 1%, less than or equal to 0.1%, or less than or equal to 0.01% of the total amount of cells, CAR+ cells, CAR+CD4+ cells, and/or CD8+CAR+ cells of a standard dose of the therapeutic cell composition. In some embodiments, a reduced dose of a therapeutic cell composition contains less than or equal to 90%, less than or equal to 80%, less than or equal to 70%, less than or equal to 60%, less than or equal to 50%, less than or equal to 40%, less than or equal to 30%, less than or equal to 20%, or less than or equal to 10%, or less than or equal to 1%, less than or equal to 0.1%, or less than or equal to 0.01% of the amount of CAR+ cells of a standard dose of the therapeutic cell composition total amount of cells per kg of subject body weight, CAR+ cells per kg of subject body weight, CAR+CD4+ cells per kg of subject body weight, and/or CD8+CAR+ cells per kg of subject body weight.

In some embodiments, a subject that is determined to be at risk or likely at risk of developing a toxicity following administration of the cell therapy is administered a dose of genetically engineered cells, e.g. CAR-T cells, that is from or from about 2×10⁶ to 5×10⁷ total recombinant receptor-expressing cells, inclusive, or any range or number between any of the foregoing. In some embodiments, a subject that is determined to be at risk or likely at risk of developing a toxicity following administration of the cell therapy is administered a dose of genetically engineered cells, e.g. CAR-T cells, that is is from or from about 2×10⁵ cells/kg to 5×10⁵ cells/kg total recombinant receptor-expressing cells, inclusive, or any range or number between any of the foregoing.

In certain embodiments, if the risk, probability, and/or likelihood that the subject will experience and/or develop a toxicity following administration of or associated with the cell therapy is determined to be low, reduced, or decreased by one or more of the methods provided in Section-I, then the subject is administered a standard dose of the cell therapy. In some embodiments, a subject that is determined not to be at risk or likely not to be at risk of developing a toxicity following administration of the cell therapy is administered a dose of genetically engineered cells, e.g. CAR-T cells, that is from or from about 1×10⁷ to 2.0×10⁸ total recombinant receptor-expressing cells, inclusive, such as any number or range between any of the foregoing. In some embodiments, a subject that is determined not to be at risk or likely not to be at risk of developing a toxicity following administration of the cell therapy is administered a dose of genetically engineered cells, e.g. CAR-T cells, that is from or from about 1×10⁶ cells/kg to 2×10⁶ cells/kg total recombinant receptor-expressing cells, inclusive, such as any number or range between any of the foregoing.

In some embodiments, the subject is a subject that is known to or that has been selected based on having a Philadelphia chromosome (Ph+) and/or Ph chromosome-like (Ph-like) molecular subtype of acute lymphoblastic leukemia (ALL). In such embodiments, the subject is administered a relatively high dose. In some embodiments, a subject that has a Philadelphia chromosome (Ph+) and/or Ph chromosome-like (Ph-like) molecular subtype of acute lymphoblastic leukemia (ALL) is administered a dose of genetically engineered cells, e.g. CAR-T cells, that is from or from about 1×10⁷ to 2.0×10⁸ total recombinant receptor-expressing cells, inclusive, such as any number or range between any of the foregoing. In some embodiments, a subject that has a Philadelphia chromosome (Ph+) and/or Ph chromosome-like (Ph-like) molecular subtype of acute lymphoblastic leukemia (ALL) is administered a dose of genetically engineered cells, e.g. CAR-T cells, that is from or from about 1×10⁶ cells/kg to 2×10⁶ cells/kg total recombinant receptor-expressing cells, inclusive, such as any number or range between any of the foregoing.

In some embodiments, the dose of cells, e.g., recombinant receptor-expressing T cells, is administered to the subject as a single dose or is administered only one time within a period of two weeks, one month, three months, six months, 1 year or more.

In some aspects, the pharmaceutical compositions and formulations are provided as unit dose form compositions including the number of cells for administration in a given dose or fraction thereof. In some embodiments, the provided methods produce cells in a predictable timeline to dosing as compared to other methods of incubating (e.g., stimulating) cells. In some cases, the dose of cells for administration is determined based on the number of naïve-like cells in the input cell composition. In some embodiments, the unit dose is a standard dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some aspects, the size of a dose, e.g., standard dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent standard dose is inversely correlated with disease burden. In some aspects, as in the context of a large disease burden, the subject is administered a low number of cells. In other embodiments, as in the context of a lower disease burden, the subject is administered a larger number of cells.

In some aspects, the size of the standard dose is determined by the subject's risk for toxicity, e.g., as assessed by the expression of one or more genes in a sample such as a bone marrow aspirate sample. In some aspects, the number of cells administered in the dose is determined based on the gene expression of a sample that is collected prior to such as immediately prior to administration of the initiation of the dose of cells. In some embodiments, the size of the first and/or subsequent dose is inversely correlated with the risk for toxicity. In some aspects, as in the context of a likelihood and/or elevated risk or probability of toxicity, the subject is administered a low number of cells. In other embodiments, as in the context of a lower risk or probability, the subject is administered a larger number of cells.

In some aspects, the size of the standard dose is determined by the activity of the cells of the cell therapy, e.g., as determined by measuring one or more parameters of the cell therapy. In some aspects, the number of cells administered in the dose is determined based on the activity of the cells of the cell therapy. In some embodiments, the size of the first and/or subsequent dose is inversely correlated with the amount or level of activity in the cells. In particular embodiments, the number of cells administered in the dose is determined by the activity of the cells of the cell therapy and the risk of the subject for a toxicity. In certain embodiments, the subject is administered a standard dose of the cell therapy if the cells have a low or moderate activity as determined by measuring the one or more parameters, regardless of the risk assessment provided by the measuring the expression of one or more genes of a sample form the subject.

In some embodiments, the size of the standard dose is determined by the disease-subtype of the subject. In certain embodiments, the subject has cancer and/or a proliferative disease. In some embodiments, the cancer or proliferative disease is ALL. In certain embodiments, the subject has or is suspected of having ALL of a particular subtype. In some embodiments, the size of the first and/or subsequent dose is reduced if the ALL subtype is not Philadelphia positive and/or if cells of the bone marrow do contain a Philadelphia chromosome. In particular embodiments, the size of the first and/or subsequent dose is reduced if the ALL subtype is not the Philadelphia-like subtype and/or if cells of the bone marrow do contain a Philadelphia-like gene signature and/or expression profile. In some embodiments, the size of the first and/or subsequent dose is reduced if the ALL subtype is not the Philadelphia positive or the Philadelphia-like subtype. In particular embodiments, the first and/or subsequent dose is a standard dose if the ALL subtype is the Philadelphia positive or the Philadelphia-like subtype.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

The cells can be administered by any suitable means. The cells are administered in a dosing regimen to achieve a therapeutic effect, such as a reduction in tumor burden. Dosing and administration may depend in part on the schedule of administration of the immunomodulatory compound, which can be administered prior to, subsequent to and/or simultaneously with initiation of administration of the T cell therapy. Various dosing schedules of the T cell therapy include but are not limited to single or multiple administrations over various time-points, bolus administration, and pulse infusion. In certain embodiments, the engineered T cells express a recombinant receptor. In certain embodiments, the engineered T cells express a CAR.

In particular embodiments, the ratio of the CD4+ T cells to CD8+ T cells that are administered to the subject in the same composition, dose, or mixture is between 5:1 to 0.2:1, between 4:1 to 0.25:1, between 3:1 to 0.33:1, between 2:1 to 0.5:1, between 1.5:1 to 0.66:1, or between 1.25:1 to 0.8:1. In some embodiments, the ratio of CD4+ T cells to CD8+ T cells administered to the subject in the same composition, dose, or mixture is or is about 2.0:1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, or 0.5:1.

In particular embodiments, the ratio of recombinant receptor expressing CD4+ T cells to recombinant receptor expressing CD8+ T cells that are administered to the subject in the same composition, dose, or mixture is between 5:1 to 0.2:1, between 4:1 to 0.25:1, between 3:1 to 0.33:1, between 2:1 to 0.5:1, between 1.5:1 to 0.66:1, or between 1.25:1 to 0.8:1. In certain embodiments, the ratio of recombinant receptor expressing CD4+ T cells to recombinant receptor expressing CD8+ T cells that are administered to the subject in the same composition, dose, or mixture is or is about 2.0:1, 1.9:1, 1.8:1, 1.7:1, 1.6:1, 1.5:1, 1.4:1, 1.3:1, 1.2:1, 1.1:1, 1.0:1, 0.9:1, 0.8:1, 0.7:1, 0.6:1, or 0.5:1. In particular embodiments, ratio of the administered recombinant receptor expressing CD4+ T cells to recombinant receptor expressing CD8+ T cells is or is about 1:1. In some embodiments, the recombinant receptor is a TCR or a CAR. In particular embodiments, the recombinant receptor is a CAR.

In some embodiments, the ratio of engineered CD4+ T cells to engineered CD8+ T cells of the dose, composition, or mixture that is administered to the subject is within a certain tolerated difference or range of error of such a defined, desired, or fixed ratio. In some embodiments, the tolerated difference is within of or of about 1%, 2%, 3%, 4%, 5%, 10%, 15%, 20%, 25%, 30%, 35%, 40%, 45%, or 50%, of the target, defined, preferred, and/or fixed ratio.

In some embodiments, a composition of cells produced by the methods provided herein, e.g., an output composition, having a ratio of engineered CD4+ T cells to engineered CD8+ T cells of between 2:1 and 0.5:1 is administered to a subject in a single composition, dose, or mixture. In certain embodiments, the composition contains a ratio of engineered CD4+ T cells to engineered CD8+ T cells of or of about 1:1. In some embodiments, a cell composition produced from an input cell composition, e.g., an input cell composition described in Section I-A1, has a ratio of engineered CD4+ T cells to engineered CD8+ T cells of between 2:1 and 0.5:1 and is administered to a subject in a single composition, dose, or mixture. In particular embodiments, the cell composition produced from an input cell composition has a ratio of engineered CD4+ T cells to engineered CD8+ T cells of 1:1with a tolerated difference of 50%, 25%, 10%, or less.

### IV. INTERVENTIOND TO PREVENT, REDUCE, OR TREAT TOXICITY

Particular embodiments of the methods provided herein include one or more steps of administering an intervention or interventions to a subject to prevent, reduce, ameliorate, and/or treat neurotoxicity and/or to prevent or reduce the likelihood or probability of neurotoxicity. In certain embodiments, the subject is likely to develop toxicity, e.g., severe neurotoxicity, to a therapy, e.g., an immunotherapy or a cell therapy such as a CAR-T cell therapy, and/or has an increased risk, probability, or likelihood of developing toxicity following administration of the therapy as determined by one or more methods provided in Section I. In certain embodiments, the therapy is an immunotherapy. In particular embodiments, the therapy is a cell therapy. In particular embodiments, the cell therapy is a cell therapy that is or includes CAR expressing cells.

In some embodiments, the intervention is or includes administration of one or more agents or other treatments capable of treating, preventing, delaying, or attenuating the development of a toxicity. In some embodiments, the intervention is administered prior to and/or concurrently with administration of the therapy. In some examples, the agent or other treatment capable of treating, preventing, delaying, or attenuating the development of a toxicity is administered (i) prior to, (ii) within one, two, or three days of, (iii) concurrently with and/or (iv) at first fever following, the initiation of administration of the cell therapy to the subject. In some embodiments, the therapy is an immunotherapy or a cell therapy. In particular embodiments, the therapy is a CAR-T cell therapy.

The agent or treatments are administered following or based on the results of an assessment, in a biological sample (e.g. apheresis or leukapheresis sample) for the expression of one or more genes that are positively or negatively associated with the toxicity following administration of a therapy. In some embodiments, the one or more genes are provided in Section I-B. In particular embodiments, the subject is determined to have an elevated or increased risk of developing a toxicity, e.g., a severe neurotoxicity, based on the expression of one or more genes provided in Section 2, Table 1, Table 2, Table E2A, Table E2B, and/or Table E4 (e.g. a subset of these genes in the relevant Table which as a recited SEQ ID NO, and/or a Uniprot ID), as assessed by the methods described in herein, e.g., Section-I.

A. Toxicity

In some aspects, the provided embodiments are based on observations that the efficacy of a therapy, e.g., a cell therapy, may be limited by the development of toxicity in the subject to whom such cells are administered, which toxicity in some cases can be severe. For example, in some cases, administering a dose of cells expressing a recombinant receptor, e.g. a CAR, can result in toxicity or risk thereof, such as CRS or neurotoxicity. In some cases, while a higher dose of such cells can increase the efficacy of the treatment, for example, by increasing exposure to the cells such as by promoting expansion and/or persistence, they may also result in an even greater risk of developing a toxicity or a more severe toxicity.

In some embodiments, the provided embodiments are designed to or include features that result in a lower degree of risk of toxicity, a toxic outcome or symptom, toxicity-promoting profile, factor or property, such as a symptom or outcome associated with or indicative of cytokine release syndrome (CRS) or neurotoxicity, for example, compared to other methods in which the subject and/or a biological sample from the subject has not been assessed in a assay for safety control and/or in which the therapy has not been administered to a subject in accord with an assessment of any preexisting risk of the subject to toxicity following administration of the therapeutic cell composition.

In some aspects, the toxic outcome of a therapy, such as a cell therapy, is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS, such as severe CRS, typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF-alpha or TNFa), and/or interleukin (IL)-2. Other cytokines that may be rapidly induced in CRS are IL-1β, IL-6, IL-8, and IL-10.

CRS criteria that appear to correlate with the onset of CRS to predict which patients are more likely to be at risk for developing sCRS have been developed (see Davilla et al. Science translational medicine. 2014;6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014;124(2):188-95). In some embodiments, the criteria reflective of CRS grade are those detailed in Table 4 below.

TABLE 4
Exemplary Grading Criteria for CRS

| Grade | Description of Symptoms |
|---|---|
| 1 | Not life-threatening, require only symptomatic |
| Mild | treatment such as antipyretics and anti-emetics |
| | (e.g., fever, nausea, fatigue, headache, |
| | myalgias, malaise) |
| 2 | Require and respond to moderate intervention: |
| Moderate | Oxygen requirement <40%, or |
| | Hypotension responsive to fluids or low dose of |
| | a single vasopressor, or |
| | Grade 2 organ toxicity (by CTCAE v4.0) |
| 3 | Require and respond to aggressive intervention: |
| Severe | Oxygen requirement ≥40%, or |
| | Hypotension requiring high dose of a single |
| | vasopressor (e.g., norepinephrine ≥20 |
| | μg/kg/min, dopamine ≥10 μg/kg/min, |
| | phenylephrine ≥200 μg/kg/min, or epinephrine |
| | ≥10 μg/kg/min), or |
| | Hypotension requiring multiple vasopressors (e.g., |
| | vasopressin + one of the above agents, or |
| | combination vasopressors equivalent to ≥20 |
| | μg/kg/min norepinephrine), or |
| | Grade 3 organ toxicity or Grade 4 transaminitis |
| | (by CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 4.

In some embodiments, outcomes associated with severe CRS or grade 3 CRS or greater, such as grade 4 or greater, such as set forth in Table 4. In some embodiments, these include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNy), GM-CSF, IL-6, IL-10, Flt-3L, fracktalkine, and IL-5, and/or tumor necrosis factor alpha (TNF-alpha or TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, severe CRS encompasses a combination of (1) persistent fever (fever of at least 38 degrees Celsius for at least three days) and (2) a serum level of CRP of at least at or about 20 mg/dL. In some embodiments, severe CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

In some aspects, the toxic outcome of a therapy, such as a cell therapy, is or is associated with or indicative of neurotoxicity or severe neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to, or secondary to, administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 5. In some embodiments, a severe neurotoxicity is deemed to be a prolonged grade 3 if symptoms or grade 3 neurotoxicity last for 10 days or longer.

TABLE 5
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental |
| Moderate | activities of daily living (ADL), such as preparing |
| | meals, shopping for groceries or clothes, using the |
| | telephone, managing money |
| 3 | Presence of symptoms that limit self-care ADL, such |
| Severe | as bathing, dressing and undressing, feeding self, |
| | using the toilet, taking medications |
| 4 | Symptoms that are life-threatening, requiring |
| Life- | urgent intervention |
| threatening | |
| 5 | Death |
| Fatal | |

In some embodiments, the provided embodiments, including methods of treatment, lead to reduced symptoms associated with neurotoxicity following cell therapy compared to other methods. For example, subjects treated according to the provided methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the provided methods may have reduced symptoms associated with peripheral motor neuropathy, peripheral sensory neuropathy, dysesthesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

B. Methods for Intervention

In some embodiments, a subject with a high, elevated, and/or increased risk of a toxicity following administration of a therapeutic cell composition is administered an intervention or interventions, including by administration of agents or therapies that treat the toxicity (e.g. neurotoxicity, such as severe neurotoxicity and/or CRS, such as severe CRS) and/or that prevent, delay, or attenuate the development of or risk for developing neurotoxicity, e.g., severe neurotoxicity and/or CRS, such as severe CRS. Thus, in some embodiments, provided herein is an intervention or interventions, including agents or therapies that treat the toxicity and/or that prevent, delay, or attenuate the development of or risk for developing neurotoxicity or CRS. Also provided are compositions and formulations, e.g., pharmaceutical formulations, comprising one or more of the agents.

In some embodiments, the methods provided herein allows for selection of a subject for an intervention, e.g., administration of an agent or other treatment capable of treating, preventing, delaying, reducing or attenuating the development or risk of development of a toxicity following administration of the therapy, by identifying subject at with a likelihood and/or an increased risk, probability, or likelihood of developing or experiencing toxicity following administration of a therapy, e.g., an immunotherapy or cell therapy. In some embodiments, the intervention is administered (i) prior to, (ii) within one, two, or three days of, (iii) concurrently with and/or (iv) at first fever following, the initiation of administration of the cell therapy to the subject.

In some embodiments, the subject is not administered or provided with an intervention, e.g., an agent or treatment capable of treating, preventing, delaying, reducing or attenuating the development or risk of development of a toxicity such as described in Section IV-C, prior to the administration of the therapy, e.g., the immunotherapy and/or cell therapy. In some embodiments, the subject is not administered or provided with the intervention for a period of time following the initiation of the administration of the therapy. In some embodiments, the period of time is or is about 1, 2, 3, 4, 5, 6, 7, 8, 9, 10, 11, 12, 13, 14, or 15 days, and/or is or is about 1, 2, 3, 4, 5, 6, or longer than 6 weeks after the administration of the therapy. In some embodiments, the subject is not administered or provided with the intervention following administration of the dose, prior to or unless the subject exhibits a sign or symptom of toxicity, e.g., a sign or symptom of toxicity described in Section IV-A. In some embodiments, the subject is not administered or provided with the intervention following administration of the dose, prior to or unless the subject exhibits a sign or symptom of the toxicity other than a fever, unless the fever is a sustained fever or if the fever is not reduced by more than 1° C. after treatment with an antipyretic.

In some embodiments, the subject is administered or provided with an intervention, e.g., an agent or treatment capable of treating, preventing, delaying, reducing or attenuating the development or risk of development of a toxicity such as described in Section IV-C, prior to the administration of the therapy, e.g., the immunotherapy and/or cell therapy. In some embodiments, the subject has been determined to have a high, increased, or elevated risk for toxicity. In some embodiments, the subject is administered or provided with an intervention within period of time following the initiation of the administration of the therapy, such as at or about within 1, 2, 3, 4, 5 days after the administration of the therapy. In some embodiments, the subject is administered or provided with the intervention at the first sign or symptom of toxicity, e.g., a sign or symptom of toxicity described in Section IV-A.

In some embodiments, the agent, e.g., a toxicity-targeting agent, or treatment capable of treating, preventing, delaying, or attenuating the development of a toxicity is a steroid, is an antagonist or inhibitor of a cytokine receptor, such as IL-6 receptor, CD122 receptor (IL-2Rbeta receptor), or CCR2, or is an inhibitor of a cytokine, such as IL-6, MCP-1, IL-10, IFN-γ, IL-8, or IL-18. In some embodiments, the agent is an agonist of a cytokine receptor and/or cytokine, such as TGF-β. In some embodiments, the agent, e.g., agonist, antagonist or inhibitor, is an antibody or antigen-binding fragment, a small molecule, a protein or peptide, or a nucleic acid.

In some embodiments, a fluid bolus can be employed as an intervention, such as to treat hypotension associated with CRS. In some embodiments, the target hematocrit levels are >24%. In some embodiments, the intervention includes the use of absorbent resin technology with blood or plasma filtration. In some cases, the intervention includes dialysis, plasmapheresis, or similar technologies. In some embodiments, vasopressors or acetaminophen can be employed.

In some embodiments, the agent can be administered sequentially, intermittently, or at the same time as or in the same composition as the therapy, such as cells for adoptive cell therapy. For example, the agent can be administered before, during, simultaneously with, or after administration of the immunotherapy and/or cell therapy.

In some embodiments, the agent is administered at a time as described herein and in accord with the provided methods. In some embodiments, the toxicity-targeting agent is administered at a time that is within, such as less than or no more than, 3, 4, 5, 6, 7, 8, 9 or 10 days after initiation of the immunotherapy and/or cell therapy. In some embodiments, the toxicity-targeting agent is administered within or within about 1 day, 2 days or 3 days after initiation of administration of the immunotherapy and/or cell therapy.

In some embodiments, the agent, e.g., toxicity-targeting agent, is administered to a subject after initiation of administration of the immunotherapy and/or cell therapy at a time at which the subject does not exhibit grade 2 or higher CRS or grade 2 or higher neurotoxicity. In some embodiments, the subject has been determined to be at high risk for toxicity and/or to have an increased risk, probability or likelihood of developing toxicity following administration of the therapy, for example by the methods provided in Section I. In some aspects, the toxicity-targeting agent is administered after initiation of administration of the immunotherapy and/or cell therapy at a time at which the subject does not exhibit severe CRS or severe neurotoxicity. Thus, between initiation of administration of the immunotherapy and/or cell therapy and the toxicity-targeting agent, the subject, e.g., the subject at risk for toxicity, is one that does not exhibit grade 2 or higher CRS, such as severe CRS, and/or does not exhibit grade 2 or higher neurotoxicity, such as severe neurotoxicity.

Non-limiting examples of interventions for treating or ameliorating a toxicity, such as severe CRS (sCRS), are described in Table 6A. In some embodiments, the intervention includes tocilizumab or other toxicity-targeting agent as described, which can be at a time in which there is a sustained or persistent fever of greater than or about 38° C. or greater than or greater than about 39° C. in the subject. In some embodiments, the fever is sustained in the subject for more than 10 hours, more than 12 hours, more than 16 hours, or more than 24 hours before intervention.

TABLE 6A
Examples of interventions for treating or ameliorating a toxicity

| Symptoms related to CRS | Suggested Intervention |
|---|---|
| Fever of ≥38.3° C. | Acetaminophen (12.5 mg/kg) PO/IV up to |
| | every four hours |
| Persistent fever of ≥39° C. for 10 hours that is | Tocilizumab (8-12 mg/kg) IV |
| unresponsive to acetaminophen | |
| Persistent fever of ≥39° C. after tocilizumab | Dexamethasone 5-10 mg IV/PO up to every 6- |
| | 12 hours with continued fevers |
| Recurrence of symptoms 48 hours after initial | Tocilizumab (8-12 mg/kg) IV |
| dose of tocilizumab | |
| Hypotension | Fluid bolus, target hematocrit >24% |
| Persistent/recurrent hypotension after initial | Tocilizumab (8-12 mg/kg) IV |
| fluid bolus (within 6 hours) | |
| Use of low dose pressors for hypotension for | Dexamethasone 5-10 mg IV/PO up to every 6 |
| longer than 12 hours | hours with continued use of pressors |
| Initiation of higher dose pressors or addition of | Dexamethasone 5-10 mg IV/PO up to every 6 |
| a second pressor for hypotension | hours with continued use of pressors |
| Initiation of oxygen supplementation | Tocilizumab (8-12 mg/kg) IV |
| Increasing respiratory support with concern for | Dexamethasone 5-10 mg IV/PO up to every 6 |
| impending intubation | hours with continued use of pressors |
| Recurrence/Persistence of symptoms for which | Tocilizumab (8-12 mg/kg) IV |
| tocilizumab was given ≥48 hours after initial | |
| dose was administered | |

In some cases, the agent or treatment is administered alone or is administered as part of a composition or formulation, such as a pharmaceutical composition or formulation, as described herein. Thus, the agent alone or as part of a pharmaceutical composition can be administered intravenously or orally, or by any other acceptable known route of administration or as described herein.

In some embodiments, the dosage of agent or the frequency of administration of the agent in a dosage regimen is reduced compared to the dosage of the agent or its frequency in a method in which a subject is treated with the agent after grade 2 or higher CRS or neurotoxicity, such as after severe, e.g., grade 3 or higher, CRS or after severe, e.g., grade 3 or higher neurotoxicity, has developed or been diagnosed (e.g. after physical signs or symptoms of grade 3 or higher CRS or neurotoxicity has manifested). In some embodiments, the dosage of agent or the frequency of administration of the agent in a dosage regimen is reduced compared to the dosage of the agent or its frequency in a method in which a subject is treated for CRS or neurotoxicity greater than 3 days, 4 days, 5 days, 6 days, 1 week, 2 weeks, three weeks, or more after administration of the immunotherapy and/or cell therapy. In some embodiments, the dosage is reduced by greater than or greater than about 1.2-fold, 1.5-fold, 2-fold, 3-fold, 4-fold, 5-fold, 6-fold, 7-fold, 8-fold, 9-fold, 10-fold or more. In some embodiments, the dosage is reduced by greater than or about 10%, 20%, 30%, 40%, 50%, 60%, 70%, 80%, 90%, or more. In some embodiments, the frequency of dosing is reduced, such as the number of daily doses is reduced or the number of days of dosing is reduced.

C. Agents for Intervention

In some embodiments, the agent, e.g., toxicity-targeting agent, is an agent that treats and/or that prevents, delays, or attenuates the development of or risk for developing a toxicity following administration of an immunotherapy and/or a cell therapy. In some embodiments, the agent is a steroid, is an antagonist or inhibitor of a cytokine receptor, such as IL-6 receptor, CD122 receptor (IL-2R/IL-15Rbeta receptor), or CCR2, or is an inhibitor of a cytokine, such as IL-6, IL-15, MCP-1, IL-10, IFN-γ, IL-8, or IL-18. In some embodiments, the agent is an agonist of a cytokine receptor and/or cytokine, such as TGF-β. In some embodiments, the agent, e.g., agonist, antagonist or inhibitor, is an antibody or antigen-binding fragment, a small molecule, a protein or peptide, or a nucleic acid. In some embodiments, the agent is an anti-histamine.

In some embodiments, the intervention includes the use of absorbent resin technology with blood or plasma filtration. In some cases, the intervention includes dialysis, plasmapheresis, or similar technologies.

In some embodiments, the agent can be administered sequentially, intermittently, or at the same time as or in the same composition as cells for adoptive cell therapy. For example, the agent can be administered before, during, simultaneously with, or after administration of the cell therapy.

In some embodiments, the agent is administered at a time following administration of cell therapy when the subject has been identified or predicted to be at risk for developing neurotoxicity and/or CRS, such as severe CRS. In some embodiments, the agent is administered at a time following administration of cell therapy when the subject has been identified or predicted to be at risk for developing neurotoxicity and/or CRS, but before physical symptoms of severe neurotoxicity, such as neurotoxicity of grade 3 or above and/or severe CRS, such as a CRS of grade 3 or above, manifest.

In some embodiments, the agent is administered at a time at which a clinical risk for neurotoxicity and/or CRS is detected to be present following the administration of cell therapy. In some embodiments, the agent is administered at a time at which a biochemical readout evidencing neurotoxicity and/or CRS is detected following administration of cell therapy. In some embodiments, the agent is administered at a time at which a serum level of a factor indicative of neurotoxicity and/or CRS in the subject indicates a higher risk for the development of neurotoxicity and/or CRS as compared to the serum level of the indicator in the subject immediately prior to said administration of the cells. In some embodiments, the agent is administered at a time at which the subject does not exhibit neurotoxicity and/or CRS, does not exhibit severe neurotoxicity and/or CRS, or does not exhibit neurotoxicity and/or CRS above grade 3 following administration of cell therapy.

In some embodiments, administration of the agent reduces symptoms associated with neurotoxicity and/or CRS compared to other methods. For example, subjects treated with the agent may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects who do not receive the agent, or receive the agent at a time when physical symptoms of neurotoxicity have manifested in the subject. In some embodiments, subjects treated with the agent according to the provided methods may have reduced symptoms associated with peripheral motor neuropathy, peripheral sensory neuropathy, dysesthesia, neuralgia or paresthesia.

In some embodiments, the administration of the agent according to the provided methods reduces outcomes associated with neurotoxicity including damages to the nervous system and/or brain, such as the death of neurons. In some aspects, the administration of the agent reduces the level of factors associated with neurotoxicity such as beta amyloid (Aβ), glutamate, and oxygen radicals.

In some embodiments, the administration of the agent according to the provided methods reduces outcomes associated with CRS including fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. In some embodiments, the administration of the agent according to the provided methods reduces outcomes associated with CRS including such as fatigue, nausea, headache, seizure, tachycardia, myalgias, rash, acute vascular leak syndrome, liver function impairment, and renal failure. In some embodiments, the administration of the agent according to the provided methods reduces outcomes associated with CRS including such as an increase in one or more factors such as serum-ferritin, d-dimer, aminotransferases, lactate dehydrogenase and triglycerides, or with hypofibrinogenemia or hepatosplenomegaly.

Thus, in some embodiments, subjects administered the agent that treats, prevents, or reduces the risk of developing neurotoxicity and/or CRS have reduced symptoms, outcomes, or factors associated with neurotoxicity and/or CRS compared to subjects who are not administered the agent, and/or subjects who are administered the agent at a time at which the subject exhibits clinical signs or symptoms of neurotoxicity or severe neurotoxicity, e.g., neurotoxicity of grade 3 or higher and/or severe CRS, such as a CRS of grade 3 or above.

In some embodiments, the agent can be administered greater than 4 hours, 5 hours, 6 hours, 7 hours, 8 hours, 9 hours, 10 hours, 11 hours, 12 hours, 18 hours, 24 hours, 36 hours, 2 days, 3 days, 4 days, or 5 days or more following administration of the cell therapy. In some of such embodiments, the agent may be administered no later than 5 hours, 6 hours, 7 hours, 8 hours, 9 hours, 10 hours, 11 hours, 12 hours, 18 hours, 24 hours, 36 hours, 2 days, 3 days, 4 days, or 5 days or more following administration of the cell therapy.

In some aspects, the agent can be administered between or between about 4 hours and 5 days following administration of cell therapy, such as between or between about 4 hours and 4 days, 8 hours and 3 days, 1 day and 3 days, 2 days and 3 days, or 1 day and 2 days following administration of cell therapy. In some such cases, the agent is administered at or about 1 day, at or about 2 days, or at or about 3 days following the administration of cell therapy. In some instances, the subject is treated with the agent within 3 days, within 2 days or within 1 day after administration of the cell therapy.

In some cases, the agent is administered alone or is administered as part of a composition or formulation, such as a pharmaceutical composition or formulation, as described herein. Thus, the agent alone or as part of a pharmaceutical composition can be administered intravenously or orally, or by any other acceptable known route of administration or as described herein.

In some embodiments, the dosage of agent or the frequency of administration of the agent in a dosage regimen is reduced compared to the dosage of the agent or its frequency in a method in which a subject is treated with the agent after severe, e.g., grade 3 or higher, neurotoxicity has developed or been diagnosed (e.g. after physical signs or symptoms of grade 3 or higher neurotoxicity has manifested). In some embodiments, the dosage of agent or the frequency of administration of the agent in a dosage regimen is reduced compared to the dosage of the agent or its frequency in a method in which a subject is treated for neurotoxicity greater than 3 days, 4 days, 5 days, 6 days, 1 week 2 weeks, three weeks, or more after administration of the cell therapy. In some embodiments, the dosage is reduced by greater than or about 10%, 20%, 30%, 40%, 50%, 60%, 70%, 80%, 90%, or more. In some embodiments, the frequency of dosing is reduced, such as the number of daily doses is reduced or the number of days of dosing is reduced.

1. Steroid

In some embodiments, the agent that treats neurotoxicity and/or CRS, and/or that prevents, delays, or attenuates the development of or risk for developing severe neurotoxicity is a steroid, e.g., corticosteroid. Corticosteroids typically include glucocorticoids and mineralocorticoids.

Generally, any corticosteroid, e.g., glucocorticoid, can be used in the methods or compositions provided herein. In some embodiments, glucocorticoids include synthetic and non-synthetic glucocorticoids. Exemplary glucocorticoids include, but are not limited to: alclomethasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980).

In some examples, the glucocorticoid is selected from among cortisones, dexamethasones, hydrocortisones, methylprednisolones, prednisolones and prednisones. In a particular example, the glucocorticoid is dexamethasone.

In some embodiments, the agent is a corticosteroid and is administered in an amount that is therapeutically effective to treat, ameliorate or reduce one or more symptoms of neurotoxicity and/or CRS. In some embodiments, indicators of improvement or successful pretreatment include determination of the failure to manifest a relevant score on neurotoxicity and/or CRS grading scale, such as a score of less than 3, or a change in grading or severity on the neurotoxicity and/or CRS grading scale as discussed herein, such as a change from a score of 4 to a score of 3.

In some aspects, the corticosteroid is provided in a therapeutically effective dose. Therapeutically effective concentration can be determined empirically by testing in known in vitro or in vivo (e.g. animal model) systems. For example, the amount of a selected corticosteroid to be administered to ameliorate symptoms or adverse effects of neurotoxicity and/or CRS can be determined by standard clinical techniques. In addition, animal models can be employed to help identify optimal dosage ranges. The precise dosage, which can be determined empirically, can depend on the particular therapeutic preparation, the regime and dosing schedule, the route of administration and the seriousness of the disease.

The corticosteroid can be administered in any amount that is effective to ameliorate one or more symptoms associated with neurotoxicity and/or CRS. Thus, the corticosteroid, e.g., glucocorticoid, can be administered, for example, at an amount between at or about 0.1 and 100 mg, per dose, 0.1 to 80 mg, 0.1 to 60 mg, 0.1 to 40 mg, 0.1 to 30 mg, 0.1 to 20 mg, 0.1 to 15 mg, 0.1 to 10 mg, 0.1 to 5 mg, 0.2 to 40 mg, 0.2 to 30 mg, 0.2 to 20 mg, 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg, 0.4 to 5 mg, 0.4 to 4 mg, 1 to 20 mg, 1 to 15 mg or 1 to 10 mg, to a 70 kg adult human subject. Typically, the corticosteroid, such as a glucocorticoid is administered at an amount between at or about 0.4 and 20 mg, for example, at or about 0.4 mg, 0.5 mg, 0.6 mg, 0.7 mg, 0.75 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg or 20 mg per dose, to an average adult human subject.

In some embodiments, the corticosteroid can be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

The corticosteroid, or glucocorticoid, for example dexamethasone, can be administered orally (tablets, liquid or liquid concentrate), PO, intravenously (IV), intramuscularly or by any other known route or route described herein (e.g., with respect to pharmaceutical formulations). In some aspects, the corticosteroid is administered as a bolus, and in other aspects it may be administered over a period of time.

In some aspects, the glucocorticoid can be administered over a period of more than one day, such as over two days, over 3 days, or over 4 or more days. In some embodiments, the corticosteroid can be administered one per day, twice per day, or three times or more per day. For example, the corticosteroid, e.g., dexamethasone, may in some examples be administered at 10 mg (or equivalent) IV twice a day for three days.

In some embodiments, the dosage of corticosteroid, e.g., glucocorticoid, is administered in successively lower dosages per treatment. Hence, in some such treatment regimes, the dose of corticosteroid is tapered. For example, the corticosteroid may be administered at an initial dose (or equivalent dose, such as with reference to dexamethasone) of 4 mg, and upon each successive administration the dose may be lowered, such that the dose is 3 mg for the next administration, 2 mg for the next administration, and 1 mg for the next administration.

Generally, the dose of corticosteroid administered is dependent upon the specific corticosteroid, as a difference in potency exists between different corticosteroids. It is typically understood that drugs vary in potency, and that doses can therefore vary, in order to obtain equivalent effects. Table 6B shows equivalence in terms of potency for various glucocorticoids and routes of administration. Equivalent potency in clinical dosing is well known. Information relating to equivalent steroid dosing (in a non-chronotherapeutic manner) may be found in the British National Formulary (BNF), 37 March 1999.

TABLE 6B
Glucocorticoid administration

| Glucocorticoid (Route) | Equivalency Potency |
|---|---|
| Hydrocortisone (IV or PO) | 20 |
| Prednisone | 5 |
| Prednisolone (IV or PO) | 5 |
| Methylprednisolone sodium succinate (IV) | 4 |
| Dexamethasone (IV or PO) | 0.5-0.75 |

Thus, in some embodiments, the steroid is administered in an equivalent dosage amount of from or from about 1.0 mg to 20 mg dexamethasone per day, such as 1.0 mg to 15 mg dexamethasone per day, 1.0 mg to 10 mg dexamethasone per day, 2.0 mg to 8 mg dexamethasone per day, or 2.0 mg to 6.0 mg dexamethasone per day, each inclusive. In some cases, the steroid is administered in an equivalent dose of at or about 4 mg or at or about 8 mg dexamethasone per day.

In some embodiments, the steroid is administered if fever persists after treatment with tocilizumab. For example, in some embodiments, dexamethasone is administered orally or intravenously at a dosage of 5-10 mg up to every 6-12 hours with continued fevers. In some embodiments, tocilizumab is administered concurrently with or subsequent to oxygen supplementation.

2. Inhibitors of Microglial Cell Activity

In some embodiments, the agent is an inhibitor of a microglial cell activity. In some embodiments, the administration of the inhibitor modulates the activity of microglia. In some embodiments, the inhibitor is an antagonist that inhibits the activity of a signaling pathway in microglia. In some embodiments, the microglia inhibitor affects microglial homeostasis, survival, and/or proliferation. In some embodiments, the inhibitor targets the CSF1R signaling pathway. In some embodiments, the inhibitor is an inhibitor of CSF1R. In some embodiments, the inhibitor is a small molecule. In some cases, the inhibitor is an antibody.

In some aspects, administration of the inhibitor results in one or more effects selected from an alteration in microglial homeostasis and viability, a decrease or blockade of microglial cell proliferation, a reduction or elimination of microglial cells, a reduction in microglial activation, a reduction in nitric oxide production from microglia, a reduction in nitric oxide synthase activity in microglia, or protection of motor neurons affected by microglial activation. In some embodiments, the agent alters the level of a serum or blood biomarker of CSF1R inhibition, or a decrease in the level of urinary collagen type 1 cross-linked N-telopeptide (NTX) compared to at a time just prior to initiation of the administration of the inhibitor. In some embodiments, the administration of the agent transiently inhibits the activity of microglia activity and/or wherein the inhibition of microglia activity is not permanent. In some embodiments, the administration of the agent transiently inhibits the activity of CSF1R and/or wherein the inhibition of CSF1R activity is not permanent.

In some embodiments, the agent that reduces microglial cell activity is a small molecule, peptide, protein, antibody or antigen-binding fragment thereof, an antibody mimetic, an aptamer, or a nucleic acid molecule. In some embodiments, the method involves administration of an inhibitor of microglia activity. In some embodiments, the agent is an antagonist that inhibits the activity of a signaling pathway in microglia. In some embodiments, the agent that reduces microglial cell activity affects microglial homeostasis, survival, and/or proliferation.

In some embodiments, the agent that reduces microglial cell activation is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-KB), activates a CB₂ receptor and/or is a CB₂ agonist, a phosphodiesterase inhibitor, inhibits microRNA-155 (miR-155), upregulates microRNA-124 (miR-124), inhibits nitric oxide production in microglia, inhibits nitric oxide synthase, or activates the transcription factor NRF2 (also called nuclear factor (erythroid-derived 2)-like 2, or NFE2L2).

In some embodiments, the agent that reduces microglial cell activity targets CSF1 (also called macrophage colony-stimulating factor MCSF). In some embodiments, the agent that reduces microglial cell activity affects MCSF-stimulated phosphorylation of the M-CSF receptor (Pryer et al. Proc Am Assoc Cancer Res, AACR Abstract nr DDT02-2 (2009)). In some cases, the agent that reduces microglial cell activity is MCS110 (international patent application publication number WO2014001802; Clinical Trial Study Record Nos.:A1 NCT00757757; NCT02807844; NCT02435680; NCT01643850).

In some embodiments, the agent that reduces microglial cell activity is a small molecule that targets the CSF1 pathway. In some embodiments, the agent is a small molecule that binds CSF1R. In some embodiments, the agent is a small molecule which inhibits CSF1R kinase activity by competing with ATP binding to CSF1R kinase. In some embodiments, the agent is a small molecule which inhibits the activation of the CFS1R receptor. In some cases, the binding of the CSF-1 ligand to the CSF1R is inhibited. In some embodiments, the agent that reduces microglial cell activity is any of the inhibitors described in US Patent Application Publication Number US20160032248.

In some embodiments, the agent is a small molecule inhibitor selected from PLX-3397, PLX7486, JNJ-40346527, JNJ28312141, ARRY-382, PLX73086 (AC-708), DCC-3014, AZD6495, GW2580, Ki20227, BLZ945, PLX647, PLX5622. In some embodiments, the agent is any of the inhibitors described in Conway et al., Proc Natl Acad Sci USA, 102(44):16078-83 (2005); Dagher et al., Journal of Neuroinflammation, 12:139 (2015); Ohno et al., Mol Cancer Ther. 5(11):2634-43 (2006); von Tresckow et al., Clin Cancer Res.,21(8) (2015); Manthey et al. Mol Cancer Ther. (8(11):3151-61 (2009); Pyonteck et al., Nat Med. 19(10): 1264-1272 (2013); Haegel et al., Cancer Res AACR Abstract nr 288 (2015); Smith et al., Cancer Res AACR Abstract nr 4889 (2016); Clinical Trial Study Record Nos.: NCT01525602; NCT02734433; NCT02777710; NCT01804530; NCT01597739; NCT01572519; NCT01054014; NCT01316822; NCT02880371; NCT02673736; international patent application publication numbers WO2008063888A2, WO2006009755A2, US patent application publication numbers US20110044998, US 2014/0065141, and US 2015/0119267.

In some embodiments, the agent that reduces microglial cell activity is 4-((2-(((1R,2R)-2-hydroxycyclohexyl)amino)benzo[d]thiazol-6-yl)oxy)-N-methylpicolinamide (BLZ945) or a pharmaceutically acceptable salt thereof or derivatives thereof. In some embodiments, the agent is the following compound:

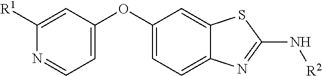

wherein R1 is an alkyl pyrazole or an alkyl carboxamide, and R2 is a hydroxycycloalkyl or a pharmaceutically acceptable salt thereof.

In some embodiments, the agent that reduces microglial cell activity is 5-((5-chloro-1H-pyrrolo[2,3-b]pyridin-3-yl)methyl)-N-((6-(trifluoromethyl)pyridin-3-yl)methyl)pyridin-2-amine, N-[5-[(5-Chloro-1H-pyrrolo[2,3-b]pyridin-3-yl)methyl]−2-pyridinyl]−6-(trifluoromethyl)-3-pyridinemethanamine) (PLX 3397) or a pharmaceutically acceptable salt thereof or derivatives thereof. In some embodiments, the agent is 5-(1H-Pyrrolo[2,3-b]pyridin-3-ylmethyl)-N-[[4-(trifluoromethyl)phenyl]methyl]−2-pyridinamine dihydrochloride (PLX647) or a pharmaceutically acceptable salt thereof or derivatives thereof. In some embodiments, the agent that reduces microglial cell activity is the following compound:

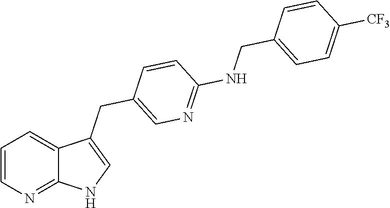 [SMILES: FC(F)(F)c1ccc(CNc2ccc(Cc3c[nH]c4ncccc34)cn2)cc1]

or a pharmaceutically acceptable salt thereof. In some embodiments, the agent that reduces microglial cell activity is the following compound:

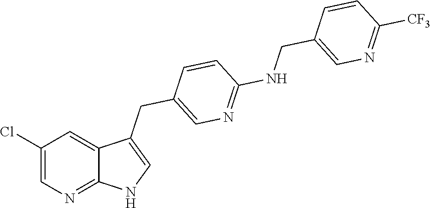 [SMILES: FC(F)(F)c1ccc(CNc2ccc(Cc3c[nH]c4ncc(Cl)cc34)cn2)cn1]

or a pharmaceutically acceptable salt thereof. In some embodiments, the agent is any of the inhibitors described in US patent number U.S. Pat. No. 7,893,075.

In some embodiments, the agent that reduces microglial cell activity is 4-cyano-N-[2-(1-cyclohexen-l-yl)-4-[1-[(dimethylamino)acetyl]−4-piperidinyl]phenyl]−1H-imidazole-2-carboxamide monohydrochloride (JNJ28312141) or a pharmaceutically acceptable salt thereof or derivatives thereof. In some embodiments, the agent is the following compound:

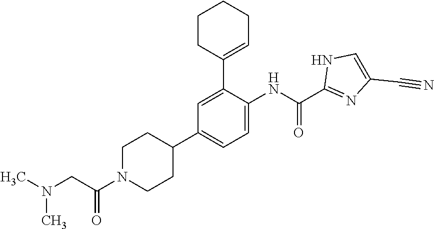 [SMILES: CN(C)CC(=O)N1CCC(c2ccc(NC(=O)c3nc(C#N)c[nH]3)c(C3=CCCCC3)c2)CC1]

or a pharmaceutically acceptable salt thereof. In some embodiments, the agent is any of the inhibitors described in US patent number U.S. Pat. No. 7,645,755.

In some embodiments, the agent that reduces microglial cell activity is 1H-Imidazole-2-carboxamide, 5-cyano-N-(2-(4,4-dimethyl-l-cyclohexen-l-yl)-6-(tetrahydro-2,2,6,6-tetramethyl-2H-pyran-4-yl)-3-pyridinyl)-, 4-Cyano-1H-imidazole-2-carboxylic acid N-(2-(4,4-dimethylcyclohex-1-enyl)-6-(2,2,6,6-tetramethyltetrahydropyran-4-yl)pyridin-3-yl)amide, 4-Cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527) or a pharmaceutically acceptable salt thereof or derivatives thereof. In some embodiments, the agent is the following compound:

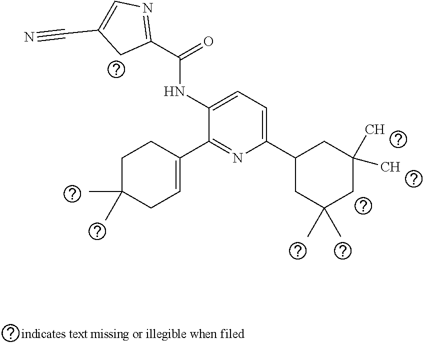 [SMILES: CC1(C)CC=C(c2nc(C3CC(C)(C)CC(C)(C)C3)ccc2NC(=O)C2=NC=C(C#N)C2)CC1]

or a pharmaceutically acceptable salt thereof.

In another embodiment, the agent that reduces microglial cell activity is 5-(3-Methoxy-4-((4-methoxybenzyl)oxy)benzyl)pyrimidine-2,4-diamine (GW2580) or a pharmaceutically acceptable salt thereof or derivatives thereof. In some embodiments, the agent is the following compound:

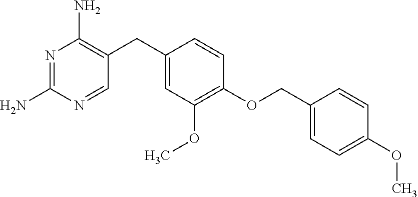 [SMILES: COc1ccc(COc2ccc(Cc3cnc(N)nc3N)cc2OC)cc1]

or a pharmaceutically acceptable salt thereof (international patent application publication number WO2009099553).

In some embodiments, the agent that reduces microglial cell activity is 4-(2,4-difluoroanilino)-7-ethoxy-6-(4-methylpiperazin-l-yl)quinoline-3-carboxamide (AZD6495) or a pharmaceutically acceptable salt thereof or derivatives thereof. In some embodiments, the agent is the following compound:

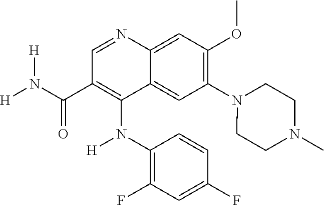 [SMILES: COc1cc2ncc(C(N)=O)c(Nc3ccc(F)cc3F)c2cc1N1CCN(C)CC1]

or a pharmaceutically acceptable salt thereof.

In some embodiments, the agent that reduces microglial cell activity is N-{4-[(6,7-dimethoxy-4-quinolyl)oxy]-2-methoxyphenyl}-N0-[1-(1,3-thiazole-2-yl)ethyl]urea (Ki20227) or a pharmaceutically acceptable salt thereof or derivatives thereof. In some embodiments, the agent is the following compound:

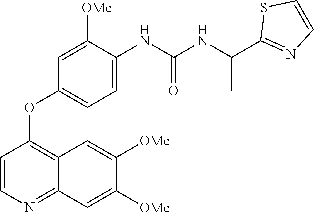 [SMILES: COc1cc(Oc2ccnc3cc(OC)c(OC)cc23)ccc1NC(=O)NC(C)c1nccs1]

or a pharmaceutically acceptable salt thereof.

In some embodiments, the agent that reduces microglial cell activation is an antibody that targets the CSF1 pathway. In some embodiments, the agent is an antibody that binds CSF1R. In some embodiments, the anti-CSF1R antibody blocks CSF1R dimerization. In some embodiments, the anti-CSF1R antibody blocks the CSF1R dimerization interface that is formed by domains D4 and D5 (Ries et al. Cancer Cell 25(6):846-59 (2014)). In some cases, the agent is selected from emactuzumab (RG7155; R05509554), Cabiralizumab (FPA-008), LY-3022855 (IMC-CS4), AMG-820, TG-3003, MCS110, H27K15, 12-2D6, 2-4A5 (Rovida and Sbarba, J Clin Cell Immunol.6:6 (2015); Clinical Trial Study Record Nos.: NCT02760797; NCT01494688; NCT02323191; NCT01962337; NCT02471716; NCT02526017; NCT01346358; NCT02265536; NCT01444404; NCT02713529, NCT00757757; NCT02807844; NCT02435680; NCT01643850).

In some embodiments, the agent that reduces microglial cell activation is a tetracycline antibiotic. For example, the agent affects IL-lb, IL-6, TNFa, or iNOS concentration in microglia cells (Yrjänheikki et al. PNAS 95(26): 15769-15774 (1998); Clinical Trial Study Record No: NCT01120899). In some embodiments, the agent is an opioid antagonist (Younger et al. Pain Med. 10(4):663-672 (2009.) In some embodiments, the agent reduces glutamatergic neurotransmission (U.S. Pat. No. 5,527,814). In some embodiments, the agent modulates NFkB signaling (Valera et al J. Neuroinflammation 12:93 (2015); Clinical Trial Study Record No: NCT00231140). In some embodiments, the agent targets cannabinoid receptors (Ramírez et al. J. Neurosci 25(8):1904-13(2005)). In some embodiments, the agent is selected from minocycline, naloxone, riluzole, lenalidomide, and a cannabinoid (optionally WIN55 or 212-2).

Nitric oxide production from microglia is believed, in some cases, to result in or increase neurotoxicity. In some embodiments, the agent modulates or inhibits nitric oxide production from microglia. In some embodiments, the agent inhibits nitric oxide synthase (NOS). In some embodiments, the NOS inhibitor is Ronopterin (VAS-203), also known as 4-amino-tetrahydrobiopterin (4-ABH4). In some embodiments, the NOS inhibitor is cindunistat, A-84643, ONO-1714, L-NOARG, NCX-456, VAS-2381, GW-273629, NXN-462, CKD-712, KD-7040, or guanidinoethyldisulfide. In some embodiments, the agent is any of the inhibitors described in Ming et al., Cell Stem Cell. 2012 Nov. 2;11(5):620-32.

In some embodiments, the agent blocks T cell trafficking, such as to the central nervous system. In some embodiments, blocking T cell trafficking can reduce or prevent immune cells from crossing blood vessel walls into the central nervous system, including crossing the blood-brain barrier. In some cases, activated antigen-specific T cells produce proinflammatory cytokines, including IFN-γ and TNF, upon reactivation in the CNS, leading to activation of resident cells such as microglia and astrocytes. See Kivisäkk et al., Neurology. 2009 Jun. 2; 72(22): 1922-1930. Thus, in some embodiments, sequestering activated T cells from microglial cells, such as by blocking trafficking and/or inhibiting the ability of such cells to cross the blood-brain barrier, can reduce or eliminate microglial activation. In some embodiments, the agent inhibits adhesion molecules on immune cells, including T cells. In some embodiments, the agent inhibits an integrin. In some embodiments, the integrin is alpha-4 integrin. In some embodiments, the agent is natalizumab (Tysabri®). In some embodiments, the agent modulates a cell surface receptor. In some embodiments, the agent modulates the sphingosine-1-phosphate (S1P) receptor, such as S1PR1 or S1PR5. In some embodiments, the agent causes the internalization of a cellular receptor, such as a sphingosine-1-phosphate (S1P) receptor, such as S1PR1 or S1PR5. In some embodiments, the agent is fingolimod (Gilenya®) or ozanimod (RPC-1063).

The transcription factor NRF2 is believed to regulate the anti-oxidant response, for example, by turning on genes that contain a cis-acting element in their promoter region. An example of such an element includes an antioxidant response element (ARE). In some embodiments, the agent activates NRF2. In some embodiments, activating NRF2 in microglial cells reduces the microglial cells' responsiveness to IFN and LPS. In some embodiments, activating NRF2 inhibits, slows, or reduces demyelination, axonal loss, neuronal death, and/or oligodendrocyte death. In some embodiments, the agent upregulates the cellular cytoprotective pathway regulated by NRF2. In some embodiments, the agent that activates NRF2 is dimethyl fumarate (Tecfidera®). In some embodiments, the agent is any of the inhibitors described in U.S. Pat. No. 8,399,514. In some embodiments, the agent is any of the inhibitors described in Hoing et al., Cell Stem Cell. 2012 Nov 2;11(5):620-32.

In some embodiments, the agent that reduces microglial cell activation is (4S,4aS,5aR,12aS)-4,7-bis(dimethylamino)-3,10,12,12a-tetrahydroxy-1,11-dioxo-1,4,4a,5,5a,6,11,12a-octahydrotetracene-2-carboxamide (Minocycline) or a pharmaceutically acceptable salt thereof or derivatives thereof. In some embodiments, the agent is any of the compounds described in US patent application publication number US20100190755. In some embodiments, the agent is the following compound:

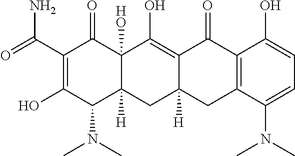 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

or a pharmaceutically acceptable salt thereof.

In some embodiments, the agent that reduces microglial cell activation is 3-(7-amino-3-oxo-1H-isoindol-2-yl)piperidine-2,6-dione (lenalidomide) or a pharmaceutically acceptable salt thereof or derivatives thereof. In some embodiments, the agent is the following compound:

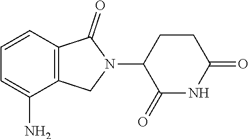 [SMILES: Nc1cccc2c1CN(C1CCC(=O)NC1=O)C2=O]

or a pharmaceutically acceptable salt thereof.

In some embodiments, the agent that reduces microglial cell activation is 4R,4aS,7aR,12bS)-4a,9-dihydroxy-3-prop-2-enyl-2,4,5,6,7a,13-hexahydro-1H-4,12-methanobenzofuro[3,2-e]isoquinoline-7-one (naloxone) or a pharmaceutically acceptable salt thereof or derivatives thereof. In some embodiments, the agent is any of the compounds described in US patent number U.S. Pat. No. 8,247,425. In some embodiments, the agent is the following compound:

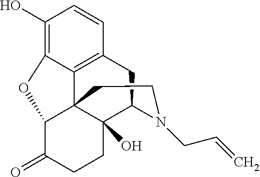 [SMILES: C=CCN1CC[C@]23c4c5ccc(O)c4O[C@H]2C(=O)CC[C@@]3(O)[C@H]1C5]

or a pharmaceutically acceptable salt thereof.

In some embodiments, the agent that reduces microglial cell activation is 2-amino-6-(trifluoromethoxy)benzothiazole, 6-(trifluoromethoxy)benzo[d]thiazol-2-amine, or 6-(trifluoromethoxy)-1,3-benzothiazol-2-amine (riluzole) or a pharmaceutically acceptable salt thereof or derivatives thereof as described in US patent number U.S. Pat. No. 5,527,814. In some embodiments, the agent is the following compound:

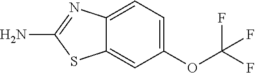 [SMILES: Nc1nc2ccc(OC(F)(F)F)cc2s1]

or a pharmaceutically acceptable salt thereof.

In some embodiments, the agent that reduces microglial cell activation is a modulator of a signaling pathway in microglia. In some cases, the agent reduces microglia singling. In some embodiments, the agent is a GM-CSF (CSF2) inhibitor. In other embodiments, the agent that reduces microglial cell activation is an ion channel blocker. In some specific embodiments, the agent is a calcium channel blocker. For example, in some specific examples, the agent is a dihydropyridine calcium channel blocker. In some embodiments, the agent is a microRNA inhibitor. For example, the agent targets miR-155. In some embodiments, the agent that reduces microglial cell activation is selected from MOR103, Nimodipine, IVIg, and LNA-anti-miR-155 (Butoxsky et al. Ann Neurol., 77(1):75-99 (2015) and Sanz et al., Br J Pharmacol. 167(8): 1702-1711 (2012); Winter et al., Ann Clin and Transl Neurol. 2328-9503 (2016); Clinical Trial Study Record Nos.: NCT01517282, NCT00750867).

In some embodiments, the agent that reduces microglial cell activation is 3-(2-methoxyethyl) 5-propan-2-yl 2,6-dimethyl-4-(3-nitrophenyl)-1,4-dihydropyridine-3,5-dicarboxylate (nimodipine) or a pharmaceutically acceptable salt thereof or derivatives thereof. In some embodiments, the agent is any of the inhibitors described in US patent number U.S. Pat. No. 3,799,934. In some embodiments, the agent is the following compound:

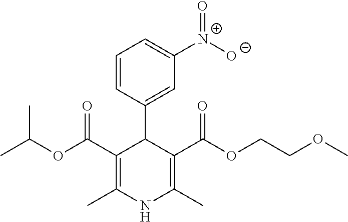 [SMILES: COCCOC(=O)C1=C(C)NC(C)=C(C(=O)OC(C)C)C1c1cccc([N+](=O)[O-])c1]

or a pharmaceutically acceptable salt thereof.

In some cases, the agent that reduces microglial cell activation is administered in a form that only affects to central nervous system and/or does not affect tumor-associated macrophages. In some embodiments, the agent promotes microglia quiescence but does not eliminate or reduce the number of microglia. In some embodiments, the method involves inhibiting microglia activity specifically in the brain such as described in Ponomarev et al., Nature Medicine, (1):64-70 (2011)

Exemplary agents that reduce microglial cell activation, and exemplary dosing regimens for administering such agents, are set forth in Table 7 below.

TABLE 7
Exemplary microglia inhibitors and dosage regimens

| Exemplary | Type of | Molecular | |
|---|---|---|---|
| Inhibitor | Molecule | Target(s) | Exemplary Dosing Regimen(s) |
| Pexidartinib | small molecule | CSF1R; c-Kit; | 200 mg tablets, twice daily for 28 days; |
| (PLX3397) | | FET3 | Administer daily as split dose regimen, |
| | | | five dose-levels possible in dose |
| | | | escalation part: 400 mg 5 days on 2 days |
| | | | off (intermittent schedule), 400 mg, 600 |
| | | | mg, 800 mg or 1000 mg; 1000 mg/day |
| | | | for 2 weeks then 800 mg/day for 22 |
| | | | weeks |
| Emactuzumab | monoclonal | CSF1R | 100-3000 mg once every 2 weeks |
| (RG1755; | antibody | | |
| RO5509554) | | | |
| Cabiralizumab | antibody | CSF1R | Intravenous infusion over 30 minutes |
| (FPA-008) | | | every 2 weeks |
| LY-3022855 | monoclonal | CSF1R | 1.25 mg/kg intravenous delivery every 2 |
| (IMC-CS4) | antibody | | weeks for 6 weeks |
| JNJ-40346527 | small molecule | CSF1R | 100 mg twice daily for 12 weeks; 100- |
| | | | 1000 mg capsule daily |
| MCS110 | antibody | MCSF (CSF1) | Up to 4 doses of 10 mg/kg MCS110 |
| | | | administered intravenously once every 4 |
| | | | weeks starting at Day 1 |
| MOR103 | antibody | GM-CSF | 6 doses of 0.5-2.0 mg/kg over 70 days |
| IVIg | immunoglobulin | Unknown | Intravenous infusion of 0.4 g/kg each |
| | | | month for 6 months |
| Minocyline | small molecule | broad spectrum | Oral dose of 100 mg of minocycline |
| | | antibiotic: IL-1b; | twice daily for 24 months |
| | | IL-6, TNF-a; | |
| | | iNOS | |
| Naloxone | small molecule | Opioid receptors | 4.5 mg naltrexone hydrochloride |
| | | | capsules once/day for 8 weeks |
| Lenalidomide/thalidomide | small molecule | NFkB signaling | 100-400 mg daily |
| Riluzole | small molecule | Glutamate release | 50 mg twice daily |
| | | by microglia | |
| Cannabinoids/ | small molecule | cannabinoid | Orally 10 mg/kg/day for 6 weeks |
| cannabidiol | | receptors | (average of 700 mg/day) |
| (e.g. WIN55, 212-2) | | | |
| Dimethyl | small molecule | Nrf2 signaling | Starting dose of 120 mg taken orally |
| fumarate | | | twice/day for 7 days. Dose increased to |
| (Tecfidera ®). | | | 240 mg taken orally twice/day thereafter |
| natalizumab | antibody | alpha-4 integrin | 300 mg infused intravenously over |
| (Tysabri ®) | | | one hour, every four weeks |
| fingolimod | small molecule | S1P receptors, | 0.5 mg orally once-daily |
| (Gilenya ®) | | including | |
| | | S1PR1 | |
| ozanimod | small molecule | S1PR1 and | 0.25 mg, 0.5 mg, or 1 mg once daily |
| (RPC-1063) | | S1PR5 | |

3. Other Agents

In some embodiments, the agent or other treatment that treats or ameliorates symptoms of a toxicity of immunotherapy and/or a cell therapy, such as CRS or neurotoxicity, is one that targets a cytokine, e.g., is an antagonist or inhibitor of a cytokine, such as transforming growth factor beta (TGF-beta), interleukin 6 (IL-6), interleukin 10 (IL-10), IL-2, MIP1β (CCL4), TNF-alpha, IL-1, interferon gamma (IFN-gamma), or monocyte chemoattractant protein-1 (MCP-1). In some embodiments, the agent that treats or ameliorates symptoms of a toxicity of an immunotherapy and/or a cell therapy, such as CRS or neurotoxicity, is one that targets (e.g. inhibits or is an antagonist of) a cytokine receptor, such as IL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1β receptor (e.g., CCR5), TNF-alpha receptor (e.g., TNFR1), IL-1 receptor (IL1-Rα/IL-1Rβ), or IL-10 receptor (IL-10R).

The amount of a selected agent that treats or ameliorates symptoms of a toxicity of an immunotherapy and/or a cell therapy, such as CRS or neurotoxicity following administration of be administered to ameliorate symptoms or adverse effects of a toxicity following administration of an immunotherapy and/or a cell therapy, such as CRS or neurotoxicity, can be determined by standard clinical techniques. Exemplary adverse events include, but are not limited to, an increase in alanine aminotransferase, an increase in aspartate aminotransferase, chills, febrile neutropenia, headache, hypotension, left ventricular dysfunction, encephalopathy, hydrocephalus, seizure, and/or tremor.

In some embodiments, the agent is administered in a dosage amount of from or from about 30 mg to 5000 mg, such as 50 mg to 1000 mg, 50 mg to 500 mg, 50 mg to 200 mg, 50 mg to 100 mg, 100 mg to 1000 mg, 100 mg to 500 mg, 100 mg to 200 mg, 200 mg to 1000 mg, 200 mg to 500 mg or 500 mg to 1000 mg.

In some embodiments, the agent is administered from or from about 0.5 mg/kg to 100 mg/kg, such as from or from about 1 mg/kg to 50 mg/kg, 1 mg/kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive. In some aspects, the agent is administered in a dosage amount of at least or at least about or about 1 mg/kg, 2 mg/kg, 4 mg/kg, 6 mg/kg, 8 mg/kg, 10 mg/kg or more. In some embodiments, the agent is administered at a dose of 4 mg/kg or 8 mg/kg.

In some embodiments, the agent is administered by injection, e.g., intravenous or subcutaneous injections, intraocular injection, periocular injection, subretinal injection, intravitreal injection, trans-septal injection, sub-scleral injection, intra-choroidal injection, intra-cameral injection, sub-conjunctival injection, sub-conjunctival injection, sub-Tenon's injection, retro-bulbar injection, peri-bulbar injection, or posterior juxta-scleral delivery. In some embodiments, they are administered by parenteral, intrapulmonary, and intranasal, and, if desired for local treatment, intra-lesional administration. Parenteral infusions include intramuscular, intravenous, intraarterial, intraperitoneal, or subcutaneous administration.

In some embodiments, the amount of the agent is administered about or approximately twice daily, daily, every other day, three times a week, weekly, every other week or once a month.

In some embodiments, the agent is administered as part of a composition or formulation, such as a pharmaceutical composition or formulation as described below. Thus, in some cases, the composition comprising the agent is administered as described below. In other aspects, the agent is administered alone and may be administered by any known acceptable route of administration or by one described herein, such as with respect to compositions and pharmaceutical formulations.

In some embodiments, the agent that treats or ameliorates symptoms of a toxicity of the immunotherapy and/or cell therapy, such as CRS or neurotoxicity, is an antibody or antigen binding fragment. In some embodiments, the agent is tocilizumab, siltuximab, sarilumab, olokizumab (CDP6038), elsilimomab, ALD518/BMS-945429, sirukumab (CNTO 136), CPSI-2634, ARGX-109, FE301, or FM101.

In some embodiments, the agent is an antagonist or inhibitor of IL-6 or the IL-6 receptor (IL-6R). In some aspects, the agent is an antibody that neutralizes IL-6 activity, such as an antibody or antigen-binding fragment that binds to IL-6 or IL-6R. For example, in some embodiments, the agent is or comprises tocilizumab (atlizumab) or sarilumab, anti-IL-6R antibodies. In some embodiments, the agent is an anti-IL-6R antibody described in U.S. Pat. No: 8,562,991. In some cases, the agent that targets IL-6 is an anti-IL-6 antibody, such as siltuximab, elsilimomab, ALD518/BMS-945429, sirukumab (CNTO 136), CPSI-2634, ARGX-109, FE301, FM101, or olokizumab (CDP6038). In some aspects, the agent may neutralize IL-6 activity by inhibiting the ligand-receptor interactions. The feasibility of this general type of approach has been demonstrated with a natural occurring receptor antagonist for interleukin-1. See Harmurn, C. H. et al., Nature (1990) 343:336-340. In some aspects, the IL-6/IL-6R antagonist or inhibitor is an IL-6 mutein, such as one described in U.S. Pat. No. 5591827. In some embodiments, the agent that is an antagonist or inhibitor of IL-6/IL-6R is a small molecule, a protein or peptide, or a nucleic acid.

In some embodiments, the agent is tocilizumab. In some embodiments, tocilizumab is administered as an early intervention in accord with the provided methods a dosage of from or from about 1 mg/kg to 12 mg/kg, such as at or about 4 mg/kg, 8 mg/kg, or 10 mg/kg. In some embodiments, tocilizumab is administered by intravenous infusion. In some embodiments, tocilizumab is administered for a persistent fever of greater than 39° C. lasting 10 hours that is unresponsive to acetaminophen. In some embodiments, a second administration of tocilizumab is provided if symptoms recur after 48 hours of the initial dose.

In some embodiments, the agent is an agonist or stimulator of TGF-β or a TGF-β receptor (e.g., TGF-β receptor I, II, or III). In some aspects, the agent is an antibody that increases TGF-β activity, such as an antibody or antigen-binding fragment that binds to TGF-β or one of its receptors. In some embodiments, the agent that is an agonist or stimulator of TGF-β and/or its receptor is a small molecule, a protein or peptide, or a nucleic acid.

In some embodiments, the agent is an antagonist or inhibitor of MCP-1 (CCL2) or a MCP-1 receptor (e.g., MCP-1 receptor CCR2 or CCR4). In some aspects, the agent is an antibody that neutralizes MCP-1 activity, such as an antibody or antigen-binding fragment that binds to MCP-1 or one of its receptors (CCR2 or CCR4). In some embodiments, the MCP-1 antagonist or inhibitor is any described in Gong et al. J Exp Med. 1997 Jul. 7; 186(1): 131-137 or Shahrara et al. J Immunol 2008; 180:3447-3456. In some embodiments, the agent that is an antagonist or inhibitor of MCP-1 and/or its receptor (CCR2 or CCR4) is a small molecule, a protein or peptide, or a nucleic acid.

In some embodiments, the agent is an antagonist or inhibitor of IFN-γ or an IFN-γ receptor (IFNGR). In some aspects, the agent is an antibody that neutralizes IFN-γ activity, such as an antibody or antigen-binding fragment that binds to IFN-γ or its receptor (IFNGR). In some aspects, the IFN-gamma neutralizing antibody is any described in Dobber et al. Cell Immunol. 1995 Feb;160(2):185-92 or Ozmen et al. J Immunol. 1993 Apr. 1; 150(7):2698-705. In some embodiments, the agent that is an antagonist or inhibitor of IFN-γ/IFNGR is a small molecule, a protein or peptide, or a nucleic acid.

In some embodiments, the agent is an antagonist or inhibitor of IL-10 or the IL-10 receptor (IL-10R). In some aspects, the agent is an antibody that neutralizes IL-10 activity, such as an antibody or antigen-binding fragment that binds to IL-10 or IL-10R. In some aspects, the IL-10 neutralizing antibody is any described in Dobber et al. Cell Immunol. 1995 February; 160(2):185-92 or Hunter et al. J Immunol. 2005 Jun 1;174(11):7368-75. In some embodiments, the agent that is an antagonist or inhibitor of IL-10/IL-10R is a small molecule, a protein or peptide, or a nucleic acid.

In some embodiments, the agent is an antagonist or inhibitor of IL-1 or the IL-1 receptor (IL-1R). In some aspects, the agent is an IL-1 receptor antagonist, which is a modified form of IL-1R, such as anakinra (see, e.g., Fleischmann et al., (2006) Annals of the rheumatic diseases. 65(8):1006-12). In some aspects, the agent is an antibody that neutralizes IL-1 activity, such as an antibody or antigen-binding fragment that binds to IL-1 or IL-1R, such as canakinumab (see also EP 2277543). In some embodiments, the agent that is an antagonist or inhibitor of IL-1/IL-1R is a small molecule, a protein or peptide, or a nucleic acid.

In some embodiments, the agent is an antagonist or inhibitor of a tumor necrosis factor (TNF) or a tumor necrosis factor receptor (TNFR). In some aspects, the agent is an antibody that blocks TNF activity, such as an antibody or antigen-binding fragment that binds to a TNF, such as TNFα, or its receptor (TNFR, e.g., TNFRp55 or TNFRp75). In some aspects, the agent is selected from among infliximab, adalimumab, certolizumab pegol, golimumab and etanercept. In some embodiments, the agent that is an antagonist or inhibitor of TNF/TNFR is a small molecule, a protein or peptide, or a nucleic acid. In some embodiments, the agent is a small molecule that affects TNF, such as lenalidomide (see, e.g., Muller et al. (1999) Bioorganic & Medicinal Chemistry Letters. 9 (11):1625).

In some embodiments, the agent is an antagonist or inhibitor of signaling through the Janus kinase (JAK) and two Signal Transducer and Activator of Transcription (STAT) signaling cascade. JAK/STAT proteins are common components of cytokine and cytokine receptor signaling. In some embodiments, the agent that is an antagonist or inhibitor of JAK/STAT, such as ruxolitinib (see, e.g., Mesa et al. (2012) Nature Reviews Drug Discovery. 11(2):103-104), tofacitinib (also known as Xeljanz, Jakvinus tasocitinib and CP-690550) , Baricitinib (also known as LY-3009104, INCB-28050), Filgotinib (G-146034, GLPG-0634), Gandotinib (LY-2784544), Lestaurtinib (CEP-701), Momelotinib (GS-0387, CYT-387), Pacritinib (SB1518), and Upadacitinib (ABT-494). In some embodiments, the agent is a small molecule, a protein or peptide, or a nucleic acid.

In some embodiments, the agent is a kinase inhibitor. In some embodiments, the agent is an inhibitor of Bruton's tyrosine kinase (BTK). In some embodiments, the inhibitor is or comprises ibrutinib or acalabrutinib (see, e.g., Barrett et al., ASH 58^{th} Annual Meeting San Diego, Calif. Dec. 3-6, 2016, Abstract 654; Ruella et al., ASH 58^{th} Annual Meeting San Diego, Calif. Dec. 3-6, 2016, Abstract 2159). In some embodiments, the agent is an inhibitor as described in U.S. Pat. Nos. 7,514,444; 8,008,309; 8,476,284; 8,497,277; 8,697,711; 8,703,780; 8,735,403; 8,754,090; 8,754,091; 8.957,079; 8,999,999; 9,125,889; 9,181,257; or 9,296,753.

In some embodiments, a device, such as absorbent resin technology with blood or plasma filtration, can be used to reduce cytokine levels. In some embodiments, the device used to reduce cytokine levels is a physical cytokine absorber, such as an extracorporeal cytokine absorber. In some embodiments, a physical cytokine absorber can be used to eliminate cytokines from the bloodstream in an ex vivo, extracorporeal manner. In some embodiments, the agent is a porous polymer. In some embodiments, the agent is CytoSorb (see, e.g., Basu et al. Indian J Crit Care Med. (2014) 18(12): 822-824).

### V. KITS AND ARTICLES OF MUNUFACTURE

In some embodiments, provided here are kits and/or articles of manufacture that are useful for performing any of the methods provided herein. In particular embodiments, instructions are included for performing the methods with the kits and/or articles of manufacture.

In certain embodiments, provided herein are kits that are useful for determining, estimating, and/or assess the risk, probability and/or likelihood that a subject will experience a toxicity following administration of a therapy. In some embodiments, provided herein are kits that are useful for detecting a gene signature associated with a risk of toxicity following administration of a therapy. In particular embodiments, the kits are useful for detecting the expression of one or more genes that are associated with a toxicity following administration of a therapy. In certain embodiments, the therapy is a treatment with a therapeutic cell composition. In particular embodiments, the therapeutic cell composition is a cell composition that expresses a recombinant receptor, e.g., a CAR. In some embodiments the toxicity is neurotoxicity. In particular embodiments the toxicity is severe neurotoxicity, e.g., neurotoxicity of grade 4 or 5 and/or of a prolonged grade 3. In certain embodiments, the therapy is for treating a cancer, e.g., acute lymphoblastic leukemia (ALL).

In some embodiments, the kit includes one or more reagents for measuring, detecting, assessing, and/or quantifying one or more gene products or portions thereof that are associated with a risk of developing a toxicity, e.g., neurotoxicity, to a therapy. In particular embodiments, the gene products are an RNA gene product and/or protein gene products. In some embodiments, the gene products are expressed by any of the genes provided herein, e.g., genes of Section I, such as Section-IB and Section-IE. In particular embodiments, the kit contains a reagent for measuring, detecting, assessing, and/or quantifying one or more gene products that negatively correlate to a risk of developing toxicity. In particular embodiments, the kit contains one or more reagents for measuring, detecting, assessing, and/or quantifying one or more gene products positively that correlate to a risk of developing toxicity. In certain embodiments, the kit contains one or more reagents for detecting one or more gene products expressed and/or encoded by a gene listed in Table 1, Table 2, Table 3, Table E2A, Table E2B, or Table E4 (e.g. a subset of these genes in the relevant Table which as a recited SEQ ID NO, and/or a Uniprot ID). In some embodiments, the gene products are or include one or more gene products expressed and/or encoded by ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A. In various embodiments, the gene products are or include one or more gene products expressed and/or encoded by CCL17, CA6, JCHAIN, PTP4A3, IFITM1, CRLF2, ENAM, GBP5, ABCA9, SV2C, SLC37A3, IL15, IL2RA, and SEMA6A. In particular embodiments, the gene products are or include one or more gene products expressed and/or encoded by ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28. In various embodiments, the gene products are or include one or more gene products expressed and/or encoded by PCDHGA12, PCDHGB6, PCDHGB5, PCDHGA9, PINLYP, ASAP2, TTC28, PTCH1, and FMNL1. In some embodiments, the gene products are or include one or more gene products encoded by CCL17, CCR6, GAS6, GL12, PTP4A3, or IL2RA.

In certain embodiments, the kit contains one or more reagents for measuring, detecting, assessing, and/or quantifying one or more gene products expressed by one or more genes listed in Table 1 and/or Table E2A and/or one or more genes listed in Table 2 and/or Table E2B (e.g. a subset of these genes in the relevant Table which as a recited SEQ ID NO, and/or a Uniprot ID). In some embodiments, the kit contains one or more reagents for measuring, detecting, assessing, and/or quantifying one or more gene products from one or more of ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53ITP1, TNF, TRIM47, UST, WNT9A, ENG, SELE, ICAM3, or IL6R, and/or one or more of ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415.

In certain embodiments, the kit contains one or more reagents for measuring, detecting, assessing, and/or quantifying one or more gene products expressed by one or more of ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, and WNT9A and/or one or more of ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28.

In certain embodiments, the kit contains one or more reagents for measuring, detecting, assessing, and/or quantifying one or more gene products expressed by one or more of IGJ (JCHAIN), MUC4, CA6, WNT9A, ADGRF1 and CCL17 and/or one or more of PINLYP, ASAP2, FMNL2, PTCH1, TTC28, PCDHGA6, PCDHGB6 and PCDHGA12. In particular embodiments, the kit contains one or more reagents for measuring, detecting, assessing, and/or quantifying one or more gene products expressed by one or more of IGJ (JCHAIN), MUC4, CA6, WNT9A, ADGRF1 and CCL17 and/or one or more of PINLYP and PCDHGA12.

In some embodiments, the kit includes and/or contains one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-five, at least forty, at least fifty, at least sixty, at least seventy, at least eighty, at least ninety, or at least one hundred reagents for measuring, detecting, assessing, and/or quantifying one or more gene products. In some embodiments, reagents for measuring, detecting, assessing, and/or quantifying one or more gene products may include, but are not limited to, oligonucleotides, polynucleotides, polypeptides, antibodies or an antigen binding fragments thereof, and/or small molecules that are capable of recognizing, binding, or otherwise indicating the presence of the gene product, or portion thereof, in a sample. In certain embodiments, the reagent has an attached, e.g., covalently attached detectable label, e.g., a fluorescent label.

In some embodiments, the kit also contains one or more reagents for assessing, measuring, detecting, and/or quantifying a parameter of the therapeutic cell composition. In certain embodiments, the kit contains a reagent that is useful for stimulating a receptor, such as a recombinant receptor or a CAR, that are expressed by at least some of the cells in the therapeutic cell composition. In particular embodiments, kit contains one or more reagents for measuring, detecting, assessing, and/or quantifying proinflammatory cytokines. In some embodiments, the kit contains one or more reagents for measuring, detecting, assessing, and/or quantifying one or more of IL-2, IL-13, IFN-gamma, or TNF-alpha.

In some embodiments, provided herein is a kit for administering a therapeutic T cell composition to a subject. In certain embodiments, the kit contains the therapeutic T cell composition and instructions for administering the therapeutic T cell composition to a subject. In certain embodiments, the instructions are for administering the therapeutic T cell composition to a subject by any method provided herein. In certain embodiments, the instructions for use indicate that if the expression of one or more gene products or portions thereof in a sample from the subject, in relation to a gene reference value, indicates that the subject is not likely to develop toxicity, e.g., severe neurotoxicity, associated with the therapeutic T cell composition, then the therapeutic T cell composition is administered to the subject. In particular embodiments, the instructions for use indicate that if the expression of one or more gene products or portions thereof in a sample from the subject indicates that the subject is likely to develop toxicity, e.g., severe neurotoxicity, then an alternative therapeutic treatment is administered to the subject. In some embodiments, an alternative therapeutic treatment is a therapeutic treatment other than the therapeutic T cell composition, a low dosage amount of the therapeutic T cell composition and/or a combination of the therapeutic T cell composition and one or more interventions for ameliorating the risk of developing neurotoxicity.

Also provided herein are articles of manufacture, wherein the articles of manufacture includes a kit provided herein and instructions for use. In some embodiments, the instructions described any of the methods provided herein.

In some embodiments, the articles of manufacture contain an immunotherapy and instructions for administering the immunotherapy. In some embodiments, the immunotherapy is an immunotherapy described in Section II, e.g., an immune cell activator described in Section II-A or a cell therapy described in Section in Section II-B. In some embodiments, the immunotherapy is a cell therapy, such as a CAR-T cell therapy. In particular embodiments, the instructions specify that the immunotherapy is administered to a subject that has been selected or identified as having a PH+ and/or Ph-like molecular subtype of ALL. In some embodiments, the instructions specify that the immunotherapy is not administered to a subject that has been selected or identified to have a molecular subtype of ALL that is not PH+ or Ph-like. In some embodiments, the instructions specify that the immunotherapy is administered to a subject that is selected or identified as having a PH+ or Ph-like molecular phenotype of ALL by a method as described in Section I-D.

In some embodiments, the articles of manufacture contain an immunotherapy and instructions for administering the immunotherapy to a subject. In some embodiments, the instructions specify having a positive, increased, and/or elevated expression of a gene listed in in Table 1 and/or Table E2A (e.g. a subset of these genes in the relevant Table which as a recited SEQ ID NO, and/or a Uniprot ID). In particular embodiments, the instructions specify that the immunotherapy is administered to a subject having reduced, decreased, and/or negative expression of one or more genes listed in Table 2 and/or Table E2B (e.g. a subset of these genes in the relevant Table which as a recited SEQ ID NO, and/or a Uniprot ID). In some embodiments, the instructions specify that the immunotherapy is not administered to a subject having reduced, decreased, and/or negative expression of one or more genes listed in Table 1 and/or Table E2A (e.g. a subset of these genes in the relevant Table which as a recited SEQ ID NO, and/or a Uniprot ID). In some embodiments, the instructions specify that the immunotherapy is not administered to a subject having increased, elevated, and/or positive expression of one or more genes listed in Table 2 and/or Table E2B (e.g. a subset of these genes in the relevant Table which as a recited SEQ ID NO, and/or a Uniprot ID).

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

As used herein, “percent (%) sequence identity” and “percent identity” when used with respect to a nucleotide sequence (reference nucleotide sequence) or amino acid sequence (reference amino acid sequence) is defined as the percentage of nucleotide residues or amino acid residues, respectively, in a candidate sequence that are identical with the residues in the reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe

Non-conservative amino acid substitutions will involve exchanging a member of one of these classes for another class.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” refers to within ±25%, ±20%, ±15%, ±10%, ±5%, or ±1% of the value or parameter.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### VII. EXEMPLARY EMBODIMENTS

Among the provided embodiments are:

1. A method of assessing a risk of a toxicity or a toxicity-related outcome, following administration of an immunotherapy, the method comprising:

(1) assessing the presence, absence or level of expression of one or more gene products or portions thereof in a sample from a subject that is a candidate for receiving a immunotherapy for treatment of a disease or condition, wherein:
the one or more gene products is associated with a risk of developing neurotoxicity following administration of the immunotherapy; and
the sample does not comprise the immunotherapy and/or is obtained from the subject prior to receiving the immunotherapy; and

(2) comparing the presence, absence or level of expression of the one or more gene products or portions thereof to a gene reference value, wherein the comparison indicates the risk or likely risk of the subject developing a neurotoxicity, optionally a specified grade or severity of neurotoxicity, following administration of the therapy to the subject.

2. The method of embodiment 1, wherein each of the one or more gene products is individually compared to a gene reference value for the respective gene product.

3. The method of embodiment 1 or embodiment 2, wherein:

(a) at least one of the one or more gene products is from a first group of gene products that negatively correlate to a risk of developing neurotoxicity; and/or

(b) at least one of the one or more gene products is from a second group of gene products that positively correlates to a risk of developing neurotoxicity.

4. The method of embodiment 3, wherein the at least one gene product is from (a) and is selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof.

5. The method of embodiment 3 or embodiment 4, wherein the at least one gene product is from (b) and is selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or is a portion or fragment thereof.

6. A method of assessing a risk of toxicity following administration of a immunotherapy, the method comprising:

(1) assessing the presence, absence or level of expression of one or more gene products or a portion thereof in a sample from a subject that is a candidate for receiving a immunotherapy for treating a disease or condition, wherein (a) at least one of the one or more gene products is selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or (b) at least one of the one or more gene products is selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or is a portion or fragment thereof; and

(2) comparing the presence, absence or level of expression of the one or more gene product to a gene reference value, wherein the comparison indicates whether the subject is or is likely at risk of developing a neurotoxicity or grade or severity thereof following administration of the immunotherapy when administered to the subject.

7. The method of embodiment 6, wherein each of the one or more gene products is individually compared to a gene reference value for the respective gene product.

8. The method of any of embodiments 1-7, wherein the immunotherapy is a cell therapy or is a T cell-engaging therapy, optionally wherein the cell therapy comprises cells engineered to express a recombinant receptor.

9. The method of embodiment 6, 7 or 8, wherein the sample does not comprise the immunotherapy, and/or is obtained from the subject prior to receiving the cell therapy.

10. The method of any of embodiments 1-9, wherein the sample does not contain cells genetically engineered with the recombinant receptor.

11. The method of any of embodiments 3-10, wherein the presence, absence or level of the one or more gene products is associated with a risk of developing neurotoxicity following administration of the immunotherapy, optionally a cell therapy.

12. The method of embodiment 11, wherein: expression of the at least one or more gene products from (a) negatively correlate to a risk of developing neurotoxicity; and/or expression of the at least one or more gene products from (b) positively correlates to a risk of developing neurotoxicity.

13. The method of any of embodiments 3-12, wherein: the comparison indicates the subject is or is likely at risk of developing neurotoxicity if the at least one gene product of (a) is at or below a gene reference value and/or the at least one gene product of (b) is at or above a gene reference value; or

the comparison indicates the subject is not or is likely not at risk of developing neurotoxicity if the at least one gene product of (a) is above a gene reference value and/or the at least one gene product of (b) is below a gene reference value.

14. The method of any of embodiment 3-13, wherein if the comparison indicates the subject is or is likely to develop neurotoxicity, selecting the subject for administration of a therapeutic regimen, the therapeutic regimen comprising administering to the subject:

i. an agent or other treatment capable of treating, preventing, delaying, reducing or attenuating the development or risk of development of a toxicity and the immunotherapy, wherein administration of the agent is to be administered (i) prior to, (ii) within one, two, or three days of, (iii) concurrently with and/or (iv) at first fever following, the initiation of administration of the immunotherapy to the subject;

ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy; and/or

iii. the immunotherapy in an in-patient setting and/or with admission to the hospital for one or more days, optionally wherein the immunotherapy is otherwise to be administered to subjects on an outpatient basis or without admission to the hospital for one or more days; or
iv. an alternative therapeutic treatment other than the immunotherapy.

15. The method of any of embodiments 1-13, wherein if the comparison indicates the subject is not or is likely not at risk of developing neurotoxicity, selecting the subject for administration of a therapeutic regimen, the therapeutic regimen comprising administering to the subject:

i. the immunotherapy, optionally at a non-reduced dose, optionally on an outpatient basis or without admission to the hospital for one or more days;

ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or

iii. the immunotherapy in an outpatient setting and/or without admission of the subject to the hospital overnight or for one or more consecutive days and/or is without admission of the subject to the hospital for one or more days.

16. The method of embodiment 14 or embodiment 15, further comprising administering the therapeutic regimen to the selected subject.

17. A method of treatment, the method comprising administering a therapeutic regimen to a subject that is a candidate for receiving an immunotherapy for treatment of a disease or condition, wherein the administration is carried out following or based on the results of assessing the presence, absence or level of expression, from a sample from the subject, of one or more gene products or portion thereof, wherein:

(a) at least one of the one or more gene products is selected from ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS 1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or

(b) at least one of the one or more gene products is selected from ASAP2, ATP9A, CCNA1, CDHR3, CECR2, DLX1, DPYSL3, EHD4, FMNL2, GGA2, HHIPL1, HMX3, IGF2BP1, IL3RA, IRX5, KCNIP1, KIAA1644, LINC00092, LINC01483, MIB1, MMP14, NOM1, OTOA, PCDHGA12, PCDHGA4, PCDHGA6, PCDHGBS, PCDHGB6, PINLYP, PPM1E, PRKD1, PROKR2, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TNKS1BP1, TTC28, PCDHGA9, FMNL1, or ZNF415 or is a portion or fragment thereof.

18. The method of embodiment 17, wherein the immunotherapy is a cell therapy or is a T cell-engaging therapy, optionally wherein the cell therapy comprises cells engineered to express a recombinant receptor.

19. The method of embodiment 17 or 18, wherein the sample is obtained from the subject prior to receiving the immunotherapy and/or the sample does not comprise the immunotherapy.

20. The method of any of embodiments 17-19, wherein the presence, absence or level of the one or more gene products is associated with a risk of developing neurotoxicity following administration of the immunotherapy.

21. The method of any of embodiments 17-20, wherein the results of assessing the presence, absence or level of expression of the one or more gene products or portions thereof comprises a comparison to a gene reference value, wherein the comparison indicates the risk or likely risk of the subject developing neurotoxicity following administration of the immunotherapy when administered to the subject.

22. The method of embodiment 21, wherein each of the one or more gene products is individually compared to a gene reference value for the respective gene product.

23. The method of any of embodiments 20-22, wherein:

expression of the at least one or more gene products from (a) negatively correlate to a risk that the subject is or is likely to develop neurotoxicity following administration of the immunotherapy when it is administered; and/or

expression of the at least one or more gene products from (b) positively correlates to a risk that the subject is or is likely to develop neurotoxicity following administration of the immunotherapy when it is administered.

24. The method of any of embodiments 17-23, wherein if the assessing indicates the subject is or is likely to develop neurotoxicity following administration of the immunotherapy, the therapeutic regimen comprises administering to the subject:

i. an agent or other treatment capable of treating, preventing, delaying, reducing or attenuating the development or risk of development of a toxicity and (2) the immunotherapy, wherein administration of the agent is to be administered (i) prior to, (ii) within one, two, or three days of, (iii) concurrently with and/or (iv) at first fever following, the initiation of administration of the immunotherapy to the subject;

ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy; and/or

iii. the immunotherapy in an in-patient setting and/or with admission to the hospital for one or more days, optionally wherein the immunotherapy is otherwise to be administered to subjects on an outpatient basis or without admission to the hospital for one or more days; or

iv. an alternative therapeutic treatment other than the immunotherapy.

25. The method of any of embodiments 17-24, wherein if the assessing indicates the subject is not or is likely not to develop neurotoxicity following administration of the immunotherapy, the therapeutic regimen comprises administering to the subject:

i. the immunotherapy, optionally at a non-reduced dose, optionally on an outpatient basis or without admission to the hospital for one or more days;

ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or

iii. the immunotherapy in an outpatient setting and/or without admission of the subject to the hospital overnight or for one or more consecutive days and/or is without admission of the subject to the hospital for one or more days.

26. The method of any of embodiments 1-25, wherein the disease or condition is a cancer, optionally a myeloma, lymphoma or leukemia.

27. The method of any of embodiments 1-26, wherein the disease or condition is a B cell malignancy.

28. The method of embodiment 27, wherein the B cell malignancy is selected from acute lymphoblastic leukemia (ALL), chronic lymphoblastic leukemia (CLL), acute myelogenous leukemia (AML), chronic myelogenous leukemia (CML), non-Hodgkin lymphoma (NHL), or Diffuse Large B-Cell Lymphoma (DLBCL), or a subtype of any of the foregoing.

29. The method of any of embodiments 1-28, wherein the disease or condition is acute lymphoblastic leukemia (ALL) or a subtype thereof.

30. The method of any of embodiments 1-29, wherein the at least one gene product is from (a) and is a gene product associated with a PH+ or Ph-like molecular subtype of ALL.

31. The method of embodiment 30, wherein the at least one gene product is selected from ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, L0645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A, or is a portion or fragment of any of the foregoing.

32. A method of treatment, the method comprising:

selecting a subject that exhibits a Philadelphia chromosome (Ph+) and/or Ph chromosome-like (Ph-like) molecular subtype of acute lymphoblastic leukemia (ALL); and administering to the subject a immunotherapy that binds to an antigen associated with the ALL.

33. The method of embodiment 32, wherein the immunotherapy is a cell therapy or is a T cell-engaging therapy, optionally wherein the cell therapy comprises cells engineered to express a recombinant receptor.

34. The method of embodiment 32 or 33, wherein the subject is selected based on results of cytogenetic or molecular genetic analysis.

35. The method of embodiment 32 or 33, wherein the analysis comprises karyotype analysis, fluorescence in situ hybridization (FISH), multicolor FISH, polymerase chain reaction (PCR), a tyrosine kinase inhibitor assay, gene expression profiling or microarray or an immunoassay, optionally an ELISA.

36. The method of any of embodiments 32-35, wherein:

the selected subject exhibits one or more of the (9;22)(q34;q11) chromosomal abnormality; deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or comprises a Ph-like gene expression signature;

the subject is selected based on one or more of the presence of the (9;22)(q34;q11) chromosomal abnormality, deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or the presence of a Ph-like gene expression signature.

37. The method of any of embodiments 33-36, wherein the presence of the Ph-like gene signature is based on comparison of the presence, absence or level of expression, in a sample from the subject, of at least one gene product to a reference gene value, said at least one gene product is selected from (a) ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, LO645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A or a portion or fragment of any of the foregoing and/or said at least one gene product is selected from (b) ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing, whereby the comparison indicates whether the subject exhibits a Ph-like molecular subtype of ALL.

38. The method of embodiment 37, wherein each of the one or more gene products is individually compared to a gene reference value for the respective gene product.

39. The method of any of embodiment 37 or embodiment 38, wherein the subject exhibits a Ph-like molecular subtype of ALL if the comparison indicates the at least one gene product of (a) is above a gene reference value and/or the at least one gene product of (b) is below a gene reference value.

40. The method of any of embodiments 30, 31 and 37-39, wherein the at least one gene product selected from (a) is ADGRF1, BMPR1B, CA6, CD99, CHN2, CRLF2, DENND3, ENAM, GBP5, GLI2, IFITM1, IGJ (JCHAIN), LDB3, L0645744, MDF1C, MUC4, NRXN3, PON2, S100Z, SEMA6A, SLC37A3, SLC2A5, SPATS2L, TMEM154, TP53INP1, TTYH2 or WNT9A, or is a portion of fragment of any of the foregoing.

41. The method of any of embodiments 3-40, wherein the at least one gene product selected from (a) is ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A or is a portion or fragment of any of the foregoing.

42. The method of any of embodiments 3-41, wherein the at least one gene product selected from (b) is ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing.

43. The method of any of embodiments 1-42, wherein the subject is a human and/or the one or more gene products is human.

44. The method of any of embodiments 3-31 and 37-43, wherein at least one of the one or more gene products is from (a) and at least one of the one or more gene products is from (b).

45. The method of any of embodiments 1-44, wherein the one or more genes comprises at least one gene from (a) that is IGJ (JCHAIN), MUC4, CA6, WNT9A, ADGRF1 or CCL17, or a portion or fragment of any of the foregoing.

46. The method of any of embodiments 1-45, wherein the one or more genes comprises at least one gene from (a) that is CCL17 or a portion or fragment thereof.

47. The method of any of embodiments 1-46, wherein the one or more genes comprises at least one gene from (b) that is PINLYP, ASAP2, FMNL2, PTCH1, TTC28, PCDHGA6, PCDHGB6 or PCDHGA12, or a portion or fragment of any of the foregoing.

48. The method of any of embodiments 1-47, wherein the one or more genes comprises at least one gene from (b) that is PINLYP or PCDHGA12 or a portion or fragment of any of the foregoing.

49. The method of any of embodiments 16-48, wherein:

greater than or greater than about 30%, 35%, 40%, or 50% of the subjects treated according to the method do not exhibit any grade of cytokine release syndrome (CRS) or neurotoxicity; and/or

at least at or about 45, 50, 60, 65, 70, 75, 80, 85, 90, 95% or about 100% of subjects treated according to the method do not exhibit severe CRS, optionally grade 3 or higher, prolonged grade 3 or higher or grade 4 or 5 CRS; and/or

at least at or about 45, 50, 60, 65, 70, 75, 80, 85, 90, 95% or about 100% of subjects treated according to the method do not exhibit severe neurotoxicity, optionally grade 3 or higher, prolonged grade 3 or higher or grade 4 or 5 neurotoxicity; and/or

at least at or about 45, 50, 60, 65, 70, 75, 80, 85, 90, 95% or about 100% of subjects treated according to the method do not exhibit cerebral edema.

50. The method of any of embodiments 16-49, wherein:

prior to initiation of administration of the dose of cells, the subject has not been administered an agent or treatment capable of treating, preventing, delaying, reducing or attenuating the development or risk of development of a toxicity; and/or

the subject is not administered an agent or treatment for the treatment or prevention or reduction or attenuation of a neurotoxicity and/or a cytokine release syndrome or risk thereof, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or

the subject is not administered an agent or treatment for the treatment or prevention or reduction or attenuation of a neurotoxicity and/or a cytokine release syndrome or risk thereof, following administration of the dose, prior to or unless the subject exhibits a sign or symptom of the toxicity and/or prior to or unless the subject exhibits a sign or symptom of the toxicity other than a fever, optionally wherein the fever is not a sustained fever or the fever is or has been reduced or reduced by more than 1° C. after treatment with an antipyretic; and/or

the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

51. The method of any of embodiments 16-50, wherein:

prior to initiation of administration of the dose of cells, the subject has not been administered an anti-IL-6 or anti-IL-6R antibody, optionally tocilizumab or siltuximab, and/or has not been administered a steroid, optionally dexamethasone

the subject is not administered an anti-IL-6 or anti-IL-6R antibody, optionally tocilizumab or siltuximab, and/or has not been administered a steroid, optionally dexamethasone, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or

the subject is not administered an anti-IL-6 or anti-IL-6R antibody, optionally tocilizumab or siltuximab, and/or has not been administered a steroid, optionally dexamethasone, following administration of the cell dose, prior to, or unless, the subject exhibits a sign or symptom of a toxicity, optionally a neurotoxicity or CRS, and/or prior to, or unless, the subject exhibits a sign or symptom of a toxicity, optionally a neurotoxicity or CRS, other than a fever, optionally wherein the fever is not a sustained fever or the fever is or has been reduced or reduced by more than 1° C. after treatment with an antipyretic; and/or the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

52. The method of any of embodiments 16-51, wherein:

the administration is carried out on an outpatient basis and/or without requiring admission to or an overnight stay at a hospital; and

if the subject exhibits a sustained fever or a fever that is or has not been reduced or not reduced by more than 1° C. after treatment with an antipyretic, the subject is admitted to the hospital or to an overnight stay at a hospital and/or is administered an agent or treatment for the treatment or prevention or reduction or attenuation of a neurotoxicity and/or a cytokine release syndrome or risk thereof.

53. The method of any of embodiments 1-52, wherein the neurotoxicity comprises severe neurotoxicity, optionally at or above grade 4 or grade 5 or at least prolonged grade 3 neurotoxicity.

54. The method of any of embodiments 1-53, wherein the neurotoxicity is associated with cerebral edema.

55. The method of any of embodiments 1-54, wherein the ALL is adult ALL or pediatric ALL.

56. The method of any of embodiments 1-55, wherein the sample is a tumor sample and/or the sample comprises or is likely to comprise tumor cells.

57. The method of any of embodiments 1-56, wherein the sample is or comprises a bone marrow sample, blood sample, plasma sample, or serum sample.

58. The method of any of embodiments 1-57, wherein the sample is or comprises a bone marrow sample.

59. The method of embodiment 58, wherein the sample is or comprises a bone marrow aspirate.

60. The method of any of embodiments 1-59, wherein the presence, absence or level of expression of one, two, three, four, five, six, seven, eight, nine, ten or more gene products is assessed or compared.

61. The method of any of embodiments 1-60, wherein the one or more gene products or portion or fragment thereof is a polynucleotide or a portion thereof.

62. The method of embodiment 61, wherein the polynucleotide is an RNA.

63. The method of embodiment 61 or embodiment 62, wherein the one or more gene products or portions thereof is an messenger RNA (mRNA) transcript or a partial transcript thereof.

64. The method of embodiment 63, wherein the one or more gene products is assessed from complementary DNA (cDNA), optionally based on the mRNA transcript or partial transcript thereof.

65. The method of embodiment 64, wherein the cDNA is prepared by PCR amplification, optionally by RT-PCR or quantitative PCR, of the mRNA transcript or partial transcript thereof.

66. The method of any of embodiments 1-63, wherein the presence, absence or level of expression of the one or more gene products or portions thereof is assessed by polymerase chain reaction (PCR), northern blotting, microarray, and/or a sequencing technique.

67. The method of any of embodiments 1-58, wherein the one or more gene products or portions thereof comprise a protein or a portion thereof.

68. The method of embodiment 67, wherein the presence, absence or level of expression of the one or more gene products or portions thereof is measured by an immunoassay, nucleic acid-based or protein-based aptamer techniques, high precision liquid chromatography (HPLC), peptide sequencing, and/or mass spectrometry.

69. The method of embodiment 67 or embodiment 68, wherein the presence, absence or level of the one or more gene products or portions thereof is measured by immunoassay and the immunoassay is selected from enzyme-linked immunosorbent assay (ELISA), enzyme immunoassay (EIA), radioimmunoassay (RIA), surface plasmon resonance (SPR), Western Blot, Lateral flow assay, immunohistochemistry, protein array or immuno-PCR iPCR).

70. The method of any of embodiments 1-69, wherein the gene reference value, or each of the gene reference values individually for each of the at least one or more gene product, is determined by application of an algorithm to the level, concentration or amount of expression in a control sample, or the average of such level, concentration or amount of expression among a plurality of control samples.

71. The method of any of embodiments 1-70, wherein the gene reference value for the at least one gene product of (a), or each of the gene reference values individually for each of the at least one or more gene product of (a), is a value that:

i) is within 25%, within 20%, within 15%, within 10%, or within 5% above the average level, concentration or amount, and/or is within a standard deviation above the average level, concentration or amount, of the at least one gene product in a plurality of control samples;

ii) is above the highest level, concentration or amount of the at least one gene product, optionally within 50%, within 25%, within 20%, within 15%, within 10%, or within 5% above such highest level, concentration or amount, measured in at least one sample from among a plurality of control samples; and/or

iii) is above the highest level, concentration or amount of the at least one gene product as measured among more than 75%, 80%, 85%, 90%, or 95%, or 98% of samples from a plurality of control samples;

wherein the plurality of control samples are a plurality of biological samples obtained from a group of subjects prior to receiving a immunotherapy for treating a disease or condition, optionally ALL, wherein each of the subjects of the group went on to develop severe neurotoxicity, optionally grade 3 or higher, prolonged grade 3 or higher or grade 4 or 5 neurotoxicity, after receiving the immunotherapy for treating the same disease or condition.

72. The method of embodiment 71, further wherein the gene reference value for the at least one gene product of (a), or each of the gene reference values individually for each of the at least one or more gene product of (a), is:

below the lowest level, concentration, or amount, optionally within 50%, within 25%, within 20%, within 15%, within 10%, or within 5% below the lowest level, concentration or amount, of the at least one gene product observed in a sample from among a second plurality of control samples; and/or

below the level, concentration or amount of the at least one gene product measured as measured in more than 75%, 80%, 85%, 90%, 95%, or 98% of samples from among a second plurality of control samples

wherein the second plurality of control samples is obtained from a group of subjects prior to receiving the same immunotherapy for treating the same disease or condition, wherein each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition.

73. The method of any of embodiments 1-70, wherein the gene reference value for the at least one gene product of (a), or each of the gene reference values individually for each of the at least one or more gene product of (a), is a value that:

i) is within 25%, within 20%, within 15%, within 10%, or within 5% above the average level, concentration or amount, and/or is within a standard deviation above the average level, concentration or amount, of the at least one gene product in a plurality of control samples;

ii) is above the highest level, concentration or amount of the at least one gene product, optionally within 50%, within 25%, within 20%, within 15%, within 10%, or within 5% above such highest level, concentration or amount, as measured in at least one sample from among a plurality of control samples; and/or

iii) is above the highest level, concentration or amount of the at least one gene product measured among more than 75%, 80%, 85%, 90%, 95%, or 98% of samples from a plurality of control samples;

wherein the plurality of control samples are a plurality of biological samples obtained from a group of subjects prior to receiving a immunotherapy for treating a disease or condition, optionally ALL, wherein each of the subjects of the group has ALL that is not Philadelphia chromosome positive (PH+) or Philadelphia-like (Ph-like) subtype of ALL.

74. The method of embodiment 73, further wherein the gene reference value for the at least one gene product of (a), or each of the gene reference values individually for each of the at least one or more gene product of (a), is:

below the lowest level, concentration, or amount, optionally within 50%, within 25%, within 20%, within 15%, within 10%, or within 5% below the lowest level, concentration or amount, of the at least one gene product observed in a sample from among a second plurality of control samples; and/or

below the level, concentration or amount measured in more than 75%, 80%, 85%, 90%, 95%, or 98% of samples from among a plurality of control samples,

wherein the second plurality of control samples is obtained from a group of subjects prior to receiving the same immunotherapy for treating the same disease or condition, wherein each of the subjects has ALL that is Philadelphia chromosome positive (Ph+) or Philadelphia-like (Ph-like) subtype of ALL.

75. The method of any of embodiments 1-74, wherein the gene reference value for the at least one gene product of (b), or each of the gene reference values individually for each of the at least one or more gene product of (b), is a value that:

i) is within 25%, within 20%, within 15%, within 10%, or within 5% below the average level, concentration or amount, and/or is within a standard deviation below the average level, concentration or amount, of the at least one gene product in a plurality of control samples;

ii) is below the lowest level, concentration or amount of the at least one gene product, optionally within 50%, within 25%, within 20%, within 15%, within 10%, or within 5% below the lowest level, concentration or amount, as measured in at least one sample from among a plurality of control samples;

iii) is below the lowest level, concentration or amount of the at least one gene product measured among more than 75%, 80%, 85%, 90%, 95%, or 98% of samples from a plurality of control samples;

wherein the plurality of control samples are a plurality of biological samples obtained from a group of subjects prior to receiving a immunotherapy for treating a disease or condition, wherein each of the subjects of the group went on to develop severe neurotoxicity, optionally grade 3 or higher, prolonged grade 3 or higher or grade 4 or 5 neurotoxicity, after receiving the immunotherapy for treating the same disease or condition.

76. The method of embodiment 75, further wherein the gene reference value for the at least one gene product of (b), or each of the gene reference values individually for each of the at least one or more gene product of (b) is:

above the highest level, concentration, or amount of the at least one gene product, optionally within 50%, within 25%, within 20%, within 15%, within 10%, or within 5% above such level, concentration or amount, measured in at least one sample from among a second plurality of control samples; and/or

above the level, concentration or amount of the at least one gene product measured in more than 75%, 80%, 85%, 90%, 95%, or 98% of samples from among a second plurality of control samples,

wherein the second plurality of control samples are a plurality of control samples obtained from a group of subjects prior to receiving the immunotherapy for treating the disease or condition, wherein each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition.

77. The method of any of embodiments 1-74, wherein the gene reference value for the at least one gene product of (b), or each of the gene reference values individually for each of the at least one or more gene product of (b), is a value that:

i) is within 25%, within 20%, within 15%, within 10%, or within 5% below the average level, concentration or amount, and/or is within a standard deviation below the average level, concentration or amount, of the at least one gene product in a plurality of control samples;

ii) is below the lowest level, concentration or amount of the at least one gene product, optionally within 50%, within 25%, within 20%, within 15%, within 10%, or within 5% below such lowest level, concentration or amount, as measured in at least one sample from among a plurality of control samples;

iii) is below the lowest level, concentration or amount of the at least one gene product measured among more than 75%, 80%, 85%, 90%, 95%, or 98% of samples from a plurality of control samples;

wherein the plurality of control samples are a plurality of biological samples obtained from a group of subjects prior to receiving a immunotherapy for treating a disease or condition, optionally ALL, wherein each of the subjects of the group has ALL that is not Philadelphia chromosome positive (PH+) or Philadelphia-like (Ph-like) subtype of ALL.

78. The method of embodiment 77, further wherein the gene reference value for the at least one gene product of (b), or each of the gene reference values individually for each of the at least one or more gene product of (b) is:

above the highest level, concentration, or amount of the at least one gene product, optionally within 50%, within 25%, within 20%, within 15%, within 10%, or within 5% above such level, concentration or amount, measured in at least one sample from among a second plurality of control samples; and/or

is above the level, concentration or amount of the at least one gene product measured among more than 75%, 80%, 85%, 90%, 95%, or 98% of samples from a second plurality of control samples,

wherein the second plurality of control samples are a plurality of control samples obtained from a group of subjects prior to receiving the immunotherapy for treating the same disease or condition, wherein each of the subjects has ALL that is Philadelphia chromosome positive (Ph+) or Philadelphia-like (Ph-like) subtype of ALL.

79. The method of any of embodiments 70-78, wherein the control sample or the plurality of control samples are obtained from one or more subjects who have ALL.

80. The method of any of embodiments 70-79, wherein the control sample or each of the plurality of control samples is from the same biological sample being assessed, optionally is a bone marrow sample.

81. The method of any of embodiments 70-80, wherein the plurality of control samples comprises at least 3, at least 10, at least 20, at least 50, or at least 100 control samples.

82. The method of any of embodiments 1-81, wherein the immunotherapy is a cell therapy comprising cells engineered to express a recombinant receptor, optionally a CAR, wherein the risk or likely risk of the subject developing neurotoxicity following administration of the cell therapy is further based on the value of a parameter that indicates or correlates with the degree of recombinant receptor-dependent, optionally CAR-dependent, activity of the composition, wherein if the value of the parameter is at or greater than a threshold value the subject is at risk of developing neurotoxicity following administration of the immunotherapy when administered to the subject.

83. The method of embodiment 82, wherein the recombinant receptor-dependent activity comprises a measure of the production or accumulation of one or more of a proinflammatory cytokine, or a normalized value thereof 84. The method of embodiment 83, wherein the proinflammatory cytokine is TNF-alpha, IFN-gamma, IL-2, IL-10, or a combination thereof.

85. The method of embodiment 83 or 84, wherein the measure is in an assay involving culture or incubation for a fixed time, optionally 24 hours, of cells of the immunotherapy or sample thereof in the presence of an antigen that binds to the recombinant receptor, cells expressing an antigen that binds to the recombinant receptor, and/or an agent that binds to a recombinant receptor.

86. The method of embodiment 85, wherein the assay is an ELISA.

87. The method of any of embodiment 83-86, wherein the measure of the proinflammatory cytokine is:

(i) a concentration, relative concentration, amount, or relative amount of the cytokine; or

(ii) an amount or relative amount of the cytokine per number of cells of the therapeutic T cell composition, optionally the number of CAR+ T cells of the therapeutic T cell composition; or

(iii) an amount or relative amount of the cytokine per unit of input cells of the given composition per unit of time, optionally one hour; or

(iv) a level indicative of any of (i)-(iii).

88. The method of any of embodiments 82-87, wherein the threshold value of the recombinant receptor-dependent activity is:

i) within 25%, within 20%, within 15%, within 10%, or within 5% below the measure of the recombinant receptor-dependent activity, and/or is within a standard deviation below the such measure, in a plurality of reference compositions;

ii) is below the lowest measure of the recombinant receptor-dependent activity, optionally within 50%, within 25%, within 20%, within 15%, within 10%, or within 5% below such lowest measure, in a composition from among a plurality of reference compositions;

iii) is below the measure of the lowest recombinant receptor-dependent activity among more than 75%, 80%, 85%, 90%, 95%, or 98% of samples from a plurality of reference compositions;

wherein the plurality of reference compositions are a plurality of compositions of the cell therapy comprising T cells expressing the recombinant receptor, optionally the CAR, from among a group of subjects that went on to receive the cell therapy for treating the same a disease or condition, optionally the same disease or condition, optionally ALL, those subjects in the group that went on to develop severe neurotoxicity, optionally grade 3 or higher, prolonged grade 3 or higher or grade 4 or 5 neurotoxicity.

89. The method of any of embodiments 1-88, wherein the immunotherapy specifically binds to an antigen associated with the disease or condition or expressed in cells of the environment of a lesion associated with the disease or condition.

90. The method of embodiment 89, wherein the disease or condition is a cancer.

91. The method of embodiment 89 or 90, wherein the disease or condition is a myeloma, leukemia or lymphoma.

92. The method of any of embodiments 89-91, wherein the disease or condition is a B cell malignancy and/or is acute lymphoblastic leukemia (ALL), adult ALL, chronic lymphoblastic leukemia (CLL), non-Hodgkin lymphoma (NHL), and Diffuse Large B-Cell Lymphoma (DLBCL).

93. The method of any of embodiments 89-92, wherein the disease or condition is acute lymphoblastic leukemia (ALL), optionally adult ALL or pediatric ALL.

94. The method of any of embodiments 89-93, wherein the target antigen is selected from among Trophoblast glycoprotein (TPBG also known as 5T4), 8H9, avb6 integrin, B7-H3, B7-H6, B cell maturation antigen (BCMA), carbonic anhydrase 9 (CA9, also known as CAIX or G250), a cancer-testes antigen, carbonic anhydrase 9 (CAIX), C-C Motif Chemokine Ligand 1 (CCL-1), CD19, CD20, CD22, CEA, hepatitis B surface antigen, CD23, CD24, CD30, CD33, CD38, CD44, CD44v6, CD44v7/8, CD123, CD133, CD138, CD171, carcinoembryonic antigen (CEA), CE7, a cyclin, cyclin A2, c-Met, dual antigen, chondroitin sulfate proteoglycan 4 (CSPG4), epidermal growth factor protein (EGFR), epithelial glycoprotein 2 (EPG-2), epithelial glycoprotein 40 (EPG-40), ephrin receptor A2 (EPHa2), ephrinB2, erb-B2, erb-B3, erb-B4, erbB dimers, epidermal growth factor protein (EGFR vIII), estrogen receptor, fetal acetylcholine receptor (Fetal AchR), folate receptor alpha, folate binding protein (FBP), ephrin receptor A2 (FCRL5; also known as Fc receptor homolog 5 or FCRH5), fetal acetylcholine receptor, G250/CAIX, ganglioside GD2, ganglioside GD3, glycoprotein 100 (gp100), glypican-3 (GPC3), G Protein Coupled Receptor 5D (GPRC5D), Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen Al (HLA-A1), Human leukocyte antigen A2 (HLA-A2), IL-22 receptor alpha(IL-22R-alpha), IL-13 receptor alpha 2 (IL-13Ra2), kinase insert domain receptor (kdr), kappa light chain, Lewis Y, L1-cell adhesion molecule (L1-CAM), CE7 epitope of L1-CAM, Leucine Rich Repeat Containing 8 Family Member A (LRRC8A), Lewis Y, Melanoma-associated antigen (MAGE)-Al, MAGE-A3, MAGE-A6, MAGE-A10, melan A (MART-1), mesothelin (MSLN), c-Met, murine CMV, mucin 1 (MUC1), MUC16, neural cell adhesion molecule (NCAM), natural killer group 2 member D (NKG2D), NKG2D ligands, cancer/testis antigen 1B (CTAG, also known as NY-ESO-1 and LAGE-2), 0-acetylated GD2 (OGD2), oncofetal antigen, Preferentially expressed antigen of melanoma (PRAME), prostate stem cell antigen (PSCA), progesterone receptor, a prostate specific antigen, prostate specific membrane antigen (PSMA), survivin, Receptor Tyrosine Kinase Like Orphan Receptor 1 (ROR1), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGF receptors or VEGFR), vascular endothelial growth factor receptor 2 (VEGF-R2), Wilms Tumor 1 (WT-1), a pathogen-specific antigen.

95. The method of any of embodiment 1-94, wherein the immunotherapy is a T cell-engaging therapy comprising a bispecific antibody, wherein at least one binding portion specifically binds to a T cell antigen.

96. The method of embodiment 95, wherein the at least one binding portion specifically binds to CD3.

97. The method of embodiment 95 or 96, wherein a second binding portion of the bispecific antibody binds to an antigen, optionally wherein the antigen is CD19.

98. The method of any of embodiments 1-97, wherein the immunotherapy is a cell therapy.

99. The method of any of embodiments 1-94 or 98, wherein the immunotherapy is a T cell therapy comprising genetically engineered cells expressing a recombinant receptor.

100. The method of any of embodiments 82-87, 98, or 99, wherein the recombinant receptor is a T cell receptor or a functional non-T cell receptor.

101. The method of any of embodiments 82-87, 98, or 99, wherein the recombinant receptor is a chimeric antigen receptor (CAR).

102. The method of any of embodiments 89-96, wherein the recombinant receptor is an anti-CD19 CAR.

103. The method of embodiment 101 or 102, wherein the CAR comprises an extracellular antigen-recognition domain that specifically binds to the antigen and an intracellular signaling domain comprising an ITAM, wherein optionally, the intracellular signaling domain comprises an intracellular domain of a CD3-zeta (CD3) chain; and/or wherein the CAR further comprises a costimulatory signaling region, which optionally comprises a signaling domain of CD28 or 4-1BB.

104. The method of any of embodiments 82-87 or 99-103, wherein the genetically engineered cells comprise T cells or NK cells.

105. The method of any of embodiments 82-87 or 99-104, wherein the engineered cells comprise T cells, and the T cells comprise CD4+ and/or CD8+ T cells.

106. The method of embodiment 105, wherein the T cells are primary T cells obtained from a subject.

107. The method of any of embodiments 1-106, wherein the immunotherapy comprises the administration of from or from about 1×105 to 1×108 total recombinant receptor-expressing cells, total T cells, or total peripheral blood mononuclear cells (PBMCs), from or from about 5×105 to 1×107 total recombinant receptor-expressing cells, total T cells, or total peripheral blood mononuclear cells (PBMCs) or from or from about 1×106 to 1×107 total recombinant receptor-expressing cells, total T cells, or total peripheral blood mononuclear cells (PBMCs), each inclusive.

108. The method of any of embodiments 1-107, wherein the immunotherapy comprises the administration of no more than 1×108 total recombinant receptor-expressing cells, total T cells, or total peripheral blood mononuclear cells (PBMCs), no more than 1×107 total recombinant receptor-expressing cells, total T cells, or total peripheral blood mononuclear cells (PBMCs), no more than 0.5×107 total recombinant receptor-expressing cells, total T cells, or total peripheral blood mononuclear cells (PBMCs), no more than 1×106 total recombinant receptor-expressing cells, total T cells, or total peripheral blood mononuclear cells (PBMCs), no more than 0.5×106 total recombinant receptor-expressing cells, total T cells, or total peripheral blood mononuclear cells (PBMCs).

109. A kit, comprising reagents for detecting the expression of two or more gene products or portions thereof in a sample, wherein the two or more gene products are encoded by two or more of ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, ZNF415, ENG, SELE, ICAM3, PCDHGA9, FMNL1, or IL6R, or a portion or a fragment of any of the forgoing.

110. The kit of embodiment 109, wherein the two or more gene products are human gene products.

111. The kit of embodiment 109 or 110, wherein the kit comprises reagents for detecting the expression of at least 3, at least 4, at least 5, at least 6, at least 7, at least 8, at least 9, at least 10, at least 11, at least 12, at least 13, at least 14, at least 15, at least 20, at least 25, at least 30, at least 40, or at least 50 gene products.

112. The kit of any of embodiments 109-111, wherein the kit comprises reagents for detecting the expression of at least 3 gene products.

113. The kit of any of embodiments 109-112, wherein the kit comprises reagents for detecting the expression of at least 5 gene products.

114. The kit of any of embodiments 109-113, wherein at least one of the two or more gene products is from a first group of gene products that negatively correlate to a risk of developing neurotoxicity, wherein the first group comprises gene products encoded by ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, and portions or a fragments of any of the forgoing.

115. The kit of any of embodiments 109-114, wherein at least one of the two or more gene products is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing.

116. The kit of any of embodiments 109-115, wherein at least one of the two or more gene products is a gene product encoded by JCHAIN, MUC4, CA6, WNT9A, ADGRF1 or CCL17, or a portion or fragment of any of the foregoing.

117. The kit of any of embodiments 109-116, wherein at least one of the two or more gene products is a gene product encoded by CCL17 or is a portion or fragment thereof.

118. The kit of any of embodiments 109-117, wherein at least one of the two or more gene products is from a second group of gene products that negatively correlate to a risk of developing neurotoxicity, wherein the second group comprises gene products encoded by ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, and ZNF415, or portions or a fragments of any of the forgoing.

119. The kit of any of embodiments 109-118, wherein at least one of the two or more gene products is a gene product encoded by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

120. The kit of any of embodiments 109-119, wherein at least one of the two or more gene products is a gene product encoded by PINLYP, ASAP2, FMNL2, PTCH1, TTC28, PCDHGA6, PCDHGB6 or PCDHGA12, or a portion or fragment of any of the foregoing.

121. The kit of any of embodiments 109-120, wherein at least one of the two or more gene products is a gene product encoded by PINLYP or PCDHGA12, or a portion or fragment of any of the foregoing.

122. The kit of any of embodiments 109-121 wherein at least one of the two or more gene products is a gene product, or a portion or fragment thereof, from the first group of gene products that negatively correlate to a risk of developing neurotoxicity and at least one of the gene products is a gene product, or a portion or fragment thereof, from the second group of gene products that positively correlate to a risk of developing neurotoxicity.

123. The kit of embodiment 122, wherein the at least one of the gene products from the first group is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing.

124. The kit of embodiment of embodiment 122 or 123, wherein the at least one of the gene products from the first group is a gene product encoded by JCHAIN, MUC4, CA6, WNT9A, ADGRF1 or CCL17, or a portion or fragment of any of the foregoing.

125. The kit of any of embodiments 122-124, wherein the at least one of the gene products from the first group is a gene product encoded by CCL17 or is a portion or fragment thereof.

126. The kit of any of embodiments 122-125, wherein the at least one of the gene products from the second group is a gene product encoded by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

127. The kit of any of embodiments 122-126, wherein the at least one of the gene products from the second group is a gene product encoded by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

128. The kit of any of embodiments 122-127, wherein the at least one of the gene products from the second group is a gene product encoded by PINLYP, ASAP2, FMNL2, PTCH1, TTC28, PCDHGA6, PCDHGB6 or PCDHGA12, or a portion or fragment of any of the foregoing.

129. The kit of any of embodiments 122-128, wherein the at least one of the gene products from the second group is a gene product encoded by PINLYP or PCDHGA12, or a portion or fragment of any of the foregoing.

130. The kit of embodiment 122, wherein the at least one of the gene products from the first group is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing; and wherein the at least one of the gene products from the second group is a gene product encoded by ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

131. The kit of embodiment 122 or 130, wherein the at least one of the gene products from the first group is a gene product encoded by JCHAIN, MUC4, CA6, WNT9A, ADGRF1 or CCL17, or a portion or fragment of any of the foregoing; and wherein the at least one of the gene products from the second group is a gene product encoded by PINLYP, ASAP2, FMNL2, PTCH1, TTC28, PCDHGA6, PCDHGB6 or PCDHGA12, or a portion or fragment of any of the foregoing.

132. The kit of any of embodiments 122, 130, 131, wherein the at least one of the gene products from the first group is a gene product encoded by CCL17 or is a portion or fragment thereof; and

wherein the at least one of the gene products from the second group is a gene product encoded by PINLYP or PCDHGA12, or a portion or fragment thereof.

133. The kit of any of embodiments 109-132, wherein the two or more gene products are or comprise mRNA.

134. The kit of embodiment 133, further comprising a reverse transcriptase, a DNA polymerase, and/or dNTPs wherein the two or more gene products are detected by a PCR reaction, optionally RT-PCR or quantitative PCR

135. The kit of any of embodiments 109-122, wherein at least one of the two or more gene products is a gene product expressed by CCL17, CCR6, GAS6, GL12, PTP4A3, or IL2RA, or a portion or fragment thereof.

136. The kit of embodiment 134 or 135, wherein the reagents comprise oligonucleotide primers that are complementary to, bind to, and/or are capable of binding to the one or more mRNA gene products.

137. The kit of any of embodiments 135-136, wherein the oligonucleotide primers are between 10 and 30 nucleotides in length, optionally between 15 and 25 nucleotides in length.

138. The kit of any of clams 133-137, wherein the reagents comprise one or more oligionucleotide and/or polynucleotide probes that are to, bind to, and/or are capable of binding to the one or more mRNA gene products.

139. The kit of any of embodiments 134-138, wherein probes are between 25 and 1,000 nucleotides in length, optionally between 50 and 500 nucleotides in length, between 50 and 200 nucleotides in length, between 100-300 nucleotides in length, or between 300 and 700 nucleotides in length.

140. The kit of any of embodiments 134-139, wherein the probes are tagged with a label, and wherein the label is detectable when the probe is bound to the mRNA gene product and/or cDNA derived from an RNA gene product.

141. The kit of any of embodiments 134-140, wherein the rt-PCR reaction is a multiplex rt-PCR reaction, optionally wherein the multiplex reaction detects two, three, four, five, or more than five RNA gene products, and/or cDNA derived from the RNA gene products.

142. The kit of embodiment 133, wherein the two or more RNA gene products are detected by a microarray.

143. The kit of embodiment 142, comprising an array, wherein the array comprises at least 5 reagents immobilized on a solid support, wherein each of the reagents:

are or comprise nucleic acid probes that bind to, are capable of binding to, and/or are complementary to the mRNA gene product and/or cDNA derived from an RNA gene product;

have a length of between 20 to 500 nucleotides. 144. The kit of embodiment 133, wherein the two or more RNA gene products are detected by RNA-seq.

145. The kit of any of embodiments 109-132, wherein the two or more gene products are or comprise proteins or variants or fragments thereof.

146. The kit of embodiment 145, wherein the protein gene product are detected by an immunoassay, optionally an immunoassay selected from enzyme-linked immunosorbent assay (ELISA), enzyme immunoassay (EIA), radioimmunoassay (RIA), surface plasmon resonance (SPR), Western Blot, Lateral flow assay, immunohistochemistry, protein array or immuno-PCR iPCR).

147. The kit of embodiments 145 or 146, wherein the reagents are or comprise antibodies or antigen binding fragments or variants thereof, wherein the antibodies or the antigen binding fragments or variants thereof bind to and/or are capable of binding to the protein gene products.

148. The kit of embodiment 147, the antibodies or the antigen binding fragments or variants thereof each comprise a bound or attached detectable label.

149 The kit of any of embodiment 148, wherein the detectable label is a fluorescent label, optionally wherein the fluorescent label comprises Alexa Fluor 346, Alexa Fluor 405, Alexa Fluor 430, Alexa Fluor 488, Alexa Fluor 647, Alexa Fluor 555, Alexa Fluor 568, Alexa Fluor 700, R-phycoerythrin, Aqua, Texas-Red, FITC, rhodamine, a rhodamine derivative, fluorescein, a fluorescein derivative, cascade blue, Cy5 or Cy3.

150. The kit of any of embodiments 109-149, further comprising an antibody or antigen-binding fragment thereof that binds to and/or is capable of binding to a proinflammatory cytokine.

151. The method of embodiment 150, wherein the proinflammatory cytokine is TNF-alpha, IFN-gamma, IL-2, IL-10, or a combination thereof.

152. The kit of embodiment 150 or 151, wherein proinflammatory cytokine is TNF-alpha.

153. The kit of any embodiments 150-152, wherein the proinflammatory cytokine is detected with ELISA; optionally wherein the kit further comprises a solid support comprising a plurality of wells, and wherein the antibody or antigen-binding fragment thereof that binds the proinflammatory cytokine is bound to the solid support, optionally at the inside of the wells.

154. The kit of any of embodiments 109-153, further comprising an immunotherapy.

155. The kit of embodiment 154, wherein the immunotherapy is a cell therapy or is a T cell-engaging therapy, optionally wherein the cell therapy comprises cells engineered to express a recombinant receptor.

156. The kit of embodiment 154 or 155, wherein the immunotherapy specifically binds to an antigen associated with a disease or condition and/or expressed in cells associated with the disease or condition.

157. The kit of embodiment 156, wherein the disease or condition is a cancer.

158. The kit of embodiment 156 or 157, wherein the disease or condition is a myeloma, leukemia or lymphoma.

159. The kit of any of embodiments 156-158, wherein the disease or condition is a B cell malignancy and/or is acute lymphoblastic leukemia (ALL), adult ALL, chronic lymphoblastic leukemia (CLL), non-Hodgkin lymphoma (NHL), and Diffuse Large B-Cell Lymphoma (DLBCL).

160. The kit of any of embodiments 156-159, wherein the target antigen is selected from among Trophoblast glycoprotein (TPBG also known as 5T4), 8H9, avb6 integrin, B7-H3, B7-H6, B cell maturation antigen (BCMA), carbonic anhydrase 9 (CA9, also known as CAIX or G250), a cancer-testes antigen, carbonic anhydrase 9 (CAIX), C-C Motif Chemokine Ligand 1 (CCL-1), CD19, CD20, CD22, CEA, hepatitis B surface antigen, CD23, CD24, CD30, CD33, CD38, CD44, CD44v6, CD44v7/8, CD123, CD133, CD138, CD171, carcinoembryonic antigen (CEA), CE7, a cyclin, cyclin A2, c-Met, dual antigen, chondroitin sulfate proteoglycan 4 (CSPG4), epidermal growth factor protein (EGFR), epithelial glycoprotein 2 (EPG-2), epithelial glycoprotein 40 (EPG-40), ephrin receptor A2 (EPHa2), ephrinB2, erb-B2, erb-B3, erb-B4, erbB dimers, epidermal growth factor protein (EGFR vIII), estrogen receptor, fetal acetylcholine receptor (Fetal AchR), folate receptor alpha, folate binding protein (FBP), ephrin receptor A2 (FCRL5; also known as Fc receptor homolog 5 or FCRH5), fetal acetylcholine receptor, G250/CAIX, ganglioside GD2, ganglioside GD3, glycoprotein 100 (gp100), glypican-3 (GPC3), G Protein Coupled Receptor 5D (GPRC5D), Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22R-alpha), IL-13 receptor alpha 2 (IL-13Ra2), kinase insert domain receptor (kdr), kappa light chain, Lewis Y, L1-cell adhesion molecule (L1-CAM), CE7 epitope of Ll-CAM, Leucine Rich Repeat Containing 8 Family Member A (LRRC8A), Lewis Y, Melanoma-associated antigen (MAGE)-A1, MAGE-A3, MAGE-A6, MAGE-A10, melan A (MART-1), mesothelin (MSLN), c-Met, murine CMV, mucin 1 (MUC1), MUC16, neural cell adhesion molecule (NCAM), natural killer group 2 member D (NKG2D), NKG2D ligands, cancer/testis antigen 1B (CTAG, also known as NY-ESO-1 and LAGE-2), 0-acetylated GD2 (OGD2), oncofetal antigen, Preferentially expressed antigen of melanoma (PRAME), prostate stem cell antigen (PSCA), progesterone receptor, a prostate specific antigen, prostate specific membrane antigen (PSMA), survivin, Receptor Tyrosine Kinase Like Orphan Receptor 1 (ROR1), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGF receptors or VEGFR), vascular endothelial growth factor receptor 2 (VEGF-R2), Wilms Tumor 1 (WT-1), a pathogen-specific antigen.

161. The kit of any of embodiments 156-160, wherein the disease or condition is ALL.

162. The kit of any of embodiments 156-162, wherein the immunotherapy is a T cell-engaging therapy comprising a bispecific antibody, wherein at least one binding portion specifically binds to a T cell antigen.

163. The kit of embodiment 161 or 162, wherein the at least one binding portion specifically binds to CD3

164. The kit of any of embodiments 161-163, wherein a second binding portion of the bispecific antibody binds to an antigen, optionally wherein the antigen is CD19.

165. The kit of any of embodiments 156-161, wherein the immunotherapy is a cell therapy.

166. The kit of any of embodiments156-161 or 165, wherein the immunotherapy is a T cell therapy comprising genetically engineered cells expressing a recombinant receptor that specifically binds to a ligand.

167. The kit of embodiment 166, wherein the recombinant receptor is a T cell receptor or a functional non-T cell receptor.

168. The kit of embodiment 167 or 168, wherein the recombinant receptor is a chimeric antigen receptor (CAR).

169. The kit of embodiment 168, wherein the CAR comprises an extracellular antigen-recognition domain that specifically binds to the antigen and an intracellular signaling domain comprising an ITAM, wherein optionally, the intracellular signaling domain comprises an intracellular domain of a CD3-zeta (CD3) chain; and/or wherein the CAR further comprises a costimulatory signaling region, which optionally comprises a signaling domain of CD28 or 4-1BB.

170. The kit of any of embodiments 109-169, further comprising an agent or other treatment capable of treating, preventing, delaying, reducing or attenuating the development or risk of development of a toxicity.

171. The kit of embodiment 170, wherein the agent or other treatment is or comprises one or more of a steroid; an antagonist or inhibitor of a cytokine receptor or cytokine selected from among IL-10, IL-10R, IL-6, IL-6 receptor, IFNy, IFNGR, IL-2, IL-2R/CD25, MCP-1, CCR2, CCR4, MIP1β, CCRS, TNF-alpha, TNFR1, IL-1, and IL-1Ralpha/IL-1beta; or an agent capable of preventing, blocking or reducing microglial cell activity or function.

172. The kit of embodiment 171, wherein the antagonist or inhibitor is or comprises an agent selected from among an antibody or antigen-binding fragment, a small molecule, a protein or peptide and a nucleic acid.

173. The kit of any of embodiments 170-172, wherein the agent or other treatment is an anti-IL-6 antibody or an anti-IL6 receptor antibody.

174. The kit of any of embodiments 170-173, wherein the agent or other treatment is or comprises an agent selected from among tocilizumab, siltuximab, clazakizumab, sarilumab, olokizumab (CDP6038), elsilimomab, ALD518/BMS-945429, sirukumab (CNTO 136), CPSI-2634, ARGX-109, FE301 and FM101.

175. The kit of any of embodiments 170-174, wherein the agent or other treatment is or comprises tocilizumab.

176. The kit of any of embodiments 170-175, wherein the agent or other treatment is or comprises siltuximab.

177. The article of manufacture of embodiment 171, wherein the steroid is or comprises dexamethasone.

178. The kit of embodiment 171, wherein the agent capable of preventing, blocking or reducing microglial cell activity or function is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-KB), activates a CB2 receptor and/or is a CB2 agonist, a phosphodiesterase inhibitor, inhibits microRNA-155 (miR-155) or upregulates microRNA-124 (miR-124).

179. The kit 178, wherein the agent capable of preventing, blocking or reducing microglial cell activation or function is a small molecule, peptide, protein, antibody or antigen-binding fragment thereof, an antibody mimetic, an aptamer, or a nucleic acid molecule.

180. The kit of embodiment 178 or 179, wherein the agent is selected from minocycline, naloxone, nimodipine, Riluzole, MOR103, lenalidomide, a cannabinoid (optionally WIN55 or 212-2), intravenous immunoglobulin (IVIg), ibudilast, anti-miR-155 locked nucleic acid (LNA), MCS110, PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945, emactuzumab, IMC-CS4, FPA008, LY-3022855, AMG-820 and TG-3003.

181. The kit of any of embodiments 178-180, wherein the agent is an inhibitor of colony stimulating factor 1 receptor (CSF1R).

182. The kit of any of embodiments 178-181, wherein the inhibitor is selected from:

PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;

emactuzumab, IMC-CS4, FPA008, LY-3022855, AMG-820 and TG-3003 or is an antigen-binding fragment thereof;

or a combination of any of the foregoing.

183. The kit of any of embodiments 178-181, wherein the inhibitor is PLX-3397.

184. An article of manufacture, comprising a kit of any one of embodiments 109-183, and instructions for using the reagents to assay a biological sample from a subject that is a candidate for treatment, optionally with an immunotherapy.

185. The article of manufacture of embodiment 184, wherein the instructions specify assessing the presence, absence or level of expression of the two or more gene products or portions thereof in the sample, and comparing the presence, absence, or level of expression of the two or more gene products or portions thereof to gene reference values, wherein the comparison indicates the risk or likely risk of the subject developing neurotoxicity following administration of the immunotherapy when administered to the subject.

186. The article of manufacture of embodiment of embodiment 185, wherein the instructions specify each of the one or more gene products is individually compared to a gene reference value for the respective gene product.

187. The article of manufacture of any of embodiments 184-186, wherein the instructions specify the sample does not comprise cells genetically engineered with the recombinant receptor and/or is obtained from the subject prior to receiving the immunotherapy.

188. The article of manufacture of any of embodiments 184-187, wherein the instructions specify the sample is a tumor sample and/or the sample comprises or is likely to comprise tumor cells.

189. The article of manufacture of any of embodiments 184-188, wherein the sample is or comprises a bone marrow sample, blood sample, plasma sample, or serum sample.

190. The article of manufacture of any of embodiments 184-189, wherein the sample is or comprises a bone marrow sample, optionally a bone marrow aspirate.

191. The article of manufacture of any of embodiments 184-190, wherein the instructions specify the comparison indicates the subject is or is likely at risk of developing neurotoxicity if the at least one gene product from the first group of gene products that negatively correlate to a risk of developing neurotoxicity is at or below a gene reference value and/or the at least one gene product from the second group of gene products that negatively correlate to a risk of developing neurotoxicity is at or above a gene reference value; or the instructions the comparison indicates the subject is not or is likely not at risk of developing neurotoxicity if the at least one gene product of (a) is above a gene reference value and/or the at least one gene product of (b) is below a gene reference value.

192. The article of manufacture of 191, wherein the instructions specify if the comparison indicates the subject is or is likely to develop neurotoxicity, selecting the subject for administration of a therapeutic regimen, the therapeutic regimen comprising administering to the subject:

i. an agent or other treatment capable of treating, preventing, delaying, reducing or attenuating the development or risk of development of a toxicity and (2) the immunotherapy, wherein administration of the agent is to be administered (i) prior to, (ii) within one, two, or three days of, (iii) concurrently with and/or (iv) at first fever following, the initiation of administration of the immunotherapy to the subject;ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy; and/or

iii. the immunotherapy in an in-patient setting and/or with admission to the hospital for one or more days, optionally wherein the immunotherapy is otherwise to be administered to subjects on an outpatient basis or without admission to the hospital for one or more days; or

iv. an alternative therapeutic treatment other than the immunotherapy. 193. The article of manufacture of 192, wherein the instructions specify if the comparison indicates the subject is not or is likely not at risk of developing neurotoxicity, selecting the subject for administration of a therapeutic regimen, the therapeutic regimen comprising administering to the subject:

i. the immunotherapy, optionally at a non-reduced dose, optionally on an outpatient basis or without admission to the hospital for one or more days;

ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or

iii. the immunotherapy in an outpatient setting and/or without admission of the subject to the hospital overnight or for one or more consecutive days and/or is without admission of the subject to the hospital for one or more days.

194. The article of manufacture of embodiment 192 or 193, wherein the instructions specify the results of assessing the presence, absence or level of expression of the one or more gene products or portions thereof comprises a comparison to a gene reference value, wherein the comparison indicates the risk or likely risk of the subject developing neurotoxicity following administration of the immunotherapy when administered to the subject.

195. The article of manufacture of embodiment 194, wherein the instructions specify each of the one or more gene products is individually compared to a gene reference value for the respective gene product.

196. The article of manufacture of embodiment 195 or 196, wherein the instructions specify:

expression of the at least one or more gene products from the first group of gene products negatively correlate to a risk that the subject is or is likely to develop neurotoxicity following administration of the immunotherapy when it is administered; and/or expression of the at least one or more gene products from the second group of gene products positively correlates to a risk that the subject is or is likely to develop neurotoxicity following administration of the immunotherapy when it is administered.

197. The article of manufacture of any of embodiments 184-196, wherein the instructions specify if the assessing indicates the subject is or is likely to develop neurotoxicity following administration of the immunotherapy, the therapeutic regimen comprises administering to the subject:

i. the agent or other treatment capable of treating, preventing, delaying, reducing or attenuating the development or risk of development of a toxicity and (2) the immunotherapy, wherein administration of the agent is to be administered (i) prior to, (ii) within one, two, or three days of, (iii) concurrently with and/or (iv) at first fever following, the initiation of administration of the immunotherapy to the subject;

ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy; and/or

iii. the immunotherapy in an in-patient setting and/or with admission to the hospital for one or more days, optionally wherein the immunotherapy is otherwise to be administered to subjects on an outpatient basis or without admission to the hospital for one or more days; or

iv. an alternative therapeutic treatment other than the immunotherapy.

198. The article of manufacture of any of embodiments 184-197, wherein the instructions specify if the assessing indicates the subject is not or is likely not to develop neurotoxicity following administration of the immunotherapy, the therapeutic regimen comprises administering to the subject:

i. the immunotherapy, optionally at a non-reduced dose, optionally on an outpatient basis or without admission to the hospital for one or more days;

ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or iii. the immunotherapy in an outpatient setting and/or without admission of the subject to the hospital overnight or for one or more consecutive days and/or is without admission of the subject to the hospital for one or more days.

199. The article of manufacture of embodiment 184,wherein the instructions specify:

selecting a subject that exhibits a Philadelphia chromosome (Ph+) and/or Ph chromosome-like (Ph-like) molecular subtype of acute lymphoblastic leukemia (ALL); and

administering to the subject a immunotherapy comprising a dose of cells expressing a recombinant receptor that binds to an antigen associated with the ALL.

200. The article of manufacture of embodiment 199, wherein the instructions specify the subject exhibits a PH+ and/or a Ph-like molecular subtype of ALL if the comparison indicates the at least one gene product of the first group of gene products is above a gene reference value and/or the at least one gene product of the second group of gene products is below a gene reference value.

201. The article of manufacture of any of embodiments 184-200, wherein the instructions specify:

prior to initiation of administration of the dose of cells, the subject has not been administered an agent or treatment capable of treating, preventing, delaying, reducing or attenuating the development or risk of development of a toxicity; and/or

the subject is not administered an agent or treatment for the treatment or prevention or reduction or attenuation of a neurotoxicity and/or a cytokine release syndrome or risk thereof, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or

the subject is not administered an agent or treatment for the treatment or prevention or reduction or attenuation of a neurotoxicity and/or a cytokine release syndrome or risk thereof, following administration of the dose, prior to or unless the subject exhibits a sign or symptom of the toxicity and/or prior to or unless the subject exhibits a sign or symptom of the toxicity other than a fever, optionally wherein the fever is not a sustained fever or the fever is or has been reduced or reduced by more than 1° C. after treatment with an antipyretic; and/or

the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

202. The article of manufacture of any of embodiments 184-201, wherein the instructions specify:

prior to initiation of administration of the dose of cells, the subject has not been administered an anti-IL-6 or anti-IL-6R antibody, optionally tocilizumab or siltuximab, and/or has not been administered a steroid, optionally dexamethasone

the subject is not administered an anti-IL-6 or anti-IL-6R antibody, optionally tocilizumab or siltuximab, and/or has not been administered a steroid, optionally dexamethasone, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or

the subject is not administered an anti-IL-6 or anti-IL-6R antibody, optionally tocilizumab or siltuximab, and/or has not been administered a steroid, optionally dexamethasone, following administration of the cell dose, prior to, or unless, the subject exhibits a sign or symptom of a toxicity, optionally a neurotoxicity or CRS, and/or prior to, or unless, the subject exhibits a sign or symptom of a toxicity, optionally a neurotoxicity or CRS, other than a fever, optionally wherein the fever is not a sustained fever or the fever is or has been reduced or reduced by more than 1° C. after treatment with an antipyretic; and/or the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

203. The article of manufacture of any of embodiments 184-202, wherein the instructions specify:

the administration is carried out on an outpatient basis and/or without requiring admission to or an overnight stay at a hospital; and

if the subject exhibits a sustained fever or a fever that is or has not been reduced or not reduced by more than 1° C. after treatment with an antipyretic, the subject is admitted to the hospital or to an overnight stay at a hospital and/or is administered an agent or treatment for the treatment or prevention or reduction or attenuation of a neurotoxicity and/or a cytokine release syndrome or risk thereof.

204. The article of manufacture of any of embodiments 184-203, wherein the instructions specify neurotoxicity comprises severe neurotoxicity, optionally at or above grade 4 or grade 5 or at least prolonged grade 3 neurotoxicity.

205. The article of manufacture of any of embodiments 184-204, wherein the instructions specify the neurotoxicity is associated with cerebral edema.

206. The article of manufacture of any of embodiments 184-205, wherein the instructions specify the presence, absence or level of expression of one, two, three, four, five, six, seven, eight, nine, ten or more gene products is assessed or compared.

207. The article of manufacture of any of embodiments 184-206, wherein the instructions specify the one or more gene products or portion or fragment thereof is a polynucleotide or a portion thereof, optionally a messenger RNA (mRNA) transcript or a partial transcript thereof.

208. The article of manufacture of embodiment 207, wherein the instructions specify cDNA is prepared by PCR amplification of the mRNA transcript or partial transcript thereof.

209. The article of manufacture of embodiment 207 or 208, wherein the instructions specify the presence, absence or level of expression of the one or more gene products or portions thereof is assessed by polymerase chain reaction (PCR), northern blotting, microarray, and/or a sequencing technique

210. The article of manufacture of any of embodiments 184-206, wherein the instructions specify the one or more gene products or portions thereof comprise a protein or a portion thereof.

211. The article of manufacture of embodiment 210, wherein the instructions specify the presence, absence or level of expression of the one or more gene products or portions thereof is measured by an immunoassay, nucleic acid-based or protein-based aptamer techniques, high precision liquid chromatography (HPLC), peptide sequencing, and/or mass spectrometry.

212. The article of manufacture of embodiment 211, wherein the instructions specify the presence, absence or level of the one or more gene products or portions thereof is measured by immunoassay and the immunoassay is selected from enzyme-linked immunosorbent assay (ELISA), enzyme immunoassay (EIA), radioimmunoassay (RIA), surface plasmon resonance (SPR), Western Blot, Lateral flow assay, immunohistochemistry, protein array or immuno-PCR iPCR).

213. The article of manufacture of any of embodiments 184-212, wherein the instructions specify the gene reference value, or each of the gene reference values individually for each of the at least one or more gene product, is determined by application of an algorithm to the level, concentration or amount of expression in a control sample, or the average of such level, concentration or amount of expression among a plurality of control samples.

214. The article of manufacture of any of embodiments 184-213, wherein the instructions specify the gene reference value, or each of the gene reference values individually for each of the at least one or more gene product is a determined value.

215. An article of manufacture comprising an immunotherapy and instructions for administering the immunotherapy to a subject that exhibits a Philadelphia chromosome (Ph+) and/or Ph chromosome-like (Ph-like) molecular subtype of acute lymphoblastic leukemia (ALL).

216. The article of manufacture of embodiment 215, wherein the instructions specify the subject exhibits the PH+ and/or the Ph-like molecular subtype based on results of cytogenetic or molecular genetic analysis.

217. The article of manufacture of embodiment 215 or 216, wherein the immunotherapy is a immunotherapy or is a T cell-engaging therapy, optionally wherein the immunotherapy comprises cells engineered to express a recombinant receptor.

218. The article of manufacture of any of embodiments 215-217, wherein the instructions specify the analysis comprises karyotype analysis, fluorescence in situ hybridization (FISH), multicolor FISH, polymerase chain reaction (PCR), a tyrosine kinase inhibitor assay, gene expression profiling or microarray or an immunoassay, optionally an ELISA.

219. The article of manufacture of any of embodiments 215-218, wherein the instructions specify:

the selected subject exhibits one or more of the (9;22)(q34;q11) chromosomal abnormality; deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or comprises a Ph-like gene expression signature;

the subject is selected based on one or more of the presence of the (9;22)(q34;q11) chromosomal abnormality, deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or the presence of a Ph-like gene expression signature.

220. The article of manufacture of any of embodiments 215-219, wherein the instructions specify the presence of the Ph-like gene signature is based on comparison of the presence, absence or level of expression, in a sample from the subject, of at least one gene product to a reference gene value, said at least one gene product is selected from (a) ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, L0645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A or a portion or fragment of any of the foregoing and/or said at least one gene product is selected from (b) ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing, whereby the comparison indicates whether the subject exhibits a Ph-like molecular subtype of ALL.

221. The article of manufacture of embodiment 220, wherein the instructions specify each of the one or more gene products is individually compared to a gene reference value for the respective gene product.

222. The article of manufacture of any of embodiment 220 or embodiment 221, wherein the instructions specify the subject exhibits a Ph-like molecular subtype of ALL if the comparison indicates the at least one gene product of (a) is above a gene reference value and/or the at least one gene product of (b) is below a gene reference value.

223. The article of manufacture of any of embodiments 220-222, wherein the instructions specify the at least one gene product selected from (a) is ADGRF1, BMPR1B, CA6, CD99, CHN2, CRLF2, DENND3, ENAM, GBP5, GLI2, IFITM1, IGJ (JCHAIN), LDB3, LO645744, MDF1C, MUC4, NRXN3, PON2, S100Z, SEMA6A, SLC37A3, SLC2A5, SPATS2L, TMEM154, TP53INP1, TTYH2 or WNT9A, or is a portion of fragment of any of the foregoing.

224. The article of manufacture of any of embodiments 220-223, wherein the instructions specify the at least one gene product selected from (a) is ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A or is a portion or fragment of any of the foregoing.

225. The article of manufacture of any of embodiments 220-224, wherein the instructions specify the at least one gene product selected from (b) is ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing.

226. An article of manufacture comprising an immunotherapy, and instructions for administering a therapeutic regimen to a subject that is a candidate for receiving an immunotherapy for treatment of a disease or condition, wherein the instructions specify the administration is carried out following or based on the results of assessing the presence, absence or level of expression, from a sample from the subject, of one or more gene products or portion thereof, wherein:

(a) at least one of the one or more gene products is selected from ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, CRLF2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS 1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or

(b) at least one of the one or more gene products is selected from ASAP2, ATP9A, CCNA1, CDHR3, CECR2, DLX1, DPYSL3, EHD4, FMNL2, GGA2, HHIPL1, HMX3, IGF2BP1, IL3RA, IRX5, KCNIP1, KIAA1644, LINC00092, LINC01483, MIB1, MMP14, NOM1, OTOA, PCDHGA12, PCDHGA4, PCDHGA6, PCDHGBS, PCDHGB6, PINLYP, PPM1E, PRKD1, PROKR2, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TNKS1BP1, TTC28, PCDHGA9, FMNL1, or ZNF415 or is a portion or fragment thereof.

227. The article of manufacture of embodiment 226, wherein the immunotherapy is a cell therapy or is a T cell-engaging therapy, optionally wherein the cell therapy comprises cells engineered to express a recombinant receptor.

228. The article of manufacture of embodiment 226 or 227, wherein at least one of the one or more gene products is from (a) and at least one of the one or more gene products is from (b).

229. The article of manufacture of any of embodiments 226-228, wherein the instructions specify the sample is obtained from the subject prior to receiving the immunotherapy and/or the sample does not comprise cells expressing a recombinant receptor.

230. The article of manufacture of any of embodiments 226-229, wherein the instructions specify the presence, absence or level of the one or more gene products is associated with a risk of developing neurotoxicity following administration of the immunotherapy.

231. The article of manufacture of any of embodiments 226-230, wherein the instructions specify the results of assessing the presence, absence or level of expression of the one or more gene products or portions thereof comprises a comparison to a gene reference value, wherein the comparison indicates the risk or likely risk of the subject developing neurotoxicity following administration of the immunotherapy when administered to the subject.

232. The article of manufacture of embodiment 231, wherein the instructions specify each of the one or more gene products is individually compared to a gene reference value for the respective gene product.

233. The article of manufacture of any of embodiments 226-232, wherein the instructions specify:

expression of the at least one or more gene products from (a) negatively correlate to a risk that the subject is or is likely to develop neurotoxicity following administration of the immunotherapy when it is administered; and/or

expression of the at least one or more gene products from (b) positively correlates to a risk that the subject is or is likely to develop neurotoxicity following administration of the immunotherapy when it is administered.

234. The article of manufacture of any of embodiments 226-233, wherein the instructions specify if the assessing indicates the subject is or is likely to develop neurotoxicity following administration of the immunotherapy, the therapeutic regimen comprises administering to the subject:

i. an agent or other treatment capable of treating, preventing, delaying, reducing or attenuating the development or risk of development of a toxicity and (2) the immunotherapy, wherein administration of the agent is to be administered (i) prior to, (ii) within one, two, or three days of, (iii) concurrently with and/or (iv) at first fever following, the initiation of administration of the immunotherapy to the subject;

ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy; and/or

iii. the immunotherapy in an in-patient setting and/or with admission to the hospital for one or more days, optionally wherein the immunotherapy is otherwise to be administered to subjects on an outpatient basis or without admission to the hospital for one or more days; or

iv. an alternative therapeutic treatment other than the immunotherapy.

235. The article of manufacture of any of embodiments 226-234, wherein the instructions specify if the assessing indicates the subject is not or is likely not to develop neurotoxicity following administration of the immunotherapy, the therapeutic regimen comprises administering to the subject:

i. the immunotherapy, optionally at a non-reduced dose, optionally on an outpatient basis or without admission to the hospital for one or more days;

ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or

iii. the immunotherapy in an outpatient setting and/or without admission of the subject to the hospital overnight or for one or more consecutive days and/or is without admission of the subject to the hospital for one or more days.

236. The article of manufacture of any of embodiments 215-235, wherein the disease or condition is a cancer, optionally a myeloma, lymphoma or leukemia.

237. The article of manufacture of any of embodiments 215-236, wherein the disease or condition is a B cell malignancy.

238. The article of manufacture of embodiment 237, wherein the B cell malignancy is selected from acute lymphoblastic leukemia (ALL), chronic lymphoblastic leukemia (CLL), acute myelogenous leukemia (AML), chronic myelogenous leukemia (CML), non-Hodgkin lymphoma (NHL), or Diffuse Large B-Cell Lymphoma (DLBCL), or a subtype of any of the foregoing.

239. The article of manufacture of any of embodiments 215-238, wherein the disease or condition is acute lymphoblastic leukemia (ALL) or a subtype thereof

240. The article of manufacture of any of embodiments 215-239, wherein the at least one gene product is from (a) and is a gene product associated with a PH+ or Ph-like molecular subtype of ALL.

241. The article of manufacture of embodiment 240, wherein the at least one gene product is selected from ADGRF1, BMPR1B, CA6, CCL17, CCR6, CD99, CHN2, CRLF2, DENND3, ENAM, GAS6, GBP5, GLI2, IFITM1, IGJ (JCHAIN), LDB3, L0645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A, or is a portion or fragment of any of the foregoing.

242. The method of embodiment 215-241, wherein the immunotherapy is a T cell-engaging therapy comprising a bispecific antibody, wherein at least one binding portion specifically binds to a T cell antigen.

243. The method of embodiment 242, wherein the at least one binding portion specifically binds to CD3.

244. The method of embodiment 242 or 243, wherein a second binding portion of the bispecific antibody binds to an antigen, optionally wherein the antigen is CD19.

245. The method of any of embodiments 215-241, wherein the immunotherapy is a cell therapy.

246. The method of any of embodiments 215-241 or 245, wherein the immunotherapy is a T cell therapy comprising genetically engineered cells expressing a recombinant receptor that specifically binds to a ligand.

247. The article of manufacture of any of embodiments 215-248, wherein the subject is a human and/or the one or more gene products is human.

### VIII. EXAMPLES

The following examples are included for illustrative purposes only and are not intended to limit the scope of the invention.

### Example 1

### Observed Correlation Between Gene Expression in Tumor From Bone Marrow Aspirate and Development of Neurotoxicity in Subjects After Administration of CAR+ T Cell Therapy

Bone marrow aspirates (BMA), obtained from subjects prior to administration of a T cell composition containing T cells genetically engineered with a chimeric antigen receptor, were collected and analyzed by RNA sequencing (RNA-Seq) to assess expression of genes that correlated to the development of neurotoxicity in subjects administered a CAR+ T cell composition.

The BMA samples assessed were from subjects (N=31) having relapsed or refractory Acute Lymphoblastic Leukemia (ALL) that were subsequently administered a cell therapy involving a T cell composition containing autologous T cells engineered to express an anti-CD19 chimeric antigen receptor (CAR). The CAR+ T cell composition had been generated by a process including immunoaffinity-based selection of T cells (including CD4+ and CD8+ cells) from leukapheresis samples from the individual subjects, followed by activation and transduction with a viral vector encoding the anti-CD19 CAR, expansion and cryopreservation. The CAR contained an anti-CD19 scFv that was derived from the murine antibody SJ25C1; a region of CD28 including an extracellular region, a transmembrane domain and a costimulatory region; and a CD3-zeta intracellular signaling domain. The cryopreserved cell compositions were thawed at bedside prior to intravenous administration.

The CAR+ T cell composition was administered to subjects at a first target dose of approximately 1×10⁶ CD3+CAR+cells/kg (subject body weight). Some subjects received a second dose, at approximately between 14 and 28 days following the initial dose. Before administration of autologous CAR-expressing cells, subjects had been administered a preconditioning lymphodepleting chemotherapy containing either a single dose of cyclophosphamide (cy; about 1-3 g/m²) only or cyclophosphamide (30-60 mg/kg) and fludarabine (flu/cy; 25 mg/m²-30 mg/m², administered daily over three days).

After administration of the T cell composition, subjects were monitored for clinical response and adverse outcomes such as neurotoxicity, e.g., severe neurotoxicity. Subjects were assessed and monitored for neurotoxicity (neurological complications including symptoms of confusion, aphasia, encephalopathy, myoclonus seizures, convulsions, lethargy, and/or altered mental status), graded on a 0-5 scale, according to the National Cancer Institute—Common Toxicity Criteria (CTCAE) scale, version 4.03 (NCI-CTCAE v4.03). Common Toxicity Criteria (CTCAE) scale, version 4.03 (NCI-CTCAE v4.03). See Common Terminology for Adverse Events (CTCAE) Version 4, U.S. Department of Health and Human Services, Published: May 28, 2009 (v4.03: June 14, 2010); and Guido Cavaletti & Paola Marmiroli Nature Reviews Neurology 6, 657-666 (December 2010). The number of individual BMA samples associated with the highest grade of neurotoxicity experienced by the subject following administration of the CAR T cell therapy is shown in Table E1.

TABLE E1
Number of BMA samples associated with each neurotoxicity grade

Neurotoxicity Grade

| | Total | 0 | 1 | 2 | 3 | 4 | 5 |
|---|---|---|---|---|---|---|---|
| Samples | 31 | 6 | 4 | 3 | 11 | 2 | 5 |

Transcriptome analysis was carried out by RNA-Seq, FPKM and FPKQ values were determined; FPKQ values were log-transformed (log 2).

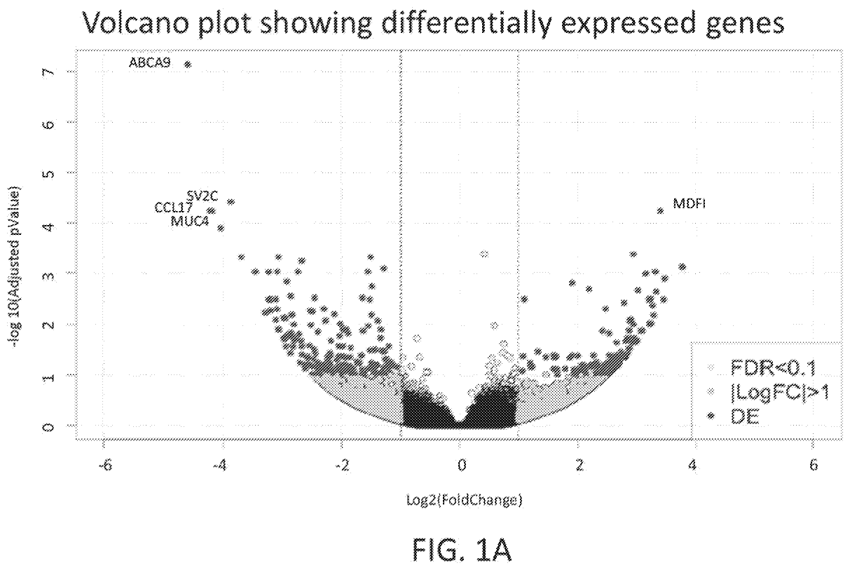

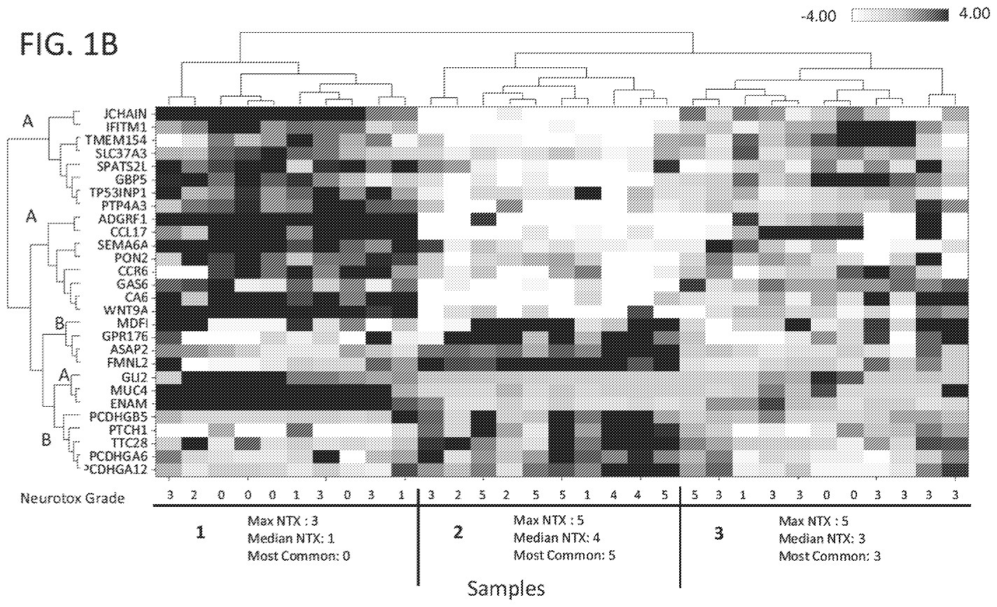

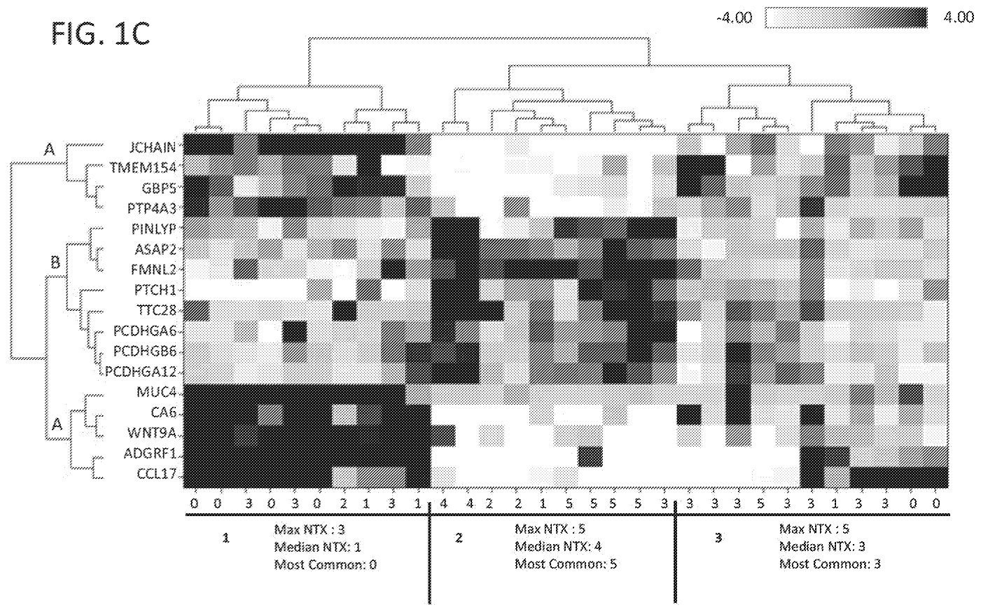

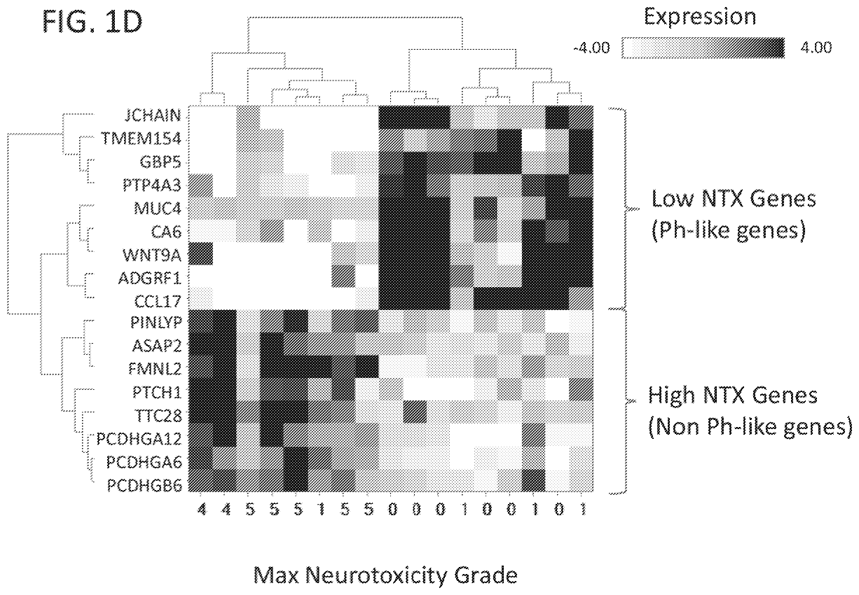

Expression of approximately 30,000 genes by RNA-Seq from BMA samples were correlated, post facto, to the development of neurotoxicity in subjects following administration of the autologous therapeutic CAR-T cell composition. Genes were identified that were significantly expressed in BMA samples from either conditions in which (1) subjects went on to develop grade of 4 or 5 neurotoxicity after receiving the cell therapy (n=7); or (2) subjects that developed grades of 0 and 1 neurotoxicity after receiving the cell therapy (n=10). Gene products were identified that were different among the two groups based on analysis of a volcano plot depicting statistical significance of expression of the gene products with the 1og 2 fold-change of expression of each gene product between the two conditions (FIG. 1A). Genes with greater than a 1og 2 fold change in gene expression of 1.0 or less than a negative 1og 2 fold change of −1.0 and having a −log 10 adjusted p value of less than 0.1 between samples associated with neurotoxicity grades 0-1 and grades 4-5 were identified as differentially expressed (DE). Among the transcripts, 119 DE genes were identified.

Table E2A sets forth DE genes that were expressed at higher levels in samples associated with development of grade 0 or 1 neurotoxicity compared to grade 4 or 5 neurotoxicity after administration of the T cell composition to subjects (designated “A” genes) and Table E2B sets forth genes that were expressed at higher levels in samples associated with development of grade 4 or 5 neurotoxicity compared to grade 0 or 1 neurotoxicity after administration of the T cell composition to subjects (designated “B” genes).

TABLE E2A
“A” genes and transcripts with elevated expression
in BMA Samples associated with grade 0-1 neurotoxicity

| Gene Symbol | Full name | Uniprot No. |
|---|---|---|
| ABCA9 | ATP-binding cassette sub-family A member 9 | Q8IUA7 |
| ADAMTSL4 | ADAMTS-like protein 4 | Q6UY14 |
| ADGRA2 | Adhesion G protein-coupled receptor A2 | Q96PE1 |
| ADGRF1 | adhesion G protein-coupled receptor F1 | Q5T601 |
| AK5 | Adenylate kinase isoenzyme 5 | Q9Y6K8 |
| APOL1 | Apolipoprotein L1 | O14791 |
| ARHGAP27 | Rho GTPase-activating protein 27 | Q6ZUM4 |
| ARID3B | AT-rich interactive domain-containing protein 3B | Q8IVW6 |
| CA6 | carbonic anhydrase 6 | P23280 |
| CABP7 | Calcium-binding protein 7 | Q86V35 |
| CCDC152 | Coiled-coil domain-containing protein 152 | Q4G0S7 |
| CCL17 | C-C motif chemokine ligand 17 | Q92583 |
| CCR1 | C-C chemokine receptor type 1 | P32246 |
| CCR6 | C-C motif chemokine receptor 6 | P51684 |
| CEP85L | Centrosomal protein of 85 kDa-like | Q5SZL2 |
| CISH | Cytokine-inducible SH2-containing protein | Q9NSE2 |
| CR2 | Complement receptor type 2 | P20023 |
| CRLF2 | Cytokine receptor-like factor 2 | Q9HC73 |
| ENAM | enamelin | Q9NRM1 |
| EPHA4 | Ephrin type-A receptor 4 | P54764 |
| FTH1P11 | ferritin heavy chain 1 pseudogene 11 | — |
| FTH1P2 | ferritin heavy chain 1 pseudogene 2 | — |
| FTH1P8 | ferritin heavy chain 1 pseudogene 8 | — |
| ENPP2 | Ectonucleotide pyrophosphatase/phosphodiesterase | Q13822 |
| | family member 2 | |
| GADD45A | Growth arrest and DNA damage-inducible protein | P24522 |
| | GADD45 alpha | |
| GAS6 | growth arrest specific 6 | Q14393 |
| GBP3 | Guanylate-binding protein 3 | Q9H0R5 |
| GBP5 | Guanylate binding protein 5 | Q96PP8 |
| GBP6 | Guanylate-binding protein 6 | Q6ZN66 |
| GIMAP1- | GIMAP1-GIMAP5 readthrough | A0A087WTJ2 |
| GIMAP5 | | |
| GLI2 | GLI family zinc finger 2 | P10070 |
| GPA33 | Cell surface A33 antigen | Q99795 |
| GPRIN3 | G protein-regulated inducer of neurite outgrowth 3 | Q6ZVF9 |
| HSPA1A | Heat shock 70 kDa protein 1A | P0DMV8 |
| IFITM1 | interferon induced transmembrane protein 1 | P13164 |
| IFITM3 | Interferon-induced transmembrane protein 3 | Q01628 |
| IL15 | Interleukin-15 | P40933 |
| IL2RA | Interleukin-2 receptor subunit alpha | P01589 |
| JCHAIN | joining chain of multimeric IgA and IgM | P01591 |
| KIAA1257 | Uncharacterized protein KIAA1257 | Q9ULG3 |
| LA16c-390H2.4 | LOC101929732 | |
| LAMB1 | Laminin subunit beta-1 | P07942 |
| LDB3 | LIM domain-binding protein 3 | O75112 |
| LINC00623 | long intergenic non-protein coding RNA 623 | |
| LST1 | Leukocyte-specific transcript 1 protein | O00453 |
| LTB | Lymphotoxin-beta | Q06643 |
| LY6E | Lymphocyte antigen 6E | Q16553 |
| MAS1 | Proto-oncogene Mas | P04201 |
| MUC4 | Mucin 4 | Q99102 |
| NLRC3 | NLR family CARD domain-containing protein 3 | Q7RTR2 |
| PLXNA4 | Plexin-A4 | Q9HCM2 |
| PON2 | paraoxonase 2 | Q15165 |
| PTGES3P1 | prostaglandin E synthase 3 pseudogene 1 | |
| PTP4A3 | protein tyrosine phosphatase type IVA, member 3 | O75365 |
| RNU1-1 | RNA, U1 small nuclear 1 | |
| RP11-345J4.6 | | |
| RP11-421N8.1 | | |
| RP11-51J9.5 | | |
| RP11-51O6.1 | | |
| RP11-552F3.9 | | |
| RP11-686D22.9 | | |
| RP11-723D22.3 | | |
| RP11-723O4.6 | | |
| RP13-512J5.1 | | |
| RP4-620F22.2 | | |
| RP5-940J5.9 | | |
| RP6-109B7.5 | | |
| RPL21P75 | | |
| RYR2 | Ryanodine receptor 2 | Q92736 |
| SAMD9L | Sterile alpha motif domain-containing protein 9-like | Q8IVG5 |
| SEMA6A | semaphorin 6A | Q9H2E6 |
| SLC37A3 | solute carrier family 37 member 3 | Q8NCC5 |
| SNRPEP4 | small nuclear ribonucleoprotein polypeptide E | |
| | pseudogene 4 | |
| SOCS1 | Suppressor of cytokine signaling 1 | O15524 |
| SPATS2L | SPATS2-like protein | Q9NUQ6 |
| SPON1 | Spondin-1 | Q9HCB6 |
| SV2C | Synaptic vesicle glycoprotein 2C | Q496J9 |
| TMEM154 | transmembrane protein 154 | Q6P9G4 |
| TNF | Tumor necrosis factor | P01375 |
| TP53INP1 | Tumor protein p53-inducible nuclear protein 1 | Q96A56 |
| TRIM47 | Tripartite motif-containing protein 47 | Q96ED4 |
| UST | Uronyl 2-sulfotransferase | Q9Y2C2 |

TABLE E2B
“B” genes and transcripts with elevated expression in
BMA Samples associated with grade 4 or 5 neurotoxicity

| Gene symbol | Full name | Uniprot No. |
|---|---|---|
| ASAP2 | ArfGAP with SH3 domain, ankyrin repeat and PH | O43150 |
| | domain 2 | |
| ATP8B1 | Phospholipid-transporting ATPase IC | O43520 |
| ATP9A | ATPase phospholipid transporting 9A | Q2NLD0 |
| CCNA1 | Cyclin-A1 | P78396 |
| CDHR3 | Cadherin-related family member 3 | Q6ZTQ4 |
| CECR2 | Cat eye syndrome critical region protein 2 | Q9BXF3 |
| CELF4 | CUGBP Elav-like family member 4 | Q9BZC1 |
| DLX1 | Homeobox protein DLX-1 | P56177 |
| DPYSL3 | dihydropyrimidinase like 3 | Q14195 |
| EHD4 | EH domain-containing protein 4 | Q9H223 |
| FMNL2 | formin like 2 | Q96PY5 |
| GGA2 | ADP-ribosylation factor-binding protein GGA2 | Q9UJY4 |
| GPR176 | G protein-coupled receptor 176 | Q14439 |
| HHIPL1 | HHIP-like protein 1 | Q96JK4 |
| HMX3 | Homeobox protein HMX3 | A6NHT5 |
| IGF2BP1 | insulin like growth factor 2 mRNA binding protein 1 | Q9NZI8 |
| HOXA7 | Homeobox protein Hox-A7 | P31268 |
| IL3RA | interleukin 3 receptor subunit alpha | P26951 |
| IRX3 | Iroquois-class homeodomain protein IRX-3 | P78415 |
| IRX5 | iroquois homeobox 5 | P78411 |
| KCNIP1 | Kv channel-interacting protein 1 | Q9NZI2 |
| KIAA1644 | Uncharacterized protein KIAA1644 | Q3SXP7 |
| LINC00092 | long intergenic non-protein coding RNA 92 | |
| LINC01483 | long intergenic non-protein coding RNA 1483 | |
| MDFI | MyoD family inhibitor | Q99750 |
| MIB1 | E3 ubiquitin-protein ligase MIB1 | Q86YT6 |
| MMP14 | Matrix metalloproteinase-14 | P50281 |
| NOM1 | Nucleolar MIF4G domain-containing protein 1 | Q5C9Z4 |
| OTOA | Otoancorin | Q7RTW8 |
| PCDHGA12 | protocadherin gamma subfamily A, 12 | O60330 |
| PCDHGA4 | Protocadherin gamma-A4 | Q9Y5G9 |
| PCDHGA6 | protocadherin gamma subfamily A, 6 | Q9Y5G7 |
| PCDHGB1 | Protocadherin gamma-B1 | Q9Y5G3 |
| PCDHGB5 | protocadherin gamma subfamily B, 5 | Q9NZI8 |
| PCDHGB6 | protocadherin gamma subfamily B, 6 | Q9Y5G7 |
| PINLYP | phospholipase A2 inhibitor and LY6/PLAUR domain | A6NC86 |
| | containing | |
| PPM1E | Protein phosphatase 1E | Q8WY54 |
| PRKD1 | Serine/threonine-protein kinase D1 | Q15139 |
| PROKR2 | Prokineticin receptor 2 | Q8NFJ6 |
| PRSS12 | Neurotrypsin | P56730 |
| PRTG | Protogenin | Q2VWP7 |
| PTCH1 | Protein patched homolog 1 | Q13635 |
| RFX8 | DNA-binding protein RFX8 | Q6ZV50 |
| RP11-146B14.1 | | |
| RP11-3P17.5 | | |
| RP11-41O4.1 | | |
| RP11-713N11.4 | | |
| RP4-568B10.1 | | |
| SERF1A | Small EDRK-rich factor 1 | O75920 |
| SEZ6L | Seizure 6-like protein | Q9BYH1 |
| SMURF1 | E3 ubiquitin-protein ligase SMURF1 | Q9HCE7 |
| TBC1D30 | TBC1 domain family member 30 | Q9Y2I9 |
| TCF12 | Transcription factor 12 | Q99081 |
| TCP11 | T-complex protein 11 homolog | Q8WWU5 |
| TM9SF3 | Transmembrane 9 superfamily member 3 | Q9HD45 |
| TMPRSS15 | transmembrane protease, serine 15 | P98073 |
| TMSB15A | Thymosin beta-15A | P0CG34 |
| TNKS1BP1 | 182 kDa tankyrase-1-binding protein | Q9C0C2 |
| TREM2 | Triggering receptor expressed on myeloid cells 2 | Q9NZC2 |
| TTC28 | tetratricopeptide repeat domain 28 | Q96AY4 |
| ZNF415 | zinc finger protein 415 | Q09FC8 |

Hierarchical clustering and heat map analysis of the differentially expressed “A” and “B” genes shown in Tables E2A and E2B were carried out and associated with the identified molecular ALL subtypes of the BMA samples. The analysis indicated that a subset of both the “A” and “B” genes were differentially highly expressed in various molecular subtypes of ALL. It was observed that the list of “A” genes overlapped with components of a genetic signature associated with a high-risk subtype of pediatric ALL, and that a subset of “B” genes were expressed at higher levels in other known molecular ALL subtypes. This gene expression profile may be referred to as Philadelphia like (also referred to as Ph-like or BCR-ABL-like), based on its association in subjects with the Philadelphia chromosome (Ph+), an abnormal chromosome 22 that incorporates of a portion of chromosome 9 (Harvey et al. (2010) Blood, 116:4874-4884; Harvey et al. (2013) Blood, 122:826; International PCT Publ. No. WO2013090419). Analysis the subjects of the clinical study described in above classified 16 patients as having PH+ or Ph-like gene expression and 15 patients having non Ph-like expression.

Table E3 summarizes the clinical factors of Ph+/Ph-like and non-Ph-like among treated subjects. A significant association was observed between subtype and whether the subject had prior CNS disease or had been treated with both cyclophosphamide (30-60 mg/kg) and fludarabine (flu/cy). No association between subtype and clinical factors previously identified as being correlated with neurotoxicity, (number of prior treatments, tumor burden, and age) were observed.

TABLE E3
Clinical factors associated with molecular subtype

| | | Non | Ph+/ | |
|---|---|---|---|---|
| | Total | Ph-like | Ph-like | p-value |
| Prior CNS disease | 31 | 0 yes | 6 yes | 0.018 |
| Conditioning Cy vs | 31 | 6 Flu/Cy | 1 Flu/Cy | 0.037 |
| Flu/Cy | | | | |
| Prior Blintumamab | 31 | 5 Yes | 10 Yes | 0.160 |
| Sex | 31 | 2 F | 6 F | 0.220 |
| Prior treatments < 2 | 31 | 11 Yes | 6 Yes | 0.458 |
| Tumor Burden | 31 | 53% (Mean) | 47% (Mean) | 0.635 |
| Age | 31 | 39 (Mean) | 42 (Mean) | 0.722 |
| Weight | 31 | 89 kg (Mean) | 90 kg (Mean) | 0.929 |

Exemplary subsets of the “A” and “B” genes that associated with molecular ALL subtypes are shown in Table E4.

TABLE E4
Subsets of “A” and “B” genes associated
with molecular subtypes of ALL.

| Subset A Genes | Subset of B Genes |
|---|---|
| (Ph-like subtype) | (other molecular subtypes) |
| ADGRF1 (GPR110) | ASAP2 |
| CA6 | FMNL2 |
| CCL17 | GPR176 |
| CCR6 | MDFI |
| CRLF2 | PCDHGA12 |
| ENAM | PCDHGA6 |
| GAS6 | PCDHGB5 |
| GBP5 | PCDHGB6 |
| GLI2 | PINLYP |
| IFITM1 | PTCH1 |
| IGJ (JCHAIN) | TTC28 |
| MUC4 | ATP9A |
| PON2 | HMX3 |
| PTP4A3 | DPYSL3 |
| SEMA6A | ZNF415 |
| SEC37A3 | IRX5 |
| SPATS2L | TMPRSS15 |
| TMEM154 | IL3RA |
| TP53INP1 | IGF2BP1 |
| WNT9A | |
| IL2RA | |

A hierarchical clustering and heat map analysis of the “A” and “B” genes identified in Table E4 is shown in FIG. 1B in which expression of each gene is grouped by the association to the degree of neurotoxicity observed in the subjects after administration of the CAR+ T cell composition. FIG. 1C sets forth results from a subset of the genes in FIG. 1B but also including PINLYP. Specifically, genes signatures of the BMA samples clustered into three groups. A first group of BMA samples contained a gene signature in which, among subjects that were subsequently administered the CAR+ T cell composition, the exemplary “A” genes were expressed at high levels and “B” genes expressed at lower levels in BMA samples from subjects that went on to develop low neurotoxicity (median grade 1). A second group of BMA samples, which were from subjects that went on to develop a higher degree neurotoxicity (median grade of 4, with the most common grade being a maximum grade of 5) to the administered therapeutic T cell composition, contained a gene signature in which the “A” genes were generally expressed at lower levels and the “B” genes were generally expressed at high levels. A further group of BMA samples generally involved a mixed expression of “A” genes and “B” genes, which was found in BMA samples from subjects that went on to develop moderate neurotoxicity (median grade 3, with the most common grade being 3 and maximum grade of 5) to the administered therapeutic T cell composition. FIG. 1D displays gene expression data of exemplary “A” genes and “B” genes measured in BMA samples from 17 subjects who experienced grade 0-1 or 4-5 neurotoxicity following administration of the CAR+ T cell composition. In agreement with the hierarchical clustering results described above, samples associated with grades 0-1 neurotoxicity displayed high expression of “A” genes and low expression of “B” genes, while samples associated with grades 4-5 neurotoxicity displayed high expression of “B” genes and low expression of “A” genes.

### Example 2

### Multi-Dimensional Analysis of Gene Expression and CAR-Induced Activity of T Cell Therapy With Association to Development of Neurotoxicity

Gene expression for exemplary “A” genes and exemplary “B” genes were plotted in two dimensions based on RNA expression results described in Example 1. The RNA-seq gene expression values were further plotted as a function of the CAR-antigen-dependent pro-inflammatory cytokine production of the therapeutic T cell composition. Therapeutic compositions that had been administered to individual subjects were separately assessed for information regarding parameters; for example, cells of the therapeutic compositions were incubated in the presence of CD19-expressing target cells and accumulated amounts (pg/mL) of TNF-alpha were assessed.

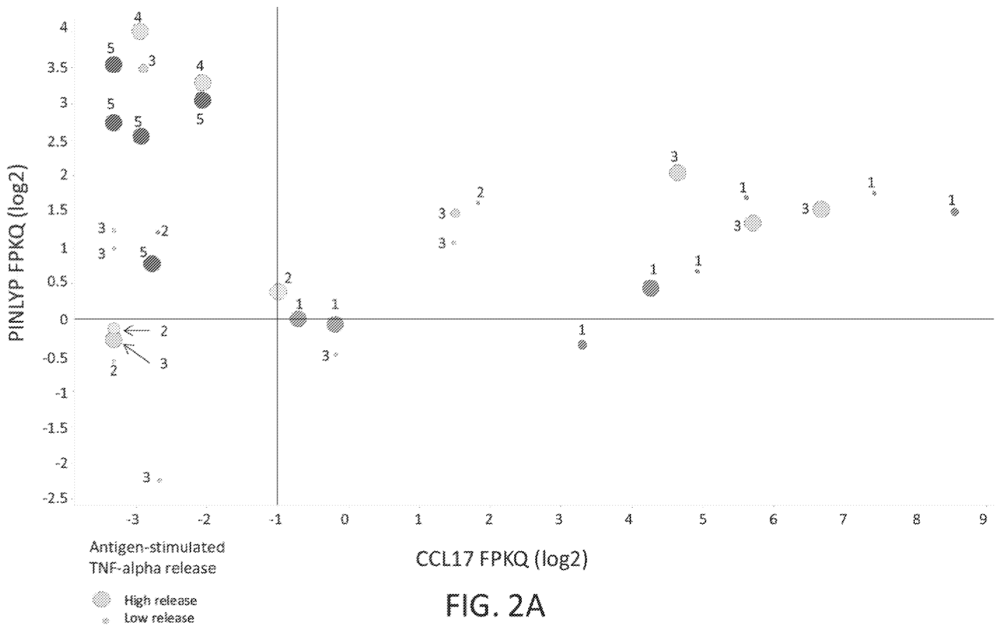

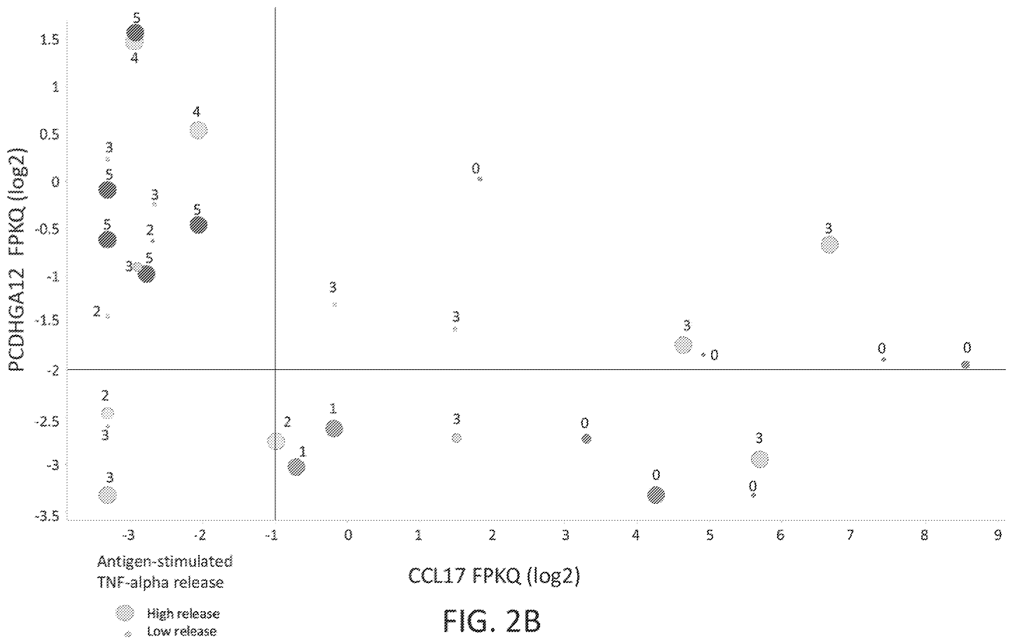

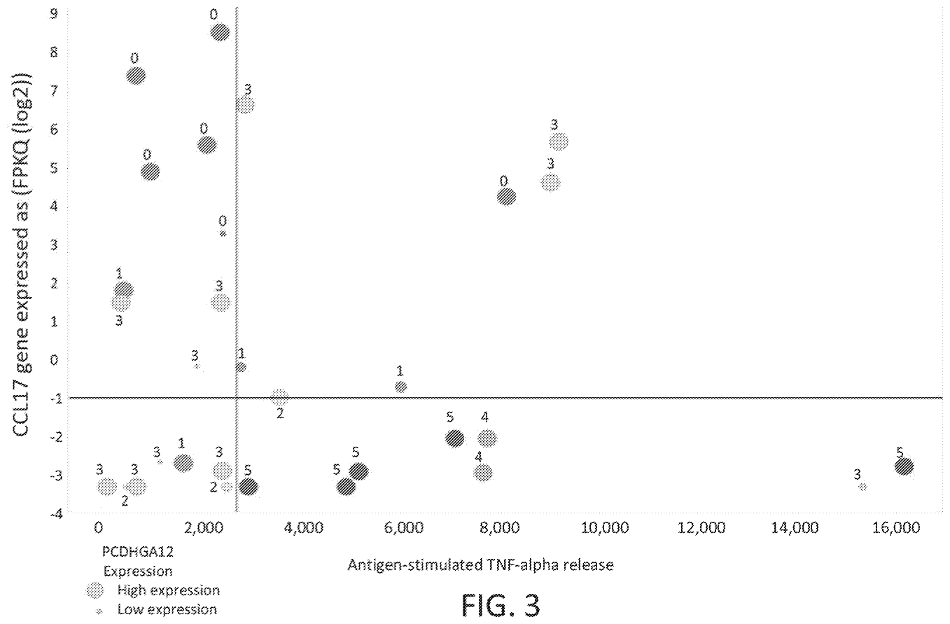

FIG. 2A plots FPKQ values (log 2 scale) for expression of CCL17 (A gene) and PINLYP (B gene). FIG. 2B plots FPKQ values (log 2 scale) for expression of CCL17 (A gene) and PCDHGA12 (B gene). In both plots, the size of each plotted data point represents the relative amount of TNF-alpha release TNF-alpha accumulation in culture following culture of cells of the therapeutic composition with cells expressing the antigen for which the CAR was specific, with the largest sized points indicating the highest levels of CAR antigen-specific accumulation of TNF-alpha observed in this assay. Degree of neurotoxicity or absence thereof (grade 0-5) observed in the individual subjects following administration of the CAR+ therapeutic T cell composition is shown next to the each data point. In FIG. 3, RNA-seq-based gene expression level (FPKQ) of an A gene (in this case CCL17) is plotted against the CAR antigen-specific TNF accumulation value (Antigen-stimulated TNF-alpha accumulation), with the size of each plotted data point representing the RNA-seq-based expression of PINLYP (a B gene).

Results from post-facto analysis of subjects administered the anti-CD19 CAR+ T cell composition indicated that for all assessed BMA samples from subjects who had gone on to develop either grade 4 or grade 5 neurotoxicity following administration of the therapeutic T cell composition, the FPKQ determined for the A gene CCL17 was below a certain level (−1 on the log 2 scale) , the determined FPKQ for the B gene PINLYP was above a certain level (e.g., above 0 or above 0.5 on the log 2 scale shown), and the determined FPKQ for the B gene PCDHGA12 was above a certain level (−2 on the log scale). In general, therapeutic compositions administered to subjects that went on to develop grade 4 or grade 5 neurotoxicity also were observed to have relatively higher levels of the parameter indicative of CAR antigen-specific accumulation of TNF-alpha; no subject with a composition observed with a value for this parameter below a threshold level went on to develop grade 4 or grade 5 neurotoxicity. As shown in FIG. 3, all subjects for which BMA samples were observed to be above the noted level for CCL17 gene FPKQ values did not develop grade 4 or grade 5 neurotoxicity, even those for which administered therapeutic compositions exhibited levels of the threshold value indicative of CAR antigen-specific accumulation of TNF-alpha.

In some embodiments, expression levels for particular genes or panels of genes, and/or gene expression profiles are determined, for biological samples such as BMA samples (such as those containing tumor cells). In some embodiments, such levels and/or profiles are used to assess the presence, absence and/or level of risk that a patient or patients may go on to experience an adverse event such as neurotoxicity or grade thereof, such as grade 4 or 5, following administration of an immunotherapy such as an engineered cell therapy. In some embodiments, such information or expression levels or profiles, in combination with levels of one or more parameters of therapeutic compositions, such as those indicative of CAR-induced/dependent and/or antigen-specific function, may be used to assess such presence/absence or level of risk. In some embodiments, the combination of such attributes may provide a more accurate predictive value for such risk or outcome.

### Example 3

### Analysis of Gene Signature in Acute Lymphoblastic Leukemia (ALL) Samples

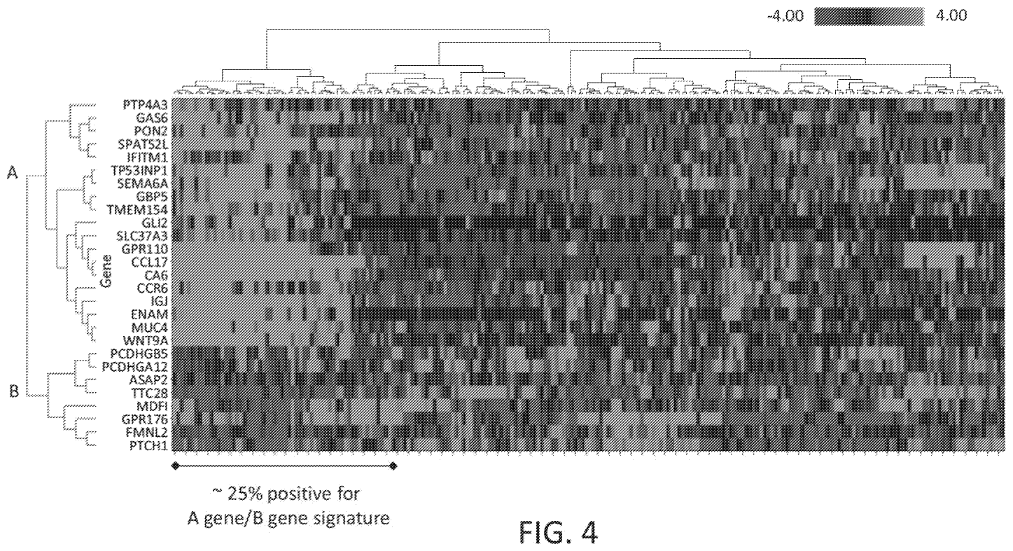

Analysis of gene expression for genes identified in Example 1 was assessed on RNA-seq data from 250 tumor samples from subjects having childhood precursor B-cell ALL (B-ALL) (see TARGET initiative managed by the National Cancer Institute (NCI); RNA-seq data set and clinical information for the TARGET ALL project used for this analysis are available at the database of Genotypes and Phenotypes (dbGaP) under accession phs000218.v16.p6). FIG. 4 shows hierarchical clustering and heat map of gene expression data from the TARGET dataset for exemplary genes described in Example 1 that were found to be associated with risk of developing grade 4 or 5 neurotoxicity. As shown, approximately 25% of the assessed data set exhibited a gene signature generally exhibiting high expression of “A” genes and low expression of “B” genes, which is the same signature observed in subjects identified in Example 1 that were less likely to develop grade 4 or 5 neurotoxicity after receiving the therapeutic T cell composition.

Statistical analyses were carried out to assess the degree of correlation of gene expression of various genes with expression of CCL17, an exemplary “A” gene. As shown in Table ES, other “A” list genes were among the genes that were most highly correlated to expression of CCL17. These results demonstrated that the gene signature identified in Example 1 as having high expression of “A” genes and low expression of “B” genes could be identified in another data set.

TABLE E5
Gene Expression Correlated to CCL17
Expression in Pediatric ALL Samples

| Gene Symbol | Correlation (R²) | Category |
|---|---|---|
| CA6 | 0.772 | A |
| ADGRF1 | 0.7133 | A |
| IGJ (JCHAIN) | 0.6612 | A |
| MUC4 | 0.6610 | A |
| WNT9A | 0.6270 | A |
| ADARB2-AS1 | 0.6177 | — |
| SLFN12L | 0.6061 | — |
| TMEM154 | 0.5723 | A |
| SPATS2L | 0.5512 | A |
| ABCA9 | 0.5723 | A |
| SLC37A3 | 0.5457 | A |
| PTP4A3 | 0.5435 | A |
| A2MP1 | 0.5381 | — |
| AK311257 | 0.5340 | — |
| GAS6 | 0.5318 | A |
| NRXN3 | 0.5316 | — |
| RASSF8 | 0.5309 | — |
| LAIR1 | 0.5284 | — |
| CKMT2 | 0.5283 | — |
| ENAM | 0.5163 | A |
| VWA2 | 0.5099 | — |
| STAG3 | 0.5097 | — |
| SOCS2-AS1 | 0.5095 | — |
| MYO1B | 0.5058 | — |
| CD99 | 0.5047 | — |
| GDPD4 | 0.5041 | — |
| TP53INP1 | 0.5038 | A |
| RNF157 | 0.4999 | — |
| GLI2 | 0.4966 | A |
| ECEL1 | 0.4949 | — |
| SLC8A1-AS1 | 0.4941 | — |
| LDB3 | 0.4935 | — |
| CABP7 | 0.4929 | A |
| PRX | 0.4935 | — |
| SOCS2 | 0.4929 | — |
| TMEM236 | 0.4860 | — |
| RASSF8-AS1 | 0.4853 | — |
| PTGDR | 0.4847 | — |
| RPS6KA2 | 0.4814 | — |
| HHIP | 0.4787 | — |
| BMPR1B | 0.4778 | — |
| HDAC7 | 0.4756 | — |
| SEMA6A | 0.4742 | A |

A clustering analysis of “A” genes was assessed in samples from the TARGET database and from a second dataset, which included microarray data from samples from subjects having B-ALL (GSE79533; described in Hirabayashi et al. Haematologica (2017) 102(1):118-129. PMID: 27634205, see also www.ncbi.nlm.nih.gov/geo/query/acc.cgi?acc=GSE79533). The results indicated that in both the TARGET and GSE79533 data sets, the “A” genes clustered the samples by molecular subtype, with “A” genes highly expressed in PH+ and Ph-like samples.

The identity of the molecular subtype of ALL was available from clinical information for 12 of the subjects in the clinical trial described in Example 1. Gene fusion analysis from the RNA-seq data collected as described in Example 1 identified fusions corresponding to molecular subtypes in an additional 6 subjects. A machine learning algorithm trained on the TARGET data set was used to classify the samples with a known subtype as Ph-like or non Ph-like based on gene expression. The gene expression prediction was concordant with the known subtype in 94% of the samples.

### Example 4

### Correlation of Acute Lymphoblastic Leukemia (ALL) Molecular Subtype

With Development of Neurotoxicity Following CAR+ T cell therapy

The severity of neurotoxicity following administration of a CAR+ T composition to subjects with a Philadelphia chromosome (Ph+) molecular subtype of ALL versus Philadelphia chromosome (Ph−) molecular subtype ALL was assessed from among subjects administered CAR-expressing T cells in three different clinical trials, including the clinical trial described in Example 1(Trial #1). All clinical trials included treatment of subjects with B-ALL with a T cell composition containing autologous T cells engineered to express an anti-CD19 CAR.

In one of the further clinical trials designated Trial #2, the cells expressed the same anti-CD19 chimeric antigen receptor as employed in the clinical trial described in Example 1, but were manufactured by a different process. In subjects in Trial #2, two days after lymphodepletion, a split dose infusion of CAR-T cells at one of three dose levels: 3×10⁶, 1 x 10⁷, and 3×10⁷ CAR+ T cells/kg of subject weight was administered.

In another clinical trial (Trial #3), T cells expressing an anti-CD19 CAR were administered that differed in the antigen-binding domain, spacer and the costimulatory signaling domain of the CAR described in each of the clinical trials above, whereby the CAR contained an anti-CD19 scFv that was derived from the murine antibody FMC63, an immunoglobulin spacer, a transmembrane domain derived from CD28, a costimulatory region derived from 4-1BB, and a CD3-zeta intracellular signaling domain. In Trial #3, the subjects were administered one of three different doses, 2×10⁵, 2×10⁶, or 2×10⁷ CAR+ T cells /kg of subject weight, 48 to 96 hours after lymphodepleting chemotherapy. In the different clinical trials, the subjects were monitored for clinical response and adverse outcomes. Neurotoxicity was graded on a 0-5 scale as described in Example 1. The presence or absence of the Philadelphia chromosome was assessed. In the three clinical trials examined, 100 Ph− subjects and 29 PH+ subjects were identified.

The number of subjects with a PH+ or Ph− molecular subtype of ALL that did or did not go on to develop indicated degrees of neurotoxicity is indicated in Table E6A, with total numbers from all three clinical trials shown in Tables E6B and E6C, respectively. Subjects with Ph− molecular subtype ALL generally were observed to develop higher degrees of neurotoxicity following administration of the CAR+ T cells, as opposed to the PH+ subjects. As shown in Table E6-B, a smaller percentage of PH+ subjects went on to develop a neurotoxicity of grade of 3 or higher compared to subjects with Ph− molecular subtype, and no incidents of grade 4 or 5 neurotoxicity were observed in subjects with PH+ molecular subtype ALL, as compared to a rate of 14% in subjects with Ph− molecular subtype. The results indicated an association of Ph− status and development of grade 4 or 5 neurotoxicity after administration of the CAR+ T cell composition in this study (p<0.05).

TABLE E6A
Number of Ph− and Ph+ subjects experiencing
the indicated grade of neurotoxicity

| | | Maximum Neurotoxicity Grade | | | | | |
|---|---|---|---|---|---|---|---|
| TRIAL | Status | 0 | 1 | 2 | 3 | 4 | 5 |
| Trial #1 | Ph− | 5 | 3 | 2 | 9 | 2 | 5 |
| | Ph+ | 1 | 1 | 1 | 2 | 0 | 0 |
| Trial #2 | Ph− | 14 | 5 | 0 | 14 | 3 | 0 |
| | Ph+ | 6 | 3 | 2 | 4 | 0 | 0 |
| Trial #3 | Ph− | 19 | 4 | 3 | 8 | 2 | 2 |
| | Ph+ | 3 | 2 | 2 | 2 | 0 | 0 |
| Combined | Ph− | 38 | 12 | 5 | 31 | 7 | 7 |
| | Ph+ | 10 | 6 | 5 | 8 | 0 | 0 |

TABLE E6B
Number of Ph− and Ph+ subjects experiencing
the indicated grade of neurotoxicity

| | Neurotoxicity Grade | | % Grade |
|---|---|---|---|
| Status | 0-2 | 3-5 | 3-5 |
| Ph− | 55 | 45 | 45% |
| Ph+ | 21 | 8 | 8% |

TABLE E6C
Number of Ph− and Ph+ subjects experiencing
the indicated grade of neurotoxicity

| | Neurotoxicity Grade | | % Grade |
|---|---|---|---|
| Status | 0-3 | 4-5 | 3-5 |
| Ph− | 86 | 14 | 14% |
| Ph+ | 29 | 0 | 0% |

Development of grades of neurotoxicity following CAR-T cell administration was assessed for ALL subjects of Trial #1 and an additional clinical trial (Trial #4) who were or were not determined to be either positive for a Ph-like gene profile (via BMA gene expression analysis) or determined to be of the PH+ molecular subtype (such subjects exhibiting these features designated Ph+/Ph-like; other subjects designated Non-Ph). Trial #4 included children and young adults having recurrent or refractory CD19+ ALL that were administered autologous T cell compositions containing cells expressing the same anti-CD19 CAR as used in Trial #3. Subjects in Trial #4 received a dose of 0.5×10⁶, 1×10⁶, 5×10⁶, or 10×10⁶ total cells per kg of subject weight at a ratio of 1:1 CD4+ cells to CD8+ cells.

The subjects of both trials were monitored for clinical response and adverse outcomes, and neurotoxicity was graded on a 0-5 scale as described in Example 1. Results are set forth in Tables E7 and E8. In both trials, fewer of the Ph+/Ph-like subjects went on to develop neurotoxicity of grades 3 or greater (3+) following treatment, as compared to the non-Ph subjects. No grade 4 or grade 5 neurotoxicity was observed in Ph+/Ph-like subjects in either clinical trial. The lack of observed grade 4 or 5 neurotoxicity events in PH+ or Ph-like subjects of Trials #2-4 was consistent with the low risk associated with neurotoxicity in PH+ or Ph-like subjects extending beyond the clinical trial described in Example 1.

TABLE E7
Number of Ph+/Ph-like and non-Ph subjects from Trial #1 experiencing
the indicated grade of neurotoxicity

| | Neurotoxicity Grade | | | Neurotoxicity Grade | | | |
|---|---|---|---|---|---|---|---|
| | No | | | No | | | |
| Status | (0-2) | 3+ | P value | (0-3) | 4-5 | 5 | P value |
| non-Ph | 3 | 12 (80%) | 0.03 | 8 | 7 | 5 | 0.0002 |
| Ph+/ | 10 | 6 (38%) | | 16 | 0 | 0 | |
| Ph-like | | | | | | | |

TABLE E8
Number of Ph+/Ph-like and non-Ph subjects from Trial #4 experiencing
the indicated neurotoxicity grades

Neurotoxicity Grade

| Status | 0-2 | 3-5 | 0-3 | 4-5 |
|---|---|---|---|---|
| non-Ph | 27 | 8 | 32 | 3 |
| Ph+/Ph-like | 7 | 1 | 8 | 0 |

The low incidence of grade 3 or higher neurotoxicity among Ph-like or PH+ subjects was consistent across the different trials examined that spanned adult and pediatric ALL subjects, and different anti-CD19 CARs products containing different costimulatory domains, spacers, and binding domains, and produced by different processes. Across all of the 172 total subjects from these four clinical trials, no grade of 4 or 5 neurotoxicity was observed in PH+ or Ph-like subjects. These results are consistent with different degrees of risk for anti-CD19 CAR T associated neurotoxicity among subjects with different B-ALL molecular subtypes, with Ph-like subjects being at significantly lower risk than non Ph-like patients. These results are also in agreement with a role of stratification by molecular subtype or gene expression signature in identifying patients at elevated risk for severe NT.

### Example 5

### Association of Protein Analytes in Plasma With Development of Neurotoxicity Following CAR+ T Cell Therapy

Plasma samples, collected from a group of the subjects with ALL described in Example 1, at one day prior to infusion with the anti-CD19 CAR-T cell composition (pre-dose), and two, four, and seven days subsequent to (post-dose) the administration of the anti-CD19 CAR-T cell composition, were assessed for levels of various factors. As described in Example 1, following administration of the CAR+ T cell composition, subjects had been monitored for clinical response and adverse outcomes, and neurotoxicity graded on a 0-5 scale. Plasma samples were analyzed with an immunoassay (Meso Scale Discovery) to assess amounts of soluble versions of the proteins listed in Table E9.

TABLE E9
Proteins assessed by immunoassay

| | |
|---|---|
| Angiopoietin 1 | MCP-4 |
| Angiopoietin 2 | MDC |
| Brain derived | Mesothelin |
| neurotropic factor | |
| CTACK (CCL27) | MIP-1α |
| Endoglin | MIP-1β |
| Eotaxin | MIP-3α |
| Eotaxin-3 | MIP-3β |
| EPO | MMP-9 |
| E-Selectin | NSE |
| FAS | Osteocalcin |
| FasL | PRDX6 |
| Fractalkine | P-Selectin |
| GRO-α | RANTES |
| I-309 | Resistin |
| ICAM-1 | S100β |
| ICAM-3 | SAA |
| IL-1Ra | SDF-1α |
| IL-6R | CCL17 (TARC) |
| IP-10 | Thrombomodulin |
| I-TAC | VCAM-1 |
| MCP-1 | vWF |
| MCP-3 | YKL-40 |

Expression levels of each of the proteins in plasma samples of individual subjects were assessed, post facto, and compared among subjects based on development (or lack thereof) of neurotoxicity; specifically, expression levels in subjects that developed no (grade 0) or grade 1 neurotoxicity were compared to those observed in subjects that had neurotoxicity of grade 4 or 5. Wilcoxon statistical comparisons were performed for each protein at each time point.

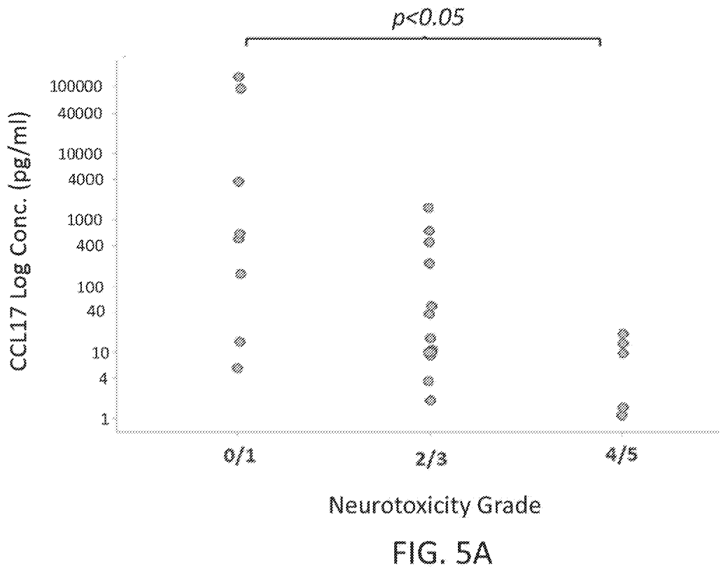

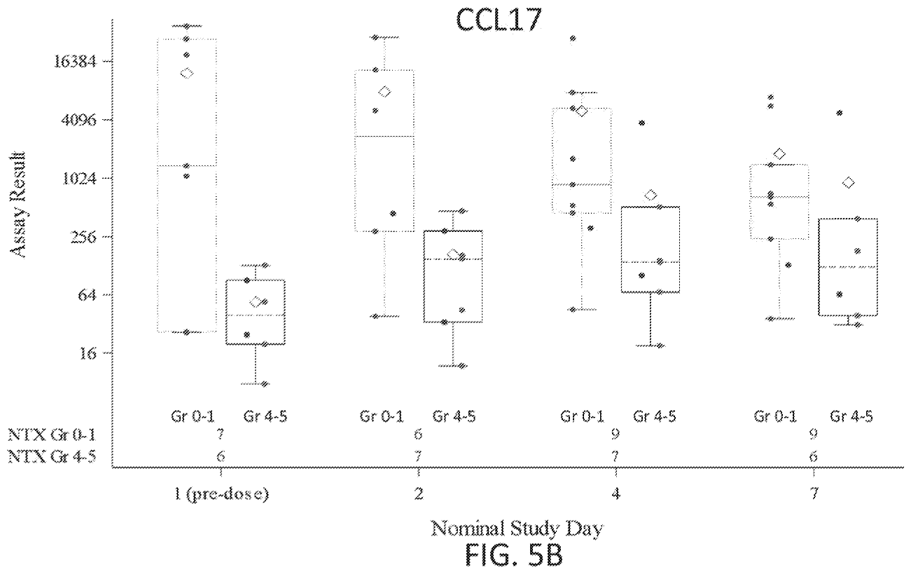

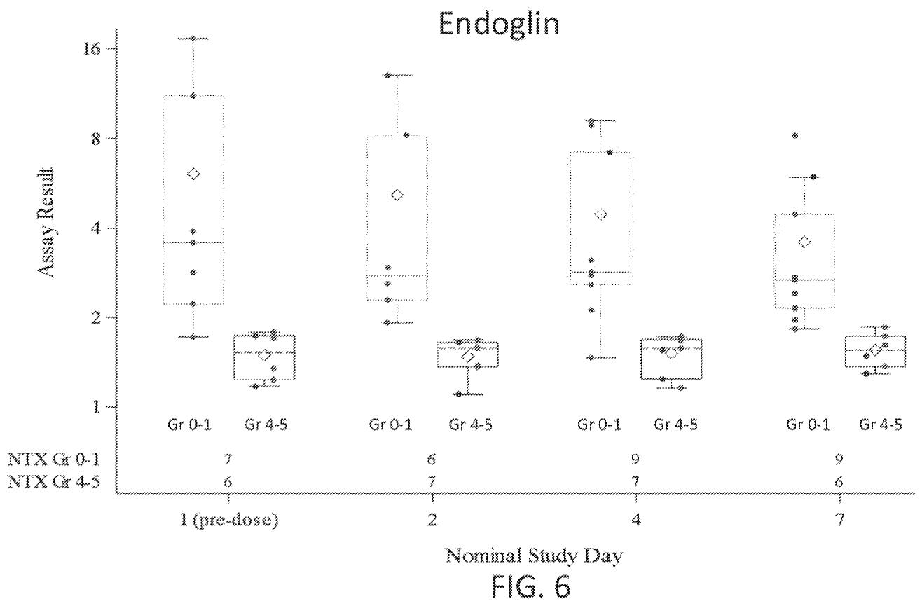

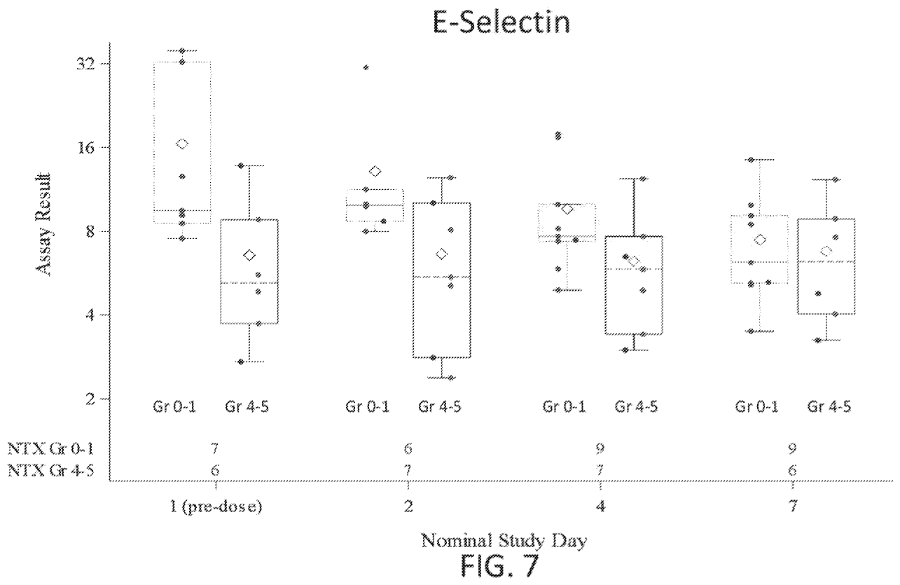

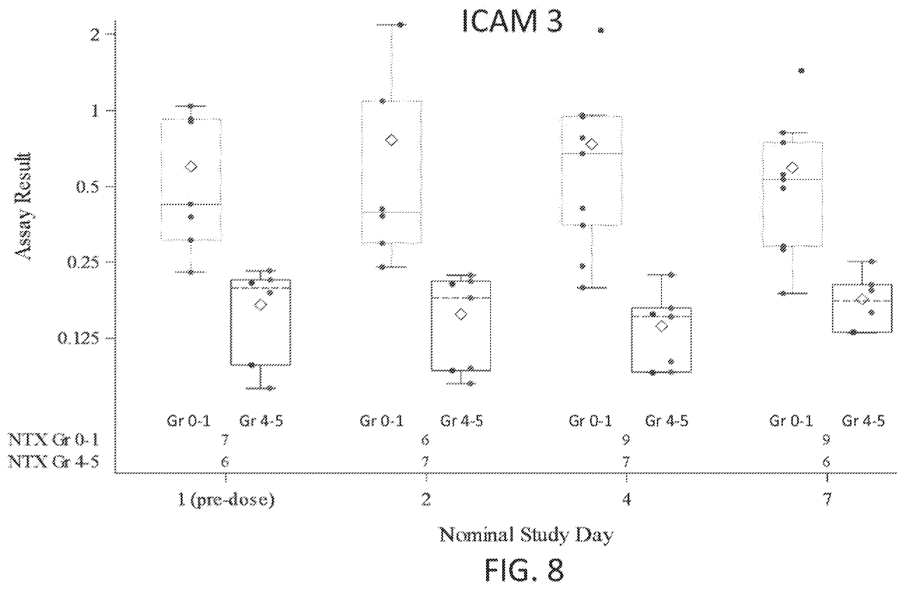

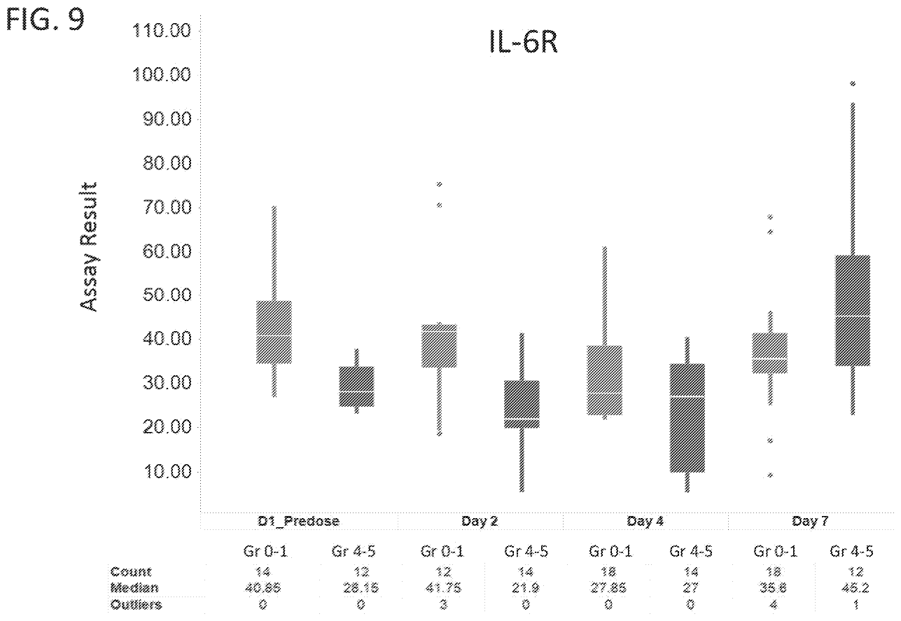

As shown in Table E10, a significant difference between protein levels of certain assessed plasma analytes was observed in subjects who went on to develop grade 0-1 neurotoxicity as compared to those who went on to develop grade 4-5 neurotoxicity at various assessed time points. As shown in FIGS. 5-9, protein levels in plasma of soluble versions of CCL17/TARC, which can play a role in T cell trafficking and activation (FIGS. 5A and 5B), Endoglin (FIG. 6), E-selectin (FIG. 7), ICAM-3 (FIG. 8), IL-6R (FIG. 9) were observed in pre-dose samples to negatively correlate to the development of higher grades of neurotoxicity (higher expression in pre-dose plasma samples from subjects with grade 0-1 neurotoxicity versus grade 4-5 neurotoxicity). Higher levels of Endoglin and E-Selectin were observed in samples from subjects with grade 0-1, as opposed to grade 4-5, neurotoxicity. Without wishing to be bound by theory, each of these analytes in some aspects is expressed on vascular endothelial cells and/or is associated with endothelial cell activation, vascular permeability and/or angiogenesis. In some embodiments, provided are compositions and methods for predicting risk of neurotoxicity involving assessing one or more analytes; in some aspects, one or more of the analytes is expressed on vascular endothelial cells and/or is associated with endothelial cell activation, vascular permeability and/or angiogenesis, and increased levels of such analyte(s) is associated with an increased risk of developing a toxicity or a severe form thereof, such as grade 4 or higher neurotoxicity.

Table E10 lists p values reflective of differences observed (p values indicated) among the different groups of subjects (grade 0-1 vs grade 4-5 neurotoxicity) in plasma levels of the indicated exemplary proteins, for plasma samples taken pre-dose and/or the indicated post-treatment time-points.

For example, plasma levels of Endoglin and ICAM-3 were observed to be different among these groups when assessed pre-dose, and were also observed to be different in post-dose plasma taken at various time-points (in each case, higher values were observed in subjects experiencing grade 0-1 versus those experiencing grade 4-5 neurotoxicity). Pre-dose plasma levels of CCL17, a chemokine highly expressed in samples associated with low neurotoxicity in the clinical trial described in Example 1 and in Ph-like B-ALL datasets, were also significantly higher in subjects experiencing grade 0 and 1 versus those experiencing grade 4 and 5 neurotoxicity.

TABLE E10
Proteins with significant differences between samples
associated grade 0-1 and grade 4-5 neurotoxicity

| Analyte | Pre dose | Day 2 | Day 4 | Day 7 |
|---|---|---|---|---|
| CCL27 | | | 0.044 | |
| Endoglin | 0.0082 | 0.0034 | 0.0059 | 0.0027 |
| E-Selectin | 0.05 | | | |
| FAS | | | | 0.039 |
| Fractalkine | | | | |
| I-309 | | | 0.044 | |
| ICAM-3 | 0.0053 | 0.0034 | 0.0015 | 0.0056 |
| IL-6R | 0.027 | | | |
| NSE | | | | 0.051 |
| P-Selectin | | | | 0.011 |
| Resistin | | | | 0.039 |
| S100β | | | | 0.052 |
| CCL17 (TARC) | 0.038 | | 0.057 | |
| Thrombomodulin | | | 0.011 | 0.011 |
| vWF | | | | 0.068 |

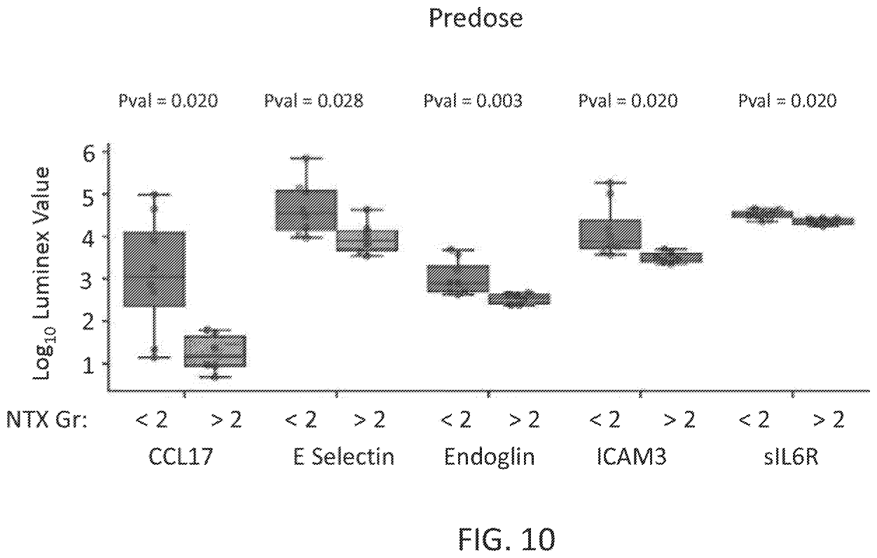

Pre-dose plasma samples were also analyzed with a Luminex multiplex assay to assess amounts of CCL17, E-selectin, Endoglin, ICAM-3, and soluble IL-6R (sIL6R). Amounts of each protein measured from subjects that developed no or grade 1 neurotoxicity (<2) were compared to those observed in subjects that had neurotoxicity of grades 3 or greater (>2). Consistent with the results obtained from the Meso Scale Discovery immunoassay, significantly higher amounts of each protein were observed in plasma sample collected from subjects that developed <2 neurotoxicity (FIG. 10).

### Example 6

### Additional Baseline Factors Associated With Development of Neurotoxicity Following CAR+ T Cell Therapy

Additional baseline factors, measured prior to treatment with CAR-T cell compositions in a number of different clinical studies for B cell malignancies such as ALL and NHL, were assessed and compared among subjects that had and that had not developed grade 3 or higher neurotoxicity. Data from 3 clinical trials were analyzed (including the study described in Example 1, that involved anti-CD19 CAR-T treatment in subjects with ALL (Trial 1), a clinical study involving a similar anti-CD19 CAR treatment administered to subjects with relapsed or refractory adult ALL (Trial 2), and an additional clinical trial in which subjects with B-cell malignancies, including ALL and non-Hodgkin lymphoma (NHL), were treated with cells expressing a different anti-CD19 CAR(Trial 3)). Subjects were administered anti-CD19 CAR-T cell compositions and monitored for clinical response and adverse outcomes. Neurotoxicity was graded on a 0-5 scale as described in Example 1.

Baseline factors were analyzed, post facto, and compared among subjects using a number of statistical analyses. Exemplary baseline factors that were observed to be associated with grade 3 or greater neurotoxicity in the subjects are listed in Table Ell below, including levels of serum IL-15 and platelet cell counts prior to administration of CAR-T cells, disease type (non-Hodgkin lymphoma (NHL) as compared to ALL), and disease burden as defined by percentages of blasts in the bone marrow.

TABLE E11
Exemplary baseline factors associated with Grade 3 or greater neurotoxicity

| Baseline | | | | | | |
|---|---|---|---|---|---|---|
| Factor | Indication | Criterion | Gr ≥ 3 NTX | | P value^{a} | Source |
| Inflammatory | B-Cell | High IL-15 | 26/45 | (58%) | <.0001 | Trial 1 |
| marker: | malignancies | Low IL-15 | 19/109 | (17%) | | Trial 3 |
| Serum IL-15 | | | | | | |
| Platelet Count | ALL | <120 K | 60/162 | (37%) | .002 | Trail 1 |
| | | >120 K | 9/60 | (15%) | | Trial 2 |
| | | | | | | Trial 3 |
| Disease Type | B-Cell | ALL | 14/47 | (30%) | .003 | Trial 3 |
| | malignancies | NHL | 8/62 | (13%) | | |
| Disease | ALL | ≥5% BM | 40/94 | (43%) | .02 | Trail 1 |
| Burden | | blasts | | | | Trial 2 |
| | | <5% BM | 9/42 | (21%) | | Trial 3 |
| | | blasts | | | | |

^{a}Two sided Fisher's exact test
^{b}High IL-15 ≥ 30 pg/mL.

Blood samples collected from subjects at various time points after administration of CAR-T cells in Trial 1 were analyzed for CAR+-T cell counts. Serum levels of cytokines, e.g., IL-15, from the subjects were assessed at pre-treatment (screening) and various post-treatment time-points (day 2, day 4, and day 7). The results demonstrated that prolonged and fatal neurotoxicity correlated with early and rapid rise in levels of IL-15, a T cell promoting cytokine, and CAR T cell expansion.

More rapid CAR-T expansion and earlier peak expansion of CAR-expressing T cells was observed in the blood of subjects that went on to develop more severe neurotoxicity, and in particular those who developed grade 5 neurotoxicity and cerebral edema. For example, results indicated that these subjects had exhibited a detectable level of CAR+cells per μl of blood within 4-7 days after the first dose of cells, with greater than 20 CAR+cells per μl of blood observed in blood of all such subjects within seven days after cells were administered. In subjects with less severe neurotoxicity, peak expansion of CAR+ T cells generally was observed later and with a lower peak number of CAR+ T cells per microliter (FIG. 10).

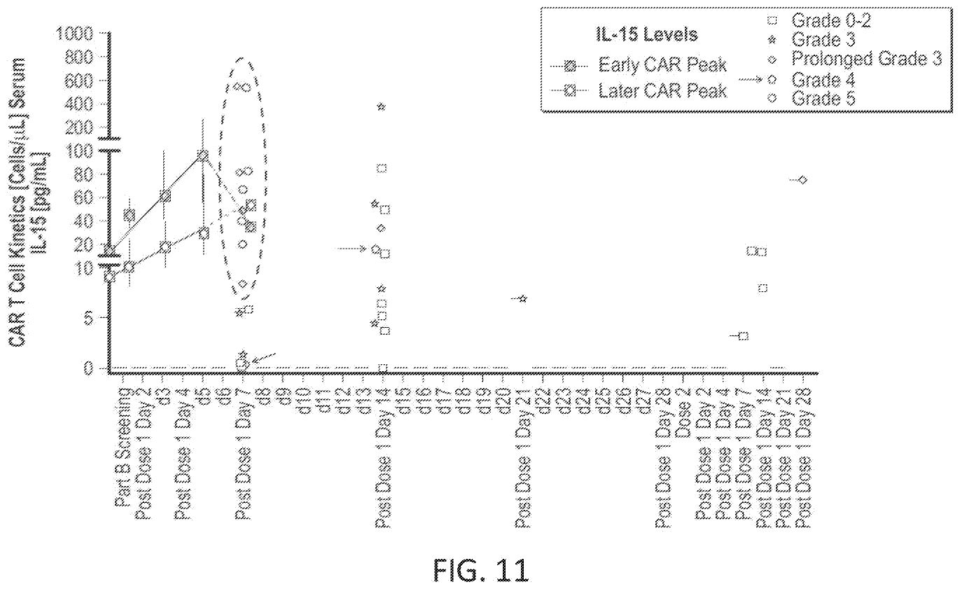

For the group of subjects that had experienced earlier peak CAR-T cell expansion (peak CAR+-T numbers by day 7), higher medium baseline serum IL-15 levels and a steeper increase in serum IL-15 through day 4 post-administration were observed, as compared to subjects with later peak CAR T cell expansion (FIG. 11). By the next time-point (day 7) at which serum IL-15 levels were measured, serum IL-15 levels in these subjects with rapid peak expansion had declined and were approximately comparable to or below levels in subjects with later peak expansion. The results were consistent with a conclusion that baseline serum IL-15 levels and/or higher or rapid increases in IL-15 levels, may indicate or correlate with potential risk for neurotoxicity or cerebral edema or severity thereof, following CAR-T cell treatment. Lymphodepleting and bridging chemotherapies are also observed to be associated with an increase in T cell proliferation-promoting cytokines such as IL-15.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Summary of Sequences

| # | ANNOTATION |
|---|---|
| 1 | carbonic anhydrase 6 (isoform 1) |
| 2 | carbonic anhydrase 6 (isoform 2) |
| 3 | carbonic anhydrase 6 (isoform 3) |
| 4 | carbonic anhydrase 6 (isoform 4) |
| 5 | C-C motif chemokine ligand 17 |
| 6 | C-C motif chemokine receptor 6 |
| 7 | enamelin |
| 8 | growth arrest specific 6 |
| 9 | guanylate binding protein 5 |
| 10 | GLI family zinc finger 2 |
| 11 | adhesion G protein-coupled receptor F1 (Isoform I) |
| 12 | adhesion G protein-coupled receptor F1 (Isoform 2) |
| 13 | interferon induced transmembrane protein 1 |
| 14 | joining chain of multimeric IgA and IgM |
| 15 | Mucin 4 (isoform A) |
| 16 | Mucin 4 (isoform D) |
| 17 | Mucin 4 (isoform E) |
| 18 | Mucin 4 (isoform F) |
| 19 | paraoxonase 2 (isoform 1) |
| 20 | paraoxonase 2 (isoform 2) |
| 21 | protein tyrosine phosphatase type IVA, member 3 (isoform 1) |
| 22 | protein tyrosine phosphatase type IVA, member 3 (isoform 2) |
| 23 | semaphorin 6A |
| 24 | solute carrier family 37 member 3 |
| 25 | Spermato-genesis associated serine rich 2 like |
| 26 | Trans-membrane protein 154 |
| 27 | tumor protein p53 inducible nuclear protein 1 (isoform a) |
| 28 | tumor protein p53 inducible nuclear protein 1 (isoform b) |
| 29 | Wnt family member 9A |
| 30 | ArfGAP with SH3 domain, ankyrin repeat and PH domain 2 |
| 31 | formin like 2 |
| 32 | G protein-coupled receptor 176 (isoform 1) |
| 33 | G protein-coupled receptor 176 (isoform 2) |
| 34 | G protein-coupled receptor 176 (isoform 3) |
| 35 | MyoD family inhibitor (isoform 1) |
| 36 | MyoD family inhibitor (isoform 2) |
| 37 | protocadherin gamma subfamily A, 12 (isoform 1) |
| 38 | protocadherin gamma subfamily A, 12 (isoform 2) |
| 39 | protocadherin gamma subfamily A, 6 (isoform 1) |
| 40 | protocadherin gamma subfamily A, 6 (isoform 2) |
| 41 | protocadherin gamma subfamily B, 6 (isoform 1) |
| 42 | protocadherin gamma subfamily B, 6 (isoform 2) |
| 43 | phospholipase A2 inhibitor and LY6/PLAUR domain |
| | containing (isoform 1) |
| 44 | phospholipase A2 inhibitor and LY6/PLAUR domain |
| | containing (isoform 2) |
| 45 | Protein patched homolog 1 (Isoform L) |
| 46 | Protein patched homolog 1 (Isoform M) |
| 47 | Protein patched homolog 1 (Isoform S) |
| 48 | tetratricopeptide repeat domain 28 |
| 49 | carbonic anhydrase 6 (isoform 1) |
| 50 | carbonic anhydrase 6 (isoform 2) |
| 51 | carbonic anhydrase 6 (isoform 3) |
| 52 | carbonic anhydrase 6 (isoform 4) |
| 53 | C-C motif chemokine ligand 17 |
| 54 | C-C motif chemokine receptor 6 |
| 55 | enamelin |
| 56 | growth arrest specific 6 |
| 57 | guanylate binding protein 5 |
| 58 | GLI family zinc finger 2 |
| 59 | adhesion G protein-coupled receptor F1 (Isoform I) |
| 60 | adhesion G protein-coupled receptor F1 (Isoform 2) |
| 61 | interferon induced transmembrane protein 1 |
| 62 | joining chain of multimeric IgA and IgM |
| 63 | Mucin 4 (isoform A) |
| 64 | Mucin 4 (isoform D) |
| 65 | Mucin 4 (isoform E) |
| 66 | Mucin 4 (isoform F) |
| 67 | paraoxonase 2 (isoform 1) |
| 68 | paraoxonase 2 (isoform 2) |
| 69 | protein tyrosine phosphatase type IVA, member 3 (isoform 1) |
| 70 | protein tyrosine phosphatase type IVA, member 3 (isoform 2) |
| 71 | semaphorin 6A |
| 72 | solute carrier family 37 member 3 |
| 73 | spermatogenesis associated serine rich 2 like |
| 74 | transmembrane protein 154 |
| 75 | tumor protein p53 inducible nuclear protein 1 (isoform a) |
| 76 | tumor protein p53 inducible nuclear protein 1 (isoform b) |
| 77 | Wnt family member 9A |
| 78 | ArfGAP with SH3 domain, ankyrin repeat and PH domain 2 |
| 79 | formin like 2 |
| 80 | G protein-coupled receptor 176 (isoform 1) |
| 81 | G protein-coupled receptor 176 (isoform 2) |
| 82 | G protein-coupled receptor 176 (isoform 3) |
| 83 | MyoD family inhibitor (isoform 1) |
| 84 | MyoD family inhibitor (isoform 2) |
| 85 | Protocadherin gamma subfamily A, 12 (isoform 1) |
| 86 | Protocadherin gamma subfamily A, 12 (isoform 2) |
| 87 | Protocadherin gamma subfamily A, 6 (isoform 1) |
| 88 | Protocadherin gamma subfamily A, 6 (isoform 2) |
| 89 | Protocadherin gamma subfamily B, 6 (isoform 1) |
| 90 | Protocadherin gamma subfamily B, 6 (isoform 2) |
| 91 | phospholipase A2 inhibitor and LY6/PLAUR domain |
| | containing (isoform 1) |
| 92 | phospholipase A2 inhibitor and LY6/PLAUR domain |
| | containing (isoform 2) |
| 93 | Protein patched homolog 1 (Isoform L) |
| 94 | Protein patched homolog 1 (Isoform M) |
| 95 | Protein patched homolog 1 (Isoform S) |
| 96 | TTC28 |
| 97 | IL2RA isoform 1 precursor mRNA transcript |
| 98 | IL2RA isoform 2 precursor mRNA transcript |
| 99 | IL2RA isoform 3 precursor mRNA transcript |
| 100 | ATP9A mRNA |
| 101 | HMX3 mRNA |
| 102 | DPYSL3 isoform 1 mRNA |
| 103 | DPYSL3 isoform 2 mRNA |
| 104 | ZNF415 isoform 1 mRNA |
| 105 | ZNF415 isoform 2 mRNA |
| 106 | ZNF415 isoform 3 mRNA |
| 107 | ZNF415 isoform 4 mRNA |
| 108 | ZNF415 isoform 5 mRNA |
| 109 | ZNF415 isoform 6 mRNA |
| 110 | ZNF415 isoform 7 mRNA |
| 111 | ZNF415 isoform 8 mRNA |
| 112 | ZNF415 isoform 9 mRNA |
| 113 | IRX5 isoform 1 mRNA |
| 114 | IRX5 isoform 2 mRNA |
| 115 | TMPRSS15 mRNA |
| 116 | IL3RA isoform 1 mRNA |
| 117 | IL3RA isoform 2 mRNA |
| 118 | IGF2BP1 isoform 1 mRNA |
| 119 | IGF2BP1 isoform 2 mRNA |
| 120 | IL2RA Protein isoform 1 precursor |
| 121 | IL2RA Protein isoform 2 precursor |
| 122 | IL2RA Protein isoform 3 precursor |
| 123 | ATP9A protein |
| 124 | HMX3 protein |
| 125 | DPYSL3 isoform 1 protein |
| 126 | DPYSL3 isoform 2 protein |
| 127 | ZNF415 isoform 1 protein |
| 128 | ZNF415 isoform 2 protein |
| 129 | ZNF415 isoform 3 protein |
| 130 | ZNF415 isoform 4 protein |
| 131 | ZNF415 isoform 5 protein |
| 132 | ZNF415 isoform 6 protein |
| 133 | ZNF415 isoform 7 protein |
| 134 | ZNF415 isoform 8 protein |
| 135 | ZNF415 isoform 9 protein |
| 136 | IRX5 isoform 1 protein |
| 137 | IRX5 isoform 2 protein |
| 138 | TMPRSS15 protein |
| 139 | IL3RA isoform 1 protein |
| 140 | IL3RA isoform 2 protein |
| 141 | IGF2BP1 isoform 1 protein |
| 142 | IGF2BP1 isoform 2 protein |
| 143 | PCDHGB5 isoform 1 mRNA |
| 144 | PCDHGB5 Isoform 2 mRNA |
| 145 | PCDHGB5 isoform 1 Protein |
| 146 | PCDHGB5 Isoform 2 Protein |
| 147 | spacer (IgG4hinge) (aa) Homo sapiens |
| 148 | spacer (IgG4hinge) (nt) Homo sapiens |
| 149 | Hinge-CH3 spacer Homo sapiens |
| 150 | Hinge-CH2-CH3 spacer Homo sapiens |
| 151 | IgD-hinge-Fc Homo sapiens |
| 152 | T2A artificial |
| 153 | tEGFR artificial |
| 154 | CD28 (amino acids 153-179 of Accession No. |
| | P10747)Homo sapiens |
| 155 | CD28 (amino acids 114-179 of Accession No. P10747) |
| | Homo sapiens |
| 156 | CD28 (amino acids 180-220 of P10747)Homo sapiens |
| 157 | CD28 (LL to GG)Homo sapiens |
| 158 | 4-1BB (amino acids 214-255 of Q07011.1) Homo sapiens |
| 159 | CD3 zeta Homo sapiens |
| 160 | CD3 zeta Homo sapiens |
| 161 | CD3 zeta Homo sapiens |
| 162 | tEGFR artificial |
| 163 | 2A element T2A |
| 164 | 2A element P2A |
| 165 | 2A element P2A |
| 166 | 2A element E2A |
| 167 | 2A element F2A |
| 168 | Exemplary linker sequence |
| 169 | Exemplary linker sequence |
| 170 | Exemplary linker sequence |
| 171 | Exemplary linker sequence |
| 172 | Exemplary spacer derived from Ig hinge |
| 173 | Exemplary spacer derived from Ig hinge |
| 174 | Exemplary spacer derived from Ig hinge |
| 175 | Exemplary spacer derived from Ig hinge |
| 176 | Exemplary spacer derived from Ig hinge |
| 177 | Exemplary spacer derived from Ig hinge |
| 178 | Exemplary spacer derived from Ig hinge |
| 179 | Exemplary spacer derived from Ig hinge |
| 180 | FMC63 LC-CDR3 |
| 181 | FMC63 CDR L1 |
| 182 | FMC63 CDR L2 |
| 183 | FMC63 CDR L3 |
| 184 | FMC63 CDR H1 |
| 185 | FMC63 CDR H2 |
| 186 | FMC63 CDR H3 |
| 187 | FMC63 VH |
| 188 | FMC63 VL |
| 189 | FMC63 scFv |
| 190 | SJ25C1 CDR L1 |
| 191 | SJ25C1 CDR L2 |
| 192 | SJ25C1 CDR L3 |
| 193 | SJ25C1 CDR H1 |
| 194 | SJ25C1 CDR H2 |
| 195 | SJ25C1 CDR H3 |
| 196 | SJ25C1 VH |
| 197 | SJ25C1 VL |
| 198 | Linker |
| 199 | SJ25C1 scFv |
| 200 | FMC63 HC-CDR3 |
| 201 | FMC63 LC-CDR2 |
| 202 | Linker |
| 203 | Sequence encoding scFv |

What is claimed:

1. A method of assessing a risk of a toxicity or a toxicity-related outcome, following administration of an immunotherapy, the method comprising:
(1) assessing the presence, absence or level of expression of one or more gene products or portions thereof in a sample from a subject that is a candidate for receiving a immunotherapy for treatment of a disease or condition, wherein the disease or condition is acute lymphoblastic leukemia (ALL) or a subtype thereof, wherein:
the one or more gene products is associated with a risk of developing neurotoxicity following administration of the immunotherapy; and
the sample does not comprise the immunotherapy and/or is obtained from the subject prior to receiving the immunotherapy; and
(2) comparing the presence, absence or level of expression of the one or more gene products or portions thereof to a gene reference value, wherein the comparison indicates the risk or likely risk of the subject developing a neurotoxicity, optionally a specified grade or severity of neurotoxicity, following administration of the therapy to the subject.

2. The method of claim 1, wherein each of the one or more gene products is individually compared to a gene reference value for the respective gene product.

3. The method of claim 1 or claim 2, wherein:
(a) at least one of the one or more gene products is from a first group of gene products that negatively correlate to a risk of developing neurotoxicity; and/or
(b) at least one of the one or more gene products is from a second group of gene products that positively correlates to a risk of developing neurotoxicity.

4. The method of claim 3, wherein:
the at least one gene product is from (a) and is selected from CCL17, ABCA9, ADAMTSL4, ADGRA2, ADGRF1, AK5, APOL1, ARHGAP27, ARID3B, CA6, CABP7, CCDC152, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS 1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or
the at least one gene product is from (b) and is selected from PINLYP, PCDHGA12, ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA4, PCDHGA6, PCDHGB1, PCDHGB5, PCDHGB6, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or is a portion or fragment thereof.

5. A method of assessing a risk of toxicity following administration of an immunotherapy, the method comprising:
(1) assessing the presence, absence or level of expression of one or more gene products or a portion thereof in a sample from a subject that is a candidate for receiving a immunotherapy for treating a disease or condition, wherein the disease or condition is acute lymphoblastic leukemia (ALL) or a subtype thereof, wherein:
(a) at least one of the one or more gene products is selected from CCL17, ABCA9, ADAMTSL4, ADGRA2, ADGRF1, AKS, APOL1, ARHGAP27, ARID3B, CA6, CABP7, CCDC152, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof, optionally wherein said one or more gene products negatively correlate to a risk of developing neurotoxicity; and/or
(b) at least one of the one or more gene products is selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, or ZNF415 or is a portion or fragment thereof, optionally wherein said one or more gene products positively correlates to a risk of developing neurotoxicity; and
(2) comparing the presence, absence or level of expression of the one or more gene product to a gene reference value, wherein the comparison indicates whether the subject is or is likely at risk of developing a neurotoxicity or grade or severity thereof following administration of the immunotherapy when administered to the subject.

6. The method of claim 5, wherein each of the one or more gene products is individually compared to a gene reference value for the respective gene product.

7. The method of any of claims 1-6, wherein the immunotherapy is a cell therapy or is a T cell-engaging therapy, optionally wherein the cell therapy comprises cells engineered to express a recombinant receptor.

8. The method of claim 5, claim 6 or claim 7, wherein the sample does not comprise the immunotherapy, and/or is obtained from the subject prior to receiving the immunotherapy, optionally wherein the immunotherapy is a cell therapy.

9. The method of any of claims 1-8, wherein the sample does not contain cells genetically engineered with the recombinant receptor.

10. The method of any of claims 3-9, wherein:
the comparison indicates the subject is or is likely at risk of developing neurotoxicity if the at least one gene product of (a) is at or below a gene reference value and/or the at least one gene product of (b) is at or above a gene reference value; or the comparison indicates the subject is not or is likely not at risk of developing neurotoxicity if the at least one gene product of (a) is above a gene reference value and/or the at least one gene product of (b) is below a gene reference value.

11. The method of claim 10, wherein if the comparison indicates the subject is or is likely to develop neurotoxicity, selecting the subject for administration of a therapeutic regimen, the therapeutic regimen comprising administering to the subject:
i. an agent or other treatment capable of treating, preventing, delaying, reducing or attenuating the development or risk of development of a toxicity and the immunotherapy, wherein administration of the agent is to be administered (i) prior to, (ii) within one, two, or three days of, (iii) concurrently with and/or (iv) at first fever following, the initiation of administration of the immunotherapy to the subject;
ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy;
iii. the immunotherapy in an in-patient setting and/or with admission to the hospital for one or more days, optionally wherein the immunotherapy is otherwise to be administered to subjects on an outpatient basis or without admission to the hospital for one or more days; or iv. an alternative therapeutic treatment other than the immunotherapy.

12. The method of claim 10, wherein if the comparison indicates the subject is not or is likely not at risk of developing neurotoxicity, selecting the subject for administration of a therapeutic regimen, the therapeutic regimen comprising administering to the subject:
i. the immunotherapy, optionally at a non-reduced dose, optionally on an outpatient basis or without admission to the hospital for one or more days;
ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or
iii. the immunotherapy in an outpatient setting and/or without admission of the subject to the hospital overnight or for one or more consecutive days and/or is without admission of the subject to the hospital for one or more days.

13. The method of claim 11 or claim 12, further comprising administering the therapeutic regimen to the selected subject.

14. A method of treatment, the method comprising administering a therapeutic regimen to a subject that is a candidate for receiving an immunotherapy for treatment of a disease or condition, wherein the disease or condition is acute lymphoblastic leukemia (ALL) or a subtype thereof, wherein the administration is carried out following or based on the results of assessing the presence, absence or level of expression, from a sample from the subject, of one or more gene products or portion thereof, wherein:
(a) at least one of the one or more gene products is selected from CCL17, ABCA9, ADAMTSL4, ADGRA2, ADGRF1, AK5, APOL1, ARHGAP27, ARID3B, CA6, CABP7, CCDC152, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAP5, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS 1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, or is a portion or fragment thereof; and/or
(b) at least one of the one or more gene products is selected from ASAP2, ATP9A, CCNA1, CDHR3, CECR2, DLX1, DPYSL3, EHD4, FMNL2, GGA2, HHIPL1, HMX3, IGF2BP1, IL3RA, IRX5, KCNIP1, KIAA1644, LINC00092, LINC01483, MIB1, MMP14, NOM1, OTOA, PCDHGA12, PCDHGA4, PCDHGA6, PCDHGB5, PCDHGB6, PINLYP, PPM1E, PRKD1, PROKR2, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TNKS1BP1, TTC28, PCDHGA9, FMNL1, or ZNF415 or is a portion or fragment thereof,
wherein the presence, absence or level of the one or more gene products from (a) negatively correlate to a risk that the subject is or is likely to develop neurotoxicity following administration of the immunotherapy when it is administered, and expression of the at least one or more gene products from (b) positively correlates to a risk that the subject is or is likely to develop neurotoxicity following administration of the immunotherapy when it is administered.

15. The method of claim 14, wherein the immunotherapy is a cell therapy or is a T cell-engaging therapy, optionally wherein the cell therapy comprises cells engineered to express a recombinant receptor.

16. The method of claim 14 or 15, wherein the sample is obtained from the subject prior to receiving the immunotherapy and/or the sample does not comprise the immunotherapy.

17. The method of any of claims 14-16, wherein the results of assessing the presence, absence or level of expression of the one or more gene products or portions thereof comprises a comparison to a gene reference value, wherein the comparison indicates the risk or likely risk of the subject developing neurotoxicity following administration of the immunotherapy when administered to the subject.

18. The method of claim 17, wherein each of the one or more gene products is individually compared to a gene reference value for the respective gene product.

19. The method of any of claims 14-18, wherein if the assessing indicates the subject is or is likely to develop neurotoxicity following administration of the immunotherapy, the therapeutic regimen comprises administering to the subject:
i. an agent or other treatment capable of treating, preventing, delaying, reducing or attenuating the development or risk of development of a toxicity and the immunotherapy, wherein administration of the agent is to be administered (i) prior to, (ii) within one, two, or three days of, (iii) concurrently with and/or (iv) at first fever following, the initiation of administration of the immunotherapy to the subject;
ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy;
iii. the immunotherapy in an in-patient setting and/or with admission to the hospital for one or more days, optionally wherein the immunotherapy is otherwise to be administered to subjects on an outpatient basis or without admission to the hospital for one or more days; or iv. an alternative therapeutic treatment other than the immunotherapy.

20. The method of any of claims 14-18, wherein if the assessing indicates the subject is not or is likely not to develop neurotoxicity following administration of the immunotherapy, the therapeutic regimen comprises administering to the subject:
i. the immunotherapy, optionally at a non-reduced dose, optionally on an outpatient basis or without admission to the hospital for one or more days;
ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or
iii. the immunotherapy in an outpatient setting and/or without admission of the subject to the hospital overnight or for one or more consecutive days and/or is without admission of the subject to the hospital for one or more days.

21. The method of any of claims 3-20, wherein the at least one gene product is from (a) and is a gene product associated with a PH+ or Ph-like molecular subtype of ALL.

22. The method of claim 21, wherein the at least one gene product is selected from CCL17, ADGRF1, BMPR1B, CA6, CCR6, CD99, CHN2, CRLF2, DENND3, ENAM, GAS6, GBP5, GLI2, IFITM1, IGJ (JCHAIN), LDB3, L0645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A, or is a portion or fragment of any of the foregoing.

23. A method of treatment, the method comprising:
selecting a subject that exhibits a Philadelphia chromosome (Ph+) and/or Ph chromosome-like (Ph-like) molecular subtype of acute lymphoblastic leukemia (ALL); and
administering to the subject an immunotherapy that binds to an antigen associated with the ALL.

24. The method of claim 23, wherein the immunotherapy is a cell therapy or is a T cell-engaging therapy, optionally wherein the cell therapy comprises cells engineered to express a recombinant receptor.

25. The method of claim 23 or claim 24, wherein:
the selected subject exhibits one or more of (9;22)(q34;q11) chromosomal abnormality; deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or comprises a Ph-like gene expression signature; or
the subject is selected based on one or more of the presence of (9;22)(q34;q11) chromosomal abnormality, deletion or mutation of IKZF1 transcription factor; a kinase-activating alteration, optionally a rearrangement involving ABL1, ABL2, CRLF2, CSF1R, EPOR, JAK2, NTRK3, PDGFRB, PTK2B, TSLP, or TYK2; a sequence mutation involving FLT3, IL7R, SH2B3, TYK2, IL2RB, NTRK3, DGKH, KRAS, NRAS, PTPN11, NF1; and/or the presence of a Ph-like gene expression signature.

26. The method of claim 25, wherein the Ph-like gene signature is based on comparison of the presence, absence or level of expression, in a sample from the subject, of at least one gene product to a reference gene value, said at least one gene product is selected from:
(a) CCL17, ADGRF1, BMPR1B, CA6, CCR6, CD99, CHN2, CRLF2, DENND3, ENAM, GAS6, GBP5, GLI2, IFITM1, IGJ (JCHAIN), LDB3, L0645744, MDF1C, MUC4, NRXN3, PON2, PTP4A3, S100Z, SEMA6A, SLC37A3, SLC2A5, SPATS2L, TMEM154, TP53INP1, TTYH2, IL2RA, or WNT9A or a portion or fragment of any of the foregoing; and/or
(b) ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing,
wherein the comparison indicates the subject exhibits a Ph-like molecular subtype of ALL if the at least one gene product of (a) is above a gene reference value and/or the at least one gene product of (b) is below a gene reference value.

27. The method of claim 26, wherein each of the one or more gene products is individually compared to a gene reference value for the respective gene product.

28. The method of any of claims 21, 22, 26 and 27, wherein the at least one gene product selected from (a) is ADGRF1, BMPR1B, CA6, CD99, CHN2, CRLF2, DENND3, ENAM, GBP5, GLI2, IFITM1, IGJ (JCHAIN), LDB3, L0645744, MDF1C, MUC4, NRXN3, PON2, S100Z, SEMA6A, SLC37A3, SLC2A5, SPATS2L, TMEM154, TP53INP1, TTYH2 or WNT9A, or is a portion of fragment of any of the foregoing.

29. The method of any of claims 3-22 and 26-28, wherein the at least one gene product selected from (a) is ADGRF1, CA6, CCL17, CCR6, ENAM, GAS6, GBP5, GLI2, IFITM1, IGJ (JCHAIN), MUC4, PON2, PTP4A3, SEMA6A, SLC37A3, SPATS2L, TMEM154, TP53INP1, IL2RA, or WNT9A or is a portion or fragment of any of the foregoing.

30. The method of any of claims 3-22 and 26-29, wherein the at least one gene product selected from (b) is ASAP2, FMNL2, GPR176, MDFI, PCDHGA12, PCDHGA6, PCDHGB5, PCDHGB6, PINLYP, PTCH1, ATP9A, HMX3, DPYSL3, ZNF415, IRX5, TMPRSS15, IL3RA, IGF2BP1, or TTC28 or is a portion or fragment of any of the foregoing.

31. The method of any of claims 1-30, wherein the subject is a human and/or the one or more gene products is human.

32. The method of any of claims 3-22 and 26-31, wherein at least one of the one or more gene products is from (a) and at least one of the one or more gene products is from (b).

33. The method of any of claims 3-22 and 26-32, wherein the one or more gene product comprises at least one gene product from (a) that is IGJ (JCHAIN), MUC4, CA6, WNT9A, ADGRF1 or CCL17, or a portion or fragment of any of the foregoing.

34. The method of any of claims 3-22 and 26-33, wherein the one or more gene products comprises at least one gene product from (a) that is CCL17 or a portion or fragment thereof.

35. The method of any of claims 3-22 and 26-34, wherein the one or more gene products comprises at least one gene product from (b) that is PINLYP, ASAP2, FMNL2, PTCH1, TTC28, PCDHGA6, PCDHGB6 or PCDHGA12, or a portion or fragment of any of the foregoing.

36. The method of any of claims 3-22 and 26-35, wherein the one or more gene products comprise at least one gene product from (b) that is PINLYP or PCDHGA12 or a portion or fragment of any of the foregoing.

37. The method of any of claims 13-36, wherein:
greater than or greater than about 30%, 35%, 40%, or 50% of the subjects treated according to the method do not exhibit any grade of cytokine release syndrome (CRS) or neurotoxicity; and/or
at least at or about 45, 50, 60, 65, 70, 75, 80, 85, 90, 95% or about 100% of subjects treated according to the method do not exhibit severe CRS, optionally grade 3 or higher, prolonged grade 3 or higher or grade 4 or 5 CRS; and/or
at least at or about 45, 50, 60, 65, 70, 75, 80, 85, 90, 95% or about 100% of subjects treated according to the method do not exhibit severe neurotoxicity, optionally grade 3 or higher, prolonged grade 3 or higher or grade 4 or 5 neurotoxicity; and/or
at least at or about 45, 50, 60, 65, 70, 75, 80, 85, 90, 95% or about 100% of subjects treated according to the method do not exhibit cerebral edema.

38. The method of any of claims 13-37, wherein:
prior to initiation of administration of the dose of cells, the subject has not been administered an agent or treatment capable of treating, preventing, delaying, reducing or attenuating the development or risk of development of a toxicity; and/or
the subject is not administered an agent or treatment for the treatment or prevention or reduction or attenuation of a neurotoxicity and/or a cytokine release syndrome or risk thereof, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or
the subject is not administered an agent or treatment for the treatment or prevention or reduction or attenuation of a neurotoxicity and/or a cytokine release syndrome or risk thereof, following administration of the dose, prior to or unless the subject exhibits a sign or symptom of the toxicity and/or prior to or unless the subject exhibits a sign or symptom of the toxicity other than a fever, optionally wherein the fever is not a sustained fever or the fever is or has been reduced or reduced by more than 1° C. after treatment with an antipyretic; and/or
the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

39. The method of any of claims 13-38, wherein:
prior to initiation of administration of the dose of cells, the subject has not been administered an anti-IL-6 or anti-IL-6R antibody, optionally tocilizumab or siltuximab, and/or has not been administered a steroid, optionally dexamethasone;
the subject is not administered an anti-IL-6 or anti-IL-6R antibody, optionally tocilizumab or siltuximab, and/or has not been administered a steroid, optionally dexamethasone, within a period of time following administration of the dose, which period of time is optionally at or about 1, 2, 3, 4, 5 days or is optionally at or about 6, 7, 8, 9, 10, 11 days or is optionally 1 or 2 or 3 or 4 weeks; and/or
the subject is not administered an anti-IL-6 or anti-IL-6R antibody, optionally tocilizumab or siltuximab, and/or has not been administered a steroid, optionally dexamethasone, following administration of the cell dose, prior to, or unless, the subject exhibits a sign or symptom of a toxicity, optionally a neurotoxicity or CRS, and/or prior to, or unless, the subject exhibits a sign or symptom of a toxicity, optionally a neurotoxicity or CRS, other than a fever, optionally wherein the fever is not a sustained fever or the fever is or has been reduced or reduced by more than 1° C. after treatment with an antipyretic; and/or
the administration and any follow-up is carried out on an outpatient basis and/or without admitting the subject to a hospital and/or without an overnight stay at a hospital and/or without requiring admission to or an overnight stay at a hospital, optionally unless or until the subject exhibits a sustained fever or a fever that is or has not been reduced or not reduced by more than 1° C. after treatment with an antipyretic.

40. The method of any of claims 13-39, wherein:
the administration is carried out on an outpatient basis and/or without requiring admission to or an overnight stay at a hospital; and
if the subject exhibits a sustained fever or a fever that is or has not been reduced or not reduced by more than 1° C. after treatment with an antipyretic, the subject is admitted to the hospital or to an overnight stay at a hospital and/or is administered an agent or treatment for the treatment or prevention or reduction or attenuation of a neurotoxicity and/or a cytokine release syndrome or risk thereof.

41. The method of any of claims 1-40, wherein the neurotoxicity comprises severe neurotoxicity, optionally at or above grade 4 or grade 5 or at least prolonged grade 3 neurotoxicity.

42. The method of any of claims 1-41, wherein the ALL is adult ALL or pediatric ALL.

43. The method of any of claims 1-42, wherein the sample is or comprises a bone marrow sample, blood sample, plasma sample, or serum sample.

44. The method of any of claims 1-43, wherein the sample is or comprises a bone marrow aspirate.

45. The method of any of claims 1-43, wherein the sample is or comprises a serum or plasma sample.

46. The method of any of claims 1-45, wherein the presence, absence or level of expression of one, two, three, four, five, six, seven, eight, nine, ten or more gene products is assessed or compared.

47. The method of any of claims 1-46, wherein the one or more gene products or portion or fragment thereof is a polynucleotide or a portion thereof.

48. The method of claim 47, wherein the polynucleotide is an RNA.

49. The method of any of claims 1-46, wherein the one or more gene products or portions thereof comprise a protein or a portion thereof.

50. The method of claim 49, wherein the one or more gene products is a gene product from (s) selected from CCL17, ENG, SELE, ICAM3, or IL6R.

51. The method of any of claims 3-50, wherein the gene reference value for the at least one gene product of (a), or each of the gene reference values individually for each of the at least one or more gene product of (a), is a value that:
i) is within 25%, within 20%, within 15%, within 10%, or within 5% above the average level, concentration or amount, and/or is within a standard deviation above the average level, concentration or amount, of the at least one gene product in a plurality of control samples;
ii) is above the highest level, concentration or amount of the at least one gene product, optionally within 50%, within 25%, within 20%, within 15%, within 10%, or within 5% above such highest level, concentration or amount, measured in at least one sample from among a plurality of control samples; and/or
iii) is above the highest level, concentration or amount of the at least one gene product as measured among more than 75%, 80%, 85%, 90%, or 95%, or 98% of samples from a plurality of control samples;
wherein the plurality of control samples are a plurality of biological samples obtained from a group of subjects prior to receiving a immunotherapy for treating ALL, wherein each of the subjects of the group went on to develop severe neurotoxicity, optionally grade 3 or higher, prolonged grade 3 or higher or grade 4 or 5 neurotoxicity, after receiving the immunotherapy for treating the same disease or condition.

52. The method of claim 51, further wherein the gene reference value for the at least one gene product of (a), or each of the gene reference values individually for each of the at least one or more gene product of (a), is:
below the lowest level, concentration, or amount, optionally within 50%, within 25%, within 20%, within 15%, within 10%, or within 5% below the lowest level, concentration or amount, of the at least one gene product observed in a sample from among a second plurality of control samples; and/or
below the level, concentration or amount of the at least one gene product measured as measured in more than 75%, 80%, 85%, 90%, 95%, or 98% of samples from among a second plurality of control samples
wherein the second plurality of control samples is obtained from a group of subjects prior to receiving the same immunotherapy for treating the same disease or condition, wherein each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition.

53. The method of any of claims 3-70, wherein the gene reference value for the at least one gene product of (a), or each of the gene reference values individually for each of the at least one or more gene product of (a), is a value that:
i) is within 25%, within 20%, within 15%, within 10%, or within 5% above the average level, concentration or amount, and/or is within a standard deviation above the average level, concentration or amount, of the at least one gene product in a plurality of control samples;
ii) is above the highest level, concentration or amount of the at least one gene product, optionally within 50%, within 25%, within 20%, within 15%, within 10%, or within 5% above such highest level, concentration or amount, as measured in at least one sample from among a plurality of control samples; and/or
iii) is above the highest level, concentration or amount of the at least one gene product measured among more than 75%, 80%, 85%, 90%, 95%, or 98% of samples from a plurality of control samples;
wherein the plurality of control samples are a plurality of biological samples obtained from a group of subjects prior to receiving a immunotherapy for treating ALL, wherein each of the subjects of the group has ALL that is not Philadelphia chromosome positive (Ph+) or Philadelphia-like (Ph-like) subtype of ALL.

54. The method of claim 53, further wherein the gene reference value for the at least one gene product of (a), or each of the gene reference values individually for each of the at least one or more gene product of (a), is:
below the lowest level, concentration, or amount, optionally within 50%, within 25%, within 20%, within 15%, within 10%, or within 5% below the lowest level, concentration or amount, of the at least one gene product observed in a sample from among a second plurality of control samples; and/or
below the level, concentration or amount measured in more than 75%, 80%, 85%, 90%, 95%, or 98% of samples from among a plurality of control samples,
wherein the second plurality of control samples is obtained from a group of subjects prior to receiving the same immunotherapy for treating the same disease or condition, wherein each of the subjects has ALL that is Philadelphia chromosome positive (Ph+) or Philadelphia-like (Ph-like) subtype of ALL.

55. The method of any of claims 3-54, wherein the gene reference value for the at least one gene product of (b), or each of the gene reference values individually for each of the at least one or more gene product of (b), is a value that:
i) is within 25%, within 20%, within 15%, within 10%, or within 5% below the average level, concentration or amount, and/or is within a standard deviation below the average level, concentration or amount, of the at least one gene product in a plurality of control samples;
ii) is below the lowest level, concentration or amount of the at least one gene product, optionally within 50%, within 25%, within 20%, within 15%, within 10%, or within 5% below the lowest level, concentration or amount, as measured in at least one sample from among a plurality of control samples;
iii) is below the lowest level, concentration or amount of the at least one gene product measured among more than 75%, 80%, 85%, 90%, 95%, or 98% of samples from a plurality of control samples;
wherein the plurality of control samples are a plurality of biological samples obtained from a group of subjects prior to receiving a immunotherapy for treating ALL, wherein each of the subjects of the group went on to develop severe neurotoxicity, optionally grade 3 or higher, prolonged grade 3 or higher or grade 4 or 5 neurotoxicity, after receiving the immunotherapy for treating the same disease or condition.

56. The method of claim 55, further wherein the gene reference value for the at least one gene product of (b), or each of the gene reference values individually for each of the at least one or more gene product of (b) is:
above the highest level, concentration, or amount of the at least one gene product, optionally within 50%, within 25%, within 20%, within 15%, within 10%, or within 5% above such level, concentration or amount, measured in at least one sample from among a second plurality of control samples; and/or
above the level, concentration or amount of the at least one gene product measured in more than 75%, 80%, 85%, 90%, 95%, or 98% of samples from among a second plurality of control samples,
wherein the second plurality of control samples are a plurality of control samples obtained from a group of subjects prior to receiving the immunotherapy for treating the disease or condition, wherein each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition.

57. The method of any of claims 3-54, wherein the gene reference value for the at least one gene product of (b), or each of the gene reference values individually for each of the at least one or more gene product of (b), is a value that:
i) is within 25%, within 20%, within 15%, within 10%, or within 5% below the average level, concentration or amount, and/or is within a standard deviation below the average level, concentration or amount, of the at least one gene product in a plurality of control samples;
ii) is below the lowest level, concentration or amount of the at least one gene product, optionally within 50%, within 25%, within 20%, within 15%, within 10%, or within 5% below such lowest level, concentration or amount, as measured in at least one sample from among a plurality of control samples;
iii) is below the lowest level, concentration or amount of the at least one gene product measured among more than 75%, 80%, 85%, 90%, 95%, or 98% of samples from a plurality of control samples;
wherein the plurality of control samples are a plurality of biological samples obtained from a group of subjects prior to receiving a immunotherapy for treating ALL, wherein each of the subjects of the group has ALL that is not Philadelphia chromosome positive (PH+) or Philadelphia-like (Ph-like) subtype of ALL.

58. The method of claim 57, further wherein the gene reference value for the at least one gene product of (b), or each of the gene reference values individually for each of the at least one or more gene product of (b) is:
above the highest level, concentration, or amount of the at least one gene product, optionally within 50%, within 25%, within 20%, within 15%, within 10%, or within 5% above such level, concentration or amount, measured in at least one sample from among a second plurality of control samples; and/or
is above the level, concentration or amount of the at least one gene product measured among more than 75%, 80%, 85%, 90%, 95%, or 98% of samples from a second plurality of control samples,
wherein the second plurality of control samples are a plurality of control samples obtained from a group of subjects prior to receiving the immunotherapy for treating the same disease or condition, wherein each of the subjects has ALL that is Philadelphia chromosome positive (Ph+) or Philadelphia-like (Ph-like) subtype of ALL.

59. The method of any of claims 51-58, wherein the control sample or each of the plurality of control samples is the same type of biological sample being assessed from the subject, optionally is a bone marrow sample or aspirate or is a plasma sample.

60. The method of any of claims 51-59, wherein the plurality of control samples comprises at least 3, at least 10, at least 20, at least 50, or at least 100 control samples.

61. A method of assessing a risk of a toxicity or a toxicity-related outcome, following administration of an immunotherapy, the method comprising
(a) assessing the level or amount of one or more proteins or portions thereof in a biological sample from a subject that is a candidate for receiving a immunotherapy for treatment of a disease or condition, wherein the disease or condition is acute lymphoblastic leukemia (ALL) or a subtype thereof, wherein at least one of the one the one or more proteins or portions thereof are selected from CCL17, ENG, SELE, ICAM3, or IL6R; and
(b) comparing the level or amount of the one or more proteins or portions thereof to a reference value, wherein:
the comparison indicates the subject is or is likely at risk of developing neurotoxicity if the at least one of the one or more proteins or portions thereof is at or below the reference value; or
the comparison indicates the subject is not or is likely not at risk of developing neurotoxicity if at least one of the one or more protein or portions thereof is above the reference value.

62. The method of claim 61, wherein the biological sample is a plasma sample.

63. The method of claim 61 or claim 62, wherein at least one of the one or more proteins or portions thereof is CCL17.

64. The method of any of claims 61-63, wherein the immunotherapy is a cell therapy or is a T cell-engaging therapy, optionally wherein the cell therapy comprises cells engineered to express a recombinant receptor.

65. The method of any of claims 61-64, wherein the sample does not comprise the immunotherapy, and/or is obtained from the subject prior to receiving the immunotherapy.

66. The method of any of claims 61-65, wherein the reference value for the one or more protein or portion thereof, or each of the reference values individually for each of the one or more protein or portion thereof, is a value that:
i) is within 25%, within 20%, within 15%, within 10%, or within 5% above the average level, concentration or amount, and/or is within a standard deviation above the average level, concentration or amount, of the one or more protein or portion thereof in a plurality of control samples;
ii) is above the highest level, concentration or amount of the one or more protein or portion thereof, optionally within 50%, within 25%, within 20%, within 15%, within 10%, or within 5% above such highest level, concentration or amount, measured in at least one sample from among a plurality of control samples; and/or
iii) is above the highest level, concentration or amount of the one or more protein or portion thereof as measured among more than 75%, 80%, 85%, 90%, or 95%, or 98% of samples from a plurality of control samples;
wherein the plurality of control samples are a plurality of biological samples obtained from a group of subjects prior to receiving a immunotherapy for treating ALL, wherein (1) each of the subjects of the group went on to develop severe neurotoxicity, optionally grade 3 or higher, prolonged grade 3 or higher or grade 4 or 5 neurotoxicity, after receiving the immunotherapy for treating the same disease or condition; or (2) each of the subjects of the group has ALL that is not Philadelphia chromosome positive (Ph+) or Philadelphia-like (Ph-like) subtype of ALL.

67. The method of claim 66, further wherein the reference value for the one or more protein or portion thereof, or each of the gene reference values individually for each of the one or more protein or portion thereof, is:
below the lowest level, concentration, or amount, optionally within 50%, within 25%, within 20%, within 15%, within 10%, or within 5% below the lowest level, concentration or amount, of the one or more protein or portion thereof observed in a sample from among a second plurality of control samples; and/or
below the level, concentration or amount of the one or more protein or portion thereof as measured in more than 75%, 80%, 85%, 90%, 95%, or 98% of samples from among a second plurality of control samples,
wherein the second plurality of control samples is obtained from a group of subjects prior to receiving the same immunotherapy for treating the ALL, wherein (1) each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition, or (2) each of the subjects has ALL that is Philadelphia chromosome positive (Ph+) or Philadelphia-like (Ph-like) subtype of ALL.

68. The method of claim 66 or claim 67, wherein the control sample or each of the plurality of control samples is the same type of biological sample being assessed from the subject, optionally is a plasma sample.

69. The method of any of claims 66-68, wherein the plurality of control samples comprises at least 3, at least 10, at least 20, at least 50, or at least 100 control samples.

70. The method of any of claims 61-69, wherein if the comparison indicates the subject is or is likely to develop neurotoxicity, selecting the subject for administration of a therapeutic regimen, the therapeutic regimen comprising administering to the subject:
i. an agent or other treatment capable of treating, preventing, delaying, reducing or attenuating the development or risk of development of a toxicity and the immunotherapy, wherein administration of the agent is to be administered (i) prior to, (ii) within one, two, or three days of, (iii) concurrently with and/or (iv) at first fever following, the initiation of administration of the immunotherapy to the subject;
ii. the immunotherapy at a reduced dose or at a dose that is not associated with risk of developing toxicity or severe toxicity, or is not associated with a risk of developing a toxicity or severe toxicity in a majority of subjects, and/or a majority of subjects having a disease or condition that the subject has or is suspected of having, following administration of the immunotherapy;
iii. the immunotherapy in an in-patient setting and/or with admission to the hospital for one or more days, optionally wherein the immunotherapy is otherwise to be administered to subjects on an outpatient basis or without admission to the hospital for one or more days; or iv. an alternative therapeutic treatment other than the immunotherapy.

71. The method of any of claims 61-69, wherein if the comparison indicates the subject is not or is likely not at risk of developing neurotoxicity, selecting the subject for administration of a therapeutic regimen, the therapeutic regimen comprising administering to the subject:
i. the immunotherapy, optionally at a non-reduced dose, optionally on an outpatient basis or without admission to the hospital for one or more days;
ii. the immunotherapy, wherein administration of the immunotherapy does not comprise administering, prior to or concurrently with administering the immunotherapy and/or prior to the development of a sign or symptom of toxicity other than fever, an agent or treatment capable of treating, preventing, delaying, or attenuating the development of the toxicity; or
iii. the immunotherapy in an outpatient setting and/or without admission of the subject to the hospital overnight or for one or more consecutive days and/or is without admission of the subject to the hospital for one or more days.

72. The method of any of claims 61-71, further comprising administering the therapeutic regimen to the selected subject.

73. A method of assessing a risk of a toxicity or a toxicity-related outcome, following administration of an immunotherapy, the method comprising
(a) assessing the level or amount of one or more proteins or portions thereof in a biological sample from a subject that received an immunotherapy for treatment of a disease or condition, wherein:
at least one of the one the one or more proteins or portions thereof are selected from CCL27, ENG, FAS, 1-309, ICAM3, NSE, P-Selectin, Resistin, S100(3, Thrombomodulin or vWF; and
(b) comparing the level or amount of the one or more proteins or portions thereof to a reference value, wherein:
the comparison indicates the subject is or is likely at risk of developing neurotoxicity if the at least one of the one or more proteins or portions thereof is at or below the reference value; or
the comparison indicates the subject is not or is likely not at risk of developing neurotoxicity if at least one of the one or more protein or portions thereof is above the reference value.

74. The method of claim 73, wherein the biological sample is a plasma sample.

75. The method of claim 73 or claim 74, wherein the biological sample is obtained or collected from the subject no more than 4 days, no more than 3 days, no more than 2 days or no more than 1 day, after initiation of administration of the immunotherapy and/or before the subject exhibits a sign or symptom of the toxicity and/or before the subjects develops a sustained fever.

76. The method of any of claims 73-75, wherein at least one of the one or more proteins or portions thereof is ENG or ICAM3.

77. The method of any of claims 73-76, wherein the immunotherapy is a cell therapy or is a T cell-engaging therapy, optionally wherein the cell therapy comprises cells engineered to express a recombinant receptor.

78. The method of any of claims 73-77, wherein the reference value for the one or more protein or portion thereof, or each of the reference values individually for each of the one or more protein or portion thereof, is a value that:
i) is within 25%, within 20%, within 15%, within 10%, or within 5% above the average level, concentration or amount, and/or is within a standard deviation above the average level, concentration or amount, of the one or more protein or portion thereof in a plurality of control samples;
ii) is above the highest level, concentration or amount of the one or more protein or portion thereof, optionally within 50%, within 25%, within 20%, within 15%, within 10%, or within 5% above such highest level, concentration or amount, measured in at least one sample from among a plurality of control samples; and/or
iii) is above the highest level, concentration or amount of the one or more protein or portion thereof as measured among more than 75%, 80%, 85%, 90%, or 95%, or 98% of samples from a plurality of control samples;
wherein the plurality of control samples are a plurality of biological samples obtained from a group of subjects after receiving a immunotherapy for treating ALL, wherein (1) each of the subjects of the group went on to develop severe neurotoxicity, optionally grade 3 or higher, prolonged grade 3 or higher or grade 4 or 5 neurotoxicity, after receiving the immunotherapy for treating the same disease or condition; or (2) each of the subjects of the group has ALL that is not Philadelphia chromosome positive (Ph+) or Philadelphia-like (Ph-like) subtype of ALL.

79. The method of claim 78, further wherein the reference value for the one or more protein or portion thereof, or each of the gene reference values individually for each of the one or more protein or portion thereof, is:
below the lowest level, concentration, or amount, optionally within 50%, within 25%, within 20%, within 15%, within 10%, or within 5% below the lowest level, concentration or amount, of the one or more protein or portion thereof observed in a sample from among a second plurality of control samples; and/or
below the level, concentration or amount of the one or more protein or portion thereof as measured in more than 75%, 80%, 85%, 90%, 95%, or 98% of samples from among a second plurality of control samples,
wherein the second plurality of control samples is obtained from a group of subjects after receiving the same immunotherapy for treating the ALL, wherein (1) each of the subjects of the group did not develop severe neurotoxicity, optionally wherein each of the subjects developed grade 3 or less, grade 2 or less, or grade 1 or 0 neurotoxicity, after receiving the immunotherapy for treating the same disease or condition, or (2) each of the subjects has ALL that is Philadelphia chromosome positive (Ph+) or Philadelphia-like (Ph-like) subtype of ALL.

80. The method of claim 78 or claim 79, wherein the control sample or each of the plurality of control samples is the same type of biological sample being assessed from the subject, optionally is a plasma sample.

81. The method of any of claims 78-80, wherein the control sample or each of the plurality of control samples had been obtained or collected from the subject no more than 4 days, no more than 3 days, no more than 2 days or no more than 1 day, after initiation of administration of the immunotherapy and/or before the subject exhibits a sign or symptom of the toxicity and/or before the subjects develops a sustained fever.

82. The method of any of claims 78-81, wherein the plurality of control samples comprises at least 3, at least 10, at least 20, at least 50, or at least 100 control samples.

83. The method of any of claims 73-83, wherein if the comparison indicates the subject is or is likely to develop neurotoxicity, administering to the subject an agent or other treatment capable of treating, preventing, delaying, reducing or attenuating the development or risk of development of a toxicity and the immunotherapy.

84. The method of claim 83, wherein administration of the agent is to be administered within one, two, or three days of and/or at first fever following, the initiation of administration of the immunotherapy to the subject.

85. The method of any of claims 1-88, wherein the immunotherapy specifically binds to an antigen associated with the disease or condition or expressed in cells of the environment of a lesion associated with the disease or condition.

86. The method of claim 85, wherein the antigen is CD19, CD20, CD22 or CD123.

87. The method of any of claim 1-86, wherein the immunotherapy is a T cell-engaging therapy comprising a bispecific antibody, wherein at least one binding portion specifically binds to a T cell antigen and a second binding portion binds to the antigen associated with the disease or condition or expressed in cells of the environment of a lesion associated with the disease or condition.

88. The method of claim 87, wherein the T cell antigen is CD3.

89. The method of claim 87 or 88, wherein the second binding portion binds CD19.

90. The method of any of claims 87-89, wherein the bispecific antibody is blinatumomab.

91. The method of any of claims 1-86, wherein the immunotherapy is a cell therapy, wherein the cell therapy comprises genetically engineered cells expressing a recombinant receptor.

92. The method of claim 91, wherein the genetically engineered cells comprise T cells or NK cells.

93. The method of claim 91 or claim 92, wherein the engineered cells comprise T cells.

94. The method of any of claims 1-86 and 91-93, wherein the immunotherapy is a T cell therapy comprising genetically engineered T cells expressing a recombinant receptor.

95. The method of claim 94, wherein the T cells comprise CD4+ and/or CD8+ T cells

96. The method of any of claims 91-95, wherein the recombinant receptor is a T cell receptor or a functional non-T cell receptor.

97. The method of any of claims 91-96, wherein the recombinant receptor is a chimeric antigen receptor (CAR).

98. The method of any of claims 91-97, wherein the recombinant receptor is an anti-CD19 CAR.

99. The method of claim 97 or 98, wherein the CAR comprises an extracellular antigen-recognition domain that specifically binds to the antigen and an intracellular signaling domain comprising an ITAM, wherein optionally, the intracellular signaling domain comprises an intracellular domain of a CD3-zeta (CDζ) chain; and/or wherein the CAR further comprises a costimulatory signaling region, which optionally comprises a signaling domain of CD28 or 4-1BB.

100. The method of any of claims 91-99, wherein the risk or likely risk of the subject developing neurotoxicity following administration of the cell therapy is further based on the value of a parameter that indicates or correlates with the degree of recombinant receptor-dependent, optionally CAR-dependent, activity of the composition, wherein if the value of the parameter is at or greater than a threshold value the subject is at risk of developing neurotoxicity following administration of the immunotherapy when administered to the subject.

101. The method of claim 100, wherein the recombinant receptor-dependent activity comprises a measure of the production or accumulation of one or more of a proinflammatory cytokine, or a normalized value thereof.

102. The method of claim 101, wherein the proinflammatory cytokine is TNF-alpha, IFN-gamma, IL-2, IL-10, or a combination thereof.

103. The method of any of claims 91-103, wherein the immunotherapy comprises the administration of from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive.

104. The method of claim 19 or claim 70, wherein the immunotherapy is a cell therapy, said cell therapy comprising genetically engineered cells expressing a recombinant receptor, and the subject is administered a dose that is from or from about 2×10⁶ to 5×10⁷ total recombinant receptor-expressing cells, inclusive, or that is from or from about 2×10⁵ cells/kg to 5×10⁵ cells/kg total recombinant receptor-expressing cells, inclusive.

105. The method of claim 20 or claim 71, wherein the immunotherapy is a cell therapy, said cell therapy comprising genetically engineered cells expressing a recombinant receptor, and the subject is administered a dose that is from or from about 1×10⁷ to 2.0×10⁸ total recombinant receptor-expressing cells, inclusive, or that is from or from about 1×10⁶ cells/kg to 2×10⁶ cells/kg total recombinant receptor-expressing cells, inclusive.

106. The method of claim 23-30, wherein the immunotherapy is a cell therapy, said cell therapy comprising genetically engineered cells expressing a recombinant receptor, and the subject is administered a dose that is from or from about 1×10⁷ to 2.0×10⁸ total recombinant receptor-expressing cells, inclusive, or the subject is administered a dose that is from or from about 1×10⁶ cells/kg to 2×10⁶ cells/kg total recombinant receptor-expressing cells, inclusive.

107. The method of any of claims 1-106, wherein the subject is an adult human subject.

108. The method of any of claims 1-106, wherein the subject is a pediatric human subject.

109. A kit, comprising reagents for detecting the expression of two or more gene products or portions thereof in a sample, wherein the two or more gene products are encoded by two or more of CCL17, ABCA9, ADAMTSL4, ADGRA2, ADGRF1, AKS, APOL1, ARHGAP27, ARID3B, CA6, CABP7, CCDC152, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGB5, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, ZNF415, ENG, SELE, ICAM3, PCDHGA9, FMNL1, or IL6R, or a portion or a fragment of any of the forgoing.

110. The kit of claim 109, wherein the two or more gene products are human gene products.

111. The kit of claim 109 or 110, wherein the kit comprises reagents for detecting the expression of at least 3, at least 4, at least 5, at least 6, at least 7, at least 8, at least 9, at least 10, at least 11, at least 12, at least 13, at least 14, at least 15, at least 20, at least 25, at least 30, at least 40, or at least 50 gene products.

112. The kit of any of claims 109-111, wherein at least one of the two or more gene products is from a first group of gene products that negatively correlate to a risk of developing neurotoxicity, wherein the first group comprises gene products encoded by CCL17, ABCA9, ADAMTSL4, ADGRA2, ADGRF1, AK5, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, and portions or a fragments of any of the forgoing.

113. The kit of any of claims 109-112, wherein at least one of the two or more gene products is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing.

114. The kit of any of claims 109-113, wherein at least one of the two or more gene products is a gene product encoded by JCHAIN, MUC4, CA6, WNT9A, ADGRF1 or CCL17, or a portion or fragment of any of the foregoing.

115. The kit of any of claims 109-112, wherein at least one of the two or more gene products is a gene product encoded by CCL17, ENG, SELE, ICAM3, or IL6R, and portions or a fragments of any of the forgoing.

116. The kit of any of claims 109-115, wherein at least one of the two or more gene products is a gene product encoded by CCL17 or is a portion or fragment thereof.

117. The kit of any of claims 109-116, wherein at least one of the two or more gene products is from a second group of gene products that positively correlate to a risk of developing neurotoxicity, wherein the second group comprises gene products encoded by ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, and ZNF415, or portions or a fragments of any of the forgoing.

118. The kit of any of claims 109-117, wherein at least one of the two or more gene products is a gene product encoded by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

119. The kit of any of claims 109-118, wherein at least one of the two or more gene products is a gene product encoded by PINLYP, ASAP2, FMNL2, PTCH1, TTC28, PCDHGA6, PCDHGB6 or PCDHGA12, or a portion or fragment of any of the foregoing.

120. The kit of any of claims 109-119, wherein at least one of the two or more gene products is a gene product encoded by PINLYP or PCDHGA12, or a portion or fragment of any of the foregoing.

121. The kit of any of claims 109-119 wherein:
at least one of the two or more gene products is a gene product, or a portion or fragment thereof, from a first group of gene products that negatively correlate to a risk of developing neurotoxicity selected from CCL17, ABCA9, ADAMTSL4, ADGRA2, ADGRF1, AKS, APOL1, ARHGAP27, ARID3B, CA6, CABP7, CCDC152, CCL17, CCR1, CCR6, CEP85L, CISH, CR2, ENAM, ENPP2, EPHA4, FTH1P11, FTH1P2, FTH1P8, GADD45A, GAS6, GBP3, GBP5, GBP6, GIMAP1-GIMAPS, GLI2, GPA33, GPRIN3, HSPA1A, IFITM1, IFITM3, IL15, IL2RA, JCHAIN, KIAA1257, LA16c-390H2.4, LAMB1, LDB3, LINC00623, LST1, LTB, LY6E, MAS1, MUC4, NLRC3, PLXNA4, PON2, PTGES3P1, PTP4A3, RNU1-1, RP11-345J4.6, RP11-421N8.1, RP11-51J9.5, RP11-51O6.1, RP11-552F3.9, RP11-686D22.9, RP11-723D22.3, RP11-723O4.6, RP13-512J5.1, RP4-620F22.2, RP5-940J5.9, RP6-109B7.5, RPL21P75, RYR2, SAMD9L, SEMA6A, SLC37A3, SNRPEP4, SOCS1, SPATS2L, SPON1, SV2C, TMEM154, TP53INP1, TNF, TRIM47, UST, WNT9A, ENG, SELE, ICAM3, or IL6R, and portions or a fragments of any of the forgoing; and
at least one of the gene products is a gene product, or a portion or fragment thereof, from a second group of gene products that positively correlate to a risk of developing neurotoxicity selected from ASAP2, ATP8B1, ATP9A, CCNA1, CDHR3, CECR2, CELF4, DLX1, DPYSL3, EHD4, FMNL2, GGA2, GPR176, HHIPL1, HOXA7, HMX3, IGF2BP1, IL3RA, IRX3, IRX5, KCNIP1, KIAA1644, LINC00092, LINC01483, MDFI, MIB1, MMP14, NOM1, OTOA, PCDHGA12, PCDHGA4, PCDHGA6, PCDHGB1, PCDHGBS, PCDHGB6, PINLYP, PPM1E, PRKD1, PROKR2, PRSS12, PRTG, PTCH1, RFX8, RP11-146B14.1, RP11-3P17.5, RP11-41O4.1, RP11-713N11.4, RP4-568B10.1, SERF1A, SEZ6L, SMURF1, TBC1D30, TCF12, TCP11, TM9SF3, TMPRSS15, TMSB15A, TNKS1BP1, TREM2, TTC28, PCDHGA9, FMNL1, and ZNF415, or portions or a fragments of any of the forgoing.

122. The kit of claim 121, wherein the at least one of the gene products from the first group is a gene product encoded by ADGRF1, CA6, CCL17, CCR6, ENAM, GAS6, GBP5, GLI2, IFITM1, JCHAIN, MUC4, PON2, PTP4A3, SEMA6A, SLC37A3, SPATS2L, TMEM154, TP53INP1, IL2RA, or WNT9A, or is a portion or a fragment of any of the forgoing;
and wherein the at least one of the gene products from the second group is a gene product encoded by ASAP2, FMNL2, GPR176, MDFI, PCDHGA12, PCDHGA6, PCDHGBS, PCDHGB6, PINLYP, PTCH1, ATP9A, HMX3, DPYSL3, ZNF415, IRX5, TMPRSS15, IL3RA, IGF2BP1, or TTC28, or is a portion or fragment of any of the foregoing.

123. The kit of claim 121 or 122, wherein the at least one of the gene products from the first group is a gene product encoded by JCHAIN, MUC4, CA6, WNT9A, ADGRF1 or CCL17, or a portion or fragment of any of the foregoing; and
wherein the at least one of the gene products from the second group is a gene product encoded by PINLYP, ASAP2, FMNL2, PTCH1, TTC28, PCDHGA6, PCDHGB6 or PCDHGA12, or a portion or fragment of any of the foregoing.

124. The kit of any of claims 121-123, wherein:
the at least one of the gene products from the first group is a gene product encoded by CCL17 or is a portion or fragment thereof; and
the at least one of the gene products from the second group is a gene product encoded by PINLYP or PCDHGA12, or a portion or fragment thereof.

125. The kit of any of claims 109-124, wherein the two or more gene products are or comprise mRNA.

126. The kit of claim 125, wherein the reagents comprise one or more oligonucleotide and/or polynucleotide probes that are to, bind to, and/or are capable of binding to the one or more mRNA gene products.

127. The kit of any of claims 109-124, wherein the two or more gene products are or comprise proteins or variants or fragments thereof.

128. The kit of claim 127, wherein the two or more gene products are selected from CCL17, ENG, SELE, ICAM3, or IL6R, and portions or a fragments of any of the forgoing.

129. The kit of claim 128, wherein the reagents are or comprise antibodies or antigen binding fragments or variants thereof, wherein the antibodies or the antigen binding fragments or variants thereof bind to and/or are capable of binding to the protein gene products.

130. The kit of any of claims 109-129, further comprising an immunotherapy.

131. The kit of claim 130, wherein the immunotherapy is a cell therapy or is a T cell-engaging therapy, optionally wherein the cell therapy comprises cells engineered to express a recombinant receptor.

132. The kit of any of claims 109-131, for use in connection the method of any of claims 1-108.

133. An article of manufacture, comprising a kit of any one of claims 109-134, and instructions for using the reagents to assay a biological sample from a subject that is a candidate for treatment, optionally with an immunotherapy.

134. The article of manufacture of claims 133, wherein the instructions specify carrying out the method of any of claims 1-108.

135. An article of manufacture comprising an immunotherapy and instructions for administering the immunotherapy to a subject that exhibits a Philadelphia chromosome (Ph+) and/or Ph chromosome-like (Ph-like) molecular subtype of acute lymphoblastic leukemia (ALL).

136. The article of manufacture of claim 135, wherein the immunotherapy is a immunotherapy or is a T cell-engaging therapy, optionally wherein the immunotherapy comprises cells engineered to express a recombinant receptor.
